(19) United States
(12) Patent Application Publication
(10) Pub. No.: US 2025/0264455 A1
(43) Pub. Date: Aug. 21, 2025

(54) GENETICALLY CONTROLLED NANOSCOPY CONTRAST-GENERATING UNITS, GENETICALLY CONTROLLED STRUCTURAL ELEMENTS, GENETICALLY CONTROLLED SCAFFOLDS, NANOBIOMATERIAL BASED THEREON, AND USE THEREOF IN NANOSOCOPY METHODS

(71) Applicants: Gil Gregor WESTMEYER, Starnberg (DE); Mohammad ELGAMACY, Tübingen (DE); HELMHOLTZ ZENTRUM MÜNCHEN- DEUTSCHES FORSCHUNGSZENTRUM FÜR GESUNDHEIT UND UMWELT GMBH, Neuherberg (DE); TECHNISCHE UNIVERSITÄT MÜNCHEN - TUM, Munich (DE)

(72) Inventors: Gil Gregor WESTMEYER, Starnberg (DE); Alberto PIOVESAN, Munich (DE); Felix SIGMUND, Munich (DE); Dong-Jiunn Jeffery TRUONG, Freising (DE); Oleksandr BEREZIN, Munich (DE); Niklas ARMBRUST, Freising (DE); Mohammad ELGAMACY, Tübingen (DE)

(21) Appl. No.: 18/839,717

(22) PCT Filed: Feb. 17, 2023
(86) PCT No.: PCT/EP2023/054113

§ 371 Date: Aug. 19, 2024

(30) Foreign Application Priority Data
Feb. 18, 2022 (EP) 22157586.3

(51) Int. Cl. G01N 33/50; B82Y 15/00; C07K 14/415; C07K 14/825; G01N 33/58

(52) U.S. Cl. CPC G01N 33/5035; C07K 14/415; C07K 14/825; G01N 33/582; B82Y 15/00; C07K 2319/60

(57) ABSTRACT

The present invention relates to a genetically controlled nanoscopy contrast-generating unit comprising a metal interactor, wherein the metal interactor is compatible with nanoscopy fixation protocols, nanoscopy post-fixation protocols, and nanoscopy metal staining protocols and wherein the metal interactor is a molecule to which metal ions can bind to or react with. The present invention also relates to a genetically controlled structural element, wherein said genetically controlled structural element organizes the genetically controlled nanoscopy contrast-generating unit. The genetically controlled structural element can be an encapsulin and the genetically controlled nanoscopy contrast-generating unit can be one or two murine metallothionein-3, or three chimeric metallothioneins. The present invention also relates to a genetically controlled scaffold, wherein said genetically controlled scaffold spatially organizes the genetically controlled structural elements. The present invention also relates to the use of such genetically controlled nanoscopy contrast-generating unit, such genetically controlled structural element, and/or such genetically controlled scaffold for nanoscopy detection methods. The present invention also relates to a nanobiomaterial consisting of the isolated genetically controlled structural elements, and/or genetically controlled scaffolds. The present invention also relates to vectors comprising a nucleic acid encoding a genetically controlled nanoscopy contrast-generating unit, a genetically controlled structural element, and/or a genetically controlled scaffold.

### CROSS-REFERENCE TO RELATED APPLICATIONS

This application is a national stage entry under 35 U.S.C. § 371 of International Patent Application PCT/EP2023/052908, filed Feb. 7, 2023 and published as International Patent Publication WO 2023/148392 A1 on Aug. 10, 2023, which claims the benefit of and priority to European Patent Application EP 22155466.0, filed on Feb. 7, 2022, the contents of each are hereby incorporated by reference.

### REFERENCE TO A SEQUENCE LISTING

This application contains a Sequence Listing in computer readable form. The computer readable form is incorporated herein by reference.

### FIELD OF THE INVENTION

The present invention relates to a genetically controlled nanoscopy contrast-generating unit comprising a metal interactor, wherein the metal interactor is compatible with nanoscopy fixation protocols, nanoscopy post-fixation protocols, and nanoscopy metal staining protocols and wherein the metal interactor is a molecule to which metal ions can bind to or react with. The metal interactor is not a ferroxidase and is not an enzymatic product from an exogenous substrate. The present invention also relates to a genetically controlled structural element, wherein said genetically controlled structural element organizes the genetically controlled nanoscopy contrast-generating unit. The present invention also relates to a genetically controlled scaffold, wherein said genetically controlled scaffold spatially organizes the genetically controlled structural elements. The present invention also relates to the use of such genetically controlled nanoscopy contrast-generating unit, such genetically controlled structural element, and/or such genetically controlled scaffold for nanoscopy detection methods. The present invention also relates to a nanobiomaterial consisting of the isolated genetically controlled structural elements, and/or genetically controlled scaffolds. The present invention also relates to vectors comprising a nucleic acid encoding a genetically controlled nanoscopy contrast-generating unit, a genetically controlled structural element, and/or a genetically controlled scaffold.

### INTRODUCTION

Biomedical research generates an increasing need for nanoscale mapping of cell-to-cell contacts as well as the intracellular distribution of organelles and biomolecules to decode, for example, structure-function relationships in tissue. Genetically-encodable fluorescent reporters have revolutionized biomedical research thanks to their ease of use and multi-color readout of cellular processes (Rodriguez et al., Trends in Biochemical Sciences, 2017). However, equivalent multiplexable genetic reporters for electron microscopy (EM) that are convenient to use while providing optimal resolution are not available yet.

Generally, it is desirable to achieve nanometer resolution to map, for example, cell-to-cell contacts. One example in which robust nanometer resolution is crucial is mapping cell-to-cell contacts of neuronal cells connected in neuronal networks. Here, the nanometer resolution has to be maintained over imaging volumes with edge lengths ranging from micro- to millimeters unequivocally disentangle intertwined neuronal processes.

Sparse labeling approaches range from silver-based stains used in conjunction with light microscopy (Pannese, Journal of the History of the Neurosciences, 1999) to fluorescent microscopy approaches such as transsynaptic viral tracing (Wickersham et al., Neuron, 2017) transsynaptic fluorescent protein complementation, and randomized combinatorial expression of fluorescent proteins. However, diffraction-limited fluorescence microscopy does not allow high-resolution imaging of, for example, the dense packing of neuronal processes in sub(-cortical) brain regions. Further sparse labeling approaches include super-resolution light microscopy (SRLM) and expansion microscopy (Schermelleh et al., Nature Cell Biology, 2019, Chen et al., Science 2015). SRLM can resolve synaptic contacts but involves detailed, cumbersome local analysis of selected structures labeled with dedicated fluorophores. Expansion microscopy can spatially separate selected molecular targets by pulling them apart via selective affinity handles connecting to an expanding polymer grid. This technology enables an effective sub-diffraction limit resolution with standard diffraction-limited microscopy. It thus allows high-throughput imaging with appropriate engineering solutions to automatize the clearance and isotropic expansion of the chosen targets, including membrane-bound targets to provide anatomical context.

In distinction to sparse labeling approaches, heavy metal stains for volumetric electron microscopy (Hua et al., Nature Communications, 2015) are used that densely label various biomolecules including lipids, proteins, and nucleic acids, which allows automated semantic segmentation of cellular membranes, organelles, protein complexes such as ribosomes, at nanometer-scale resolution over orders of magnitude larger volumes of interest in for example, brains of model organisms such as C. elegans, Drosophila melanogaster, Danio rerio, Danionell translucida, Mus musculus, as well as from human subjects. Established methods for volume electron microscopy include serial mechanical sectioning, collection, and arraying of individual slices, followed by either automated TEM or SEM/multi-SEM, or ion-based milling by either focused ion beam (FIB) or Gas Cluster prior to multiSEM. However, despite the rich anatomical information contained in the heavy-metal stained tissue, molecular information on the genetic identity and expression profile of cells of interest has to be filled-in via molecule-specific contrast, which is usually obtained via labor-intense correlation to antibody-targeted, or genetically expressed fluorophores. The 3D-distribution of these conventional markers can then be obtained via multiphoton microscopy pre-fixed tissue, followed by post-fixation, heavy-metal staining, and volume EM.

Antibody-coated gold nanoparticles have been used as non-genetic markers that work directly in EM without additional optical microscopy due to their precise site and strong EM contrast (Hainfeld, Science, 1987). However, there are technical problems related to tissue penetration and biodistribution, artificial clustering, and epitope masking due to fixation. Locally triggered polymerization of 3,3′-Diaminobenzidine (DAB) with subsequent heavy metal binding has also been used to generate EM contrast. While this technology has been successfully used in, for example, proximity labeling, specialized protocols for diffusing a polymerizing substrate and an oxidant (3,3′-Diaminobenzidine (DAB), H₂O₂) are required, and the polymerization may lead to spatially unbounded contrast. Accumulating metals directly on genetically encoded proteins bearing tetracysteins, or metallothioneins have also been attempted, but these systems necessitate laborious workflows and incubation with toxic metals or gold nanoparticles.

Therefore, there is an increasing demand for providing robust EM contrast compatible with high-throughput fixation, staining, and acquisition protocols for volume EM of cells, organoids, and tissues. Ideally, said EM contrast is both genetically defined and geometric multiplexed EM and, in addition, also provides robust signals for light microscopy.

The inventors have previously shown that encapsulins can be robustly and non-toxically expressed in mammalian cells as self-assembling nanocompartments that can be visualized in EM, if the native ferroxidase (IMEF) is co-expressed, which oxidizes ferrous iron and leads to the formation of iron-oxide biominerals inside the encapsulin lumen. However, the contrast achievable in most cell types, tissues, or model organisms, such as Drosophila melanogaster, is insufficient. This is most likely the case because ferrous iron is limiting.

To obtain sufficiently strong EM contrast with standard fixation, post-fixation, and heavy-metal stain protocols, the inventors have surprisingly found genetically controlled nanoscopy contrast-generating units comprising fixation-stable metal interactors, which can be spatially organized by genetically controlled structural elements to yield unique shapes, in particular rotationally invariant concentric barcodes, which can themselves be spatially organized by genetically controlled scaffolds to produce distinct geometric patterns. The inventors demonstrate inter alia how these distinct contrast geometries can be used to augment anatomical EM maps with multiplexed genetically controlled information, to map the subcellular distribution of biomolecules of interest, to sense cellular activation states, and to actuate cellular states and processes biomechanically or biochemically, or via the deposition of electromagnetic energy or magnetic gradients under nanoscopy control in cell culture and biomedical organisms.

### SUMMARY OF THE INVENTION

In one aspect, a genetically controlled nanoscopy contrast-generating unit comprising a metal interactor is claimed. The metal interactor is compatible with nanoscopy fixation protocols, nanoscopy post-fixation protocols, and nanoscopy metal staining protocols, wherein the metal interactor is a molecule to which metal ions can bind to or react with. Such metal interactor is not a ferroxidase and not an enzymatic product from an exogenous substrate.

In one embodiment, the metal interactor comprised in the genetically controlled nanoscopy contrast-generating unit comprises at least one of the following: a metal-interacting or metal-binding peptide or protein, a lipid-binding protein, an RNA-binding protein, a DNA or RNA molecule, a polymerizing or synthesizing enzyme, a biomineralizing enzyme, a bioconjugation tag, or a combination thereof.

In another embodiment, the metal interactor comprised in the genetically controlled nanoscopy contrast-generating unit comprises at least one of the following: a metallothionein, osmiophilic amino acids, a lead binder, a lanthanide binder, a uranyl-binding protein, a vanadium binder, a copper and silver binder, a fatty-acid-binding protein, preferably FABP, a lipid-binding domain of a cytochrome P450, preferably the lipid- and heme-binding domain of the cytochrome p450 BM3h (BMh3), an RNA aptamer binder:RNA aptamer pair, a programmable RNA binding protein, a polymerizing kinase, a phytochelatine synthase, or a tyrosinase, or mixtures thereof.

In yet another embodiment, the genetically controlled nanoscopy contrast-generating unit further comprises a fluorophore or chromophore. The fluorophore is a fluorescent protein, a co-factor in a fluorescent protein, or an exogenous fluorophore. The chromophore is a chromoprotein, a co-factor in a chromoprotein, or an exogenous chromophore. The exogenous fluorophore or the exogenous chromophore can bind directly to the metal interactor.

In one embodiment, the metal interactor comprised in the genetically controlled nanoscopy contrast-generating unit comprises at least one metallothionein, at least one fatty-acid binding protein, at least one lipid-binding domain, or at least one RNA molecule, and a fluorophore, wherein the fluorophore is a fluorescent protein.

In one embodiment, the genetically controlled nanoscopy contrast-generating unit comprises at least one attachment point. Said at least one attachment point is selected from the group consisting of nanobodies, frankenbodies, coiled-coil domains, isopeptide-forming partners, bioconjugation-tags, split-inteins, calmodulin-binding peptides, phosphorylatable peptides, and conformation-changing peptides including troponins, as well as combinations thereof.

In another aspect, a genetically controlled structural element is claimed, wherein said genetically controlled structural element spatially organizes the genetically controlled nanoscopy contrast-enhancing unit, wherein the genetically controlled structural element possesses a pre-determined shape and pre-determined nanoscale size. Said genetically controlled structural element generates a shape that can be differentiated at the nanoscale, and said shape is optionally a barcode.

In one embodiment, the genetically controlled structural element comprises a member of the encapsulin family, a vault, an MS2 phage, a Qbeta bacteriophage, an AP205 phage, an engineered mono-to-polyvalent hub, a filament, a linker, or combinations or variants thereof.

In another embodiment, the genetically controlled structural element comprises at least one attachment point. Said at least one attachment point is selected from the group consisting of nanobodies, frankenbodies, coiled-coil domains, isopeptide-forming partners, bioconjugation-tags, split-inteins, calmodulin-binding peptides, phosphorylatable peptides, and conformation-changing peptides including troponins, as well as combinations thereof. In addition, the genetically-controlled nanoscopy contrast generating unit interacts or binds to the at least one attachment point.

In another embodiment, the genetically controlled structural element is an encapsulin and the genetically controlled contrast-generating units are selected from the group of metallothionein MT3, a series of different metallothionein species, fatty acid-binding proteins, preferably FABPs, and the lipid-binding domain of the cytochrome P450, or combinations thereof. In said embodiment, the lipid-binding domain of the cytochrome P450 is optionally the lipid- and heme-binding domain of the cytochrome P450 BM3 (BM3h). In said embodiment, one to three copies of the MT3 or one to three different metallothionein species are bound to the N-terminus of the encapsulin structural element. In addition, one or more MT3s, FABPs, or BM3hs are bound to the surface of the encapsulin structural element, optionally via a spacer, wherein the spacer is optionally a SasG of variable length. Such structural and spatial organization is configured to generate the distinct shape of a barcode, wherein the barcode is a concentric barcode differentiable at the nanoscale. Such concentric barcode may encode information on cellular states.

In yet another aspect, a genetically controlled scaffold is claimed. The genetically controlled scaffold comprises and spatially organizes the genetically controlled structural element(s), whereby said spatial organization generates distinct geometric patterns that can be differentiated at the nanoscale. Optionally, said distinct geometric patterns encode information on cellular states.

In another embodiment, the genetically controlled scaffold is selected from the group consisting of an endogenous cellular scaffold, a de novo designed scaffold, a SasG element, a CsgA element, an RNA or DNA structure, or combinations thereof.

In one embodiment, the genetically controlled scaffold comprises a SasG of variable length, one or more encapsulins as structural element, and at least one contrast generating unit selected from MT3 or a series of different metallothionein species. In such scaffold, the encapsulins are connected and spatially organized via SasG, and comprise the MT3, or the series of different metallothionein species.

In one embodiment, the genetically controlled nanoscopy contrast-generating unit, the genetically controlled structural element and/or the genetically controlled scaffold is compatible with intact cell systems, preferably with mammalian cells and organoids or biomedical model organisms.

In one further aspect, the present invention refers to the use of the genetically controlled structural elements and/or the genetically controlled scaffolds for nanoscopy detection, wherein nanoscopy detection comprises molecular mapping and/or geometric sensing.

In one embodiment, the nanoscopy detection is molecular mapping. Said molecular mapping provides information on the subcellular distribution of a molecule of interest within a cell or tissue via the binding of genetically controlled structural elements and/or genetically controlled scaffolds to a molecule of interest, which thus determines the subcellular localization of the genetically controlled structural elements and/or the genetically controlled scaffolds.

In another embodiment, the nanoscopy detection is geometric sensing. Said geometric sensing provides information on a specific cellular state, a cellular process, the presence or absence of an analyte or environmental parameter of interest within a cell or tissue, or the response to an external stimulus. The shape generated by the genetically controlled structural element(s) and/or the geometric pattern generated by the genetically controlled scaffold(s) changes in response to a pre-defined cellular state, a pre-defined cellular process, at least one analyte, or at least one environmental parameter, or an external stimulus.

In another embodiment, the nanoscopy detection comprising mapping and/or geometrical sensing further comprises geometric actuation. Said geometric actuation alters a cellular state or process by a biomechanical, and/or biochemical effect or via an external stimulus, or the deposition of external energy, electromagnetic radiation, mechanical energy, or magnetic gradients.

In another aspect, the invention relates a nanobiomaterial consisting of isolated genetically controlled structural element(s), and/or the genetically controlled scaffold(s). The genetically controlled structural element(s), and/or the genetically controlled scaffold(s) are thus isolatable from the source of origin, which is preferably a genetically modified cell or a cell-free system.

In yet another aspect, the invention relates to a vector comprising a nucleic acid encoding the genetically controlled nanoscopy contrast-generating unit, the genetically controlled structural element and/or the genetically controlled scaffold.

In one embodiment, the genetically controlled nanoscopy-generating unit according to any of the preceding embodiments comprises a metal interactor, wherein said metal interactor is one (SEQ ID NO: 1) or two murine metallothionein-3 (SEQ ID NO: 4), or wherein said metal interactor is a chimeric metallothionein sequence comprising three metallothionein sequences from three different species. In a preferred embodiment, the three metallothionein sequences from three different species are murine etallothionein-3 (MmT3), Synechococcus elongatus (SmtA) metallothionein, and Triticum aestivum Ec-1 metallothionein (TaEC1) (SEQ ID NO: 5). In another equally preferred embodiment, the metal interactor is one (SEQ ID NO: 1) or two murine metallothionein-3 (SEQ ID NO: 4).

In another embodiment, the fluorescent protein of the genetically controlled nanoscopy contrast-generating unit according to the preceding embodiments is eUnaG, mScarlet-I, miniSog, or eUnaG and mCherry. In a preferred embodiment, the fluorescent protein is eUnaG. In one embodiment, eUnaG is N-terminally fused to one or two murine metallothionein-3. In an alternative embodiment, eUnaG is C-terminally fused to one or two murine metallothionein-3.

In yet another embodiment, the genetically controlled structural element according to any preceding embodiment comprise a member of the encapsulin family. In a preferred embodiment, the encapsulin selected from the group consisting of Quasibacillus thermotolerans (Qt) encapsulin, Myxococcus xanthus (Mx) encapsulin, and Thermotoga maritima (Tm) encapsulin. In one embodiment, the genetically controlled structural element is a member of the encapsulin family, and the genetically controlled contrast-generating unit is one (SEQ ID: NO: 1) or two murine metallothionein-3 (SEQ ID NO: 4). In a preferred embodiment, the genetically controlled structural element is an encapsulin selected from the group consisting of Quasibacillus thermotolerans (Qt) encapsulin, Myxococcus xanthus (Mx) encapsulin, and Thermotoga maritima (Tm) encapsulin.

In another embodiment, the genetically controlled structural element is an encapsulin selected from the group consisting of Quasibacillus thermotolerans (Qt) encapsulin, Myxococcus xanthus (Mx) encapsulin, and Thermotoga maritima (Tm) encapsulin and the genetically controlled contrast-generating unit is one (SEQ ID NO: 1) or two N-terminally fused murine metallothionein-3 (SEQ ID NO: 4), or genetically controlled contrast-generating unit is a chimeric metallothionein sequence comprising three N-terminally fused metallothioneins comprising murine metallothionein-3 (MmT3), Synechococcus elongatus (SmtA) metallothionein, and Triticum aestivum Ec-1 metallothionein (TaEC1) (SEQ ID NO: 5). In another embodiment, the one or two N-terminally fused murine metallothioneins, or the chimeric metallothionein sequence comprising three N-terminally fused metallothioneins comprising murine metallothionein-3 (MmT3), Synechococcus elongatus (SmtA) metallothionein, and Triticum aestivum Ec-1 metallothionein (TaEC1) (SEQ ID NO: 5) are located on the inner surface of the encapsulin. In another embodiment, a FLAG tag is C-terminally fused.

In a preferred embodiment, the genetically controlled structural element is a Quasibacillus thermotolerans (Qt) encapsulin, wherein the genetically controlled contrast-generating unit is one N-terminally fused murine metallothionein-3, wherein a FLAG tag is C-terminally fused (SEQ ID NO: 165). In yet another embodiment, the genetically controlled structural element is a Quasibacillus thermotolerans (Qt) encapsulin, wherein the genetically controlled contrast-generating unit is two N-terminally fused murine metallothionein-3, wherein a FLAG tag is C-terminally fused.

In yet another embodiment, the genetically controlled structural element is a Quasibacillus thermotolerans (Qt) encapsulin, wherein the genetically controlled contrast-generating unit is a chimeric metallothionein sequence comprising three N-terminally fused metallothioneins comprising murine metallothionein-3 (MmT3), Synechococcus elongatus (SmtA) metallothionein, and Triticum aestivum Ec-1 metallothionein (TaEC1) (SEQ ID NO: 5), wherein a FLAG tag is C-terminally fused (SEQ ID NO: 169).

In even another embodiment, the genetically controlled structural element is a Myxococcus xanthus (Mx) encapsulin, wherein the genetically controlled contrast-generating unit is one N-terminally fused murine metallothionein-3, wherein a FLAG tag is C-terminally fused (SEQ ID NO: 166).

In one embodiment, the genetically controlled structural element is a Myxococcus xanthus (Mx) encapsulin, wherein the genetically controlled contrast-generating unit is two N-terminally fused murine metallothionein-3, and wherein a FLAG tag is C-terminally fused. In another embodiment, the genetically controlled structural element is a Myxococcus xanthus (Mx) encapsulin, wherein the genetically controlled contrast-generating unit is a chimeric metallothionein sequence comprising three N-terminally fused metallothioneins comprising murine metallothionein-3 (MmT3), Synechococcus elongatus (SmtA) metallothionein, and Triticum aestivum Ec-1 metallothionein (TaEC1), and wherein a FLAG tag is C-terminally fused. In yet another embodiment, the genetically controlled structural element is a Thermotoga maritima (Tm) encapsulin, wherein the genetically controlled contrast-generating unit is one N-terminally fused murine metallothionein-3, and wherein a FLAG tag is C-terminally fused. In yet another embodiment, the genetically controlled structural elementis a Thermotoga maritima (Tm) encapsulin, wherein the genetically controlled contrast-generating unit is two N-terminally fused murine metallothionein-3 (SEQ ID NO: 4), and wherein a FLAG tag is C-terminally fused. In one embodiment, the genetically controlled structural element is a Thermotoga maritima (Tm) encapsulin, wherein the genetically controlled contrast-generating unit is a chimeric metallothionein sequence comprising three N-terminally fused metallothioneins comprising murine metallothionein-3 (MmT3), Synechococcus elongatus (SmtA) metallothionein, and Triticum aestivum Ec-1 metallothionein (TaEC1) (SEQ ID NO: 5), wherein a FLAG tag is C-terminally fused.

In one embodiment, the genetically controlled structural element is an encapsulin selected from the group consisting of Quasibacillus thermotolerans (Qt) encapsulin, Myxococcus xanthus (Mx) encapsulin, and Thermotoga maritima (Tm) encapsulin, wherein the genetically controlled contrast-generating unit is one (SEQ ID NO: 1) or two N-terminally fused murine metallothionein-3 (SEQ ID NO: 4), or wherein the genetically controlled contrast-generating unit is a chimeric metallothionein sequence comprising three N-terminally fused metallothioneins comprising murine metallothionein-3 (MmT3), Synechococcus elongatus (SmtA) metallothionein, and Triticum aestivum Ec-1 metallothionein (TaEC1) (SEQ ID NO: 5), wherein a FLAG tag comprising a nuclear localization signal is C-terminally fused. In a preferred embodiment, the genetically controlled structural element is a Quasibacillus thermotolerans (Qt) encapsulin, wherein the genetically controlled contrast-generating unit is one N-terminally fused murine metallothionein-3, or wherein a FLAG tag comprising a nuclear localization signal is C-terminally fused (SEQ ID NO: 168). In another preferred embodiment, the genetically controlled structural element is a Quasibacillus thermotolerans (Qt) encapsulin, wherein the genetically controlled contrast-generating unit is a chimeric metallothionein sequence comprising three N-terminally fused metallothioneins comprising murine metallothionein-3 (MmT3), Synechococcus elongatus (SmtA) metallothionein, and Triticum aestivum Ec-1 metallothionein (TaEC1) (SEQ ID NO: 5), wherein a FLAG tag comprising a nuclear localization signal is C-terminally fused. In yet another embodiment, the genetically controlled structural element is a Myxococcus xanthus (Mx) encapsulin, wherein the genetically controlled contrast-generating unit is one N-terminally fused murine metallothionein-3, or wherein a FLAG tag comprising a nuclear localization signal is C-terminally fused (SEQ ID NO: 167). In even another embodiment, the genetically controlled structural element is a Quasibacillus thermotolerans (Qt) encapsulin, wherein the genetically controlled contrast-enhancing unit is a chimeric metallothionein sequence comprising three N-terminally fused metallothioneins comprising murine metallothionein-3 (MmT3), Synechococcus elongatus (SmtA) metallothionein, and Triticum aestivum Ec-1 metallothionein (TaEC1) (SEQ ID NO: 5), wherein a FLAG tag comprising a nuclear export signal is C-terminally fused.

In one aspect, a transgenic Drosophila melanogaster line expressing two genetically controlled structural elements is disclosed, wherein the first genetically controlled structural element is a Quasibacillus thermotolerans (Qt) encapsulin, wherein the genetically controlled contrast-generating unit is a chimeric metallothionein sequence comprising three N-terminally fused metallothioneins comprising murine metallothionein-3 (MmT3), Synechococcus elongatus (SmtA) metallothionein, and Triticum aestivum Ec-1 metallothionein (TaEC1) (SEQ ID NO: 5),wherein a FLAG tag comprising a nuclear localization signal is C-terminally fused and the genetically controlled structural element, and wherein the second genetically controlled structural element is a Myxococcus xanthus (Mx) encapsulin, wherein the genetically controlled contrast-generating unit is one N-terminally fused murine metallothionein-3, or wherein a FLAG tag comprising a nuclear localization signal is C-terminally fused (SEQ ID NO: 167). In an alternative embodiment, the transgenic Drosophila melanogaster line expressing two genetically controlled structural elements is disclosed, wherein the first genetically controlled structural element is a Myxococcus xanthus (Mx) encapsulin, wherein the genetically controlled contrast-generating unit is one N-terminally fused murine metallothionein-3, or wherein a FLAG tag comprising a nuclear localization signal is C-terminally fused (SEQ ID NO: 167), and wherein the second genetically controlled structural element is a Quasibacillus thermotolerans (Qt) encapsulin, wherein the genetically controlled contrast-enhancing unit is a chimeric metallothionein sequence comprising three N-terminally fused metallothioneins comprising murine metallothionein-3 (MmT3), Synechococcus elongatus (SmtA) metallothionein, and Triticum aestivum Ec-1 metallothionein (TaEC1) (SEQ ID NO: 5), wherein a FLAG tag comprising a nuclear export signal is C-terminally fused. In one embodiment, said transgenic Drosophila melanogaster line expresses the genetically controlled structural element (Quasibacillus thermotolerans (Qt) encapsulin) comprising a genetically controlled contrast-generating unit, wherein said genetically controlled contrast-generating unit is a chimeric metallothionein sequence comprising three N-terminally fused metallothioneins comprising murine metallothionein-3 (MmT3), Synechococcus elongatus (SmtA) metallothionein, and Triticum aestivum Ec-1 metallothionein (TaEC1) (SEQ ID NO: 5), wherein a FLAG tag comprising a nuclear localization signal is C-terminally fused in C3 neurons and expresses the genetically controlled structural element (Myxococcus xanthus (Mx) encapsulin) comprising a genetically controlled contrast-generating unit, wherein said genetically controlled contrast-generating unit is one N-terminally fused murine metallothionein-3, or wherein a FLAG tag comprising a nuclear localization signal is C-terminally fused (SEQ ID NO: 167) in T4-5 neurons.

In another aspect, a nanoscopy method using the genetically controlled structural element according to any one of the preceding embodiments is disclosed. In one embodiment, the genetically controlled structural element does not interfere with cellular processes. In another embodiment, the nanoscopy method is electron microscopy. In one embodiment, the electron microscopy method is selected from the group consisting of scanning electron microscopy (SEM), transmission electron microscopy (TEM) and Focused Ion Beam Scanning Electron Microscopy (FIB-SEM). In another embodiment, no further incubation steps involving DAB and hydrogen peroxide or further exogenous substrates are required in the disclosed nanoscopy method.

In another embodiment, the genetically controlled structural element is an encapsulin selected from the group consisting of Quasibacillus thermotolerans (Qt) encapsulin, Myxococcus xanthus (Mx) encapsulin, and Thermotoga maritima (Tm) encapsulin, wherein the genetically controlled contrast-generating unit is one (SEQ ID NO: 1) or two murine metallothionein-3 (SEQ ID NO: 4), or wherein the genetically controlled contrast-generating unit is a chimeric metallothionein sequence comprising three N-terminally fused metallothioneins comprising murine metallothionein-3 (MmT3), Synechococcus elongatus (SmtA) metallothionein, and Triticum aestivum Ec-1 metallothionein (TaEC1) (SEQ ID NO: 5), wherein the genetically controlled structural element further comprises the fluorescent protein eUnaG. In one preferred embodiment, the genetically controlled structural element is a Quasibacillus thermotolerans (Qt) encapsulin, wherein the genetically controlled contrast-generating unit is one murine metallothionein-3, wherein eUnaG is N-terminally fused, wherein a FLAG tag is C-terminally fused (SEQ ID NO: 155). In one embodiment, the genetically controlled structural element according to the preceeding embodiments and the following embodiments is compatible with both fluorescent microscopy and electron microscopy applications. In yet another embodiment, the genetically controlled structural element is a Quasibacillus thermotolerans (Qt) encapsulin, wherein the genetically controlled contrast-generating unit is two murine metallothionein-3, wherein eUnaG is N-terminally fused, wherein a FLAG tag is C-terminally fused (SEQ ID NO: 156). In even another embodiment, the genetically controlled structural element is a Quasibacillus thermotolerans (Qt) encapsulin, wherein the genetically controlled contrast-generating unit is one murine metallothionein-3, wherein eUnaG is C-terminally fused (SEQ ID NO: 227). In one embodiment, the genetically controlled structural element further comprises at least one attachment point according to the preceding embodiments, wherein the at least one attachment point is an intrabody, wherein said intrabody is C-terminally fused. In one embodiment, the intrabody is an anti-mCherry intrabody, wherein said anti-mCherry intrabody is expressed on the outer surface of the encapsulin. In another embodiment, the genetically controlled structural element additionally comprises at least one further attachment point according to the preceding embodiments, wherein said attachment point is a coiled-coil domain. In an alternative embodiment, said attachment point is two coiled-coil domains. In another alternative embodiment, said attachment point is a SpyTag/SpyCatcher. In another embodiment, the genetically controlled structural element additionally comprises at least one further attachment point according to the preceding embodiments, wherein said attachment point is a SpyTag/SpyCatcher, wherein an intrabody is covalently attached to the SpyTag/SpyCatcher.

In one embodiment, the genetically controlled structural element is a Quasibacillus thermotolerans (Qt) encapsulin, or Thermotoga maritima (Tm) encapsulin, wherein the genetically controlled contrast-generating unit is one or two murine metallothionein-3, wherein mScarlet-I is encapsulated to the encapsulin lumen via an encapsulation signal. In another embodiment, the genetically controlled structural element is a Quasibacillus thermotolerans (Qt) encapsulin, or Thermotoga maritima (Tm) encapsulin, wherein the genetically controlled contrast-generating unit is one or two murine metallothionein-3, wherein mScarlet-I is expressed as cargo protein in the encapsulin lumen. In yet another embodiment, the genetically controlled structural element is a Quasibacillus thermotolerans (Qt) encapsulin, or Thermotoga maritima (Tm) encapsulin, wherein the genetically controlled contrast-generating unit is one or two murine metallothionein-3, wherein APEX2 is expressed as cargo protein in the encapsulin lumen.

In one aspect, an electron microscopy method, wherein the genetically controlled structural element according to the preceding embodiment is used, wherein further incubation steps involving DAB and hydrogen peroxide are performed.

In another aspect, the two genetically controlled structutal elements are expressed in a ratio of approximately 4:1, wherein the first genetically controlled structural element is a Quasibacillus thermotolerans (Qt) encapsulin, wherein the genetically controlled contrast-generating unit is one murine metallothionein-3, wherein eUnaG is C-terminally fused (SEQ ID NO: 227), wherein the second genetically controlled structural element further comprises at least one attachment point according to any of the preceding embodiment, wherein the at least one attachment point is an intrabody, wherein said intrabody is C-terminally fused. In another embodiment, said first and second genetically controlled structural elements are expressed in a ratio of approximately 4:1.

In another embodiment, the genetically controlled structural element is a Quasibacillus thermotolerans (Qt) encapsulin, wherein the genetically controlled contrast-generating unit is one murine metallothionein-3, wherein the genetically controlled structural element comprises a programmable ribosomal read-through (rt) cassette, wherein said programmable ribosomal read-through cassette comprises a stop codon and a short read-through (rt) promoting motif at the end of the open-reading frame encoding the Quasibacillus thermotolerans (Qt) encapsulin. In a preferred embodiment, the stop codon is selected from the group consisting of TGA, TAA, TAG and the short read-through promoting motif is selected from the group consisting of rt20s (SEQ ID NO: 76), rt9s (SEQ ID NO: 74), rt9us (CTATCC). In an even more preferred embodiment, the stop codon is TGA, and the short read-through promoting motif is rt20s (SEQ ID NO: 76). In one embodiment, the genetically controlled structural element additionally comprises a C-terminally fused anti-GFP intrabody. In another embodiment, the genetically controlled structural element is co-expressed with Cx43-msfGFP or msfGFP-Cx43. In yet another embodiment, the genetically controlled structural element additionally comprises an anti-GFP intrabody.

In one aspect, a method of generating a distinct ratio of genetically controlled structural elements according to any of the preceding embodiments is disclosed, wherein said genetically controlled structural element is a Quasibacillus thermotolerans (Qt) encapsulin, wherein the Qt encapsulin comprises one N-terminally fused murine metallothionein-3,wherein at the end of the open-reading frame encoding the encapsulin Quasibacillus thermotolerans (Qt) the read-through motif rt20s (SEQ ID NO: 76) and the stop codon TGA are located, wherein the Quasibacillus thermotolerans (Qt) encapsulin comprises one C-terminally fused FLAG-tag, wherein said C-terminally fused FLAG tag is further extended by an anti-GFP intrabody upon ribosomal read-through, whereby the combination of the read-through motif rt20s (SEQ ID NO: 76) and the stop codon yields about 15% anti-GFB intrabodies, thereby serving as a surrogate marker for a translational read-through rate.

In another aspect, a cell line comprising the genetically controlled structural element of any of the preceding embodiments and the fusion protein msfGFP-Cx43 is disclosed.

In one embodiment, the genetically controlled structural element is a Quasibacillus thermotolerans (Qt), Myxococcus xanthus (Mx) encapsulin or a Thermotoga maritima (Tm) encapsulin, wherein the genetically controlled contrast-generating unit is one or two murine metallothionein-3, wherein the genetically controlled structural element further comprises the fluorescent protein mScarlet-l. In a preferred embodiment, the genetically controlled structural element is a Quasibacillus thermotolerans (Qt) encapsulin, wherein the genetically controlled contrast-generating unit is one murine metallothionein-3, wherein mScarlet-I is N-terminally fused, wherein a FLAG tag is C-terminally fused. In another preferred embodiment, the genetically controlled structural element is a Quasibacillus thermotolerans (Qt) encapsulin, wherein the genetically controlled contrast-generating unit is two murine metallothionein-3, wherein mScarlet-I is N-terminally fused, wherein a FLAG tag is C-terminally fused.

In one aspect, an AAV vector comprising the genetically controlled structural element of the previous embodiment is disclosed. In one embodiment, the AAV vector is selected from the group consisting of AAV1, AAV2, AAV3, AAV4, AAV5, AAV6, AAV7, AAV8, AAV9. In another embodiment, the AAV vector is pseudotyped. In a preferred embodiment, the AAV vector is AAV5. In another embodiment, the AAV vector comprising the genetically controlled structural element of the previous embodiment, wherein said genetically controlled structural element comprises m-Scarlet-1, wherein the AAV5 vector comprises a CAMKII promoter. In yet another embodiment, the AAV vector is for use in medicine. In even another embodiment, the AAV vector is for use in diagnostic imaging.

In another aspect, a fluorescence multichannel imaging method using the genetically controlled structural element according to the preceding embodiments is disclosed.

In yet another aspect, a method for monitoring organ, organoid or cells using the genetically controlled structural element according to the preceding embodiment is disclosed. In one embodiment, the cells are mammalian oocytes. In yet another embodiment, the mammalian oocytes are monitored. In even another embodiment, the mammalian oocytes are monitored prior to in vitro fertilization.

In one embodiment, the genetically controlled structural element is a Quasibacillus thermotolerans (Qt) encapsulin, wherein the genetically controlled contrast-generating unit is one murine metallothionein-3, wherein eUnaG is N-terminally fused, wherein an anti-mCherry intrabody is C-terminally fused.

In one aspect, a recycling endosome stain comprising the genetically controlled structural element according to the preceding embodiment is disclosed, wherein the anti-mCherry intrabody is targeted to mCherry-tagged RAB11A. In another aspect, a cargo vesicle stain comprising the genetically controlled structural element according to the preceding embodiment is disclosed, wherein the anti-mCherry intrabody is targeted to mCherry-tagged Myo5b. In yet another aspect, a centriolar microtubule-organizing stain comprising the genetically controlled structural element according to the preceding embodiment is disclosed, wherein the anti-mCherry intrabody is targeted to mCherry-tagged PLK1.

In one aspect, a scanning electron microscopy imaging method or focused ion beam scanning electron microscopy imaging method using the genetically controlled structural element of any of the preceding embodiments is disclosed.

In one embodiment, a genetically controlled scaffold according to any of the preceding embodiments is disclosed, wherein said genetically controlled scaffold is a SasG having 2, 3, 4, 5, 6, 7, 8, 9 or 10 G5 domains connected by E domains, an encapsulin selected from the group consisting of Quasibacillus thermotolerans (Qt) encapsulin, Myxococcus xanthus (Mx) encapsulin, and Thermotoga maritima (Tm) encapsulin as genetically controlled structural element, one or two murine metallothionein-3 as genetically controlled nanoscopy contrast-generating unit. In another embodiment, the genetically controlled scaffold comprises a SasG having 2, 3, 4, 5, 6, 7, 8, 9 or 10 G5 domains connected by E domains, an encapsulin selected from the group consisting of Quasibacillus thermotolerans (Qt) and Thermotoga maritima (Tm) as genetically controlled structural element, one or two murine metallothionein-3 as genetically controlled nanoscopy contrast-generating unit. In yet another embodiment, the genetically controlled scaffold comprises a SasG having 2, 3, 4, 5, 6, 7, 8, 9 or 10 G5 domains connected by E domains, said SasG having N-terminally fused sfGFP and C-terminally fused mCherry, an encapsulin selected from the group consisting of Quasibacillus thermotolerans (Qt) encapsulin, Myxococcus xanthus (Mx) encapsulin, and Thermotoga maritima (Tm) encapsulin as genetically controlled structural element, one or two murine metallothionein-3 as genetically controlled nanoscopy contrast-generating unit. In even another embodiment, the genetically controlled scaffold comprises a SasG having 2, 3, 4, 5, 6, 7, 8, 9 or 10 G5 domains connected by E domains, said SasG having N-terminally fused sfGFP and C-terminally fused mCherry, an encapsulin selected from the group consisting of Quasibacillus thermotolerans (Qt) and Thermotoga maritima (Tm) as genetically controlled structural element, one murine metallothionein-3 as genetically controlled nanoscopy contrast-generating unit. In one embodiment, the genetically controlled scaffold comprises a SasG having 2, 3, 4, 5, 6, 7, 8, or more G5 domains connected by E domains, said SasG having N-terminally fused sfGFP and C-terminally fused mCherry, a Quasibacillus thermotolerans (Qt) encapsulin as genetically controlled structural element, one murine metallothionein as genetically controlled nanoscopy contrast-generating unit, wherein said Qt encapsulin has one or more anti-GFP intrabodies on its surface. In another embodiment, the genetically controlled scaffold comprises a SasG having 2, 3, 4, 5, 6, 7, 8, or more G5 domains connected by E domains, said SasG having N-terminally fused sfGFP and C-terminally fused mCherry, a Thermotoga maritima (Tm) encapsulin as genetically controlled structural element, one murine metallothionein as genetically controlled nanoscopy contrast-generating unit, wherein said Tm encapsulin has one or more anti-mCherry intrabody on its surface. In yet another embodiment, the genetically controlled scaffold comprises a SasG having 2, 3, 4, 5, 6, 7, 8, or more G5 domains connected by E domains, said SasG having N-terminally fused sfGFP and C-terminally fused mCherry, a Quasibacillus thermotolerans (Qt) encapsulin as genetically controlled structural element, one murine metallothionein as genetically controlled nanoscopy contrast-generating unit, wherein said Qt encapsulin has one or more anti-GFP intrabody on its surface, and a Thermotoga maritima (Tm) encapsulin as genetically controlled structural element, one murine metallothionein as genetically controlled nanoscopy contrast-generating unit, wherein the Tm encapsulin has one or more anti-mCherry intrabody on its surface. In another embodiment, the genetically controlled scaffold according to any of the preceding embodiments is disclosed, wherein the Quasibacillus thermotolerans (Qt) encapsulin having one or more anti-GFP intrabodies on its surface is linked to the matching anti-mCherry intrabody on the surface of the Thermotoga maritima (Tm) encapsulin. In yet another embodiment, the genetically controlled scaffold according to any of the preceding embodiments is disclosed, wherein the Quasibacillus thermotolerans (Qt) encapsulin having one or more anti-GFP intrabody on its surface results in an approximately 40 nm ring-shaped object, wherein the Thermotoga maritima (Tm) encapsulin having one or more matching anti-mCherry intrabody on the surface results in an approximately 25 nm spherical object, wherein said spherical object surrounds said ring-shaped object.

In one embodiment, the genetically controlled nanoscopy contrast-generating unit according to any one of the preceding embodiments is disclosed, wherein the metal interactor is a uranyl binder. In a preferred embodiment, the uranyl binder is MUP2 (SEQ ID NO: 45). In another embodiment, a genetically controlled structural element according to any of the preceding embodiments is disclosed, wherein the genetically controlled structural element is an encapsulin selected from the group consisting of Quasibacillus thermotolerans (Qt) encapsulin, Myxococcus xanthus (Mx) encapsulin, and Thermotoga maritima (Tm) encapsulin, wherein the genetically controlled contrast-generating unit is MUP2. In a preferred embodiment, the genetically controlled structural element is a Quasibacillus thermotolerans (Qt) encapsulin, wherein the genetically controlled contrast-generating unit is MUP2 (SEQ ID NO: 148).

In another embodiment, the genetically controlled nanoscopy contrast-generating unit according to any one of the preceding embodiments is disclosed, wherein the metal interactor is a fatty acid-binding protein. In one embodiment, the fatty acid-binding protein is B-Fabp (SEQ ID NO: 36), B-Fabp-ko, (SEQ ID NO: 248) Bm3h (SEQ ID NO: 29), or Bm3h-B7 (SEQ ID NO: 247). In another embodiment, the genetically controlled structural element is an encapsulin selected from the group consisting of Quasibacillus thermotolerans (Qt) encapsulin, Myxococcus xanthus (Mx) encapsulin, and Thermotoga maritima (Tm) encapsulin, wherein the genetically controlled contrast-generating unit is B-Fabp, B-Fabp-ko, Bm3h, or Bm3h-B7. In a preferred embodiment, the genetically controlled structural element is a Quasibacillus thermotolerans (Qt) encapsulin, wherein the genetically controlled contrast-generating unit is B-Fabp, B-Fabp-ko, Bm3h, or Bm3h-B7.

In a further embodiment, the genetically controlled structural element is an engineered mono-to-polyvalent hub, wherein the genetically controlled contrast-generating unit is B-Fabp, B-Fabp-ko, Bm3h, or Bm3h-B7. In one embodiment, the engineered mono-to-polyvalent hub is one to ten anti-moon tags, wherein the genetically controlled contrast-generating unit is B-Fabp.

In another embodiment, the genetically controlled nanoscopy contrast-generating unit according to any one of the preceding embodiments is disclosed, wherein the metal interactor is a bioconjugation tag. In a preferred embodiment, the bionconjugation tag is SNAP or CLIP. In yet another embodiment, the genetically controlled structural element is an encapsulin selected from the group consisting of Quasibacillus thermotolerans (Qt) encapsulin, Myxococcus xanthus (Mx) encapsulin, and Thermotoga maritima (Tm) encapsulin, wherein the genetically controlled contrast-generating unit is SNAP or CLIP. In a preferred embodiment, the genetically controlled structural element is a Quasibacillus thermotolerans (Qt) encapsulin, wherein the genetically controlled contrast-generating unit is SNAP (SEQ ID NO: 260) or CLIP (SEQ ID NO: 261). In one embodiment, SNAP or CLIP is N-terminally or C-terminally fused.

In yet another embodiment, the genetically controlled structural element is an encapsulin selected from the group consisting of Quasibacillus thermotolerans (Qt) encapsulin, Myxococcus xanthus (Mx) encapsulin, and Thermotoga maritima (Tm) encapsulin, wherein the genetically controlled contrast-generating unit is one or two murine metallothionein-3, or wherein the genetically controlled contrast-generating unit is a chimeric metallothionein sequence comprising three N-terminally fused metallothioneins comprising murine metallothionein-3 (MmT3), Synechococcus elongatus (SmtA) metallothionein, and Triticum aestivum Ec-1 metallothionein (TaEC1) (SEQ ID NO: 5), wherein the genetically controlled contrast-generating unit comprises an attachment point, wherein said attachment point is a bioconjugation tag,

the genetically controlled structural element is a Quasibacillus thermotolerans (Qt) encapsulin, wherein the genetically controlled contrast-generating unit is one murine metallothionein-3, wherein the genetically controlled contrast-generating unit comprises an attachment point, wherein said attachment point is a bioconjugation tag, wherein said bioconjugation tag is SNAP or CLIP.

In even another embodiment, the genetically controlled nanoscopy contrast-generating unit according to any one of the preceding embodiments is disclosed, wherein the metal interactor is a metal-binding peptide/protein. In a preferred embodiment, the RNA aptamer-binder:RNA aptamer pair is the circularized Twister-Broccoli RNA aptamer.

In a further embodiment, a genetically controlled structural element according to any of the preceding embodiments is disclosed, wherein the genetically controlled structural element is a vault, wherein the genetically controlled contrast-generating unit is the circularized Twister-Broccoli RNA aptamer.

In one embodiment, the genetically controlled nanoscopy contrast-generating unit according to any one of the preceding embodiments is disclosed, wherein the metal interactor is a metal-binding peptide/protein. In a preferred embodiment, the metal-binding peptide/protein is H. pylori ferritin comprising at least one attachment point, and wherein said at least one attachment point is a calmodulin-binding peptide (SEQ ID NO: 134). In another embodiment, the genetically controlled structural element is Quasibacillus thermotolerans (Qt), wherein the genetically controlled contrast-generating unit is one murine metallothionein-3, wherein the genetically controlled structural element further comprises the fluorescent protein eUnaG, and wherein the genetically controlled structural element further comprises the synthetic calmodulin-binding peptide 20 (SEQ ID NO: 241). In one aspect, the genetically controlled contrast-generating unit of the preceding embodiment and the genetically controlled structural element of the preceding embodiment are for the use of any preceding embodiments. In one embodiment, the analyte for the use of any preceding embodiments is calcium.

### BRIEF DESCRIPTION OF THE DRAWINGS

FIG. 1: Metal-interacting domains and osmiophilic domains. FIG. 1A: Exemplary 3D structure of yeast metallothionein (MT) (Protein data bank (PDB) accession number: 1AQS) bound to metal ions. Based on its high density in cysteines, MTs are osmiophilic. MTs are also reported to have a high affinity to lead (Pb), which is contained in standard EM stains. Exemplary sequences for these constructs are SEQ ID NO: 1-5 FIG. 1B: Contrast-to-noise ratios of individual encapsulin particles as structural elements, which were either non-modified (QtEnc^{FLAG}; SEQ ID NO: 142), fused to a tandem MT3 metallothionein domain as a contrast-generating unit (2xMT3-QtEnc^{FLAG}; SEQ ID NO: 143, or filled with iron-oxides mineralized via a ferroxidase (IMEF) (QtEnc^{FLAG}+QtIMEF) (SEQ ID NOs: 142 and 52), as native cargo protein. FIG. 1C: A strong contrast (with a certain shape) can be observed in TEM if the structural element QtEnc spatially organizes the genetically controlled nanoscopy contrast-generating unit comprising the metal-interacting protein MT3; left: TEM micrographs of HEK293T cells expressing QtEnc monomers, which assemble into structural elements N-terminally equipped with a tandem MT3 domain as a contrast-generating unit (2xMT3-QtEnc^{FLAG}); right: TEM micrographs of HEK293T cells expressing unmodified QtEnc structural elements, which form nanocompartments (QtEnc^{FLAG}). Scale bars are 100 nm. FIG. 1D: Silver-stained SDS-PAGE loaded with material from an anti-FLAG pull-down experiment from HEK293T cells expressing MT3 or tandem MT3 (2xMT3) in the context of different encapsulin shell monomers, i.e., QtEnc, TmEnc, and MxEnc. The shift towards higher molecular weights indicates that MT domains are readily expressed as contrast generating units. Since the MT3-TmEnc sample has a BC2Tag (SEQ ID NO: 144) instead of a FLAG tag, no band is seen in the gel. FIG. 1E: Schematic of genetic expression constructs for the methionine-rich protein from Pinctada fucata (30xMMKPDM; SEQ ID NO: 6) and the histidine-rich protein 2 (HRPII), which are expressed as fusion proteins to QtEnc or connected to a targeting moiety (QtSig; SEQ ID NO: 16) for QtEnc encapsulation. FIG. 1F: Preferred proteins and protein motifs with high cysteine and/or histidine abundance as osmiophilic residues, identified by screening the Protein Data Bank (specific PDB accession number given) to achieve a high concentration of osmiophilic residues. Exemplary sequences for these constructs are SEQ ID NO: 6-11.

FIG. 2: Metal-binding proteins. FIG. 2A, FIG. 2B: Selective examples for metal-binding domains, also called metal binders, which can coordinate heavy metals, such as uranyl, routinely contained in EM stains in the form of uranyl acetate. Shown are two domains into which metal binding sites were grafted, MUP2 (based on PDB:2AKF) SEQ ID NO: 45 and MUP5 (based on PDB:5APQ) SEQ ID NO: 46. The gels show a CN-PAGE (Clear Native Polyacrylamide Gel Electrophoresis) analysis of the expression of these domains as direct fusions to QtEnc encapsulin. FIG. 2C: Example of a TEM micrograph from MUP2-QtEnc (SEQ ID NO: 148) showing ring-contrasts (specific shape of a concentric barcode) in the 2D projection of the spherical QtEnc used as a structural element. Scale bar is 50 nm. FIG. 2D: BN-PAGE (Blue Native Polyacrylamide Gel Electrophoresis) analysis of encapsulin variants fused to lanthanide binding tags (LBT). The QtEnc variant fused to LBT15 yields a stable assembly (LBT15-QtEnc^{FLAG}; SEQ ID NO: 145). After adding terbium chloride, tryptophan-sensitized luminescence can be observed upon UV-illumination (UV-FL), demonstrating lanthanide binding to the structural element, also called nanosphere. This finding is supported by ICP-MS measurements (corresponding bar graph on bottom) showing Tb-enrichment in the LBT15 QtEnc variant compared to another variant (Twin-dLBT3; SEQ ID NO: 146) which worked less efficient and the empty encapsulin QtEnc^{FLAG}.

FIG. 3: Fatty acid-/lipid binding proteins. FIG. 3A: Antiparallel 1-barrel structures of L-FABP (PDB: 2LKK) and B-FABP (PDB: lfdq) with an internal binding site that binds a fatty acid in an interior cavity. FIG. 3B: TEM image of HEK cells co-expressing QtEnc-SpyTag003 (SEQ ID NO: 147) and B-FABP-SpyCatcher003 SEQ ID NO: 150, respectively, resulting in contrast enhancement (shape of concentric barcodes) depending on how many B-FABP molecules are bound to a given QtEnc. Two zoom-ins placed on the right show the localized contrast enhancement. Scale bars for the zoom-ins are 20 nm. FIG. 3C: Schematic of the genetic constructs for a two-component system to fuse B-FABP via SpyCatcher003 as an attachment point to the encapsulin surface-functionalized with the corresponding SpyTag003 as attachment point (top). Clear-native PAGE gels (bottom left) showing the slowed-down native running behavior upon co-expression of both constructs vs. the expression of only encapsulin QtEnc as a control (bottom left). The corresponding Western Blot shows an additional band for the co-expression of both constructs (bottom right). FIG. 3D: Another preferred embodiment of metal interactor comprised in the contrast-generating unit is the ligand-binding heme-domain of the cytochrome P450 BM3 (BM3h), which has been shown to selectively bind arachidonic acid (AA), a polyunsaturated fatty acid, which is a preferred interaction site for osmium tetroxide. FIG. 3E: CN-PAGE (left) and (right) analysis of the co-expression of BM3h-SpyCatcher003 (SEQ ID NO: 151) with QtEnc decorated with SpyTags (SpyTag003) (SEQ ID NO: 147). FIG. 3F: TEM images (and zoom-ins on the right) of HEK cells co-expressing the structural element QtEnc-SpyTag003 comprising SpyCatcher-Bm3h resulting in contrast enhancement depending on how many BM3h molecules, which are larger than FABPs, are bound to a given QtEnc. The ratio can be controlled by co-expressing structural element QtEnc without SpyTag, which can be achieved via transient expression or via read-through control (FIG. 15) also in stable cell lines. Scale bars for the zoom-ins are 20 nm.

FIG. 4: RNA, RNA aptamer —RNA aptamer-binding protein interactions FIG. 4A: Shown is the RNA aptamer PP7 coat dimers (SEQ ID NO: 53), which interact with PP7 loops (SEQ ID NO: 56) attached to RNA aptamers. Other preferred aptamers are MS2 (SEQ ID NO: 152), which interacts with the protein MCP (SEQ ID NO: 100), or H23 (K-turn motif) (SEQ ID NO: 206), which interacts with the protein L7ae. (SEQ ID NO: 63). Here, the RNA-binding protein functions as a CGU. In other embodiments, the RNA aptamer-binding protein functions as a structural element. The aptamers, in this case, contain FIG. 4B: PP7 stem-loop. For dual detection by EM and fluorescent microscopy, RNA aptamers such as FIG. 4C: Spinach, SEQ ID NO: 55 FIG. 4D: Dir2s, SEQ ID NO: 57 and FIG. 4E: Rho-BAST SEQ ID NO: 59 may be chosen because they bind to differently colored fluorophores and also enable super-resolution SRLM. Aptamers may also contain multiple binding sites for small fluorophores. If necessary, the aptamers can be stabilized for mammalian expression via circularization. FIG. 4F: Alternatively, or supportive to the use of RNA aptamer-binding proteins such as PCP as the CGU or structural element, protein motifs such as vertebrate RNA-recognition motif (RRM, PDB:3MDF; SEQ ID NO: 60), the very well conserved human K homology domain (KH, PDB:5WWX; SEQ ID NO: 61), or the Zinc Finger CCHC-type which binds RNA specifically (CCHC, PDB:2E5H; SEQ ID NO:62) are applied, which can bind RNA also unspecifically. FIG. 4G: In addition to relying on the unspecific interaction of heavy metals with the phosphates of RNA, specific metal-binding sites such as the one shown here (see also Kanazawa and Kondo, Journal of Inorganic Biochemistry, (2017), 176:140-143) (SEQ ID NO: 58) can be used, also in combination with fluorophore-binding RNA aptamers such that co-localization upon formation of the antiparallel duplex can also be monitored via fluorescence imaging. Other metal-binding RNA motifs, including magnetic species such as nickel and cobalt, can be taken from metal-sensing ribozymes.

FIG. 5: Polymerizing enzymes FIG. 5A: 3D structure of the tetrameric polyphosphate kinase 2 (PPK2) of F. tularensis (PDB: 5LL0). PPKs can turn over the endogenously available substrates GTP or ATP to polymerize highly negatively-charged polyphosphate chains (polyP). FIG. 5B: Schematic of DD-CgPPK2b-QtSig (SEQ ID NO: 47) targeted to/spatially organized in the structural element QtEnc lumen via the endogenous QtEnc encapsulation signal (QtSig). The QtSig encapsulation signal is “KGFTVGSLIQ” (SEQ ID NO: 16). It is the C-terminal fraction after the 2xGGGGS-Linker (SEQ ID NO: 153) in the sequence “DD-CgPPK2b-QtSig”. Cg stands for the organism of origin Corynebacterium glutamicum. DD stands for a degron motif (SEQ ID NO: 154), leading to the degradation of the cargo protein (in this case, the PPK2 kinase) unless it is encapsulated in the lumen of encapsulin. ATP/GTP, endogenously present in cells, can enter via the pores of the structural element and is polymerized into a polyP chain, which is trapped inside the encapsulin lumen FIG. 5C: Clear-Native PAGE analysis of lysates of HEK293T cells expressing QtEnc^{FLAG} (SEQ ID NO: 104) or MxEnc^{FLAG} (SEQ ID NO: 107) alone or co-expressing DD-CgPPK2b-QtSig. The gel was stained with DAPI, exhibiting a yellow shift upon binding to polyP. The UV fluorescence imaging of the gel (upper panel) reveals bands indicating intraluminal polyP accumulation. No UV fluorescent bands are observed when no cargo protein is co-expressed. FIG. 5D: TEM micrograph of HEK293T cells expressing MxEnc^{FLAG}+DD-CgPPK2b-QtSig. Inside the ˜32 nm encapsulins as structural elements, shapes generated by highly contrasted polyP cores can be observed. The scale bar is 100 nm. FIG. 5E: TEM micrograph of HEK293T cells expressing QtEnc^{FLAG}+Phytochelatine-synthase 1 fused to an encapsulation signal (AtPCS1-QtSig; SEQ ID NO: 49). “At” stands for the organism of origin Arabidopsis thaliana. The highly contrasted areas indicate heavy metal-binding to the intraluminal phytochelatins. The scale bar represents 100 nm. FIG. 5F: Clear native PAGE loaded with lysates of HEK293T cells co-expressing TmEnc comprising the coiled-coil P2 (SEQ ID NO: 159) as a cargo adapter fused to a bacterial tyrosinase (BmTyr; SEQ ID NO: 51) via the encapsulation signal P1. Bm stands for Bacillus megaterium as the organism of origin of the tyrosinase. Please also see preferred coiled-coil pairs for the respective encapsulins, as shown in FIG. 8c. The gels were incubated with 100 μM copper chloride and 2 mM L-Tyrosine. The dark high molecular weight bands at 1.0 MDa represent melanin-loaded TmEnc nanospheres as structural elements that also attract heavy metal ions.

FIG. 6: Biomineralizing enzyme Enzymatically generated biominerals such as iron-oxide are a resin to which heavy metals such as lead can bind to in order to intensify EM contrast. The stand-alone ferroxidase within the encapsulin system (IMEF; SEQ ID NO: 52) generates iron-oxide precipitates within the encapsulin nanocompartments as structural elements that are themselves already electron-dense and are thus also suitable for EM techniques that do not involve heavy-metal stains, such as cryo-ET. FIG. 6A: 3D chart of a QtIMEF dimer. FIG. 6B: Coomassie and Prussian-Blue stained BN-PAGE demonstrating QtEnc iron loading in mammalian cells upon supplementation of ferrous ammonium sulfate. FIG. 6C: TEM micrograph of HEK293T cells showing contrasted shapes, generated by QtEnc as structural element with biomineralized iron oxide cores. FIG. 6D: The metal biomineralization can be supported by co-expression of metal importers for ferrous iron (Zip14, SEQ ID NO:219) and manganese (DMT1, SEQ ID NO: 218) such that doping of iron biominerals with manganese can be obtained. FIG. 6E: The ferroxidase activity of IMEF can here be supported by the multicopper oxidase Mnx (SEQ ID NOs: 222, 223, 224). FIG. 6F: Alternatively, cystathionine-y-lyase (SEQ ID NO:220) can lead to the release of cysteines and subsequent formation of Sulfur clusters. Alkaline phosphatase (SEQ ID NO:221) can trigger the intraluminal formation of calcium-phosphate precipitates to which heavy metals can bind.

FIG. 7: Dual-mode contrast-generating unit for EM and SRLM FIG. 7A: Predicted 3D model of a fusion of the ultra-small engineered fluorescent protein eUnaG and MT3 yielding contrast for fluorescence techniques as well as EM. FIG. 7B: Widefield fluorescence microscopy of HEK293T cells expressing eUnaG-MT3-QtEnc^{FLAG} (SEQ ID NO: 155). FIG. 7C: CN PAGE analysis (upper blot) of lysates from HEK293 cells expressing either eUnaG-MT3-QtEnc or eUnaG-2xMT3-QtEnc^{FLAG} (SEQ ID NO: 156). The UV fluorescence image (lower blot) shows the bands corresponding to the assembled high-molecular weight encapsulin fusion emitting green light. FIG. 7D: CN PAGE loaded with lysates of HEK293T cells expressing either mGreenLantern (SEQ ID NO: 157) or CaMPARI2-MT3-QtEnc (SEQ ID NO: 158) fusions. The UV fluorescent image shows green fluorescent bands corresponding to the assembled nano-compartments as structural elements. These data show that even larger fluorescent protein cargos can be placed upstream of the EM-contrast generating unit MT3. FIG. 7E: TEM micrograph of HEK293T cells expressing eUnaG-MT3-QtEnc^{FLAG} showing highly contrasted shapes, i.e., ˜40 nm spherical barcodes generated by the encapsulin with a distinct lumenal phenotype. A contrasted internal ring with a smaller diameter is visible, as well as a bright center spot (see, zoom-in). FIG. 7F: TEM micrograph of HEK293T cells expressing eUnaG-2xMT3-QtEnc^{FLAG}. The presence of double the numbers of MT3 contrast generating units fills the lumen of the encapsulin structural element such that it can be distinguished from e) as a distinct concentric barcode. FIG. 7G: While fusions of fluorescent proteins to the C-terminus of encapsulin monomers, facing to the outer surface of the structural element, are likely to prevent proper encapsulin assembly, the inventors could reconstitute tripartite split-GFP on the outer surface of encapsulin by co-expression of C-terminal fusions of GFP S10 (SEQ ID NO: 25) and GFP S11 (SEQ ID NO: 26) to QtEnc and co-expression (or alternatively also C-terminal fusion) of Split GFP1-9 (left scheme) (SEQ ID NOs: 23, 24 respectively). Correct assembly of the fusion constructs is shown via BN-PAGE, and reconstituted fluorescence is shown via epifluorescence microscopy only if all three components are expressed (on the right and bottom panels).

FIG. 8: Modular targeting of host molecules Multiplexed targeting of guest molecules to the inside of structural elements can be achieved via bioorthogonal coiled-coil libraries. This modular targeting enables multi-modal detection in addition to EM contrast. FIG. 8A: Shown here are coiled-coils targeting different fluorescent proteins selectively to the lumen of encapsulins (with different triangulation numbers: QtEnc (T=4), MxEn (T=3), TmEnc (T=1)) harboring the corresponding coiled-coil binding partner. The CN-PAGE panel on top shows the fluorescent signal of the cargo proteins encapsulated in either the T1, T3, or T4 encapsulins. FIG. 8A demonstrates that the given fluorescent protein (FP) fused to a given coiled-coil will only be encapsulated if the encapsulin structural element carries the cognate coiled-coil (CC) partner. Best working CC-pairs to ensure bio-orthogonal targeting to the Tm, Mx, or Qt encapsulin species have been identified and described herein. This targeting strategy allows for bio-orthogonal targeting of fluorescent proteins, which is not possible with the promiscuous native encapsulation signal of encapsulins. Exemplary sequences for this construct is provided in SEQ ID NO: 159-164. FIG. 8B: Corresponding fluorescent microscopy images show an enrichment and stabilization of the coil-coil-tagged fluorescent proteins inside the lumen of the encapsulin shells with the corresponding coiled-coil binding partners. Alternatively or in addition, the CCs can also be linked with an MT3 linker to enable EM and fluorescent detection (SEQ ID NO: 21 with an example for a cognate coiled-coil binding partner with SEQ ID NO: 22). Alternatively, exogenous fluorophores can be bioconjugated via SNAPtags or Halotags fused to the inner surface of encapsulins. Exemplary sequences (SEQ ID NO: 210, 211) FIG. 8C: Independent demonstration of the highly selective binding of the selected pairs of coiled-coils inside the lumen of encapsulin, here measured by bioluminescent photon counts from a luciferase cargo. Coiled-coils were chosen from Lebar et al., Nat.Chem.Biol. (2020); Chen et al., Nature, (2019).

FIG. 9: Encapsulins with different radii: FIG. 9A: Schematic representation of encapsulin systems with different triangulation numbers, resulting in the following radii: Q. thermotolerans (Qt; T=4, 240 sub-units, 40 nm;), M. xanthus (Mx; T=3, 180 sub-units, 32 nm;), and T. maritima (Tm; T=1, 60 sub-units, 20 nm;). FIG. 9B-FIG. 9D: Exemplary TEM micrographs of HEK293T cells expressing either MT3-QtEnc^{FLAG} (SEQ ID NO: 165), MT3-MxEnc^{FLAG} (SEQ ID NO: 166), or MT3-TmEnc^{BC2Tag} (SEQ ID NO: 144). The scale bar is 100 nm, and the scale bar in the zoom-ins is 20 nm. An exemplary sequence for this construct is provided in SEQ ID NO: 212. FIG. 9E: Frequency distribution of diameters of the encapsulin nano-spheres as structural elements shown in b)-d). FIG. 9F: CN-PAGE analysis of lysates of HEK293T cells expressing MT-fused encapsulin variants of varying radii. The MT-fused variants have the same electrophoretic mobility as the wild-type variants indicating that the MT domains (contrast-generating units) are lumenal and do not impair encapsulin assembly or change their geometric properties. FIG. 9G: Mean image (left) of 100 manually segmented instances of TEM images of MT3-MxEnc and MT3-QtEnc expressed in HEK293 cells (shown right). Intensity profiles along the indicated on the mean images on the left (center panels), showing the distinct inner diameters of the white center and the outer black ring. Importantly, the geometrically distinct EM contrast can not only be detected in TEM, capable of high magnifications, but also in SEM compatible with high-throughput analysis by volume EM. FIG. 9H: Higher-magnification TEM micrographs of Drosophila neurons in the optic lobe expressing MT3-QtEnc-FLAG-NLS (left) or MT3-Mx-FLAG-NLS (right). Scale bars are 50 nm (20 nm for inset). (Exemplary sequence for a fusion of the NLS to FLAG is provided in SEQ ID: 167). FIG. 9I: juxtaposition of TEM and SEM images taken from brains of a Drosophila melanogaster line (elav-Gal4 x UAS-QtEnc^{FLAG}-NLS; SEQ ID NO: 168), which was fixed and stained with standard protocols. Ultrathin sections were then captured either on TEM grids or on silica wafers for subsequent analysis by SEM (inverted contrast), allowing for the analysis of the identical cell. FIG. 9J: Same protocol as in h) but with MxEnc expressed in all neurons of the nervous system of Drosophila melanogaster (elav-Gal4 x UAS-MxEnc^{FLAG}-NLS; SEQ ID NO: 167). FIG. 9K: MT3-QtEnc expressed in Drosophila melanogaster neurons. The tissue was fixed, embedded, stained, and prepared for FIB milling. FIB milling uses a focused ion beam to sequentially ablate material from a sample to enable volumetric imaging. After each milling step, an SEM image is acquired, and 3D information can be reconstructed from the resulting image series. An image volume of 3696 nm×1956 nm×204 nm (image width×image height×milling length) was acquired on a Crossbeam 550 FIB-SEM (Zeiss) with a voxel size of 4 nm using an InLens detector at 1.3 kV and a working distance of 5 mm. FIB beam settings: 30 kV/700 pA. Acquisition time: 67 minutes. The scale bar in the overview image represents 400 nm, the scale bar in the cropped image represents 50 nm. Inset: Ortho-slices through selected nanospheres.

FIG. 10: Concentric barcodes Structural elements based on the concatenation of MT3 domains fused to Enc monomers produce differential concentric barcodes. FIG. 10A: Scheme showing the single, double, or triple repeat MT-domains fused to the N-terminus of QtEnc, which provides increasingly more osmiophilic cysteine residues that also reach further into the lumen of the encapsulin shell. Note that for the triple repeat, a chimeric metallothionein series from multiple organisms was used to minimize redundant DNA sequences, which can be unstable (see, e.g., SEQ ID NOs: 5, 169). FIG. 10B: TEM image of ultra-thin heavy metal-stained slices of HEK293T cells expressing MT3-QtEnc^{FLAG} resulting in an annular electron-dense ring with a bright center (left), 2xMT3-QtEnc^{FLAG} with a much smaller bright center, and 3xchimericMT3-QtEnc^{FLAG}, which adds an additional layer of contrast into the spherical structural element, i.e., an additional concentric ring in the 2D projection of the TEM, leaving no white center. FIG. 10C: Corresponding barcode shapes for the MxEnc with smaller outer diameter. Singular or tandem MT-domains fused to the N-terminus of MxEnc. FIG. 10D: Line plots through the center of an exemplary MT3-QtEnc^{FLAG} and 2xMT3-QtEnc^{FLAG} particle. Scale bar: 100 nm. FIG. 10E: Schematic showing how additional contrast generating units (CGU), such as MT3 or FABP can be added to the outer surface of encapsulins (or other structural elements) via direct fusion, SpyTag/SpyCatcher interaction, or intrabody/XFP interaction (SEQ ID NOs: 191, 192). The spacing between such additional concentric barcodes can be controlled by a rigid spacer such as SasG. The rotational invariance of the concentric barcodes ensures a robust readout in nanoscopy even if an isotropic resolution cannot be obtained. In addition to encapsulins, the concentric assembly can also be achieved by other spherical structural elements such as ferritin or viral capsids, such as MS2 (SEQ ID NO: 100), Acinetobacter phage AP205, or Qbeta phages. FIG. 10F Examples for outer contrast rings obtained by either binding the contrast-generating units MmMT3 (Mm stands for Mus musculus, SEQ ID NO: 1) or B-FABP (exemplary sequence SEQ ID NO: 36) to the outer surface of encapsulin via the isopeptide forming partners SpyTag/SpyCatcher. The construct comprising MmT3 is given as SEQ ID NO: 171. The construct comprising B-FABP is given as SEQ ID NO: 150. The TEM micrographs on the right show the corresponding concentric barcodes added to the outer surface of encapsulin, which can be modularly combined with intraluminal modifications with MT3.

FIG. 11: Vault assemblies The claimed structural elements comprise vaults. Vaults consist of 2 hemi-vaults, each consisting of 78 sub-units of the major vault protein (PDB: 6BP7). FIG. 11A: The schematic (left) and graphics (right) show several of the engineered vault constructs. The bimodal fluorescent and EM visible cargo (mEoS4b fused to APEX2; SEQ ID NO: 208) are connected to a targeting moiety (mINT, SEQ ID NO: 170), enabling cargo packaging into the vault compartment. In comparison to the wild-type vault, in which both termini face to the inside of the nanocompartment, an external attachment point is installed via insertion of palmitoylation sites contained within SNAP25 (SEQ ID NO: 78) via two helix-loop-helix motifs (antiparallel (AP) AP4 and P3 coils). Alternatively, an S-palmitoylation signal contained in Gap43 (SEQ ID NO: 209) is placed on the N-terminus to prevent the holo-vault assembly from two halves but instead target a hemi-vault to the membrane. FIG. 11B: The corresponding fluorescent images show the distribution of mEos4b as destabilized cargo to the vault compartments. Whereas the distribution of mEos4b inside the wild-type holo-vaults is homogeneous inside the cytosol, the SNAP25-modified vaults show micrometer-sized green-fluorescent arrays, while the Gap43-modified variants SEQ ID NO: 209 show intracellular membraneous clusters. FIG. 11C: TEM micrographs showing the large arrays of the SNAP25-modified vaults (left) (SEQ ID NO: 78). In comparison, the expression of vaults modified with APH peptides (using regular and truncated antiparallel CCs from GA354 (SEQ ID NO: 79) and GA355 (APH) (SEQ ID NO: 80), on their surface results in clusters of different packing. FIG. 11D: Comparison of distinct patterns generated in EM by vault constructs with external SNAP25 motif (left) and with IMEF as N-terminal fusion (right). FIG. 11E: Fluorescence microscopy images of HEK293T cells expressing the major vault protein where a small fluorescent eUnaG (enhanced UnaG) protein was fused to the internal HLH domain, and the red fluorescent protein mScarlet was targeted to the lumen via an mINT motif. An exemplary sequence for this construct is provided in SEQ ID NOs: 77-85.

FIG. 12: Nanofibrils and clusters Contrast-generating units such as MT3 can be added to naturally occurring or de novo designed fibrils or lattice-forming components to generate EM-contrasted structural elements. FIG. 12A: AlphaFold prediction of the major curlin subunit CsgA from E. coli. This protein has been shown to form long amyloid-like fibers. FIG. 12B: CsgA optimized for intracellular expression fused to a SpyTag was co-expressed with MT3 fused to the cognate SpyCatcher binding partner (SEQ ID NOs: 112, 171), yielding contrast-enhanced fibers. The TEM micrograph shows bundles of these highly contrasted fibers in the cytosol of HEK293T cells. As an alternative embodiment, de novo designed fibril forming proteins may be used for the generation of a structural element: FIG. 12C: 3D image of a cutaway view of a de novo designed filament-forming protein (DHF119) (left). Confocal fluorescence microscopy of HEK293T cells expressing DHF119-sfGFP (right) (DHF119 published in Shen et al., Science (2018)). DAPI-stained nuclei are depicted blue, whereas the green signal shows the perinuclear DHF119 filaments. The scale bar is 20 μm. FIG. 12D: Corresponding TEM micrograph of HEK293T cells expressing the contrast generating unit MT3 fused to DHF119_sfGFP filaments (SEQ ID NO: 110). FIG. 12E: Two-component lattices (Di13b2 and Di13a, both published in Ben-Sasson et al., Nature (2021)) fused to GFP, which yield lattices as shown on the confocal fluorescent microscopy image. Scale bar is 20 μm. FIG. 12F: Corresponding TEM micrograph in which MT3 is fused as a contrast-generating unit (SEQ ID NO: 1).

FIG. 13: Concatenation of contrast generating units The contrast generating units can also be concatenated via short linkers to generate nanometer-sized contrast-generating units. FIG. 13A: Shown here is the progression from a dimer, trimer, tetramer up to a decamer of FABP using its natural termini. One G-E-G repeat of SasG (^{˜}16 nm) is shown as a reference for scale (top of each image). Repeating sequences on the nucleotide level can be avoided by exploiting sequence differences between the subtypes from the large FABP family. Individual FABP contrast-generating elements can also be connected via MT3, serving as a flexible linker and, in addition, as a contrast-generating unit. Exemplary sequences for these constructs are given in SEQ ID NOs: 194, 195. FIG. 13B: To increase the geometric diversity of the resulting structural elements, FABPs can be circularly permutated (cpFABP; SEQ ID NO: 193) to generate new termini between the alpha-helices. If those termini are fused between two cpFABPs to generate dimers, the angle can be controlled by the linker used for the fusion. FIG. 13C: Via side-by-side concatenation of FABPs, cylindrical shapes can be generated that can be closed to toroidal shapes, which can be stabilized via hydrophobic moieties and further be spatially organized via, e.g., isopeptide bonds and inteins. An exemplary sequence for these constructs is given in SEQ ID NO: 196.

FIG. 14: Mono-to-polyvalent hubs as structural elements While the substantial size that can be obtained with self-assembling nanostructures is an advantage for building nanoscopy-readable structures, it comes with the natural disadvantage that the resulting structure is symmetric and multivalent. Monovalent hubs or adapters to which several contrast-generating units can bind are thus beneficial. FIG. 14A: (left) 3D image of an exemplary emFP (electron microscopy Fluorescent Protein) based on GFP showing the tag insertion points at the termini and an internal loop. (Right) Linear scheme showing the different emFP variants with the insertion points and a number of their respective intrabody-addressable tags. Exemplary sequences for these constructs are given in SEQ ID NOs: 92-99, 172-178. FIG. 14B: Schematic of the contrast unit binding to the emFPs consisting of intracellularly-expressable intrabody or scFv fragment, the respective metal interacting unit (or a tandem thereof), GB-1 (to enhance solubility), a FLAG-epitope for detection, and a C-terminal RxxG degron for background suppression. Exemplary sequences for these constructs are given in SEQ ID NOs: 172-178. FIG. 14C: Immunocytochemistry of HEK293 cells expressing LifeAct-emFPv4 (SEQ ID NO: 179) localizing to filamentous actin. The green signal (top) represents the emFPv4 (SEQ ID NO: 94) itself, whereas the red signal (bottom) corresponds to the FLAG-tag on the contrast unit, which colocalizes with the green signal indicating successful binding to the hub. FIG. 14D: Anti-FLAG western blot of whole-cell lysates from HEK293T cells co-expressing LifeAct-emFPv4 (SEQ ID NO: 179) plus the respective metal binders. The bands correspond to the expressed contrast units demonstrating their successful expression in mammalian cells. FIG. 14E: UV fluorescence clear native PAGE analysis of lysates from HEK293T cells co-expressing LifeAct emFPv4 and the respective contrast unit. The green signal at the bottom corresponds to non-bound emFPv4, whereas the higher molecular smears correspond to emFPv4 bound to several contrast units. FIG. 14F:Furthermore, a few selected viral capsids break the symmetry with portal, tails, or accessory proteins as known for picorna-, parvo-, micro-, and leviviridae. A specific example would be the maturation protein of MS2, Acinetobacter AP205 phages, or the Allolevivirus Qbeta phage. The maturation protein is a specific component in the MS2 capsid, which represents a unique single attachment point. Based on the detailed structural information, a monomeric attachment of, e.g., MS2 capsids to F-pilus motifs can be designed via the maturation protein, while the MS2 RNA (or a subset thereof harboring the critical stem-loops) can provide EM contrast. The mammalian expression vectors are shown on the bottom (see also sequences of SEQ ID NOs: 100, 101, 102, 103). FIG. 14G: A preferred instantiation for engineered mono-to-polyvalent adapters or hubs is based on coiled-coil-rich proteins such as the Anbu protease (left) (SEQ ID NO: 88) or toroid protein (right) (SEQ ID NO: 89), in which the external coiled-coils can be replaced by designed coiled-coils that have high-affinity binding partners. PDB entry numbers are given in FIG. 14G. At the termini, all of these surface-exposed coiled-coils can be augmented by CCC-tags or Suntags. CCC-tags are a linear concatenation of coiled-coils. Suntag allows for recruiting up to 24 copies via a single-chain variable fragment (scFv) antibody to which a contrast-generating unit as described above can be attached. FIG. 14H: Other preferred examples of proteins with engineerable hypervariable loops that can be replaced with epitopes, coiled coils, or isopeptide-bonds are adhesins (left) (SEQ ID NOs: 89, 90) and fiber-knob domain proteins (right) (SEQ ID NO: 91). PDB entry numbers are given in FIG. 14H.

FIG. 15: Programmable stoichiometry control using read-through motifs FIG. 15A: Structural elements can be equipped with genetically controlled internal and external attachment points to bind contrasting units, target the structures to molecules of interest, or generate geometric patterns. Protein domains might be directly fused to the termini or an internal region of the structural element. Protein cargos might be internally docked via endogenous targeting signals such as encapsulation signals in the case of encapsulin and mINT signals in the case of vaults. Furthermore, attachment points in the form of nanobodies, CC domains, SpyTag or SpyCatcher domains, SNAP/Clip-Tag domains, and Halo-Tag domains might be fused. FIG. 15B: Since most structural elements are self-assembling and highly symmetrical, programmable translational read-through can be applied to regulate the stoichiometry of (surface) modifications. The schematic shows the genetic makeup of an exemplary programmable read-through (RT) cassette consisting of a QtEnc monomer gene with a C-terminal FLAG epitope followed by a stop codon and read-through promoting sequences (e.g., SEQ ID NO: 180, including a CTATCC DNA sequence after the stop codon, which promotes ribosomal (translational) read-through). When ribosomal read-through occurs, the ribosome skips the stop codon and continues to synthesize the nascent polypeptide chain. By using different RT motifs, different degrees of read-through can be obtained, yielding encapsulin nanostructures that consist of monomers with a C-terminal FLAG epitope (immediate stop, no RT) or with a FLAG with an additional intrabody domain (stop skipped, read-through occurred). The rate of read-through is determined by the stop codon and the respective RT motif (Exemplary SEQ ID NO: 74-76). The RT rate is defined as the number of monomers with the additional intrabody, which arise from the RT divided by the total number of monomers. Another variant of the RT cassette uses a fast-cleaving intein mutant (SspDnaE; SEQ ID NO: 181), followed by a P2A peptide to yield two fully independent, unfused, scarless polypeptide chains since the middle intein module splices on the N-terminus and downstream polypeptide will be liberated via the P2A peptide (SEQ ID NO: 182). Therefore, the RT system can be used to co-express two unrelated polypeptides at different ratios depending on the RT motif used. FIG. 15C: Densitometric analysis of RT rates depending on different stop-codon RT motif combinations. The combination of stop codons with RT motifs allows for adjusting RT rates between ^{˜}1 and 20%. Another way of quantifying the RT rate is based on the complementation of a split NanoLuciferase. If RT occurs, a HiBit epitope will be translated downstream of the intrabody sequence. After cell lysis, purified LgBit protein is added, which reconstitutes a functional luciferase yielding a luminescence signal serving as a proxy for RT.

FIG. 16 Endogenous scaffolds FIG. 16A: Structural elements can be spatially patterned via naturally occurring cellular scaffolds. The TEM micrograph of HEK293T cells expressing QtEnc modified with C-terminal farnesylation signal (SEQ ID NO: 40) shows the QtEnc capsids targeted to cellular membranes. FIG. 16B: Structural elements can also be arranged on the cytoskeleton. The confocal fluorescence microscopy images show HEK293T cells expressing mScarlet-I-loaded QtEnc C-terminally modified with a KinTag peptide (Cross et al., Cell Chemical Biology (2021); SEQ ID NO: 113) mimicking a cargo adaptor connecting to endogenous motor proteins. The fluorescent signal represents the location of the encapsulins nano-compartments as structural elements within the cell, which can be seen as dotted signals in the perinuclear region and the periphery. FIG. 16C: TEM micrograph of HEK293T cells co-expressing 2xMT3-QtEnc^{antiGFPIB} (SEQ ID NO: 183) and the lysosomal marker LAMP1-GFP, which presents GFP on the outside of the lysosomes. The micrograph shows that highly contrasted QtEnc nanostructures co-localize with the lysosomes. FIG. 16D: In this example, the membrane is used as a scaffold via expressing membrane-localized GFP (GFP-CAAX) to which the anti-GFP intrabody on the surface of QtEnc binds to (left). In the control condition, GFP-CAAX is again expressed, but the intrabody is replaced with a FLAG tag (right). The contrast in both examples is provided by co-expression of the metal importer Zip14 and the ferroxidase IMEF targeted to the encapsulin via an encapsulation signal.

FIG. 17 Genetically controlled scaffolds for barcodes readable by nanoscopy FIG. 17A: (top) Schematic of the use of the microbial filamentous protein SasG, a rigid hetero-bifunctional scaffold from S. aureus, to enable nanoscopically visible shapes, preferably barcodes. Of note, SasG consists of a repeating pattern of G5 and E units. G5_1-G5_2 is an exemplary representation of the length, wherein G5_1 and G5_2 are connected via E(1). G5_1-E(1)-G5_2, for instance, yields 16 nm. G5_1-G5_4 means G5_1-E(1)G5_2-E(2)-G5_3-E(3)-G5_4 yields 30 nm. Exemplary sequences for these constructs are given in SEQ ID NOs: 114-123. The termini are fused to sfGFP (SEQ ID NO: 184) and mCherry (SEQ ID NO: 18, respectively, which can be addressed with structural elements, in this case, with QtEnc modified with an antiGFP intrabody and TmEnc with an anti-mCherry intrabody (LaM-4) (SEQ ID NOs: 186, 187). Of note, SasG is here used as a genetically controlled scaffold. (bottom) Schematic representation of modular make-up of SasG filaments consisting of alternating G5 and E units yielding different sizes. sfGFP-G51-G54-mCherry (SEQ ID NO: 188) yields a length of approximately 30 nm. FIG. 17B: Clear native PAGE analysis of lysates from HEK293T cells expressing different sfGFP-SasG-mCherry variants yielding different lengths. The CN-PAGE gel shows an sfGFP-mCherry tandem as well as different lengths of SasG as a spacer between sfGFP and mCherry. FIG. 17C: Live wide-field fluorescence microscopy imaging of HEK293T cells expressing sfGFP-SasG_G51-G52-mCherry+MT3-QtEnc^{antiGFPIB}+P2-TmEnc^{antimcherryIB} (LaM-4) (SEQ ID NOs: 114, 189, 109). The red and green fluorescent channels demonstrate the expression of the heterobifunctional scaffold. Clusters are visible, which indicates the interaction between the scaffold and the polyvalent encapsulins serving as structural elements. FIG. 17D: Corresponding TEM micrographs of HEK293T cells co-expressing sfGFP-SasG_G51-G52-mCherry+MT3-QtEnc^{antiGFPib}+P2-TmEnc^{antimcherryIB} (LaM-4). Clusters of 40 nm QtEnc structures surrounded by TmEnc capsules can be observed. The zoom-in shows the distinct spacing between the patterning of QtEnc and TmEnc, which can be used to encode multiplexed information in an analogy to a QR barcode. FIG. 17E: In distinction to d), a longer SasG with 4 G5 units (SasG-G51-G54) was co-expressed here. FIG. 17F: Here, clusters containing sfGFP-SasG_G51-G52-mCherry linkers (^{˜}16 nm) and clusters containing sfGFP-SasG_G51-G54-mCherry (^{˜}30 nm length) are juxtaposed for comparison. FIG. 17G: In addition to linear scaffolds, the rigidity of SasG also allows for more complex scaffolds. Depicted here is an example with triangular shapes in which isopeptide bonds (SpyTag/SpyCatcher, SnoopTag/SnoopCather) or inteins (SEQ ID NOs: 115-117) were used as unique connectors between the edges. The fluorescent proteins sfGFP, mCherry, and mTurquoise are also positioned at the edges to serve as binding sites for structural elements. FIG. 17H: CN-PAGE analysis of lysates from HEK293T cells expressing different combinations of the components shown in g Under fluorescence illumination, the CN-PAGE gel demonstrates the expected combination of individual elements (color-coded). The bio-orthogonal reaction of the components is apparent from the mass shifts in the gels and the color mixing (not apparent in a black/white copy of this FIG. 17H). The last lane (1+2+3) shows a high molecular weight band (dashed box) corresponding to the assembled triangle. The fluorescent proteins positioned at the vertices can be replaced by emFPs (SEQ ID NOs: 92-97) or other hubs (see, FIG. 15) or contrast generating units. FIG. 17I: Alternatively, unbounded clusters can be generated by linking together structural elements such as shown for encapsulins and multivalent de novo cages. The TEM micrograph shows HEK293T cells co-expressing iron-mineralizing QtEnc C-terminally modified with a SpyTag, which covalently clusters with 13-01 (Hsia et al., Nature (2016)), a de novo designed icosahedron, presenting 60 cognate SpyCatcher binding partners. FIG. 17J: As an alternative to the proteinaceous scaffold exemplified by SasG, RNA scaffolds can be designed via ROAD (Geary et al., Nat. Chem., (2021)) can be used but using L7ae and BIV-tat (SEQ ID NOs: 63, 64) instead of MCP and PCP as attachment points and incorporating fluorescent aptamers such as Spinach (SEQ ID NO: 55) and Dir2s (SEQ ID NO: 57).

FIG. 18 Geometric sensing via proteolytic unmasking and molecular mapping Assembly of structural elements can be controlled via proteolytic de-blocking of monomeric subunits. FIG. 18A: Schematic of calcium-dependent proteolytic unmasking of sterically blocked QtEnc monomers. The scheme shows the different genetic components, including the blocked monomer with a TEV cleavage site (3xLBT15-TEVsite-QtEnc-FLAG; SEQ ID NO: 128), the two calcium-dependent Split TEV parts (SCANR; published in O'Neil et al, ACS Chemical Biology, (2018)), a degron-tagged NanoLuc-cargo; SEQ ID NOs: 129, 130), which serves as a reporter for assembly because it will be protected upon proteolytic de-blocking, and a regular TEV protease. Upon elevated Ca²⁺ levels, the SCANR parts will be reconstituted, the re-complemented TEV protease will cleave off the sterical 3xLBT block, the encapsulin nanocompartment will assemble and encapsulate the Luciferase cargo enzyme such that it is protected from degradation and give bioluminescent signal. FIG. 18B: In this example, QtEnc monomers are blocked with a triple LBT15 domain but can be unblocked via a bio-orthogonal protease such as TEV (TEVp), which leads to the assembly of the encapsulin nanospheres as shown by CN-PAGE. The protease may be expressed constitutively, under control of a promoter of interest, or can be made responsive to a second messenger such as calcium by using an engineered calcium-sensitive protease such as SCANR. Upon co-expression of SCANR, encapsulin assembly gradually increases with calcium, as can be seen by the increasingly stronger bands for the encapsulin on CN PAGE as well as an increasing luminescence signal obtained from the gel from a co-expressed NLuc cargo, which is destabilized unless it gets encapsulated inside the encapsulin shell. FIG. 18C: SDS-PAGE of material from a FLAG pull-down from cells expressing the conditions in b). The molecular weight shift indicates calcium-dependent cleavage of the blocking domain when either TEVp or SCANR+ionophore are present. FIG. 18D: Bulk luminescence reading of the experiment described in b). FIG. 18E: TEM micrograph of HEK293T cells expressing the blocked monomer+SCNAR 24 h post ionophore treatment. Assembled MT3-QtEnc^{FLAG} nanocompartments (structural elements) are visible throughout the cytosol. FIG. 18F; and FIG. 18G: SasG filaments can also be engineered to increase in length upon a cellular signal or state. This can be achieved by using engineered split intein domains, which can be fused to fragments of SasG to reconstitute SasG filaments of different lengths. In this example, sfGFP-SasG_G51 units are reconnected to SasG_E1_G52_mCherry units via different split intein systems (VidaL in f) and Npu DnaE in g); SEQ ID NOs: 121-123 and 118-120). The reconstitution of the fragments is indicated by the yellow band running above 242 kDa on CN-PAGE, which has the same electrophoretic mobility as the sfGFP_SasG-G51_E1_G52_mCherry construct. FIG. 18H: SasG filaments can also grow conditionally by using a self-growing EG fragment that has split-intein domains on either side of the building block (SEQ ID NOs: 120, 123). The growth can then be capped off by a capping unit (SEQ ID NOs: 119, 122). FIG. 18I: This conditional growth can be made directional by using QtEnc^{antiGFPIB} as a scaffold, in which a calcium-dependent TEV (SCANR) would un-cage a caged split-intein (Gramespacher et al., 2017) and initiate growth. Upon a certain cellular signal of interest, the growth can be capped off by expressing the capping unit. FIG. 18J: Proteolytically triggered nanostructure assembly can also be realized with vaults. The schematic shows a split vault monomer, in which each half is blocked by split intein that can be activated via proteolytical cut by, e.g., TEV, thus leading to the intein-mediated (supported by a coiled-coil motif) fusion of an intact vault monomer, exposing a SNAP25 motif. FIG. 18K: The fluorescence images show the TEV-dependent agglomeration of a fluorescent cargo (mScarlet docking via the mINT binding site) compared to the condition in which non-split vault monomers are used (right column). FIG. 18L:To complement molecular mapping via direct binding to the target biomolecule of interest, a deactivated version of Trim21 (dTrim21, SEQ ID NOs: 138, 139), incapable of auto-ubiquitinylation, can be co-delivered to cells (or expressed) together with standard antibodies to a target of interest, to act as an adapter to a structural element or scaffold. Shown here is a preferred instantiation, in which a fusion of mCherry or GFP to dTrim21 is used in combination with an encapsulin as a structural element harboring an anti-mCherry (Lam4) (SEQ ID NOs: 186, 187) or anti-GFP intrabody on the surface.

FIG. 19: Geometric sensing via a change in the concentric barcode or patterning FIG. 19A: Geometric sensing can be conducted via recruitment of contrast generating units to the surface of structural elements. Shown here is the specific case of FABPs recruited via the interaction of calmodulin and calmodulin-binding peptides to encapsulins (SEQ ID NOs: 197, 198), which alters the contrast contour of the concentric barcode. Please note that this mode is the same as in FIG. 10E and FIG. 10F, except that the addition of the external concentric barcode is dependent on the calcium-driven binding of calmodulin to calmodulin-binding peptides. Bottom: In the encapsulin lumen, concentric barcodes can also be modified by using contractile elements such as engineered contractile elements from troponin that will shorten the distance to the protein shell upon binding of calcium (SEQ ID NOs: 136, 137). FIG. 19B: In another preferred embodiment, the agglomeration state of structural elements such as encapsulin can be changed via calcium-mediated interactions of calmodulin and calmodulin-binding peptides. As another polydentate kernel for agglomeration, ferritin such as Helicobacter pylori can be used as a fusion to calmodulin (SEQ ID NO: 134). The data show the formation of fluorescent clusters of encapsulins loaded with fluorescent cargo and equipped with respective calcium-binding motives after adding 10 μM calcium ionophore. An analogous system can be built for phosphorylatable peptides (e.g. SEQ ID NO 239) and phosphopetide-binding proteins such as SEQ-ID NO: 206. FIG. 19C: Alternatively, a calcium integrator by patterning can also be achieved by changing the subcellular localization of the nanostructure as a function of calcium concentration. Shown here is a preferred embodiment in which an N-myristoylation signal contained within recoverin (PDB: 1jsa; SEQ ID NO: 238) is fused to the N-terminus to enable calcium-dependent relocalization. As known for recoverin, this membrane anchor swings out in the presence of calcium, causing relocalization to membranes. As shown by the green fluorescent mEOS4b signal, the resultant protein complexes are homogeneously distributed in the cytosol (top) unless calcium influx is triggered, leading to the generation of clusters, similar to the constitutively available S-palmitoylation signal (bottom) (SEQ ID NO:209).

FIG. 20: Geometrically precise actuation via biomechanical actuation and/or deposition of local energy FIG. 20A: The genetically controlled structural elements and scaffolds can exert biomechanical forces by polyvalent interactions with their surfaces while providing monitoring of the manipulations via nanoscopy. Shown here is the clustering of endogenous glutamate receptors in Drosophila neurons via multivalent encapsulins surface-functionalized with intrabodies against GFP, which co-cluster GFP-tagged receptors. The control (left) shows the regular distribution of GluCl receptors labeled with GFP in Drosophila brains when encapsulins, surface-decorated with anti-GFP intrabodies, were not expressed via a temperature-sensitive promoter. When expression of MT3-QtEnc^{antiGFPintrabody} was permitted at 29° C. (using the established system with a temperature-sensitive allele of GAL80) either after sparing early developmental phases (middle panel) or during development (right panel), mild or strong clustering of GluCl receptors in the somata was observed (McGuire et al, Science, (2003). Thanks to the controllable spatial arrangement of polyvalent binders by scaffolds such as SasG, more complex spatial manipulations can be systematically optimized via the nanoscopy readout. FIG. 20B: Geometrically defined actuation can also be achieved via local deposition of energy through magnetic gradient fields. Shown here is the magnetic cell sorting (MACS) of HEK293 cells overexpressing encapsulins with the ferroxidase QtEnc_IMEF encapsulated as a host enzyme (and the iron importer Zip14, or mEos4b as control), which leads to the biomineralization of biomagnetic iron-oxide species. In addition, the iron-oxide-filled encapsulins are surface-functionalized with different attachment points (SpyTag, anti-GFP intrabody) to spatially organized via different co-expressed scaffolds, including the de novo cage 13_01 functionalized with a SpyCatcher (see FIG. 17i)), the filament DHF functionalized with GFP (see FIGS. 12C and 12D). In addition, a farnesylation tag (see FIG. 16a)) is used to target the encapsulins to the membrane. A FLAG tag serves as control. After expression in HEK293 cells (grown with ferrous ammonium sulfate), the cells were dissociated and passed over a commercial magnetic column (Miltenyi) placed either inside the magnetic field or outside as a control. The data (mean with SEM) show that magnetic gradients exert sufficient mechanical forces to retain entire cells that are expressing the geometrically organized magnetic and EM-visible geometric patterns. FIG. 20C: Similar to magnetic gradients, encapsulins can also be equipped with photoabsorbers such as biosynthetic pigments, which allow for energy deposition via visible or infrared electromagnetic radiation. Shown here as an example are encapsulins that have the enzyme tyrosinase encapsulated via coiled-coil pairs (P1/P2 in the case of TmEnc (SEQ ID NOs: 159, 190), other combinations are given in FIG. 20C), which generate the strongly absorbing polymer melanin from the endogenously available amino acid tyrosine. This polymerization reaction occurs inside the lumen of the nanocompartment, which prevents the product melanin from diffusing out, while the substrate tyrosine can diffuse into the lumen through the encapsulin pores. Spatially organized patterns of these photoabsorbing encapsulin species can be used to photoablate cellular structures. Monitoring can also be achieved via photoacoustic imaging, which picks up photoabsorption.

FIG. 21: de-novo designed uranyl binding proteins. FIG. 21A: A starting epitope of disjoint residues can be mapped by its backbone (i.e., sequence-agnostic) features. Inter-residue atomic distance matrices and internal dihedrals can serve as canonical fingerprints for epitope matching. Starting from a query epitope of a high-affinity uranyl-binding site, several binding sites could be identified on a compact coiled-coil miniprotein with backbone RMSD <0.3. The uranyl binding residues can thus be engrafted in place of the template's residues to create a single-domain protein with multiple binding sites. FIG. 21B: Top scoring hits all exhibited a high level of symmetry and a strong local shape similarity to the uranyl-binding epitope. C3-, C4-, C5-symmetric hits were dominant among top hits, given the number of sites/atom constraints. FIG. 21C: Three design templates were highly expressed in E. coli and showed helical spectra. Results are shown for 4bhv, and 5apq oligomeric variants as analyzed by SDS/PAGE and circular dichroism. FIG. 21D: Average radial profile plot showing a comparison between MUP2_QtEnc (n=100) and 1M_QtEnc (n=100) integrated gray value from the center of the encapsulins outwards. FIG. 21E: Normalized average particle for both MUP2_QtEnc (n=100) and 1M_QtEnc (n=100).

FIG. 22: Lipid-binding proteins as contrast-generating units (CGUs). FIG. 22A: Schematic representation of QtEncapsulin (QtEnc) fused to brain-derived Fatty Acid-Binding protein (B-Fabp) B-Fabp and a mutant of B-Fabp with ablated lipid binding (B-Fabp-ko). FIG. 22B: Average radial profile showing a comparison between QtEnc_B-Fabp and QtEnc_B-Fabp-ko integrated gray values from the center of the encapsulins outwards (n=50). FIG. 22C: Normalized average particle for both QtEnc_B-Fabp (n=50) and QtEnc_B-Fabp-ko (n=50). FIG. 22D: Background-subtracted average particles for both QtEnc_B-Fabp (n=50) and QtEnc_B-Fabp-ko (n=50) highlighting the contrast of the encapsulin shells, B-Fabp and B-Fabp-ko. The average image on the button shows the differential contrast due to lipid binding (i.e., the contrast from B-Fabp-ko subtracted from the contrast from Bfabp). FIG. 22E: Schematic representation of H. pylori ferritin fused to B-Fabp and Bm3h. FIG. 22F: Clear-Native Page with Coomassie Staining confirming the assembly of both constructs FIG. 22G: Normalized average particle for both HpFtn_B-Fabp (n=200) and HpFtn_Bm3h (n=200). FIG. 22H: Average radial profile plot showing a comparison between HpFtn_B-Fabp and HpFtn_Bm3h integrated gray values from the center of the encapsulins outwards (n=200). FIG. 22I: Schematic representations of B-Fabp, B-Fabp-ko, Bm3h, and Bm3h-B7, a Bm3h mutant which should inhibit lipid binding, fused to the FLAG peptide. FIG. 22J, FIG. 22K: Purified (anti-Flag tag) lipid-binding proteins were incubated for 15′ with 1 μM BODIPY™ FL C16 (Invitrogen). Fluorescence of the protein-bound phospholipids was detected on a 12% Tris-Glycine Gel run under native conditions with either UV illumination (left) or Coomassie Staining (right). A Fluorescent band can be seen on the UV-illuminated gel for B-Fabp_FLAG at double than expected MW, indicating the presence of a dimer. A band can be seen for both Bm3h and Bm3h-B7 at the expected MW, indicating that both have bound the BODIPY™ FL C16. FIG. 22L Genetic construct, TEM contrast of an average particle (n=200), and overview TEM contrast obtained from B-FABP-fused C-terminally onto the de-novo cage 13-01.

FIG. 23: Fluorescent encapsulins as high-contrast markers for CLEM. FIG. 23A: Targeted fluorescent encapsulins can be generated by N-terminal attachment of the small monomeric fluorescent protein (eUnaG) to 1M- and 2M-Qt harboring surface-exposed anti-mCherry intrabodies. Alternatively, encapsulin monomers fused with outwardly directed eUnaG can be co-expressed with encapsulin monomers, a portion of which can harbor an intrabody. FIG. 23B: Confocal laser scanning microscopy of fluorescent encapsulin composed of 1M-QteUnaG monomers co-expressed in a 4:1 ratio with monomers bearing anti-mCherry intrabodies (1M-Qtanti-mCherry) with and without co-expression of membrane-bound mCherry (mem-mCherryFLAG). The scale is 10 μm. FIG. 23C: Corresponding TEM micrograph of mem-mCherry-targeted 1M-Qtanti-mCherry+1M-Qtanti-mCherry (4:1). Scale bars represent 100 nm. FIG. 23D: Control over the ratios of the different encapsulin monomers can be achieved via tunable ribosomal read-through cassettes (rt) encoded on a single cistron. To this end, different combinations of stop codons and short rt-promoting motifs are combined at the end of the ORF encoding 1M-Qt. If rt occurs, the C-terminus of 1M-QtFLAG is further extended by an anti-GFP intrabody. FIG. 23E: Confocal fluorescence microscopy of HEK293T expressing the gap junction-forming protein msfGFP-Cx43 together with the 1M-QtFLAGrt20santi-GFP, resulting in ˜20% anti-GFP intrabodies on the encapsulin surface. The encapsulins variants are also rendered fluorescent by co-expression of mTagBFP2 as cargo proteins with the Qt encapsulation signal (QtSig) and an N-terminal degron (DD-N), leading to degradation of the non-encapsulated fluorescent proteins. Scale bars represent 10 μm. FIG. 23F Corresponding TEM micrograph showing 1M-Qt (with ˜20% anti-GFP intrabody) labeling a gap junction. (Scale bar represents 100 nm) FIG. 23G Confocal microscopy images showing multiplexed tagging of heterologous gap-junctions consisting of msfGFP-Cx43 and Cx26[mut]-mCherry with encapsulin assemblies consisting of 1M-PfEncFLAG-miRFP670nano/1M-PfEncFLAG-anti-GFP (Ratio 4:1) and 1M-QtFLAG+1M-Qtanti-mCherry (Ratio 4:1)+DD-N-mTagBFP2-QtSig, respectively. The scale bars represent 10 μm.

FIG. 24: Bioconjugation tags for exogenous synthetic fluorophores. FIG. 24A: SNAP-tag (New England Biolabs) was N-terminally or C-terminally appended to 1M-Q, expressed at varying ratios with unmodified 1M-Qt in HEK293T cells and loaded as native lysates on CN-PAGE (left) to ensure the assembly of encapsulins. The fluorescent SNAPtag substrate TMR star (New England Biolabs) was added to the gel for visualization of fluorescence to confirm the functionality of SNAPtag. Notice that a combination of SNAPtag-fused monomers with non-SNAPtag-fused encapsulin monomers is necessary to enable encapsulin assembly, which can be stably expressed via the read-through motifs described below. Right panel: Fluorescence from CN-PAGE derived from CLIPtag and substrate instead of SNAPtag. Instead of CLIPtag also biotin-binding avidins can be used. FIG. 24B: Combinations of SNAPtag-labeled encapsulins (as indicated in the figure) were expressed either untargeted or targeted to GFP-fused to the membrane (CAAX motif) via an anti-GFP intrabody also presented on the encapsulin surface. c) Fluorescence from TMR-star bound to SNAPtag-surface-presenting encapsulins is maintained after fixation with glutaraldehyde, 0.1% OSO₄, uranyl-acetate, dehydration over an ethanol series, and epon (Epon 812) embedding at 4 degrees celsius.

FIG. 25 Bioconjugation tags for exogenous synthetic fluorophores; fixation stable fluorescence contrast FIG. 25A: Correlative light-electron microscopy signals from 1M-QtEnc-SNAP construct expressed in HEK cells fixed, heavy-metal-stained and embedded in resin. Encapsulins were identified in the SEM image with a pixel size of 2 nm. FIG. 25B: The acquired SEM image was used to generate a binary mask of encapsulins, scaled to the FIG. 25C: wide-field fluorescence microscopy images used to identify the SNAP-TMR dye fluorescence bound to the SNAP-tag on the surface of the encapsulins. FIG. 25D: The fluorescence image was converted to a binary mask. FIG. 25E: A template-matching algorithm was used to identify the areas with the highest correlation of both masks in the fluorescence modality. FIG. 25F: The identified area was scaled to match the dimensions of the SEM image and overlaid on the top of the SEM image.

FIG. 26: Bimolecular Fluorescence Complementation (BiFC). Green fluorescence protein was re-complemented from a tripartite split with the two smaller components fused to encapuslins. Upon co-expression of the complementary beta-barrel sfGFP1-9, an agglomeration of fluorescent encapsulin assemblies can be obtained. FIG. 26A: Representation of Co-expressed QtEnc_sfGFPS10, QtEnc_sfGFPS11 and sfGFP1-9. Coexpression of QtEnc_sfGFPS10 and QtEnc_sfGFPS11 leads to the assembly of mixed capsids displaying both fusions on the surface. Reconstitutions of full sfGFP can happen in between the S10 and S11 strands fused to different encapsulins, leading to aggregation of fluorescent encapsulin assemblies FIG. 26B: TEM Micrograph showing the aforementioned aggregation. Scale bar is 250 nm.

FIG. 27: Targeting of CLEM-compatible Enc variants via coiled-coil pairs. FIG. 27A: Schematic showing of CLEM-compatible encapsulins via orthogonal coiled-coil (CC) pairs to a surrogate membrane target, including schemes of the genetic constructs. Co-targeting of mScarlet-I with an encapsulation signal is used to render encapsulin fluorescent in this experiment. FIG. 27B: Confocal fluorescence microscopy experiment showing encapsulin targeting to the membrane as depicted in a. The scale bar represents 20 μm.

FIG. 28: Multiplexed EM contrast with different numbers of concatenated metallothioneins (as CGU) and encapsulins of different diameters (as structural elements): FIG. 28A: Schematic representation of the expression in mammals of modular constructs encoding self-assembling encapsulin monomers with N-terminal fusions of metallothioneins (M) and C-terminal surface modifications for targeting (encapsulins). Expression of the encapsulin variants results in self-assembly of hollow protein nanospheres with different triangulation numbers (T), different diameters, and 1-3 concatenated copies of M facing the lumen of the porous protein envelopes. FIG. 28B: TEM micrographs (8× magnification, 0.5525 pixels/nm) following the standard protocol for fixation and heavy metal staining of HEK293T cells expressing the different encapsulin variants from a. Scale bars indicate 100 nm. The insets show the average values of the manually segmented image sections (n=1000, except n=900 for 1M-TmBC2Tag) for each condition (side length of insets: 89.2 nm (49 pixels), scale bars: 50 nm). FIG. 28C: Multiclass semantic segmentation from end-to-end U-network architecture. FIG. 28D: Multiplexed detection of different encapsulin class combinations in neighboring HEK293T cells with overlaid semantic segmentation masks. Scale bars represent 100 nm.

FIG. 29: In vivo expression of several encapsulins (as structural elements) and metallothioneins as contrast generating units (CGU) to generate concentric barcodes in TEM in vivo in Drosophila brain. Overview confocal fluorescence microscopy images of the optic nerve head (OL) of a Drosophila line with pan-neuronal expression of FIG. 29A: 1M-QtFLAG-NLS, or FIG. 29C: 1M-MxFLAG-NLS containing a nuclear localization signal (NLS). FLAG epitopes on the outer surface of encapsulins (anti-FLAG, cyan for 1M-QtFLAG-NLS and red for 1M-MxFLAG-NLS) are colocalized with cell nuclei (with DAPI in blue, scale bar 5 μm) but do not show cytoplasmic expression (anti-Bruchpilot, gray). Scale bar is 25 μm. FIG. 29B, FIG. 29D: Corresponding TEM images with semantic segmentation maps as overlays. The scale bar is 100 nm. The insets show the average of the respective encapsulin class identified in the validation dataset (n=132, bounding box: 89.2×89.2 nm), FIG. 29E: Overview image of confocal fluorescence microscopy of the central brain (CB) and optic nerve head (OL) and FIG. 29F: zoom on Drosophila optic nerve head, which contains both T4-T5 and C3 somata and expresses 1M-MxFLAG-NLS and 3M-QtFLAG-NLS encapsulins, and FIG. 29G: corresponding TEM micrograph with superimposed multiclass semantic segmentation masks, color-coded as in FIG. 28. The scale is 100 nm.

FIG. 30: Adeno-associated virus-encoded MT3-encapsulin fusion as genetically encoded EM marker in vivo in mouse brain. FIG. 30A: Schematic representation of the genetic construct for expression of 2M-QtFLAG together with mScarlet-I via AAV transduction and intracranial injection of AAVs into mouse hippocampus. FIG. 30B: Native (CN) and corresponding SDS gels after silver staining confirming assembly of 2M-QtFLAG after pull-down (PD) from the excised hippocampus. FIG. 30C: Confocal fluorescence images of a coronal section through mouse brain one month after AAV transduction, showing direct mScarlet I fluorescence in red and encapsulin expression in cyan (anti-FLAG, FITC). Scale bars are 1 mm and 200 μm for the inset, respectively. The inset shows a magnified region in the hippocampus. FIG. 30D: TEM overview image and FIG. 30E: magnification of the region bounded by the white dashed lines in d. Inset shows further magnification on the 3 encapsulins located within the bounding box (black dashed lines). Scale bars for the respective magnifications are 1 μm, 100 nm, and 50 nm. FIG. 30F, FIG. 30G, FIG. 30H: Instances of encapsulins in neuronal processes, color-coded semantic segmentations as defined in FIG. 28. [symbol] denotes membrane interruptions. Scale bars represent 100 nm.

FIG. 31: Stabilized fluorescent RNA cargo targeted to self-assembling vaults as structural elements FIG. 31A: Schematic showing the circularized Twister-Broccoli RNA aptamer(DOI: 10.1038/s41587-019-0090-6) equipped with a PP7 aptamer. The variant shows the circularized spinach Pb2+-binding aptamer (DOI: 10.1039/c5cc01526j) FIG. 31B: TEM micrograph showing vaults with Twister-Broccoli (left column) and without (right column).

FIG. 32: Mono-to-polyvalent hubs as structural elements FIG. 32A: Schematic representation of B-Fabp fused to Anti-Moon-Tag, binding to up to 10 Moon-Tags that are present on LifeAct-emFPv4. FIG. 32B: TEM micrograph of Hek293T cells coexpressing LifeAct-emFPv4 and B-Fabp Anti-Moon-Tag showing contrasted actin filaments. FIG. 32C: Clear Native page showing the binding of B-Fabp_Anti-Moon-Tag to LifeAct_emFPv4. The higher MW smear corresponds to varying copies of Bfabp-Anti-Moon-Tag binding the up to 10 motifs on LifeAct_emFPv4. At a lower MW the band corresponding to unbound B-Fabp_Anti-Moon-Tag is visible.

FIG. 33: Stoichiometry control. FIG. 33A: Example densitometric quantification of fractional read-through across different combinations of stop codons and read-through motifs. Read-through resulted in the extension of the FLAG tag with an anti-GFP intrabody (higher molecular weight band). The heterotypic encapsulins were pulled down over the FLAG tag. Note that the example SDS-PAGE shown here does not include stop codon combinations with RT9us. The more complex banding pattern in the case of TAG IntP2A indicates intein splicing patterns. The lower band can be explained by correct splicing, resulting in a 46.9 kDa band (Qtanti-GFP). The higher band can be explained by incorrect splicing, resulting in Qt-anti-GFP fused to IntP2A with a size of 68.5 kDa. FIG. 33B: Percent read-through (rt) determined from densitometric analysis of the respective SDS-PAGE bands (QtFLAG-linker-anti-GFP/QtFLAG*100), (Bars represent mean±SD). FIG. 33C: Control condition in which 100% of the anti-GFP intrabody, ie, 240 copies per encapsulin were expressed, resulting in agglomeration of msfGFP-Cx43 at encapsulin binding. The scale is 10 μm. FIG. 33D: Alternative labeling of Cx43 with C-terminal fusion of Cx43-msGFP (as opposed to N-terminal fusion as in FIG. 3e) and corresponding confocal fluorescence microscopy images. encapsulins were made fluorescent by co-expression of mScarlet-I as cargo proteins. Scale bars correspond to 10 μm.

FIG. 34: Multiplexed patterning using hetero-bifunctional SasG concatemers and encapsulins. FIG. 34A: A rigid hetero-bifunctional cross-linker was constructed from SasG capped off with sfGFP and mCherry. Different linker lengths were obtained by increasing the number of G5 domains connected by E domains (sfGFP-2G-mCherry up to sfGFP-10G-mCherry). FIG. 34B: Clear-Native (CN) PAGE under UV illumination loaded with lysates from HEK293T cells expressing sfGFP-SasG-mCherry hetero-bifunctional linkers (2-10G units) yielded discrete yellow fluorescent bands. FIG. 34C: Co-expression of 1M-Qtanti-GFP and 1M-Tmanti-mCherry with indicated crosslinkers, or no cross-linker (0G) as control, resulted in distinct encapsulin patterns with ^{˜}40 nm ring-shaped centers from 1M-Qt surrounded by a corona of ^{˜}25 nm spherical objects (1M-Tm) in TEM micrographs. The upper panel shows 400×400 nm exemplary regions showing the concentric, programmable encapsulin patterns with overlaid semantic segmentation results from the end-to-end network (8k× magnification, 0.5525 pixels/nm). Scale bars represent 50 nm. The lower panel shows averages around selected Qt centers surrounded by a layer of Tm (n=25). The bounding box represents 165 nm. FIG. 34D: Distances between the centers of 1M-Qtanti-GFP and the centers of surrounding 1M-Tmanti-mCherry for the indicated cross-linker lengths (n=30). FIG. 34E: Average radial profile plots from the center of each 1M-Qtanti-GFP (n=25) outwards via the crosslinkers towards the surrounding ring of 1M-Tmanti-mCherry color-coded for crosslinker length. The vertical lines represent contrast minima generated by the surrounding 1M-Tmanti-mCherry.

FIG. 35: Geometric sensing of Calcium via aggregation. FIG. 35A: Confocal fluorescence microscopy experiment of HEK293T cells co-expressing 1M-QteUnaG-RS20, CaM-HpFtn (see FIG. 19b) and a blue-light inducible optogenetic tool (OptoCracl-mCherry), where Calcium influx is stimulated via OptoCrac (doi:10.7554/eLife.10024). The panels show different time points of two consecutive stimulation cycles where 1M-QteUnaG-RS20 and CaM-HpFtn aggregate upon stimulation and disaggregate when the blue light stimulation is off.

FIG. 36: Protein Designs for CLEM-GECI. FIG. 36A: Schematic depiction of the genetically encoded components of the correlative super-resolution light microscopy (SRLM) EM calcium indicator (CLEM-GECI). The underlying molecular mechanism is calcium-dependent recruitment of metal-binding proteins (TEM contrast) and exchange sites for reversibly binding fluorescent proteins (PAINT contrast) to monodisperse, cytoskeleton-associated nano-scaffolds genetically expressed in cells of interest. LIVE-PAINT (Oi et al, Commun Biol, 2020) is enabled by a docking- and an imager-peptide pair. To accelerate PAINT imaging by expressing relatively high concentrations of imager-peptide-FPs while minimizing the fluorescent background from unbound imager-peptide-FPs, we will use a protein FRET pair based on the previous publication (Auer et al, Nano Letters, 2017). Specifically, the FP for recruitment to the scaffold via the CaM-binding peptide will be a FRET acceptor (such as TagRFP or stagRFP35) to elicit FRET only upon reversible binding of a FRET donor (e.g., TagGFP2) to the docking peptide. GCaMP as the FRET donor such that fluorescence background signals in the absence of calcium are further suppressed can also be used. In this mode, the donor fluorescence can be simultaneously detected to provide diffraction-limited calcium-dependent fluorescence at the full frame rate in pixels that will not be clustered via the localization events. b, CLEM-GECI variant that integrates the EM contrast under optical control by proteolytic unmasking of FABPs in a calcium-dependent fashion, such that it can bind to the scaffold. Specifically, the established scFLARE (Sanchez et al, PNAS, 2020) system containing the calcium-activated protease TEV (CaTEV) is adapted such that it cleaves a fused consensus protease site (TEVsite) but only when the light-dependent hLOV domain is extended under illumination with blue light. Since the subsequent SpyCatcher/SpyTag binding is irreversible, each nano-scaffold thus accumulates EM contrast as a function of calcium activity. The design adds optical control over an integration interval during which calcium-dependent accumulation of the EM contrast can occur. This contrast integration is accomplished by adapting the established scFLARE2 system, based on the proteolytic release of a transcription factor via a calcium-activated and selective protease (CaTEV) if the protease cleavage site (TEVsite) is exposed by illumination with 470 nm. Since SpyTag/SpyCatcher00338 form irreversible isopeptide bonds, the EM contrast accumulates on the scaffold within the photo-controlled time interval. PAINT contrast can be simultaneously localized in a separate channel by a red FP, such as stagRFP fused to the imager-peptide, which can also be a FRET acceptor for a green donor (e.g., TagGFP2).

FIG. 37: Correlative bioluminescence-EM imaging. FIG. 37A: Schematic representation of the genetically encoded components for a dual-modality (bioluminescence and EM) calcium indicator. The system is based on the strong affinity of calmodulin (CaM) for the synthetic peptide M13. In the presence of calcium ions, the conformational change of CaM enables high-affinity binding to the M13 peptide. Also fused to M13 is the large bit (LgBit) of a split nano-luciferase, which, when complemented with the small bit (SmBit) of said split nano-luciferase, for which it has very weak affinity, restores active luciferase. While the fused LgBit and M13 peptide are fused to QtEnc or another EM-appropriate scaffold, the fused SmBit and CaM are also fused to a contrasting entity for EM. When calcium ions are present, CaM binds the M13 peptide, reconstituting both luciferase halves for bioluminescence. At the same time, the CGU (e.g. FABP) is recruited to the structural element (e.g., encapsulins, ferritin, I3_01) for EM imaging. FIG. 37B: The M13 can alternatively be fused on the protein to be recruited to the structural element and CaM fused to the structural element. LgBit and SmBit, parts of the split Nanoluciferase, are optional if only EM contrast is desired. FIG. 37C: Alternative system in which all components are assembled (preferentially via an isopeptide bond via SpyCatcher/SpyTag) such that the calcium-dependent calmodulin-M13 interaction occurs within the same protein complex. Consequently, all binding partners are present at high local concentrations and at fixed maximal distances, which increases the response time of the sensor. Specifically, CaM and M13 are separated by two SasG filaments containing 10 G-domain repeats, wherein the two rigid SasG filaments are connected by a flexible linker as a joint, which can be held in the folded position in the presence of calcium. Note that the sensor mechanism depicted here can be generalized to any protein-peptide or protein-protein interactions that are modified via a cellular process of interest, e.g., based on phospho-peptide binding domains (FHA2) to sense kinase activity.

FIG. 38: Contrast from encapsulins as structural elements and metallothioneins as contrast-generating units in SEM and FIB-SEM of Drosophila neurons. SEM and corresponding TEM images of the identical sample of a Drosophila line with panneuronal expression of either FIG. 38A: 1M-QtFLAG-NLS or FIG. 38B: 1M-MxFLAG-NLS following a standard fixation and staining protocol. Ultrathin sections were imaged on either TEM grids or silica wafers for subsequent analysis by TEM and SEM (reverse contrast) so that similar sections through the same cell could be analyzed by both techniques. SEM images were acquired using a Zeiss GeminiSEM with sense BSD, tandem decel at 1.5 kV. Corresponding TEM images were acquired with a Zeiss Libra120 at 120 kV, 13 μA, 100 μrad. Insets show average values (n=30) of respective particles from manual segmentation. White arrowheads indicate the presence of encapsulin-metallothionein particles within the nucleus. FIG. 38C, FIG. 38D: Isotropic FIB-SEM image volumes (4 nm voxel size) of Drosophila brains expressing c, 1M-QtFLAG-NLS (cyan) and d, 1M-MxFLAG-NLS (red) targeted to the nucleus. Encapsulin-metallothioneins and nuclear membranes were manually segmented and rendered within the FIB-SEM volume bounded by the ortho-disks. The magnifications (right) show ortho-disks through three Encapsulin-metallothioneins. Volume acquisition was performed with an SEM beam voltage of 1.3 kV, a working distance of 5 mm, and a nominal voxel size of 4 nm using an InLens detector. The FIB Ga beam was accelerated with a voltage of 30 kV and a current of 700 μA. The scale bars in the overview and zoom-ins represent 500 nm and 50 nm, respectively.

FIG. 39: High-res TEM micrographs of encapsulation-metallothioneins expressed in HEK293T. All six classes of concentric barcodes, resulting from the distinct combination of encapsulin shells and metallothioneins, as well as wild-type controls (QtFLAG, MxFLAG), were acquired at a target image pixel size of 0.23 nm and an illumination angle of 0.800 mrad. The scale bars are 50 nm.

FIG. 40: Performance metrics of a sequential segmentation and classification pipeline. FIG. 40A: Flowchart describing the two-step semantic instance segmentation pipeline consisting of segmentation of encapsulin-metallothionein particles and background subtraction, followed by classification of the encapsulation-metallothionein instances. FIG. 40B Pixel-level Precision-recall (PR) curves for all 6 classes. FIG. 40C table showing the performance metrics. FIG. 40D:Confusion matrix of a human classification experiment where 3 blinded human evaluators classified 12 patches of TEM micrographs from HEK293T cells (400×400 pixels, 2 examples per class) to one of the six different encapsulin-metallothionein particle classes (average accuracy: 97.22%). FIG. 40E Confusion matrix for the classification results of the EfficientNetV2 on the segmented encapsulin-metallothionein particles.

FIG. 41: Napari GUIs for interactive segmentation and classification of encapsulin-metallothionein particles. FIG. 41A: Screenshot of the Napari GUI for the end-to-end semantic segmentation network, which allows for interactive label curation and interfacing with the U-net model (github.com/HelmholtzAl-Consultants-Munich/EMcapsulins_segmentation). (1) Napari image viewer, (2) import widget for loading all images as segmentation results in a selected folder, (3) napari tools for manual annotation, (4) color-code for the encapsulin-metallothionein particle classes, (5) export widget for the curated masks. FIG. 41B: Screenshot of the napari GUI for the sequential segmentation-classification pipeline (github.com/StructuralNeurobiologyLab/emcaps): (1) napari image viewer, (2) segmentation widget (3) napari tools for manual annotation (4) classification widget (5), export widget for exporting a semantic segmentation overlay. FIG. 41C:, FIG. 41D: Examples of possible automated segmentation results flagged for not meeting the imposed criteria (linear pixel size range and circularity). Manual curation of incomplete or artificially fused segmentation instances (middle panel) allows for an improved class assignment (right).

FIG. 42: Live microscopy experiments with targeted fluorescent encapsulin-metallothionein particles. FIG. 42A: Individual frames from live-cell microscopy (Airyscan LSM880) of mammalian oocytes co-expressing mCherry-RAB11A, Myo5b-mCherry, or mCherry-PLK1 without or with eUnaG-1M-Qtanti-mCherry. Insets are magnifications of regions outlined by dashed boxes. The scale bar represents 5 μm. FIG. 42B: Confocal images of live mouse oocytes microinjected with different concentrations of eUnaG-1M-Qtanti-mCherry mRNA in the absence and presence of mCherry-RAB11A. FIG. 42C, FIG. 42D: Quantification of volume (c) and speed (d) of RAB11A-positive recycling endosomes in mCherry-RAB11A-expressing mouse oocytes microinjected with different concentrations of eUnaG-1M-Qtanti-mCherry mRNA. The number of particles or tracks analyzed is specified in italics.

FIG. 43: Encapsulin-metallothionein particles targeted to subcellular compartments in mammalian cells and Drosophila cells. FIG. 43A: UV-fluorescence and Coomassie-stained BN-PAGE of lysates from HEK293T cells expressing QtEnc N-terminally fused to the small fluorescent protein miniSOG2, and C-terminally fused to a FLAG-epitope followed by an NLS (cMyc) or an NES (HIV). The bands apparent above 1.3 MDa correspond to the native assembled nanocompartments. The bands also show fluorescence, indicating that the N-terminally appended miniSOG2 was functional and did not comprise the assembly. FIG. 43B: Corresponding live-cell fluorescence microscopy showing miniSOG2-QtFLAG-NLS predominantly localized in the nucleus and miniSOG2-QFLAG-NES predominantly localized in the cytosol. FIG. 43C: The functionality of NLS constructs was confirmed in the S2R+ Drosophila cell line by co-expressing either UAS-1M-QtFLAG-NLS or UAS-1M-QtFLAG with Actin5c-Gal4. d) Widefield fluorescence microscopy showed that encapsulin-metallothionein particles with the NLS downstream of the FLAG-epitope (anti-FLAG, green signal) readily co-localized with the nucleus (DAPI, blue signal), whereas the variant without NLS was distributed throughout the cytosol.

FIG. 44: Higher-magnification TEM micrographs of encapsulin-metallothionein particles expressed in Drosophila neurons. Neurons in the optic lobe expressing 1M-QtFLAG-NLS (top) or 1M-MxFLAG-NLS (down). Scale bars are 50 nm (20 nm for inset).

FIG. 45: Detection of encapsulin-metallothionein particles in the processes of Drosophila optic lobe neurons. FIG. 45A, FIG. 45B: IHC of the optic lobe (OL) of Drosophila brains with pan-neuronal expression of either 1M-QtFLAG-NES (a) or 1M-MxFLAG-NLS (b). Anti-FLAG (Alexa 568, cyan for 1M-QtFLAG-NES and red for 1M-MxFLAG-NLS) and DAPI (blue) to show the distribution of the neuronal nuclei. Note that there is a substantial anti-FLAG signal in the neuropil only if the nuclear export signal (NES) but not the nuclear localization signal (NLS) is expressed. The scale bars represent 50 μm. FIG. 45C TEM micrograph of the optic lobe of Drosophila pan-neuronally co-expressing 1M-QtFLAG-NES and 1M-MxFLAG-NLS with the multi-class semantic segmentation results overlaid according to the color scheme in main FIG. 1. 1M-MxFLAG-NLS encapsulins are predominantly found in the nucleus. 1M-QtFLAG-NES encapsulins can be detected in the cytosol as well as in projections FIG. 45D, FIG. 45E: Scale bar in sub-panel c represents 200 nm. The scale bars in d and e represent 100 nm.

### DEFINITIONS

Unless otherwise defined, all technical and scientific terms used herein have the same meaning as commonly understood by one of ordinary skill in the art to which the present invention belongs.

The singular forms “a”, “an”, and “the” include references to the plural unless explicitly stated otherwise in the context.

The term “comprising” means “including” as well as “consisting”, e.g., a composition “comprising” X may consist exclusively of X or may include something additional, e.g., X+Y.

“Genetically controlled” means that at least one element is a genetically encoded biomolecule, such as DNA, RNA, or protein, which may interact with non-genetically encoded molecules such as, for example, certain metal-interactors or metal-binders or substrates for enzymatic activity. Genetically controlled is therefore not limited to genetic material that has been artificially introduced into the cell but comprises endogenous genetic material expressed by the cell as well. Artificially introduced (generic) material is herein described as “exogenous” material.

“Nanoscopy” refers to imaging-based methods that resolve structures with nanometer spatial resolution, such as electron microscopy (EM), super-resolution-light microscopy (SRLM), or absorbance-based methods such as optoacoustics. Preferred are high-throughput nanoscopy methods that allow for volume nanoscopy of tissue.

“Metal interactor” is a molecule to which metal ions can bind to or with which they can react.

A “metal-interacting peptide”, for instance, includes molecules, which can reversibly bind to metals or react with a metal. Osmium, for instance, reacts with osmiophilic residues. Metal-binding (metal chelation) may still occur. As an example, MT3 is osmiophilic but may still be able to bind to lead after osmium tetroxide treatment.

A “metal-binding peptide”, for instance, a lead binder, or a lanthanide binder is a molecule that (reversibly) binds to (instead of reacts with) metals.

“Exogenous” means not genetically controlled from within the biological system, e.g., a cell, but added to the biological system, e.g., the cell, from the outside as a compound or material.

“Genetically controlled nanoscopy contrast-generating unit” refers to a genetically controlled molecule which alters the interaction of electrons with matter, e.g., by the local enrichment of electron-dense elements, such as metals, for example, heavy-metals, such that contrast in electron microscopy is generated, and/or genetically encoded chromophores or fluorophores such that photons are absorbed and optionally emitted to generate a localized signal at the nanoscale, or a genetically controlled molecule that determines the local enrichment of a cofactor for a chromophore or fluorophore, or an exogenous chromophore or fluorophore, for the use in super-resolution light-microscopy methods (SRLM) or absorbance based super-resolution methods, e.g., based on optoacoustics.

“Fluorophore” refers to any type of fluorescent molecule.

“Chromophore” refers to, for example, a protein binding a chromophore as a cofactor, such as biliverdin or phycocyanobilin, or an enzymatically generated polymer such as melanin. Chromophores such as biliverdin are also comprised as well as light-absorbing, light-refracting, light-scattering, or light-reflecting parts within a molecule, which are responsible for its color.

“Genetically controlled structural element” refers to a structural element with geometrically precise nanometer-sized features, that spatially organizes contrast-generating units to sufficiently large nanoscale size, such that their spatial distribution is discernable as a distinct geometric shape by nanoscopy, e.g., EM or super-resolution-light-microscopy (SRLM).

“Spatially organized” means that the elements are arranged in a specific manner to create specific shapes or patterns.

A “geometric shape” is generated when genetically controlled nanoscopy contrast-generating units are spatially organized by a structural element.

A “barcode” or “nanoscopy-readable barcode” refers to a distinct spatial distribution of contrast, in particular via a series of contrast-rich and contrast-poor regions with defined spatial intervals, such as a series of high-contrast parallel bars with defined width and defined low-contrast intervals.

A “concentric barcode” refers to a series of contrast-rich or contrast-poor concentric spheres or rings. In nanoscopy, concentric barcodes can be read as the cross-section or intensity projection of certain spherical structural elements. A concentric barcode can be rotationally invariant and can intrinsically also encode directionality since it has a defined center and a surround such an inside-out or outside-in directionality can be defined.

“Genetically controlled scaffold” refers to a structure that can spatially organize structural elements to generate distinct geometric patterns discernable by nanoscopy.

A “geometric pattern” is a nanoscale pattern distinguishable in nanoscopy detection methods. Such geometric patterns are generated when genetically controlled structural elements are spatially organized by a scaffold.

An “attachment point” refers to a motif on a contrast-generating unit, a structural element, or a scaffold that enables specific binding between those elements or to a cellular structure or biomolecule of interest.

“Geometric multiplexing” refers to the parallel encoding of distinct pieces of information via multiple and distinct geometric shapes or patterns.

“Molecular mapping” refers to the nanoscopic visualization of the biodistribution of a molecule or molecules of interest with a genetically controlled structural element or scaffold.

“Geometric sensing” refers to the detection of an analyte or environmental parameter of interest (e.g., temperature, pH) via a change of the geometric shape or geometric pattern. Geometric sensing can also refer to the detection of multiple analytes or environmental parameters of the physiological or pathophysiological state of a cell, such as the response of a cell, ion flux in a cell, a response of the cell, a therapeutic response of the cell, an activation of the cell, or external stimulus to the cell, or combinations thereof.

“Geometric actuation” refers to the manipulation of cellular structures or processes by geometrically distinct patterns of biomolecules organized by structural elements and/or scaffold, which are also readable/visible by nanoscopy. The actuation may occur biomechanically, biochemically, and/or via deposition of external energy, e.g., via electromagnetic radiation (e.g., photoabsorption, or radiofrequency absorption), or mechanical energy (e.g., ultrasound energy), and/or magnetic gradients.

“Nanobiomaterial” refers to a nanoscale biomaterial, which is produced in an isolatable form from a biological cell or a cell-free system and comprises nanoscale genetically controlled geometric features, based on structural elements, scaffolds, attachment points, and comprising biominerals, biopolymers, or enzymatic products for use in downstream applications. A nanobiomaterial can be used to, for example, treat, strengthen or substitute a part, such as a tissue, organ, or function of a living system. Alternatively, a part of a non-living system can be strengthened or substituted, such as, for example, a prosthesis.

The term “vector” as used in this disclosure comprises the nucleic acid encoding the genetically controlled nanoscale contrast-enhancing unit, the genetically controlled structural element, and/or the genetically controlled scaffold under control of a target cell-specific promoter. Such vector is suitable to transfer the nucleic acid encoding the genetically controlled nanoscale contrast-enhancing unit, the genetically controlled structural element and/or the genetically controlled scaffold to a target cell or target tissue. Such vector may be a viral vector, a plasmid, an agrobacterium, an exosome or a virus or virus-like particle, or any other vector known in the art and suitable for the transfection of a specific target cell.

As used herein, a “eukaryotic cell”, organ or organism may be yeast, fungus, protozoa, plant, higher plant, and insect, or amphibian cells, organs or organisms, such as the brain of Drosophila melanogaster, or “mammalian cells” such as HEK293 cultured cells (in vitro), graft cells and primary cell culture (in vitro and ex vivo), and in vivo cells, and also mammalian cells including human, which are commonly used in the art, without limitation.

“SEQ ID NO.” refers to the number of the respective sequence in the sequence listing. Comprised by this invention are also sequences having 80%, 90% or 95% sequence identity to the respective sequences given in the sequence listing.

### DETAILED DESCRIPTION

### Genetically Controlled Nanoscopy Contrast-Generating Unit (CGU)

In one aspect, the invention relates to a genetically controlled nanoscopy contrast-generating unit comprising a metal interactor. The metal interactor is compatible with nanoscopy fixation protocols, nanoscopy post-fixation protocols, and nanoscopy metal staining protocols. Moreover, the metal interactor is a molecule to which metal ions can bind to or react with, wherein the metal interactor is not a ferroxidase and not an enzymatic product from an exogenous substrate, such as 3,3′-diaminobenzidine that is turned over by an enzyme such as HRP, APEX, or APEX2, or by a light-triggered reaction mediated by miniSOG variants to generate a polymer as a product with which heavy metals can interact with.

The nanoscopopy fixation protocols, nanoscopy post-fixation protocols, and nanoscopy metal staining protocols are preferably electron microscopy fixation protocols, electron microscopy post-fixation protocols and electron microscopy metal staining protocols, respectively. Thus, the genetically controlled nanoscopy contrast-generating unit is compatible with, for example, aldehydes such as formaldehyde, glutaraldehyde and mixtures thereof, which are commonly used in electron microscopy fixation protocols. The genetically controlled nanoscopy contrast-generating unit is also compatible with, for example, osmium tetroxide and tannic acid which are commonly used in electron microscopy post-fixation protocols. Furthermore, in preferred embodiments, the genetically controlled nanoscopy contrast-generating unit is also compatible with heavy metals, for example, uranium and lead which are commonly used in electron microscopy heavy metal staining protocols, as well as with lanthanides, which may be used as an alternative stain. In yet another embodiment, the genetically controlled nanoscopy contrast-generating unit mainly provides contrast by interaction with osmium tetroxide and partly by binding of a heavy-metal such as lead, after post-fixation.

Without being bound by theory, the inventors assume that osmium interferes with numerous metal-coordination sites. Thus, the genetically controlled nanoscopy contrast-generating units comprise metal interactors. Said metal-interactors interact with, for example, osmium tetroxide. It is assumed that the metal interactor provides contrast by osmiophilicity and metal binding, inter alia. For example, the metal interactor comprises one or more osmiophilic protein domains or peptides, thereby providing the desired contrast. Alternatively, a lipid-binding protein provides the desired contrast by interacting with, for example, osmium tetroxide. One example of a lipid-binding protein according to the invention is a fatty acid-binding protein. Thus, in one embodiment the metal interactor may react with osmium tetroxide such that osmium accumulates locally, whereby said locally accumulated osmium provides further interaction or binding sites for other metals. In another embodiment, the metal-interactor does not react with osmium tetroxide such that the metal-binding site provides further interaction or binding sites with heavy metals.

The metal-interactor comprised in the genetically controlled nanoscopy contrast-generating unit may comprise at least one metal-interacting peptide or a metal-interacting protein. In an embodiment, the metal-interacting peptide is a metallothionein, preferably MT3 or a series of different metallothionein species. In a preferred embodiment, the metal interactor is a metallothionein, for example a murine metallothionein. In a preferred embodiment, the metallothionein is MT3, even more preferred a eukaryotic MT3 domain, or preferably a mammalian MT3 domain. The MT3 domain may comprise one, two (tandem MT3), or three MT3 molecules. In a specific embodiment, the MT3 domain is a mouse MT3 domain, wherein the mouse MT3 domain is a single or tandem domain, in other words, comprises one (SEQ ID NO: 1) or two (SEQ ID NO: 4) mouse MT3s. In another specific embodiment, a series of three MT molecules are chosen from different species to avoid redundant sequences on the DNA level which interfere with genetic stability (e.g., SEQ ID NO: 5). An exemplary MT3 is MmMT3 having SEQ ID NO: 1. Exemplary sequence IDs for a series of MT3 molecules are SEQ ID NOs: 1-5.

In another embodiment, the metal-interacting protein may comprise a high abundance of osmiophilic amino acids. The metal interactor comprised in the genetically controlled nanoscopy contrast-generating unit may thus comprise at least one osmiophilic amino acid or one or more osmiophilic proteins, rich in cysteines, methionines, and/or tryptophanes. In a preferred embodiment, the osmiophilic amino acid/protein comprises one or more cysteine residue. An exemplary osmiophilic protein is MT3 (SEQ ID NO: 1), HRPII (e.g., SEQ ID NO: 17 fused to the encapsulation signal), or MMKPDM (SEQ ID 6). Alternatively, the metal-interacting peptide/protein may be selected from PDB entries 2m17, 4n18, 2jyt, 51ah, 3hcu.

The metal-interactor comprised in the genetically controlled nanoscopy contrast-generating unit may comprise at least one metal-binding peptide or metal-binding protein. In an embodiment, the metal-binding protein is a lead binder, such as MT3 having SEQ ID NO: 1. In an embodiment, the metal interactor is a lanthanide binder, such as lanmodulin (SEQ ID NO: 18). In another embodiment, the metal-binding protein is a uranyl binder, such as MUP2, or MUP5, having SEQ ID NOs: 45 and 46. In a preferred embodiment, the metal interactor is a super-uranyl-binding protein (SUP), a de novo designed protein having the sequence set forth in SEQ ID NO: 225. In another embodiment, the metal-binding protein is a vanadium binder, such as vanabin 2 (SEQ ID NO: 14). In another embodiment, the metal-binding protein is a copper and/or silver binder. In one embodiment, the metal interactor is one or more copper-binding peptide, such as CusF (SEQ ID NO: 15). In another embodiment, the metal interactor is one or more silver binding peptide, such as AG4 (SEQ ID NO: 199). In even another embodiment, the metal interactor is one or more copper and silver binding peptide, such as AG4 (SEQ ID NO:199).

Therefore, the difference between a metal-reacting and a metal-binding peptide/protein is that the metal-binding does not irreversibly modify the peptide/protein, i.e, it can also unbind/dissociate again.

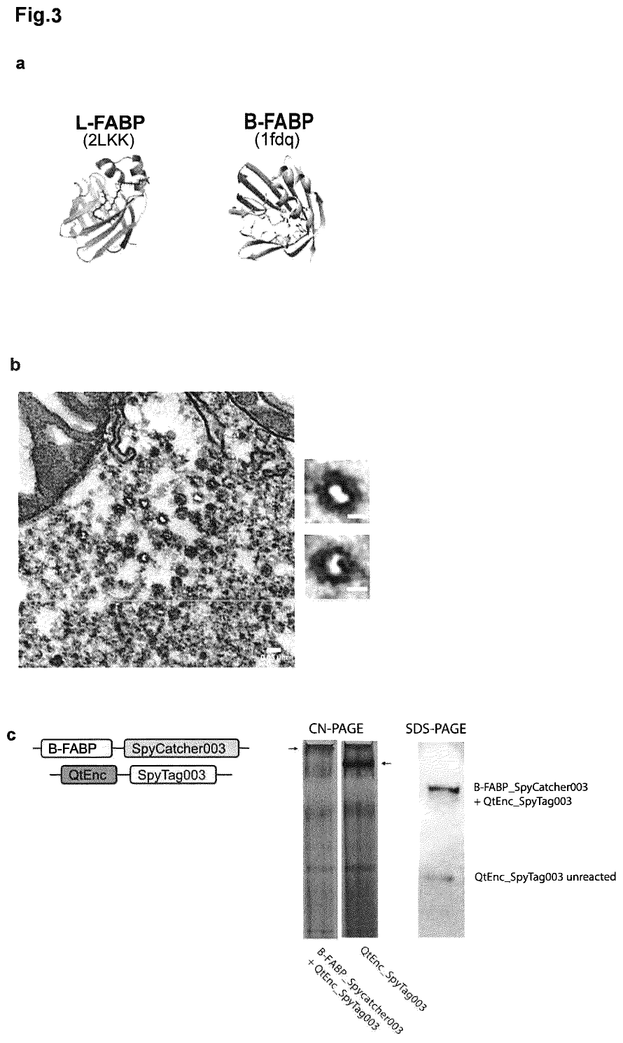

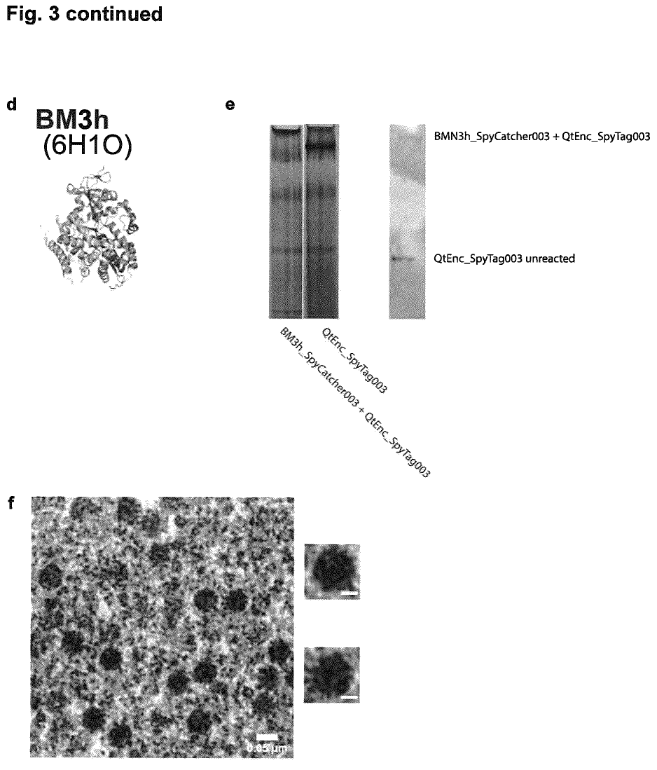

The metal-interactor comprised in the genetically controlled nanoscopy contrast-generating unit may comprise at least one lipid-binding peptide or a lipid-binding protein. The bound lipid interacts with, for example, a heavy-metal stain. In an embodiment, such lipid-binding protein is a member of the family of fatty acid-binding proteins (FABPs). Given that lipids/fatty acids are a primary target for heavy metals, in particular, the EM post-fixative and contrast agent osmium tetroxide, the inventors identified fatty acid-binding proteins (FABPs) as a class of proteins that can generate genetically controlled and spatially confined EM contrast-generating units. FABPs are a family of proteins (liver (L-), intestinal (I-), heart (H-), adipocyte (A-), epidermal (E-), ileal (II-), brain (B-), myelin (M-) and testis (T-) that share very similar three-dimensional structures (PRINTS database pattern FATTYACIDBP; accession number PR00178), containing an antiparallel p-barrel structure with an internal binding site that holds the fatty acid in an interior cavity. Exemplary sequences are given in SEQ ID NOs: 30-39, such as for brain (B-)-FABP (SEQ ID NO: 36). Variants that preferentially bind polyunsaturated fatty acids, such as PDB-lfdq and PDB-2LKK (FIG. 3a), are preferred embodiments. In a preferred embodiment, the fatty acid-binding protein is PDB: 1fdq (SEQ ID NO: 36), a protein that binds polyunsaturated fatty acids, such as arachidonic acid, known to interact with osmium tetroxide. In yet another embodiment, such lipid-binding protein is the lipid-binding domain of a cytochrome P450, such as BM3. Preferably the lipid- and heme-binding domain of the cytochrome P450 BM3 (BM3h; SEQ ID NO:29) or variants thereof, such as Bm3h-B7. Alternatively, the lipid-binding peptide/protein may be selected from the list comprising farnesylation, prenylation, and myristoylation motifs with exemplary SEQ ID NOs: 40-43. In a preferred embodiment, the lipid-binding protein is B-FABP, B-FABP-ko, Bm3h, or Bm3h-B7.

The metal interactor of the genetically-controlled nanoscopy contrast-generating unit may comprise at least one RNA or a DNA molecule. Nucleic acids interact with heavy metals that are used in EM stains. The metal interactor of the genetically-controlled nanoscopy contrast-generating unit may comprise an RNA-binding protein bound to such RNA via an RNA aptamer. Preferably, the metal interactor is an RNA aptamer-binding protein bound to RNA which is modified with aptamer motifs. RNA aptamer binder: RNA aptamer pair interaction is preferably taken from viruses. The bound RNA interacts with the heavy-metal stain. In one embodiment, the RNA-binding protein is at least one RNA-binding protein, wherein the RNA-binding protein preferably binds RNA, which comprises an RNA aptamer. A pair of an RNA aptamer: RNA aptamer-binding protein is thus preferred. In a preferred embodiment, a pair of an RNA aptamer: RNA aptamer-binding protein is, for example, PP7: PCP (SEQ ID NO: 56 and 53, 54), MS2: MCP (SEQ ID NO: 152 and 100), H23:L7ae (SEQ ID NO:206 and 63), K-turn motif:L7kk (SEQ ID NO: 206 and 63). In another embodiment, the RNA-binding protein comprises a programmable RNA-binding protein, such as dCas13 (e.g., the dCas13 of Prevotella sp, SEQ ID NO: 200), or PUF proteins/pumilios (e.g., human pumilio homology domain, SEQ ID NO: 201), or polyA-binding protein (Kuehn et al, Biochimica et Biophysica Acta (BBA, 2004). In a preferred embodiment, said bound RNA comprises a secondary structure, preferably a geometrically distinct secondary structure. In another embodiment, the RNA aptamer is selected from RNA aptamers that bind fluorophores, such as Spinach, Dir2s, and RhoBAST (SEQ ID NOs: 55, 57, 59). In another preferred embodiment, the RNA contains an engineered metal-binding with an exemplary sequence given in SEQ ID NO: 58. In another preferred embodiment, the RNA aptamer-binder: RNA aptamer pair circularized is twister-broccoli RNA aptamer.

The metal interactor comprised in the genetically controlled nanoscopy contrast-generating unit may comprise at least one polymerizing or synthesizing enzyme, by which a product is formed which in turn interacts with, for example, a heavy-metal stain. In one embodiment, the polymerizing or synthesizing enzyme comprises a kinase, a tyrosinase, a synthase, an RNA-dependent RNA polymerase transcriptase, an RNA-dependent DNA polymerase such as reverse transcriptase, a DNA polymerase, or a lipid synthetase. In a preferred embodiment, the polymerizing or synthesizing enzyme is a kinase or a tyrosinase. In one embodiment, the polymerizing enzyme may be a polymerizing kinase that can turn over, for example, GTP or ATP under the formation of highly negative-charged polyphosphate chains. In a preferred embodiment, the kinase is tetrameric polyphosphate kinase 2 (PPK2b), preferably having SEQ ID NO: 48. In a particularly preferred embodiment, the kinase is tetrameric polyphosphate kinase 2 (PPK2) of F. tularensis (PDB: 5LL0). In one embodiment, the synthesizing enzyme may be a tyrosinase. In a preferred embodiment, the tyrosinase is a bacterial tyrosinase that generates the strongly absorbing polymer melanin from the endogenously available amino acid tyrosine. Therefore, the tyrosinase may form melanin which may interact with, for example, a heavy-metal stain. In an even more preferred embodiment, the tyrosinase is a Bacillus megaterium tyrosinase (BmTyr), preferably having SEQ ID NO: 51. In yet another embodiment, the synthesizing enzyme is a phytochelatin synthase. In a preferred embodiment, the phytochelatin synthase is phytochelatin synthase 1 (AtPCS1), preferably having SEQ ID NO: 49.

The metal interactor comprised in the genetically controlled nanoscopy contrast-generating unit may comprise at least one biomineralizing enzyme. The resulting biomineral itself is electron-dense, or interacts with other electron-dense materials. In one embodiment, the biomineralizing enzyme comprises iron-oxide biominerals. In another embodiment, the biomineralizing enzyme comprises ferroxidase. In yet another embodiment, the biomineralizing enzyme is an iron-oxide biomineral such as a ferroxidase. In another embodiment, the biomineralizing enzyme is a ferroxidase from the Iron-Mineralizing Encapsulin-Associated Firmicute (IMEF, SEQ ID NO: 52) or Ferritin (PfFt, SEQ ID NO: 213, or H. pylori ferritin). In another embodiment, the biomineralizing enzyme is complemented by the iron importer Zip14 (SEQ ID NO: 219) and the manganese importer DMT1 (SEQ ID NO: 218), such as to achieve doping of the produced iron oxide biomineral with manganese. In another embodiment, the enzyme is alkaline phosphatase, in a preferred embodiment not membrane-bound (SEQ ID NO: 221), which can lead to calcium-phosphate formation. In another embodiment, the enzyme is cystathionine-y-lyase with an exemplary sequence (SEQ ID NO: 220).

The metal interactor comprised in the genetically controlled nanoscopy contrast-generating unit may comprise at least one bioconjugation tag. In one alternative embodiment, the genetically controlled nanoscopy contrast-generating unit is at least one bioconjugation tag. Without being bound by theory, the bioconjugation tag provides contrast by conjugation to metal complexes and/or exogenous fluorophores or chromophores. In one embodiment, the bioconjugation tag is, for example, SpyTag/SnoopTag-Catcher, SNAP, Clip, Halo, coiled coils, or inteins. In a preferred embodiment, an exogenous compound binding and/or reacting with the bionconjugation tag is, for example, a chromophore and/or fluorophore or a tag-compatible metal-chelator such as macrocyclic compounds, i.e., a DOTA, DO3A, DOTAM, DTPA, NOTA, NODAGA derivative or a ring or linear polymers such as dendronized polymers. In another preferred embodiment, the bioconjugation tag is a pair of coiled coils. In yet another preferred embodiment, the bionconjugation tag is SNAP. In another embodiment, the bioconjugation tags are coiled coils, and said coiled coils are utilized to generate super-resolution contrast by PAINT (points accumulation for imaging in nanoscale topography). In yet another embodiment, a low concentration of bioconjugation tags is used. A low concentration of bioconjugation tags depends on various parameters inter alia, such as the type of bioconjugation tag utilized and the type of genetically controlled contrast-enhancing unit, inter alia.

The metal interactor comprised in the genetically controlled nanoscopy contrast-generating unit may comprise a combination of at least two of said metal-interacting peptide/protein, metal-binding peptide/protein, lipid-binding protein, RNA-binding protein, RNA or DNA molecule, polymerizing or synthesizing enzyme, or bioconjugation tag. This means that all of the above-listed contrast-providing genetically controlled nanoscopy contrast-generating units can be combined.

Without being bound by theory, the metal-interactor provides contrast by osmiophilicity and metal-binding, inter alia. Furthermore, all of the contrast-generating units of the invention generate not only contrast in electron microscopy but may also be detected by fluorescence, bioluminescence, absorbance, magnetic measurements such as NMR, MRI, NanoSIMS, nitrogen-vacancy magnetometry, mass spectrometry imaging, and/or ultrasound. In one embodiment, the genetically controlled nanoscopy contrast-generating unit is detectable by both electron microscopy and fluorescence microscopy or optoacoustic microscopy techniques. In yet another embodiment, the genetically controlled nanoscopy contrast-generating unit is compatible with both electron microscopy and fluorescence microscopy techniques.

Therefore, in one preferred embodiment, the genetically controlled nanoscopy contrast-generating unit comprises a metal interactor, and further comprises a fluorophore or a chromophore. The fluorophore is, for example, a fluorescent protein, a co-factor in a fluorescent protein, or an exogenous fluorophore such as an Alexa dye or an indocyanine derivative. The fluorophore may also be a fluorophore-binding RNA aptamer. In one embodiment, the fluorescent protein is for example UnaG, preferable eUnaG (SEQ ID NO: 202), mGreenLantern (e.g, SEQ ID NO: 203), emFP (e.g, SEQ ID NO: 92-99), CaMPARI2 (e.g, SEQ ID NO: 204), mCherry (e.g, SEQ ID NO:185) or mRFP (e.g, SEQ ID NO: 205). Any other fluorescent protein known in the art and suitable for this purpose is encompassed in this embodiment. In another embodiment, the fluorescent protein is eUnaG, mScarlet, miniSog, or eUnaG and mCherry. In even another embodiment, the fluorophore-binding RNA aptamers are, for example, spinach (SEQ ID NO: 55) and/or DIR2s (SEQ ID NO: 57).

In one preferred embodiment, the genetically controlled nanoscopy contrast-generating unit comprises a metal interactor, wherein the metal interactor comprises at least one metallothionein, at least one fatty acid-binding protein, at least one lipid-binding domain or at least one RNA molecule and further comprises a fluorophore, wherein the fluorophore is a fluorescent protein.

In one embodiment, the metal interactor is an MT3 domain and the fluorophore is GreenLantern, CaMPARI2, mEos4b (SEQ ID NO:214), or mEosEM (SEQ ID NO:215); in other words, the genetically controlled nanoscopy contrast-generating unit is a GreenLantern-MT3 or a CaMPARI2-MT3, mEos4b-MT3, mEosEM-MT3 fusion protein. In another embodiment, the metal is an MT3 domain and the fluorophore is UnaG, preferably enhanced UnaG (eUnaG) (SEQ ID NO: 202); in other words, the genetically controlled nanoscopy contrast-generating unit is a eUnaG-MT3 fusion protein. In another embodiment, the MT3 domain are two MT3 domains or in other words, a tandem domain and eUnaG is N-terminally fused, resulting in a eUnaG-2xMT3 fusion protein.

In another embodiment, the metal interactor is an RNA-binding protein bound to RNA via aptamers. The RNA aptamers may be spinach, which may further bind to exogenous fluorophores. Said RNA aptamers thus allow dual detection by both electron microscopy and fluorescence microscopy.

In another embodiment, the genetically controlled nanoscopy contrast-generating unit comprises a metal interactor and a chromophore. The chromophore may be a chromoprotein, a co-factor in a chromoprotein, or an exogenous chromophore such as a dark quencher. The chromoprotein is, for example, any fluorescent protein, a protein binding a chromophore as a co-factor, such as biliverdin (SEQ ID NO: 216) or phycocyanobilin (SEQ ID NO: 217), or an enzymatically generated polymer such as melanin, or light-absorbing, light-refracting, light-scattering, or light-reflecting parts within a molecule which are responsible for its color. For instance, the genetically controlled nanoscopy contrast-generating unit comprises a eUnaG-MT3 fusion protein, wherein eUnaG binds the endogenous ligand bilirubin.

Alternatively, the genetically controlled nanoscopy contrast-generating unit comprises a metal interactor and further comprises a bioluminescent protein, for example firefly luciferase, nanoluciferase, renilla luciferase, or AkaLuc.

The genetically controlled nanoscopy contrast-generating unit may comprise at least one attachment point. Said at least one attachment point is selected from the group consisting of nanobodies, frankenbodies, coiled-coil domains, isopeptide-forming partners such as SpyTag or SpyCatcher domains, SnoopTag/SnoopCatcher, DogTag/DogCatcher, bioconjugation tags such as SNAP/Clip-Tag domains, and/or Halo-Tag domains, split-inteins, calmodulin-binding peptides, phosphorylatable peptides, conformation-changing peptides such as troponins, as well as combinations thereof. In one embodiment, the genetically controlled contrast-generating unit is H. pylori ferritin comprising at least one attachment point, wherein said at least one attachment point is a calmodulin binding peptide (SEQ ID NO. 134). In one embodiment, the genetically controlled nanoscopy contrast-generating unit comprises internal attachment points. Said internal attachment points are selected from the group consisting of nanobodies, frankenbodies, coiled-coil domains, isopeptide-forming partners such as SpyTag or SpyCatcher domains, SnoopTag/SnoopCatcher, DogTag/DogCatcher, bioconjugation tags such as SNAP/Clip-Tag domains, and/or Halo-Tag domains, split-inteins, calmodulin-binding peptides, phosphorylatable peptides, conformation-changing peptides such as troponins, as well as combinations thereof. In another embodiment, the genetically controlled nanoscopy contrast-generating unit comprises external attachment points. Said external attachment points are selected from the group consisting of nanobodies, frankenbodies, coiled-coil domains, isopeptide-forming partners such as SpyTag or SpyCatcher domains, SnoopTag/SnoopCatcher, DogTag/DogCatcher, bioconjugation tags such as SNAP/Clip-Tag domains, and/or Halo-Tag domains, split-inteins, calmodulin-binding peptides, phosphorylatable peptides, conformation-changing peptides such as troponins (Thestrup et al., Nature Methods, 2014), as well as combinations thereof. In yet another embodiment, the genetically controlled nanoscopy contrast-generating unit comprises internal attachment points and external attachment points. Said internal and external attachment points are selected from the group consisting of nanobodies, frankenbodies, coiled-coil domains, isopeptide-forming partners such as SpyTag or SpyCatcher domains, SnoopTag/SnoopCatcher, DogTag/DogCatcher, bioconjugation tags such as SNAP/Clip-Tag domains, and/or Halo-Tag domains, split-inteins, calmodulin-binding peptides, phosphorylatable peptides, conformation-changing peptides such as troponins, as well as combinations thereof.

In a preferred embodiment, the genetically controlled nanoscopy contrast-generating unit comprises MT3, FABP, or BM3h as the metal interactor as either direct fusions or via attachment points chosen from coiled-coils, isopeptide forming pairs or inteins, wherein via the attachment point the CGU can be attached to structural elements such as encapsulins, vaults, emFP, or SasG.

### Genetically Controlled Structural Elements (SEs)

In one aspect, the invention relates to a genetically controlled structural element (SE). The genetically controlled structural element comprises and spatially organizes at least one genetically controlled nanoscopy contrast-enhancing unit. The genetically controlled structural element interacts or binds to the genetically controlled nanoscopy contrast-enhancing unit and therefore, organizes the genetically controlled nanoscopy contrast-enhancing unit. In general, the genetically controlled structural element has to be of a sufficient size that is detectable by, for example, in high-throughput EM using, e.g., a pixel size of 1 to 4 nanometers. The genetically controlled structural element possesses a pre-determined shape and pre-determined nanoscale size and generates a shape that can be differentiated at the nanoscale, i.e., it can be detected and distinguished from shapes of other SEs or other (cell) structures in EM and other nanoscale detection methods. Such a distinct shape is preferably a barcode, even more preferably a concentric barcode.

The distinct shape is a function of the 3D distribution of the genetically controlled nanoscopy contrast-generating units within the genetically controlled structural element. In other words, the geometrically distinct shape is generated by the interaction of the structurally organized genetically controlled nanoscopy contrast-generating units and the genetically controlled structural element. In standard EM methods, these 3D distributions are commonly detected or presented as 2D cross-sections or projections onto a plane.

If such a shape generates a distinct spatial distribution of contrast, in particular, a series of contrast-rich and contrast-poor regions with defined spatial intervals, such as a series of high-contrast parallel bars with defined width and defined low-contrast intervals, the shape is called a “barcode”. In a preferred embodiment, the distinct shapes are concentric rings, i.e., an annular shape, which in the limit converges to a disc shape. In one embodiment, the distinct shape is, for example, a ring, a disc, a rectangle, a triangle etc.

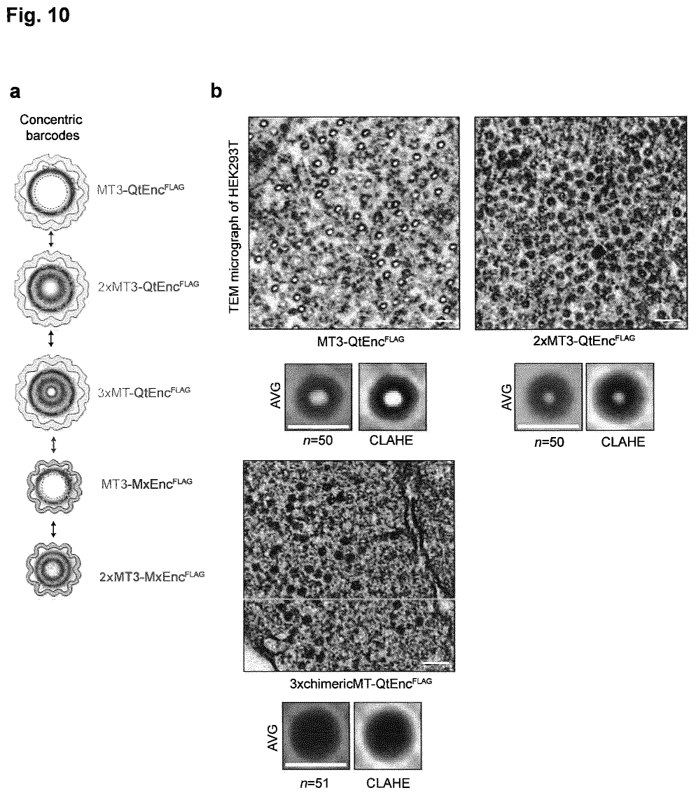

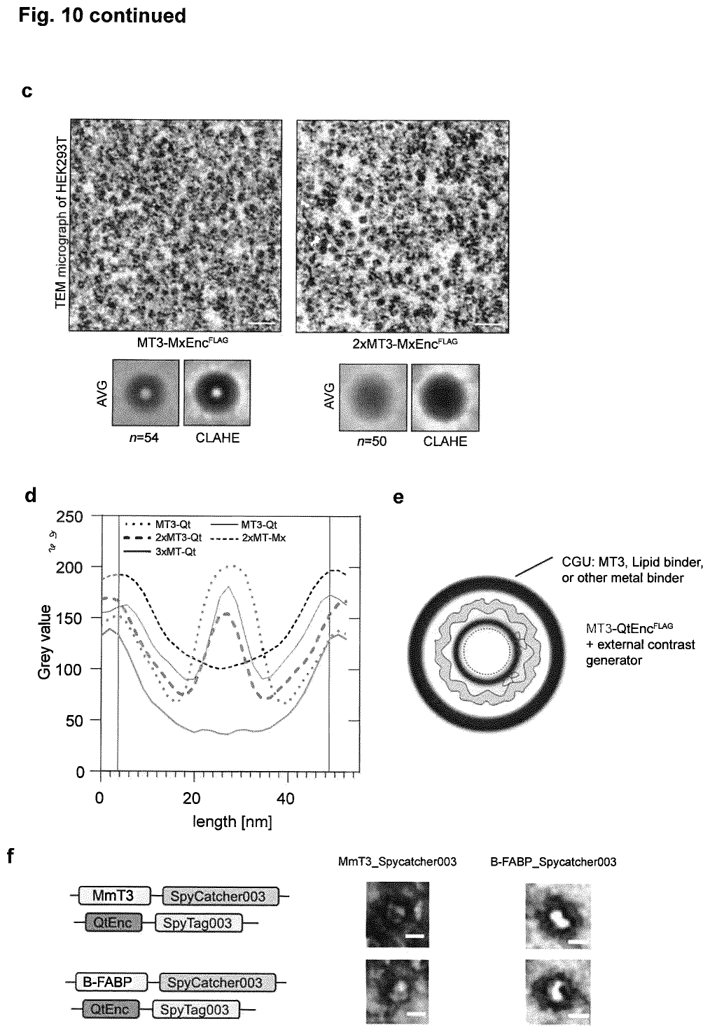

In another preferred embodiment, concatenated MT3 domains fused to encapsulin monomers produce an annular or disc shape. In one embodiment, single or tandem MT3 domains are fused to the N-terminus of QtEnc. In another embodiment, tandem MT3 domains provide twice the number of osmiophilic residues which reach further into the lumen of the encapsulin. More contrast is generated, and thus the diameter of the “inner circle” of the ring shape decreases, resulting in a disc. If three MT3 domains are fused to an encapsulin, the shape is a completely “dark” disc (see, FIG. 10b). In one embodiment, MT3-QtEnc^{FLAG} is a ring. In other words, one MT3 is fused to the interior of an encapsulin, resulting in a ring shape. In said embodiment, the ring is an annular electron-dense ring with a bright center. In another embodiment, 2xMT3-QtEnc^{FLAG} is a disc. In other words, two MT3s are fused to the interior of an encapsulin, resulting in a disc shape. Said disc has a darker appearance than the MT3-QtEnc^{FLAG} ring. In an alternative embodiment, 3xMT3-QtEnc^{FLAG} adds another layer of contrast to the lumen, which appears as a disc. In other words, three MTs from different species (3xchimericMT, SEQ ID NO: 169) are fused to the interior of an encapsulin, resulting in a disc shape. All three shapes are called “concentric barcodes,” which can be complemented by additional shapes generated by adding more layers of contrast to the outer surface of the spherical nanostructure (FIGS. 10e, 10f).

The genetically controlled structural element is preferably a self-assembling structure. A self-assembling structure has the advantage of consisting of only a few building blocks while having the disadvantage of being polyvalent, thus making it challenging to include sparse attachment points that can be used to bind to a target of interest or attach to a scaffold to generate a precise geometric pattern. In one embodiment, the genetically controlled structural element is not self-assembling. In another embodiment, the genetically controlled structural element is self-assembling. In one embodiment, the genetically controlled structural element is not symmetrical. In another embodiment, the genetically controlled structural element is symmetrical. In one embodiment, the genetically controlled structural element is not self-assembling and not symmetrical. In another embodiment, the genetically controlled structural element is self-assembling and symmetrical. An encapsulin is an example of a genetically controlled structural element, which is self-assembling, symmetrical, and spherical. A vault is an example of a genetically controlled structural element that is self-assembling and symmetrical in one axis but is not a sphere. Obtaining a low valency, let alone a single valency on the outer surface, is very challenging if a self-assembling and symmetrical structure is used. A low valency is preferred such that cross-linking of, for example, the target is avoided.

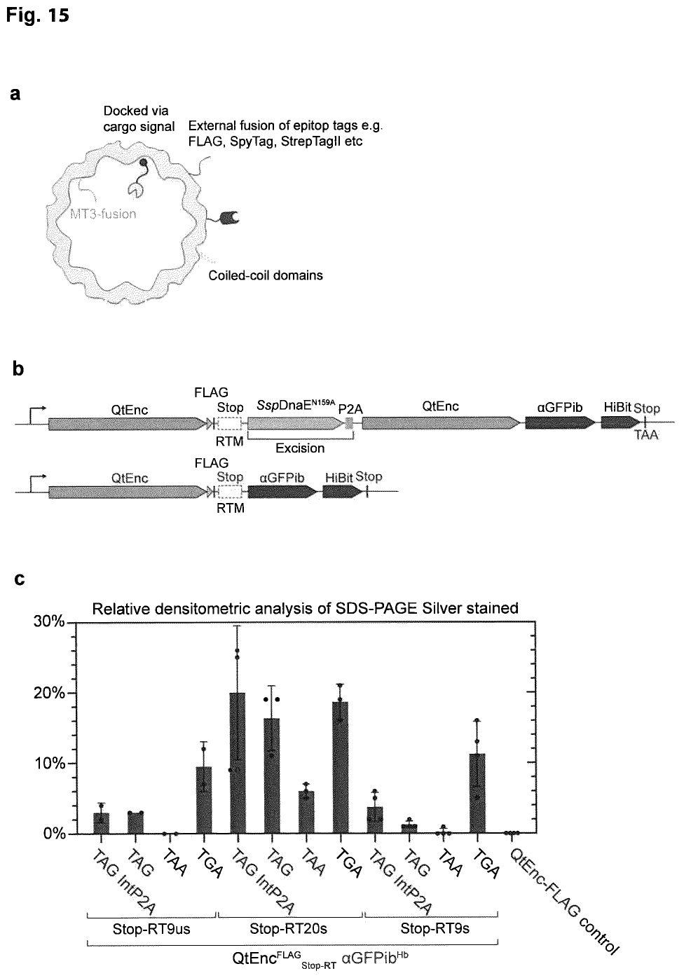

A solution to the problem is provided by genetically controlling the stoichiometry of self-assembling building blocks with or without distinct attachment points. This objective can be achieved via changing the concentration of plasmids or viruses encoding the respective genetically encoded building blocks in transient transfection or transduction systems. For a stable genetic modification, a solution is provided by a translational read-through system (FIG. 15), which uses several variants of termination codons to regulate the probability of ribosomes passing through the termination codon to prolong the translated peptide, e.g. by a terminal attachment point.

In one embodiment, the stoichiometry of surface modifications of the self-assembling and symmetrical genetically controlled structural element can thus be regulated. In one embodiment, said regulation takes place by programmable translational read-through. In a preferred embodiment, programmable translational read-through entails a programmable RT cassette comprising a structural element with a stop codon and a read-through promoting sequences. In another preferred embodiment, the programmable RT cassettes comprise a QtEnc monomer gene with a C-terminal FLAG epitope followed by a stop codon and read-through promoting sequences. In one embodiment, the programmable translational read-through is determined by the stop codon and the respective read-through motif. In one embodiment, the stop codon is selected from the group consisting of UAG, UAA, and UGA (TAG, TAA; TGA). In a preferred embodiment, the stop codon is TGA. In one embodiment, the read-through motif is selected from the group consisting of 3 SEQ ID NOs: 74-76. In one embodiment, the combination of the stop codon and read-through motif allows for adjusting read-through rates between about 1 and 20%. In one embodiment, the genetically controlled structural element is a Quasibacillius thermotolerans (Qt) encapsulin, wherein the genetically controlled contrast-generating units is one murine metallothionein-3, wherein the genetically controlled structural element comprises a programmable read-through cassette, wherein said programmable read-through cassette comprises distinct combinations of stop codons and short read-through promoting motifs at the end of the open-reading frame encoding the encapsulin Quasibacillius thermotolerans (Qt). In one embodiment, the stop codon is selected from the group consisting of TGA, TAA, TAG and wherein the short read-through promoting motif is selected from the group consisting of rt20s (SEQ ID NO: 76), rt9s (SEQ ID NO: 74), rt9us (CTATCC). In a preferred embodiment, the stop codon is TGA and the read-through promoting motif is rt20 (SEQ ID NO: 76). In another embodiment, the genetically controlled structural element additionally comprises a C-terminally fused anti-GFP intrabody. In yet another embodiment, the genetically controlled structural element is co-expressed with Cx43-msfGFP. In even another embodiment, the genetically controlled structural element is co-expressed with msfGFP-Cx43. In another embodiment, the genetically controlled structural element is co-expressed with a C-terminally fused Cx43-msfGFP. In an alternative embodiment, the genetically controlled structural element is co-expressed with a C-terminally fused msfGFP-Cx43. In even another embodiment, the genetically controlled structural element additionally comprises a N-terminally, or C-terminally fused anti-GFP intrabody. In a preferred embodiment, the additionally fused anti-GFP intrabody faces the exterior, i.e. not the lumen. In one aspect, a method of generating a distinct ratio of genetically controlled structural elements is disclosed. In one embodiment, the genetically controlled structural element is a Quasibacillius thermotolerans (Qt) encapsulin, wherein said Quasibacillius thermotolerans (Qt) encapsulin comprises one N-terminally fused murine metallothionein-3, wherein at the end of the open-reading frame encoding the encapsulin Quasibacillius thermotolerans (Qt) the read-through motif rt20s (SEQ ID NO: 76) and the stop codon TGA are located, wherein the Quasibacillius thermotolerans (Qt) encapsulin comprises one C-terminally fused FLAG-tag, wherein said C-terminally fused FLAG tag is further extended by an anti-GFP intrabody upon ribosomal read-through, whereby the combination of the read-through motif rt20s (SEQ ID NO: 76) and the stop codon yields about 15% anti-GFP intrabodies, thereby serving as a surrogate marker for a translational read-through rate. In one embodiment, distinct combinations of a read-through motif and a stop codon yield about 5%, or about 20% anti-GFP intrabodies, i.e. a read-through rate of about 5% or 20%. Thus, in another embodiment a method for the detection of translational read-through rates is also provided. In a further aspect, a cell line comprising the genetically controlled structural element and the C-terminally fused Cx43-msfGFP is provided.

In an embodiment, the genetically controlled structural element is self-assembling, symmetrical, and rotationally invariant. A member of the encapsulin family is an example of a genetically controlled structural element that is self-assembling, symmetrical, and rotationally invariant. Taking the example of encapsulins, a maximum number of internal attachment points is desirable to maximize the density of contrast-generating units.

In one embodiment, the genetically controlled structural element comprises a member of the encapsulin family, a vault, an MS2 phage, a Qbeta phage, an AP205 phage, an engineered mono-to-polyvalent hub, a filament, or a linker, or combinations or variants thereof.

The genetically controlled structural element may comprise a member of the encapsulin family. In a preferred embodiment, the genetically controlled structural element is an encapsulin, preferably from the species Q. thermotolerans (QtEnc; T=4, 40 nm in diameter), M. xanthus (MxEnc; T=3, 32 nm in diameter), and T. maritima (TmEnc; T=1, 20 nm in diameter). In another embodiment, the encapsulin has a triangulation number of four, three, or one. In even another embodiment, the encapsulin has a size of between about 20 to 45 nm, about 35 to 45 nm, about 27 to 37 nm, or about 15 to about 25 nm in diameter. In a preferred embodiment, the encapsulin has a size of about 40 nm, 30 nm or 20 nm in diameter. In another preferred embodiment, the encapsulin has a triangulation number of four and a size of about 40 nm, a triangulation number of three and a size of about 30 nm, a triangulation number of 1, and a size of about 20 nm all in diameter.

In another embodiment, the genetically controlled structural element comprises an encapsulin and at least one genetically controlled nanoscopy contrast-generating unit comprising a metal interactor wherein the encapsulin spatially organizes the genetically controlled nanoscopy contrast-generating unit. In another embodiment, the genetically controlled structural element organizing the genetically controlled nanoscopy contrast-generating unit is a fusion protein. In a preferred embodiment, the genetically controlled structural element organizing the genetically controlled nanoscopy contrast-generating unit is a fusion protein comprising an encapsulin and at least one metal interactor. In one embodiment, the metal interactor is a uranyl binder. In a preferred embodiment, the uranyl binder is MUP2 (SEQ ID NO:45), MUP5 (SEQ ID NO: 46), SUP (SEQ ID NO: 225), des_7bau (SEQ ID NO: 268), des_2akf_3bs (SEQ ID NO: 269), des_2akf_6bs (SEQ ID NO: 270), or des_5apq_6bs (SEQ ID NO: 271). In a particularly preferred embodiment, the uranyl binder is MUP2 (SEQ ID NO:45) and the genetically controlled structural element is an encapsulin selected from Quasibacillius thermotolerans (Qt), Myxococcus xanthus (Mx), and Thermotoga maritima (Tm). In a preferred embodiment, the uranyl binder is MUP2 and the genetically controlled structural element is a Quasibacillius thermotolerans (Qt) encapsulin (SEQ ID NO: 148). In a preferred embodiment, the at least one metal interactor is a metallothionein, preferably MT3. In one embodiment, said MT3 is murine MT3. In another preferred embodiment, the encapsulin is fused with a tandem MT3 domain, in other words, comprises two MT3 domains. In one embodiment, an encapsulin is N-terminally fused to a tandem mouse MT3 domain. In another preferred embodiment, the fusion protein is MT3-QtEnc (SEQ ID NO: 165), MT3-MxEnc (SEQ ID NO: 166), or MT3-TmEnc, or in other words, a genetically controlled structural element comprising a Quasibacillius thermotolerans (Qt) encapsulin, Myxococcus xanthus (Mx) encapsulin, or Thermotoga maritima (Tm) encapsulin, further comprising a genetically controlled contrast-generating unit, wherein said genetically controlled contrast-generating unit is one N-terminally fused murine metallothionein-3 (MmT3). In one embodiment, the one murine metallothionein is located on the inner surface of the encapsulin. In another embodiment, the fusion protein is 2xMT3-QtEnc, 2xMT3-MxEnc, or 3xMT3-TmEnc, or in other words, a genetically controlled structural element comprising Quasibacillius thermotolerans (Qt) encapsulin, Myxococcus xanthus (Mx) encapsuin, or Thermotoga maritima (Tm) encapsulin, further comprising a genetically controlled contrast-generating unit, wherein said genetically controlled contrast-generating unit is two N-terminally fused murine metallothionein. In one embodiment, the two murine metallothioneine are located on the inner surface of the encapsulin. In yet another embodiment, the fusion protein is 3x MT3-QtEnc, 3xMT3-MxEnc, or 3xMT3-TmEnc, or in other words, a genetically controlled structural element comprising a Quasibacillius thermotolerans (Qt) encapsulin, Myxococcus xanthus (Mx) encapsulin, or Thermotoga maritima (Tm) encapsulin, further comprising a genetically controlled contrast-generating unit, wherein said genetically controlled contrast-generating uni is a chimeric metallothionein sequence comprising three N-terminally fused metallothioneins comprising murine metallothionein-3 (MmT3), Synechococcus elongatus (SmtA) metallothionein, and Triticum aestivum Ec-1 metallothionein (TaEC1) (SEQ ID NO: 5). In one embodiment, the three N-terminally chimeric fused metallothioneins are located on the inner surface of the encapsulin.

In another embodiment, the genetically controlled structural element, comprising a Quasibacillius thermotolerans (Qt) encapsulin, Myxococcus xanthus (Mx) encapsulin, or Thermotoga maritima (Tm) encapsulin and a metallothionein according to the preceding embodiments undergoes sequential segmentation and classification, wherein the sequential segmentation and classification comprises the steps of (i) segmentation, (ii) background subtraction, (iii) classification. Table 1 below displays tabulated segmentation metrics for all 6 classes. Overall average model precision is 82.2%, recall 74.6%, DSC 0.78, and IoU 0.64.:

TABLE 1
Sequential segmentation and classification of Quasibacillius
thermotolerans (Qt) encapsulin, Myxococcus xanthus (Mx) encapsulin,
or Thermotoga maritima (Tm) encapsulin comprising one (1M) or two
murine metallothioneins (2M) or the chimeric metallothionein sequence
having three metallothioneins (3M):

| EMcapsulins | | | | |
|---|---|---|---|---|
| class | Precision | Recall | DSC | IoU |
| 1M-Qt | 0.79 | 0.89 | 0.84 | 0.72 |

[symbol]

| | | | | |
|---|---|---|---|---|
| 3M-Qt | 0.83 | 0.67 | 0.74 | 0.59 |
| 1M-Mx | 0.71 | 0.59 | 0.64 | 0.48 |
| 2M-Mx | 0.75 | 0.56 | 0.64 | 0.47 |
| 1M-Tm | 0.74 | 0.24 | 0.36 | 0.22 |

In yet another preferred embodiment, the fusion protein is MT3-QtEnc-FLAG, MT3-MxEnc-FLAG, or MT3-TmEnc-BC2Tag. In still another embodiment, the fusion protein is MT3-TmEnc-FLAG. Put differently, a genetically controlled structural element comprising a Quasibacillius thermotolerans (Qt) encapsulin, Myxococcus xanthus (Mx) encapsulin, or Thermotoga maritima (Tm) encapsulin, further comprising a genetically controlled contrast-generating unit, wherein said genetically controlled contrast-generating unit is one N-terminally fused murine metallothionein-3 (MmT3), wherein a FLAG tag is C-terminally fused is disclosed. In one embodiment, the one murine metallothionein is located on the inner surface of the encapsulin. In another embodiment, the fusion protein is 2xMT3-QtEnc-FLAG, 2xMT3-MxEnc-FLAG, or 3xMT3-TmEnc-FLAG, or in put differently, a genetically controlled structural element comprising a Quasibacillius thermotolerans (Qt) encapsulin, Myxococcus xanthus (Mx) encapsulin, or Thermotoga maritima (Tm) encapsulin, further comprising a genetically controlled contrast-generating unit, wherein said genetically controlled contrast-generating unit is two N-terminally fused murine metallothionein, wherein a FLAG-tag is C-terminally fused. In one embodiment, the two murine metallothioneine are located on the inner surface of the encapsulin. In yet another embodiment, the fusion protein is 3xMT3-QtEnc-FLAG (SEQ ID NO: 169), 3xMT3-MxEnc-FLAG, or 3xMT3-TmEnc-FLAG, or in other words, a genetically controlled structural element comprising a Quasibacillius thermotolerans (Qt) encapsulin, Myxococcus xanthus (Mx) encapsulin, or Thermotoga maritima (Tm) encapsulin), further comprising a genetically controlled contrast-generating unit, wherein said genetically controlled contrast-generating unit is a chimeric metallothionein sequence comprising three N-terminally fused metallothioneins comprising murine metallothionein-3 (MmT3), Synechococcus elongatus (SmtA) metallothionein, and Triticum aestivum Ec-1 metallothionein (TaEC1) (SEQ ID NO: 5)., wherein a FLAG-tag is C-terminally fused. In one embodiment, the three N-terminally chimeric fused metallothioneins are located on the inner surface of the encapsulin.

In even another embodiment, the genetically controlled structural element additionally comprises apart from a C-terminally fused FLAG-tag also a nuclear localization signal (NLS). In one embodiment, said NLS can be fused to said FLAG-tag via cMyc. Consequently, in one embodiment, the genetically controlled structural element is Quasibacillius thermotolerans (Qt) encapsulin, Myxococcus xanthus (Mx) encapsulin, or Thermotoga maritima (Tm) encapsulin, comprising a genetically controlled contrast-generating unit having one, or two N-terminally fused murine metallothionein-3, or wherein the genetically controlled contrast-generating units is chimeric metallothionein sequence comprising three N-terminally fused metallothioneins comprising murine metallothionein-3 (MmT3), Synechococcus elongatus (SmtA) metallothionein, and Triticum aestivum Ec-1 metallothionein (TaEC1) (SEQ ID NO: 5), wherein a FLAG tag with a nuclear localization signal is C-terminally fused. In another embodiment, the genetically controlled structural element is a Quasibacillius thermotolerans (Qt) encapuslin, wherein the genetically controlled contrast-generating unit is one N-terminally fused murine metallothionein-3, or wherein a FLAG tag with a nuclear localization signal is C-terminally fused. In even another embodiment, the genetically controlled structural element is a Quasibacillius thermotolerans (Qt) encapuslin, comprising a genetically controlled contrast-generating unit having three N-terminally fused metallothioneins comprising murine metallothionein-3 (MmT3), Synechococcus elongatus (SmtA) metallothionein, and Triticum aestivum Ec-1 metallothionein (TaEC1) (SEQ ID NO: 5), wherein a FLAG tag with a nuclear localization signal is C-terminally fused.

In another preferred embodiment, the genetically controlled structural element is an encapsulin, and the at least one metal interactor is RNA.

In yet another preferred embodiment, the genetically controlled structural element is an encapsulin, and the at least one metal interactor is an RNA-binding protein.

In even another preferred embodiment, the genetically controlled structural element is an encapsulin, and the at least one metal interactor is a lipid-binding protein, preferably a fatty-acid binding protein.

In one embodiment, the genetically controlled structural element is an encapsulin, the at least one metal interactor is RNA and the genetically controlled nanoscopy contrast-generating unit additionally comprises a kinase, IMEF, with co-expression of ZIP14, DMT1, or a tyrosinase.

In yet another preferred embodiment, the genetically controlled structural element is an encapsulin, the at least one metal interactor is an RNA-binding protein and the genetically controlled nanoscopy contrast-generating unit additionally comprises a kinase, IMEF, or a tyrosinase.

In even another preferred embodiment, the genetically controlled structural element is an encapsulin, the at least one metal interactor is a lipid-binding protein, preferably a fatty-acid binding protein and the genetically controlled nanoscopy contrast-generating unit additionally comprises a kinase, IMEF, or a tyrosinase.

In a preferred embodiment, the genetically controlled nanoscopy contrast-generating unit is the synthetic enzyme tyrosinase and the structural element is an encapsulin comprising the tyrosinase. Melanin is generated in the encapsulin lumen by the tyrosinase (an exemplary tyrosinase is BmTyr having SEQ ID NO: 51). In one embodiment, melanin binds to heavy metals. In another embodiment, the melanin is contrasted without binding to heavy metals in EM.

In another embodiment, the genetically controlled nanoscopy contrast-generating unit is the polymerizing enzyme kinase and the structural element is an encapsulin comprising the kinase, preferably PPK2. ATP or GTP enters the genetically controlled structural element via its pores, where it is polymerized into a polyphosphate chain trapped inside the encapsulin lumen. The result is highly contrasted polyphosphate pores. In this particular embodiment, the resulting structures are from about 25 to 33 nm, preferably about 32 nm, and comprise MxEnc^{FLAG}+DD-CgPPK2b-QtSig (SEQ ID NOs: 107, 47).

In another embodiment, the encapsulin comprises mineralized iron oxide via its native IMEF cargo protein. In yet another embodiment, an IMEF dimer is docked into the encapsulin shell, where ferrous ion enters through the pore in the encapsulin shell and is subsequently bio-mineralized. The resulting encapsulin is, for example, QtEnc+QtIMEF, which is a spherical structure of about 40 nm.

In another embodiment, the genetically controlled structural element organizing the genetically controlled nanoscopy contrast-generating unit is a fusion protein comprising an encapsulin, at least one metal interactor, and a fluorophore. In a preferred embodiment, the fusion protein is eUnaG-MT3-QtEnc (SEQ ID NO: 155), or put differently a genetically controlled structural element, wherein the genetically controlled structural element is a Quasibacillius thermotolerans (Qt) encapsulin, Myxococcus xanthus (Mx) encapsulin, or Thermotoga maritima (Tm) encapsulin, comprising a genetically controlled contrast-generating units having one murine metallothionein-3, wherein the genetically controlled structural element further comprises the fluorescent protein eUnaG. In a preferred embodiment, the genetically controlled structural element is Quasibacillius thermotolerans (Qt) encapsulin. In another preferred embodiment, eUnaG is N-terminally fused. In another embodiment, the genetically controlled structural element additionally comprises a C-terminally fused FLAG tag, such that the fusion protein is eUnaG-MT3-QtEnc^{FLAG}. In one aspect, a fluorescence multichannel imaging method is provided, using the genetically controlled structural element additionally comprising a C-terminally fused FLAG tag, such that the fusion protein is eUnaG-MT3-QtEnc^{FLAG}. In another aspect, a method for monitoring organ, organoid or cells is provided, using the genetically controlled structural element additionally comprising a C-terminally fused FLAG tag, such that the fusion protein is eUnaG-MT3-QtEnc^{FLAG}. In another embodiment, eUnaG is C-terminally fused. Therefore, in one embodiment, the fusion protein is MT3-QtEnc^{eUnaG}. In another aspect, the genetically controlled structural element according to the preceding embodiments is compatible with both fluorescent microscopy and electron microscopy applications. In a preferred embodiment, the genetically controlled structural element according to the preceding embodiments is used for sequential analysis, first by fluorescent microscopy, followed by electron microscopy. In another embodiment, the genetically controlled structural element further comprises at least one attachment point, wherein the at least one attachment point is an intrabody, wherein said intrabody is C-terminally fused. In one embodiment, the intrabody is an anti-mCherry intrabody. In another embodiment, the anti-mCherry intrabody is expressed on the outer surface of the encapsulin. Thus, in one embodiment, the fusion protein is MT3-QtEnc-^{an}ti-^{mcherry}. In another embodiment, the genetically controlled structural element is a Quasibacillius thermotolerans (Qt) encapsulin, comprising a genetically controlled contrast-generating unit, wherein said genetically controlled contrast-generating unit is one murine metallothionein-3, wherein eUnaG is N-terminally fused, wherein an anti-mCherry intrabody is C-terminally fused. Hence, the fusion protein according to the preceding embodiment is eUnaG-MT3-QtEnc^{anti-mcherry}. In one aspect, a fluorescence imaging method using the genetically controlled structural element, wherein the genetically controlled structural element is a Quasibacillius thermotolerans (Qt) encapsulin, comprising a genetically controlled contrast-generating unit, wherein said genetically controlled contrast-generating unit is one murine metallothionein-3, wherein eUnaG is N-terminally fused, wherein an anti-mCherry intrabody is C-terminally fused, or in other words, the fusion protein eUnaG-MT3-QtEnc^{anti-mcherry} is provided. In another aspect, a method for monitoring organ, organoid or cells using the genetically controlled structural element according to the preceding embodiment is provided. In one embodiment, the monitored cells are mammalian oocytes. In a preferred embodiment, the oocytes are murine oocytes. In another preferred embodiment, the oocytes are human oocytes. In another embodiment, the mammalian oocytes according to the preceding embodiments are monitored prior to in vitro fertilization. In one aspect, a recycling endosome stain comprising a Quasibacillius thermotolerans (Qt) encapsulin as genetically controlled structural element, further comprising a genetically controlled contrast-generating unit, wherein said genetically controlled contrast-generating unit is one murine metallothionein-3, wherein eUnaG is N-terminally fused, wherein an anti-mCherry intrabody is C-terminally fused, wherein the anti-mCherry intrabody is targeted to RAB11A is provided. In another aspect, a cargo vesicle stain comprising a Quasibacillius thermotolerans (Qt) encapsulin as genetically controlled structural element, further comprising a genetically controlled contrast-generating unit, wherein said genetically controlled contrast-generating unit is one murine metallothionein-3, wherein eUnaG is N-terminally fused, wherein an anti-mCherry intrabody is C-terminally fused, wherein the anti-mCherry intrabody is targeted to Myo5b is provided. In yet another aspect, an acentriolar microtubule-organizing stain comprising the genetically controlled structural comprising a Quasibacillius thermotolerans (Qt) encapsulin as genetically controlled structural element, further comprising a genetically controlled contrast-generating unit, wherein said genetically controlled contrast-generating unit is one murine metallothionein-3, wherein eUnaG is N-terminally fused, wherein an anti-mCherry intrabody is C-terminally fused, wherein the anti-mCherry intrabody is targeted to PLK1 is provided.

In one embodiment, MT3-QtEnc^{eUnaG} is co-expressed with MT3-QtEnc^{-anti-mcherry}. In a preferred embodiment, MT3-QtEnc^{eUnaG} is co-expressed with MT3-QtEnc^{-anti-mcherry} in a ratio of approximately 4:1. In one aspect, a subcellular labelling method is provided wherein MT3-QtEnc^{eUnaG} is co-expressed with MT3-QtEnc^{-anti-mcherry}. In a preferred embodiment, MT3-QtEnc^{eUnaG} is co-expressed with MT3-QtEnc^{-anti-mcherry} in a ratio of approximately 4:1. In another embodiment, the genetically controlled contrast-generating unit additionally comprises at least one further attachment point, wherein said attachment point is a coiled-coil domain. In a preferred embodiment, the attachment point is two coiled-coil domains. In yet another embodiment, the genetically controlled contrast-generating unit additionally comprises at least one further attachment point, wherein said attachment point is a SpyTag/SpyCatcher.

In a particularly preferred embodiment, the fusion protein is eUnaG-MT3-QtEnc or eUnaG-2xMT3-QtEnc, or in other words, a genetically controlled structural element, wherein the genetically controlled structural element is a Quasibacillius thermotolerans (Qt) encapsulin, Myxococcus xanthus (Mx) encapsulin, or Thermotoga maritima (Tm) encapsulin, comprising a genetically controlled contrast-generating units having two murine metallothionein-3, wherein the genetically controlled structural element further comprises the fluorescent protein eUnaG. In a preferred embodiment, the genetically controlled structural element is Quasibacillius thermotolerans (Qt) encapsulin. In another preferred embodiment, eUnaG is N-terminally fused. In another embodiment, the genetically controlled structural element additionally comprises a C-terminally fused FLAG tag, such that the fusion protein is eUnaG-2xMT3-QtEnc^{F}LAG. In one aspect, a fluorescence multichannel imaging method is provided, using the genetically controlled structural element additionally comprising a C-terminally fused FLAG tag, such that the fusion protein is eUnaG-2xMT3-QtEnc^{FLAG}. In another aspect, a method for monitoring organ, organoid or cells is provided, using the genetically controlled structural element additionally comprising a C-terminally fused FLAG tag, such that the fusion protein is eUnaG-2xMT3-QtEnc^{FLAG}. In another embodiment, eUnaG is C-terminally fused. In another embodiment, the genetically controlled structural element further comprises at least one attachment point, wherein the at least one attachment point is an intrabody, wherein said intrabody is C-terminally fused. In one embodiment, the intrabody is an anti-mCherry intrabody. In another embodiment, the anti-mCherry intrabody is expressed on the outer surface of the encapsulin. In another embodiment, the genetically controlled contrast-generating unit additionally comprises at least one further attachment point, wherein said attachment point is a coiled-coil domain. In a preferred embodiment, the attachment point is two coiled-coil domains. In yet another embodiment, the genetically controlled contrast-generating unit additionally comprises at least one further attachment point, wherein said attachment point is a SpyTag/SpyCatcher.

In even another embodiment, the genetically controlled structural element is a Quasibacillius thermotolerans (Qt) encapsulin, comprising a genetically controlled contrast-generating unit, wherein said genetically controlled contrast-generating unit is one murine metallothionein-3, wherein the genetically controlled structural element further comprises the fluorescent protein eUnaG, and wherein the genetically controlled structural element further comprises synthetic calmodulin-binding peptide 20 (SEQ ID NO: 241). In one embodiment, the genetically controlled structural element according to the preceding embodiment and a genetically controlled nanoscopy contrast-generating unit, wherein said genetically controlled nanoscopy contrast-generating unit comprises the metal-binding peptide/protein is H. pylori ferritin, comprising at least one attachment point, and wherein said at least one attachment point is a calmodulin-binding peptide (SEQ ID NO: 134) are used for nanoscopy detection, wherein nanoscopy detection comprises molecular mapping and/or geometric sensing. In another embodiment, the genetically controlled structural embodiment further comprising the synthetic calmodulin-binding peptide 20 (SEQ ID NO: 241), the genetically controlled contrast-generating unit of SEQ ID NO: 31 and additionally OptoCracl-mCherry are used for nanoscopy detection, wherein nanoscopy detection comprises molecular mapping and/or geometric sensing. OptoCracl-mCherry is a blue-light inducible optogenetic tool and calcium influx is stimulated via OptoCrac1 (doi:10.7754/eLife.10024). In a preferred embodiment, said molecular mapping provides information on the subcellular distribution of a molecule of interest within a cell or tissue via the binding of the genetically controlled structural element and the genetically controlled constast-generating unit according to the preceding embodiment to a molecule of interest, which thus determines the subcellular localization of the genetically controlled structural elements and the genetically controlled constast-generating unit.

In yet another embodiment, the nanoscopy detection is geometric sensing, and wherein said geometric sensing provides information on a specific cellular state, a cellular process, or the presence or absence of an analyte or environmental parameter of interest within a cell or tissue, or the response to an external stimulus, and wherein the shape generated by the genetically controlled structural element and the genetically controlled contrast-generating unit changes in response to a pre-defined cellular state, a pre-defined cellular process, at least one analyte, or at least one environmental parameter, or an external stimulus. In a preferred embodiment, the at least one analyte is calcium.

In even another embodiment, the nanoscopy detection comprises mapping and/or the geometrical sensing further comprises geometric actuation, wherein said geometric actuation alters a cellular state or process by an external stimulus. In a preferred embodiment, the external stimulus is calcium.

In one embodiment, eUnaG-MT3-QtEnc generates concentric barcodes of about 35-45 nm in diameter, preferably about 40 nm. In one embodiment, said spherical structures harbor a distinct lumenal phenotype showing a contrasted internal ring as well as a bright center spot. In one embodiment, eUnaG-MT3 is localized to the encapsulin lumen. In yet another embodiment, the fusion protein is eUnaG-2xMT3-QtEnc, and the lumen is homogeneously contrasted.

In one embodiment, UnaG-MT3-QtEnc is localized to the cytosol. In another embodiment, UnaG-2xMT3-QtEnc is localized to the cytosol.

In yet another embodiment, the fusion protein is MT3-MxEnc^{FLAG}-cMyc-NLS. In one embodiment, MT3-MxEnc-FLAG-cMyc-NLS is expressed pan-neuronally in Drosophila melanogaster. In yet another embodiment, MT3-MxEnc^{FLAG}-cMyc-NLS is expressed pan-neuronally in the optical lobe in Drosophila melanogaster. In a preferred embodiment, MT3-MxEnc^{FLAG}-cMyc-NLS is expressed in T4-T5 neurons in Drosophila melanogaster, or in other words, a genetically controlled structural element, wherein the genetically controlled structural element is a Myxococcus xanthus (Mx) encapsulin, further comprising a genetically controlled contrast-generating unit, wherein said genetically controlled contrast-generating unit is one N-terminally fused murine metallothionein-3, and wherein a FLAG tag with a nuclear localization signal is C-terminally fused via cMyc. In another aspect, a transgenic Drosophila melanogaster line is disclosed. In one embodiment, the transgenic Drosophila melanogaster line expresses MT3-MxEnc^{FLAG}-cMyc-NLS. In one aspect, MT3-MxEnc^{FLAG}-cMyc-NLS, or in other words, a genetically controlled structural element, wherein the genetically controlled structural element is a Myxococcus xanthus (Mx) encapsulin, further comprising a genetically controlled contrast-generating unit, wherein said genetically controlled contrast-generating unit is one N-terminally fused murine metallothionein-3, and wherein a FLAG tag with a nuclear localization signal is C-terminally fused via cMyc is used in a nanoscopy method. In one embodiment, the genetically controlled structural element does not interfere with cellular processes. In another embodiment, the nanoscopy method is electron microscopy. In yet another embodiment, the electron microscopy method is selected from the group consisting of scanning electron microscopy (SEM), transmission electron microscopy (TEM) and Focused Ion Beam Scanning Electron Microscopy (FIB-SEM). In even another embodiment, the electron microscopy method does not require further incubation steps involving DAB and hydrogen peroxide or further exogenous substrates.

In even another embodiment, the fusion protein is 3xMT3-QtEnc_^{FLAG}-cMyc-NLS. In one embodiment, 3xMT3-QtEnc_^{FLAG}-cMyc-NLS expressed pan-neuronally in Drosophila melanogaster. In a preferred embodiment, 3xMT3-QtEnc_^{FLAG}-cMyc-NLS is expressed in C3 neurons in Drosophila melanogaster, or in other words, a gentetically controlled structural element, wherein the genetically controlled structural element is a Quasibacillus thermotolerans (Qt) encapsulin, further comprising a genetically controlled contrast-generating unit, wherein said genetically controlled contrast-generating unit is three N-terminally fused chimeric metallothionein-3, and wherein a FLAG tag with a nuclear localization signal is C-terminally fused via cMyc. In another aspect, a transgenic Drosophila melanogaster line is disclosed. In one embodiment, the transgenic Drosophila melanogaster line expresses 3xMT3-QtEnc_^{FLAG}-cMyc-NLS. In a preferred embodiment, the transgenic Drosophila melanogaster line expresses 3xMT3-QtEnc_^{FLAG}-cMyc-NLS and MT3-MxEnc^{FLAG}-cMyc-NLS. In another preferred embodiment, the transgenic Drosophila melanogaster line expressed MT3-MxEnc^{FLAG}-cMyc-NLS in T4-T5 neurons and 3xMT3-QtEnc_^{FLAG}-cMyc-NLS in C3 neurons. In one aspect, 3xMT3-QtEnc_^{FLAG}-cMyc-NLS, or in other words, a genetically controlled structural element, wherein the genetically controlled structural element is a Myxococcus xanthus (Mx) encapsulin, further comprising a genetically controlled contrast-generating unit, wherein said genetically controlled contrast-generating unit is three N-terminally fused chimeric metallothionein-3, and wherein a FLAG tag with a nuclear localization signal is C-terminally fused via cMyc is used in a nanoscopy method. In one embodiment, the genetically controlled structural element does not interfere with cellular processes. In another embodiment, the nanoscopy method is electron microscopy. In yet another embodiment, the electron microscopy method is selected from the group consisting of scanning electron microscopy (SEM), transmission electron microscopy (TEM) and Focused Ion Beam Scanning Electron Microscopy (FIB-SEM). In even another embodiment, the electron microscopy method does not require further incubation steps involving DAB and hydrogen peroxide or further exogenous substrates.

In a preferred embodiment, MmMT3-QtEnc is from about 35 to 45 nm in diameter, preferably about 39.24±3.51 nm, most preferably about 39 nm. In one embodiment, MmMT3-MxEnc is from about 25 to 35 nm in diameter, preferably about 29.94±2.61 nm, most preferably about 30 nm. In one embodiment, MmMT3-TmEnc is from about 15 to 25 nm in diameter, preferably about 21.2±2.54 nm, most preferably about 21 nm.

In another embodiment, the genetically controlled structural element is a Quasibacillius thermotolerans (Qt) encapsulin, Myxococcus xanthus (Mx) encapsulin, or Thermotoga maritima (Tm) encapsulin, further comprising a genetically controlled contrast-generating unit, wherein said genetically controlled contrast-generating unit is one or two murine metallothionein-3, wherein the genetically controlled structural element further comprises the fluorescent protein mScarlet-l. In a preferred embodiment, the genetically controlled structural element, is a Quasibacillius thermotolerans (Qt) encapuslin, further comprising a genetically controlled contrast-generating unit, wherein said genetically controlled contrast-generating unit is one murine metallothionein-3, wherein mScarlet-I is N-terminally fused, wherein a FLAG tag is C-terminally fused. In another preferred embodiment, the genetically controlled structural element, is a Quasibacillius thermotolerans (Qt) encapuslin, further comprising a genetically controlled contrast-generating unit, wherein said genetically controlled contrast-generating unit is two murine metallothionein-3, wherein mScarlet-I is N-terminally fused, wherein a FLAG tag is C-terminally fused.

In a specifically preferred embodiment, the genetically controlled structural element is an encapsulin and the genetically controlled nanoscopy contrast-generating unit comprises a metal interactor selected from the group of metallothionein, preferably MT3 or a series of different metallothionein species, fatty acid-binding proteins (FABPs), and the lipid-binding domain of the cytochrome P450, preferably the lipid- and heme-binding domain of the cytochrome P450 BM3 (BM3h) or a combination of these metal interactors (SEQ ID NO: 229, 230, 231). Therefore, the genetically controlled structural element according to this embodiment is an encapsulin selected from the group consisting of Quasibacillius thermotolerans (Qt) encapsulin, Myxococcus xanthus (Mx) encapsulin, or Thermotoga maritima (Tm) encapsulin and the genetically contrast-generating unit is B-FABP, B-FABP-ko, Bm3h, or Bm3h-B7. In a preferred embodiment, the encapsulin is a Quasibacillius thermotolerans encapsulin (Qt). In this embodiment, if the metal interactor is one or more metallothionein, one to three copies of the MT3 or one to three different metallothionein species are bound to the N-terminus of the encapsulin. In an alternative embodiment, the genetically contrast-generating unit is the lipid-binding protein, i.e. no metallothionein is present. Moreover, one or more MT3, FABP, or BM3h are bound to the surface of the encapsulin, optionally via a spacer, wherein the spacer is optionally a SasG element of variable length. Such organized structural element is configured to generate a distinct shape of a barcode, wherein the barcode is a concentric barcode differentiable at the nanoscale (SEQ ID NO: 228, 232, 233, 234, 235, 236). Such concentric barcode may encode information about the cellular state.

In yet another embodiment, the genetically controlled structural element is a Quasibacillius thermotolerans (Qt) encapsulin, Myxococcus xanthus (Mx) encapsulin, or Thermotoga maritima (Tm) encapsulin and the genetically contrast-generating unit is a bionconjugation tag. In a preferred embodiment, the genetically controlled structural element is a Quasibacillius thermotolerans (Qt) encapsulin, Myxococcus xanthus (Mx) encapsulin, or Thermotoga maritima (Tm) encapsulin and the genetically controlled contrast-generating unit is the bioconjugation tag SNAP. In a preferred embodiment, the genetically controlled structural element is an Quasibacillius thermotolerans (Qt) encapsulin and the genetically controlled contrast-generating unit is SNAP. In one embodiment, SNAP is either N-terminally or C-terminally fused.

The genetically controlled structural element may comprise a vault. In an embodiment, the vault comprises two hemi-vaults, each comprising 39 subunits of the major vault protein. In yet another embodiment, the vault protein comprises at least one fluorophore, preferably a fluorescent protein. In a preferred embodiment, the vault protein comprises a small fluorescent protein. In a more preferred embodiment, the small fluorescent protein is eUnaG, and the fluorescent protein is mScarlet. In another preferred embodiment, the small fluorescent protein is fused covalently to the artificially introduced internal HLH domain of the vault protein and/or the fluorescent protein is targeted non-covalently via a minimal interaction (mINT) motif. In yet another preferred embodiment, the vault comprises a SNAP25 palmitoylation motif. In a preferred embodiment, the genetically controlled structural element organizing the genetically controlled nanoscopy contrast-generating unit is a fusion protein comprising a vault and at least one metal interactor. In yet another preferred embodiment, the genetically controlled structural element is a vault, and the at least one metal interactor comprises at least one metallothionein. In yet another preferred embodiment, the genetically controlled structural element is a vault, and the at least one metal interactor is at least one murine metallothionein. In even another preferred embodiment, the genetically controlled structural element is a vault, and the at least one metal interactor is tandem MT3. In another preferred embodiment, the genetically controlled structural element is a vault, and the at least one metal interactor is RNA. In yet another preferred embodiment, the genetically controlled structural element is a vault, and the at least one metal interactor is an RNA-binding protein. In another embodiment, the genetically controlled structural element is a vault and the genetically controlled constrast-gerating unit is a RNA aptamer-binder: RNA aptamer pair. In said embodiment, the RNA aptamer-binder: RNA aptamer pair is preferably the circularized twister-broccoli aptamer. In even another preferred embodiment, the genetically controlled structural element is a vault, and the at least one metal interactor is a lipid-binding protein, preferably a fatty-acid binding protein. In one embodiment, the genetically controlled structural element is a vault, the at least one metal interactor is RNA and the genetically controlled nanoscopy contrast-generating unit additionally comprises a kinase, IMEF, or a tyrosinase. In yet another preferred embodiment, the genetically controlled structural element is a vault, the at least one metal interactor is an RNA-binding protein and the genetically controlled nanoscopy contrast-generating unit additionally comprises a kinase, IMEF, or a tyrosinase. In even another preferred embodiment, the genetically controlled structural element is a vault, the at least one metal interactor is a lipid-binding protein, preferably a fatty-acid binding protein and the genetically controlled nanoscopy contrast-generating unit additionally comprises a kinase, IMEF, or a tyrosinase.

The genetically controlled structural element may comprise a bacteriophage, in other words, prokaryotic (bacterial and archaeal) viruses. In an embodiment, the genetically controlled structural element comprises a bacteriophage and at least one genetically controlled nanoscopy contrast-generating unit comprising a metal interactor wherein genetically controlled nanoscopy contrast-generating unit is spatially organized by the structural element. In a preferred embodiment, the genetically controlled structural element is selected from the group consisting of MS2 phage (SEQ ID NO: 100), Qbeta phage (SEQ ID NO: 102), AP205 phage (SEQ ID NO: 207). In yet another preferred embodiment, the genetically controlled structural element is a self-assembling and thus, symmetric and multivalent genetically controlled structural element such as for example, MS2 phage and Qbeta phage. In a preferred embodiment, the genetically controlled structural element organizing the genetically controlled nanoscopy contrast-generating unit is a fusion protein comprising a bacteriophage and at least one metal interactor. In a preferred embodiment, the at least one metal interactor is MT3. In yet another preferred embodiment, the genetically controlled structural element is a bacteriophage and the at least one metal interactor comprises at least one metallothionein. In yet another preferred embodiment, the genetically controlled structural element is a bacteriophage and the at least one metal interactor is at least one murine metallothionein. In even another preferred embodiment, the genetically controlled structural element is a bacteriophage, and the at least one metal interactor is tandem MT3. In another preferred embodiment, the genetically controlled structural element is a bacteriophage, and the at least one metal interactor is RNA. In yet another preferred embodiment, the genetically controlled structural element is a bacteriophage and the at least one metal interactor is an RNA-binding protein. In even another preferred embodiment, the genetically controlled structural element is a bacteriophage and the at least one metal interactor is a lipid-binding protein, preferably a fatty-acid binding protein. In one embodiment, the genetically controlled structural element is a bacteriophage, the at least one metal interactor is RNA and the genetically controlled nanoscopy contrast-generating unit additionally comprises a kinase, IMEF or a tyrosinase. In yet another preferred embodiment, the genetically controlled structural element is a bacteriophage, the at least one metal interactor is an RNA-binding protein and the genetically controlled nanoscopy contrast-generating unit additionally comprises a kinase, IMEF, or a tyrosinase. In even another preferred embodiment, the genetically controlled structural element is a bacteriophage, the at least one metal interactor is a lipid-binding protein, preferably a fatty-acid binding protein, and the genetically controlled nanoscopy contrast-generating unit additionally comprises a kinase, IMEF, or a tyrosinase.

The genetically controlled structural element may comprise an engineered mono-to-multivalent hub. Such engineered mono-to-multivalent hub is, for instance, a SunTag, an anti-moon tag, or CCC-tag, preferably an emFP (SEQ ID NOs: 92-99). In one embodiment, the genetically controlled structural element is an engineered mono-to-multivalent hub. In another embodiment, the genetically controlled structural element is an engineered mono-to-multivalent hub further comprising a genetically controlled contrast-generating unit, wherein said genetically controlled contrast-generating unit is B-FABP (SEQ ID NO: 36), B-FABP-ko (SEQ ID NO: 248), Bm3H (SEQ ID NO: 29), or Bm3H-B7 (SEQ ID NO: 247). In a preferred embodiment, the genetically controlled structural element is an anti-moon tag, preferably one to twenty anti-moon tags, more preferably one to fifteen anti-moon tags, more preferably one to ten anti-moon tags. In another embodiment, the genetically controlled structural element is one to ten anti-moon tags and the genetically controlled structural element further comprises a genetically controlled contrast-generating unit, wherein said genetically controlled contrast-generating unit is B-FABP. In a preferred embodiment, the genetically controlled structural element is one anti-moon tags and the genetically controlled structural element further comprises a genetically controlled contrast-generating unit, wherein said genetically controlled contrast-generating unit is B-FABP In (SEQ ID NO: 259). In yet another embodiment, the genetically controlled structural element comprises at least one mono-to-multivalent hub and at least one metal interactor by the organization of the genetically controlled nanoscopy contrast-generating unit.

The genetically controlled structural element may comprise one or more filaments. In a preferred embodiment, the one or more filament is DHF 119. In another preferred embodiment, the genetically controlled structural element organizing the genetically controlled nanoscopy contrast-generating unit is a fusion protein comprising at least one filament and at least one metal interactor. In a preferred embodiment, the metal interactor is MT3.

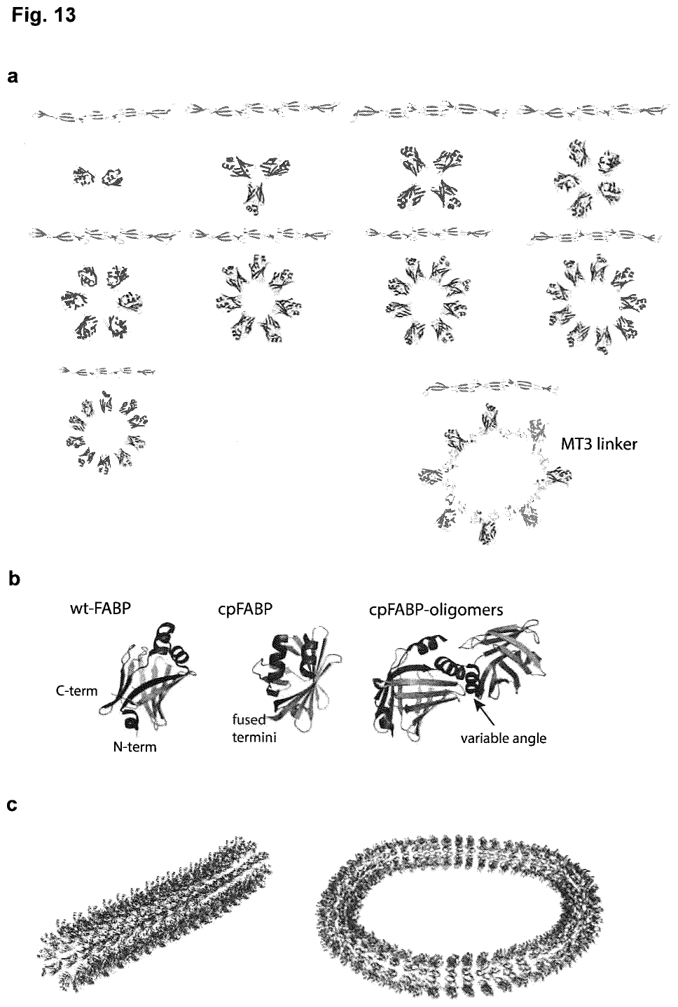

The minimal structural element can thus be constructed from just linkers, such as a GGSG linker (SEQ ID NO:237) that connect individual CGUs such as FABPs (FIG. 13).

The genetically controlled structural element may comprise at least one or more fibers. In a preferred embodiment, the genetically controlled structural element organizing the genetically controlled nanoscopy contrast-generating unit is a fiber. In a preferred embodiment, the at least one or more fibers is an amyloid-like fiber. In an even more preferred embodiment, the amyloid-like fiber is functional amyloid. In a preferred embodiment, the functional amyloid is CsgA, preferably E. coli CsgA. In another preferred embodiment, the CsgA is optimized for intracellular expression, i.e., the CsgA is codon-optimized for mammalian cells. In another embodiment, the fiber is contrast-enhanced by fusing the fiber with, e.g., to a SpyTag. In a preferred embodiment, the genetically controlled structural element organizing the genetically controlled nanoscopy contrast-generating unit is a fusion protein comprising a fiber and at least one metal interactor. In a preferred embodiment, the metal interactor is MT3, a lipid-binding protein, or RNA. In another preferred embodiment, MT3 is fused to the cognate SpyCatcher binding partner.

In another embodiment, the genetically controlled structural element is spaghetti monster fluorescent proteins and variants thereof such as emFPs (SEQ ID NOs: 92-99) into which amino acid sequences are introduced that in turn can be bound by the set of attachment points conjugated to the genetically controlled nanoscopy contrast generating units such as MT3, FABPS, BM3h, RNA, RNA aptamers, metal binders.

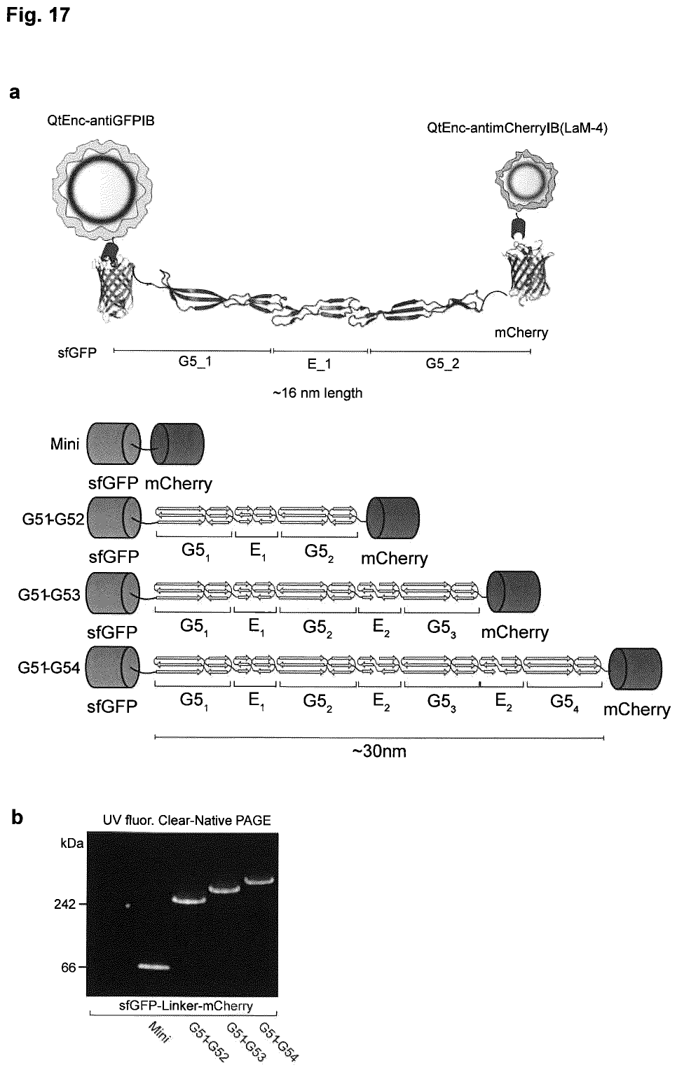

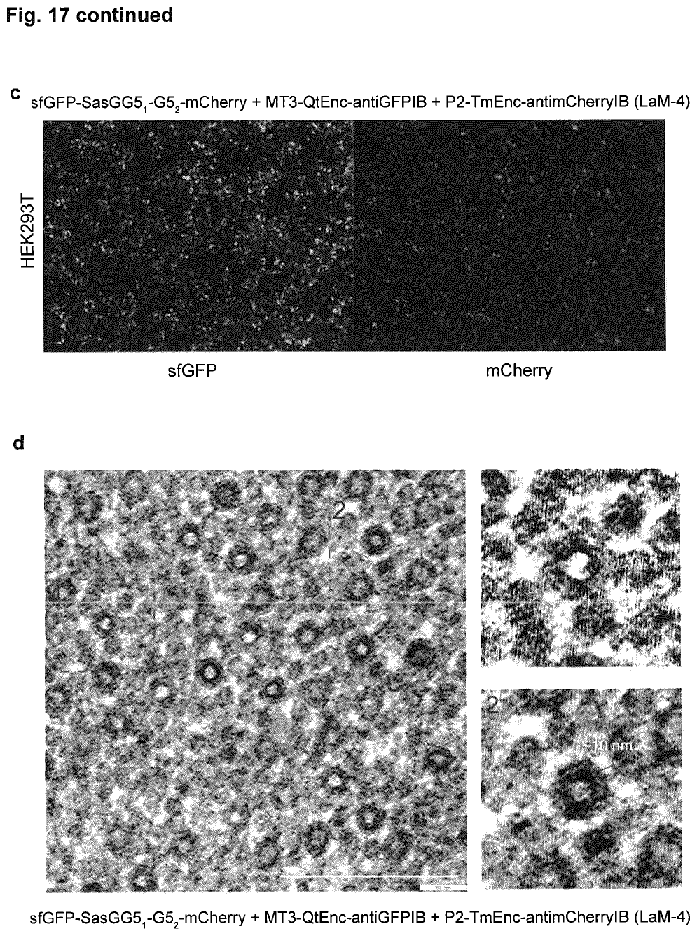

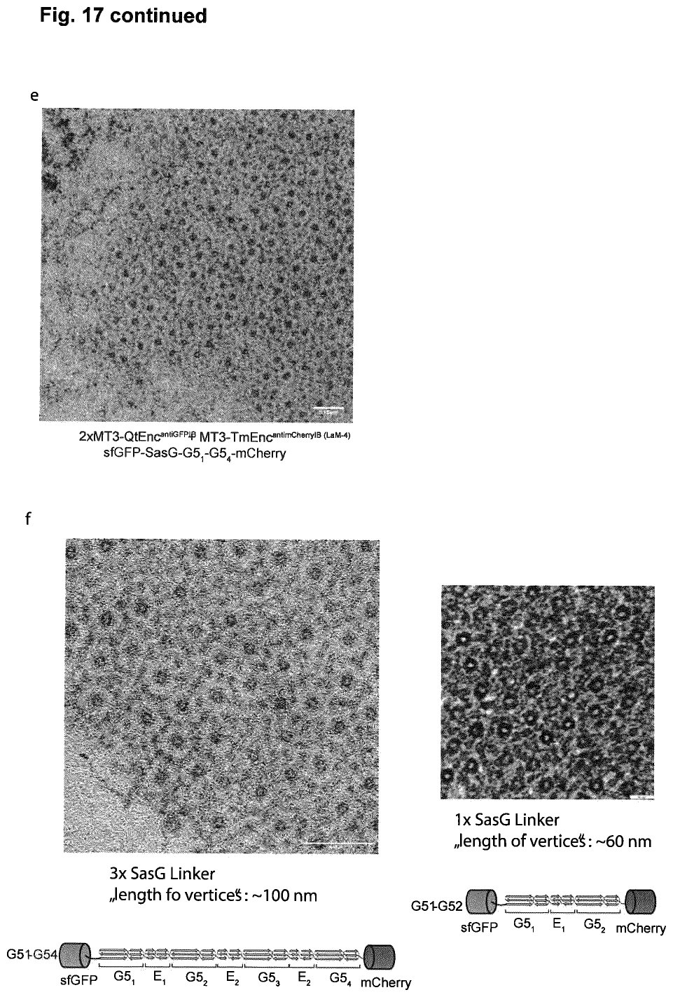

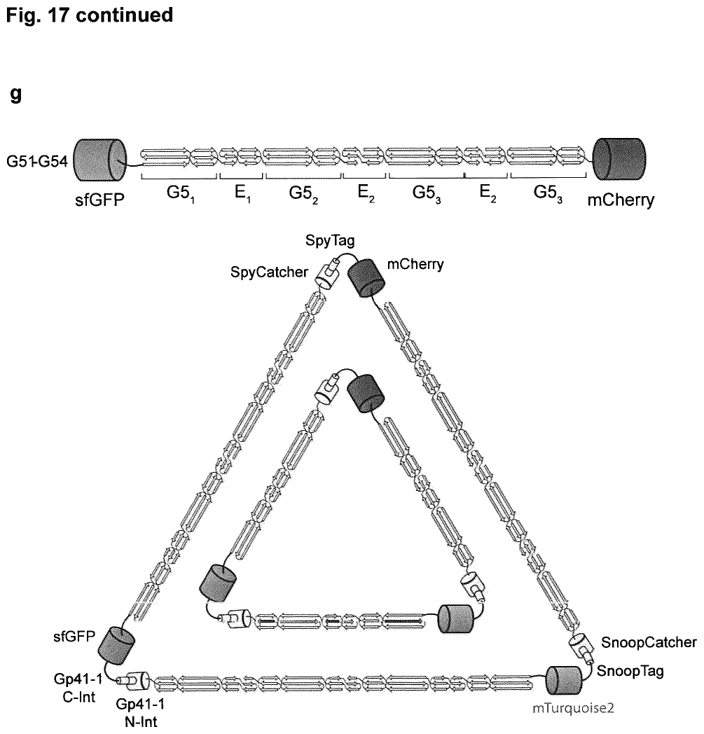

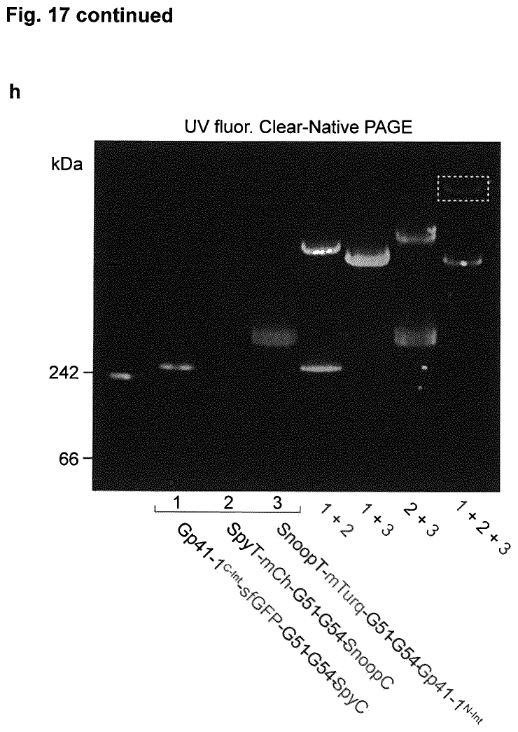

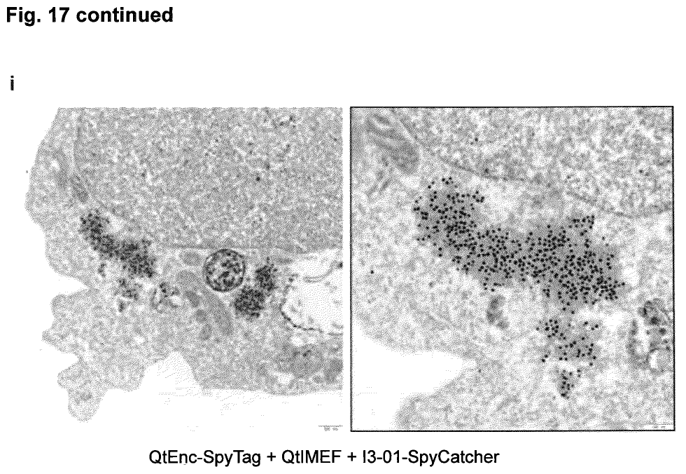

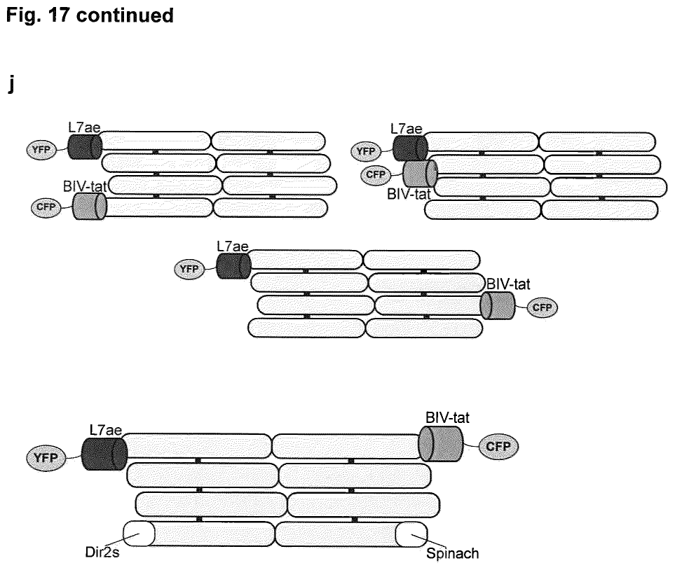

The genetically controlled structural element may be the rigid rod-like protein SasG (SEQ ID NO: 207). In a preferred embodiment, SasG comprises a linear structure composed of concatenated SasG elements. In one preferred embodiment, SasG may contain mono-to-polyvalent hubs such as emFPs SEQ ID NOs: 92-99 instead of the fluorescent proteins, e.g. sfGFP, mTurquoise2, or mCherry (in FIG. 17); alternatively, CGUs such as FABPs directly replace the fluorescent proteins (SEQ ID NO: 30-39). In one embodiment, said concatenable elements provide attachment points to genetically controlled nanoscopy contrast-generating units according to the invention. For example, in one embodiment, the genetically controlled nanoscopy contrast-generating units comprise a metallothionein, a lipid-binding protein, or RNA, and at least one RNA-binding protein.

The genetically controlled structural element may further comprise at least one attachment point. Said at least one attachment point is selected from the group consisting of nanobodies, frankenbodies, coiled-coil domains, isopeptide-forming partners such as SpyTag or SpyCatcher domains, SnoopTag/SnoopCatcher, DogTag/DogCatcher, bioconjugation tags such as SNAP/Clip-Tag domains, and/or Halo-Tag domains, split-inteins, calmodulin-binding peptides, phosphorylatable peptides, conformation-changing peptides such as troponins, as well as combinations thereof. The genetically controlled nanoscopy contrast-generating unit binds to and/or interacts with the structural element via at least one attachment point. In one embodiment, the genetically controlled structural element is an encapsulin from Quasibacillius thermotolerans (Qt), Myxococcus xanthus (Mx), or Thermotoga maritima (Tm), wherein the genetically controlled contrast-generating unit is one or two murine methallothionein, or wherein the genetically controlled contrast-generating unit is a chimeric metallothionein sequence comprising three N-terminally fused metallothioneins comprising murine metallothionein-3 (MmT3), Synechococcus elongatus (SmtA) metallothionein, and Triticum aestivum Ec-1 metallothionein (TaEC1) (SEQ ID NO: 5), wherein the genetically controlled contrast-generating unit comprises an attachment point, wherein said attachment point is a bionjungation tag, wherein said bioconjugation tag is SNAP. In a preferred embodiment, the genetically controlled structural element is a Quasibacillius thermotolerans (Qt) encapsulin, wherein the genetically controlled contrast-generating unit is one murine metallothionein, wherein the genetically controlled contrast-generating unit comprises an attachment point, wherein said attachment point is a bionjungation tag, wherein said bioconjugation tag is SNAP. As laid out above, in other embodiments the fussion protein is eUnaG-MT3-QtEnc^{FLAG} or eUnaG-2xMT3-QtEnc^{FLAG} and the genetically controlled structural element further comprises at least one attachment point, wherein the at least one attachment point is an intrabody, wherein said intrabody is C-terminally fused. In one embodiment, the intrabody is an anti-mCherry intrabody. In another embodiment, the anti-mCherry intrabody is expressed on the outer surface of the encapsulin. In another embodiment, the genetically controlled contrast-generating unit additionally comprises at least one further attachment point, wherein said attachment point is a coiled-coil domain. In a preferred embodiment, the attachment point is two coiled-coil domains. In yet another embodiment, the genetically controlled contrast-generating unit additionally comprises at least one further attachment point, wherein said attachment point is a SpyTag/SpyCatcher.

In one embodiment, the genetically controlled structural element comprises internal attachment points. Said internal attachment points are selected from the group consisting of nanobodies, frankenbodies, coiled-coil domains, SpyTag or SpyCatcher domains, SNAP/Clip-Tag domains, and/or Halo-Tag domains, split-inteins, calmodulin-binding peptides, phosphorylatable peptides, conformation-changing peptides such as troponins, as well as combinations thereof. In another embodiment, the genetically controlled structural element comprises external attachment points. Said external attachment points are selected from the group consisting of nanobodies, frankenbodies, coiled-coil domains, SpyTag or SpyCatcher domains, SnoopTag/SnoopCatcher, DogTag/DogCatcher, SNAP/Clip-Tag domains, and/or Halo-Tag domains, split-inteins, calmodulin-binding peptides, phosphorylatable peptides, conformation-changing peptides such as troponins, as well as combinations thereof. In yet another embodiment, the genetically controlled structural element comprises internal attachment points and external attachment points. Said internal and external attachment points are selected from the group consisting of nanobodies, frankenbodies, coiled-coil domains, SpyTag or SpyCatcher domains, SnoopTag/SnoopCatcher, DogTag/DogCatcher, SNAP/Clip-Tag domains, and/or Halo-Tag domains, split-inteins, calmodulin-binding peptides, phosphorylatable peptides, conformation-changing peptides such as troponins, as well as combinations thereof. In one embodiment, the genetically controlled nanoscopy contrast-generating unit interacts or binds to at least one external attachment point. In yet another embodiment, the genetically controlled nanoscopy contrast-generating unit can interact or bind to internal and/or external attachment points, whereby the interaction or binding leads to a distinct, pre-defined shape.

In one embodiment, the genetically controlled nanoscopy contrast-generating unit is N-terminally and/or C-terminally fused to the genetically controlled structural element. In a preferred embodiment, the genetically controlled nanoscopy contrast-generating unit is N-terminally fused to the genetically controlled structural element. In an even more preferred embodiment, the genetically controlled nanoscopy contrast-generating unit is MT3 and is N-terminally fused to an encapsulin. In an equally preferred embodiment, MT3 is N-terminally fused to an encapsulin, and an epitope tag is fused to the C-terminus of encapsulin. In one embodiment, the C-terminal epitope tag is a FLAG tag, or BC2 tag.

In one embodiment, the genetically controlled structural element comprises a small fluorescent protein, which provides both high contrast in EM applications and super-resolution in fluorescence applications. In another embodiment, a small fluorescent protein is N-terminally or C-terminally appended to the genetically controlled structural element. In another embodiment, a small fluorescent protein is N-terminally appended to the genetically controlled structural element. In yet another embodiment, the genetically controlled structural element is n=1, 2, 3, 4, 5, 6, 7, 8, 9, or more. In a preferred embodiment, the small fluorescent protein is UnaG, and UnaG is N-terminally appended to the genetically controlled structural element. In another preferred embodiment, the genetically controlled structural element is an encapsulin. In an even more preferred embodiment, UnaG is N-terminally appended to 1x MT3-QtEnc, resulting in UnaG-1xMT3-QtEnc. In an equally preferred embodiment, UnaG is N-terminally appended to 2xMT3-QtEnc, resulting in UnaG-2xMT3-QtEnc.

In one embodiment, the genetically controlled structural element is loaded with protein cargo. In another preferred embodiment, the genetically controlled structural element is loaded with protein cargo and targeted to an intracellular protein target by an intrabody. In a preferred embodiment, the protein cargo is mScarlet. Therefore, in one embodiment, the genetically controlled structural element is a Quasibacillius thermotolerans (Qt) encapsulin, or Thermotoga maritima (Tm) encapsulin further comprising a genetically controlled contrast-generating unit, wherein said genetically controlled contrast-generating unit is one or two murine metallothionein-3, wherein mScarlet-I is encapsulated to the encapsulin lumen via an encapsulation signal. In another embodiment, the genetically controlled structural element is a Quasibacillius thermotolerans (Qt) encapsulin, or Thermotoga maritima (Tm) encapsulin further comprising a genetically controlled contrast-generating unit, wherein said genetically controlled contrast-generating unit is one or two murine metallothionein-3, wherein mScarlet-I is expressed as cargo protein in the encapsulin lumen. In another embodiment, the protein cargo is APEX2 and APEX2 is expressed as cargo protein in the encapsulin lumen by encapsulating APEX2 via an encapsulation signal. In one aspect, an electron microscopy method is provided, wherein the genetically controlled structural element is a Quasibacillius thermotolerans (Qt) encapsulin, or Thermotoga maritima (Tm) encapsulin further comprising a genetically controlled contrast-generating unit, wherein said genetically controlled contrast-generating unit is one or two murine metallothionein-3, wherein APEX2 is expressed as cargo protein in the encapsulin lumen and further incubation steps involving DAB and hydrogen peroxide are performed.

In one embodiment, the intrabody is directly fused to the C-terminus of the genetically controlled structural element. In a preferred embodiment, the genetically controlled structural element is an encapsulin, preferably QtEnc. In an equally preferred embodiment, the intrabody is covalently attached to the genetically controlled structural element by SpyTag-SpyCatcher chemistry, wherein the SpyTag is presented on the exterior of the genetically controlled structural element, and the intrabody is fused to a SpyCatcher domain. In a preferred embodiment, the genetically controlled structural element is an encapsulin, preferably QtEnc. In another preferred embodiment, the intracellular protein target is membrane localized EGFP (EGFP-CAAX) and the intrabody is an anti-GFP intrabody. In yet another preferred embodiment, a cognate coiled-coil (CC) pair is fused to the exterior of the genetically controlled structural element; the cognate CC part P3 is N-terminally appended to the intracellular protein target. In a preferred embodiment, the genetically controlled structural element is an encapsulin, preferably QEnc; the cognate CC part P3 is N-terminally appended to the membrane-localized EGFP (EGFP-CAAX).

In one embodiment, the genetically controlled structural element is detectable by both electron microscopy and fluorescence microscopy techniques. In yet another embodiment, the genetically controlled structural element is compatible with both electron microscopy and fluorescence microscopy techniques.

In one embodiment, the genetically controlled structural element is expressed in a cell of a given tissue. Said tissue is captured on TEM grids or on silica wafers for subsequent analysis by TEM or SEM, allowing the detection of said genetically structural element in the identical cell. In a preferred embodiment, the genetically controlled structural element is QtEnc-FLAG-NLS. In another preferred embodiment, the genetically controlled structural element can be detected by both TEM and SEM.

In another embodiment, the genetically controlled structural element is detected by FIB-SEM (focused ion beam scanning electron microscopy). In yet another embodiment, MT3-QtEnc is detected by FIB-SEM. In a preferred embodiment, MT3-QtEnc is expressed in Drosophila melanogaster neurons and detected by FIB-SEM. In another preferred embodiment, the genetically controlled structural element can be detected by TEM, SEM including FIB-SEM, and fluorescence microscopy.

In one embodiment, the genetically controlled structural element can be directed to the nucleus. In another embodiment, the genetically controlled structural element can be directed to the cytosol. In one embodiment, the genetically controlled structural element is an encapsulin. In a preferred embodiment, the encapsulin is QtEnc. In a preferred embodiment, the genetically controlled structural element is QtEnc N-terminally fused to a fluorescent protein. In an even more preferred embodiment, the genetically controlled structural element is QtEnc, and the N-terminally fused fluorescent protein is miniSOG2. In an equally preferred embodiment, QtEnc is N-terminally fused to miniSOG2, and C-terminally fused to a FLAG epitope, followed by a mammalian NLS (c-myc) or a NES (HIV). In another embodiment, the N-terminally fused fluorescent protein does not compromise the assembly of the genetically controlled structural element. In a preferred embodiment, the N-terminally fused miniSOG2 does not compromise the assembly of QtEnc.

Another preferred embodiment is the CC-MT3-QtEnc construct, for which an exemplary sequence is given in SEQ ID NO: 21, with a CC-binding partner given in SEQ ID NO: 22.

The genetically controlled structural element may be of any combination of the above listed structural elements in any combination with any genetically controlled nanoscopy contrast generating unit disclosed herein. The structural element may comprise one or more contrast generating units comprising one or more of a metal-interacting polypeptide/protein, metal-binding peptide/protein, lipid-binding protein, RNA-binding protein, RNA or DNA molecule, polymerizing or synthesizing enzyme, or bioconjugation tag. This means, all of the above-listed contrast-providing genetically controlled nanoscopy contrast-generating units, can be combined with each other and can be spatially organized in any one or more of the above-listed structural elements. The genetically controlled structural elements and the genetically controlled nanoscopy contrast-generating units disclosed herein are thus to be seen as modules that may be combinable in form of modular combinatorics.

### Genetically Controlled Scaffolds

In yet another aspect, the invention refers to a genetically controlled scaffold. The genetically controlled scaffold comprises and spatially organizes one or more genetically controlled structural elements. Such spatial organization generates distinct geometric patterns that can be distinguished at the nanoscale. Preferably, such distinct geometric patterns encode information on cellular states.

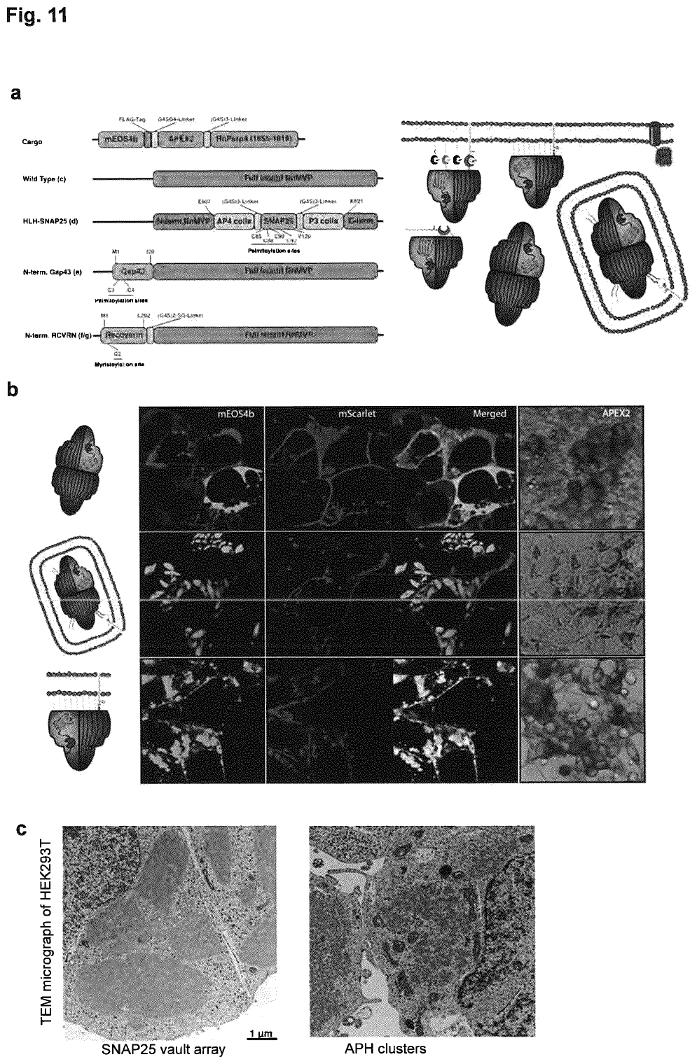

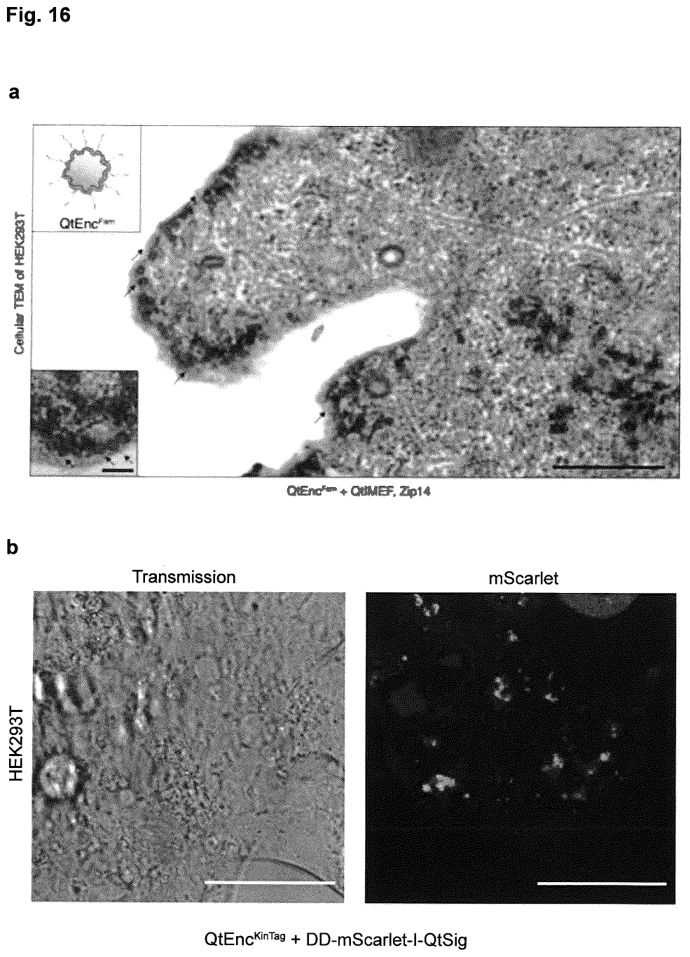

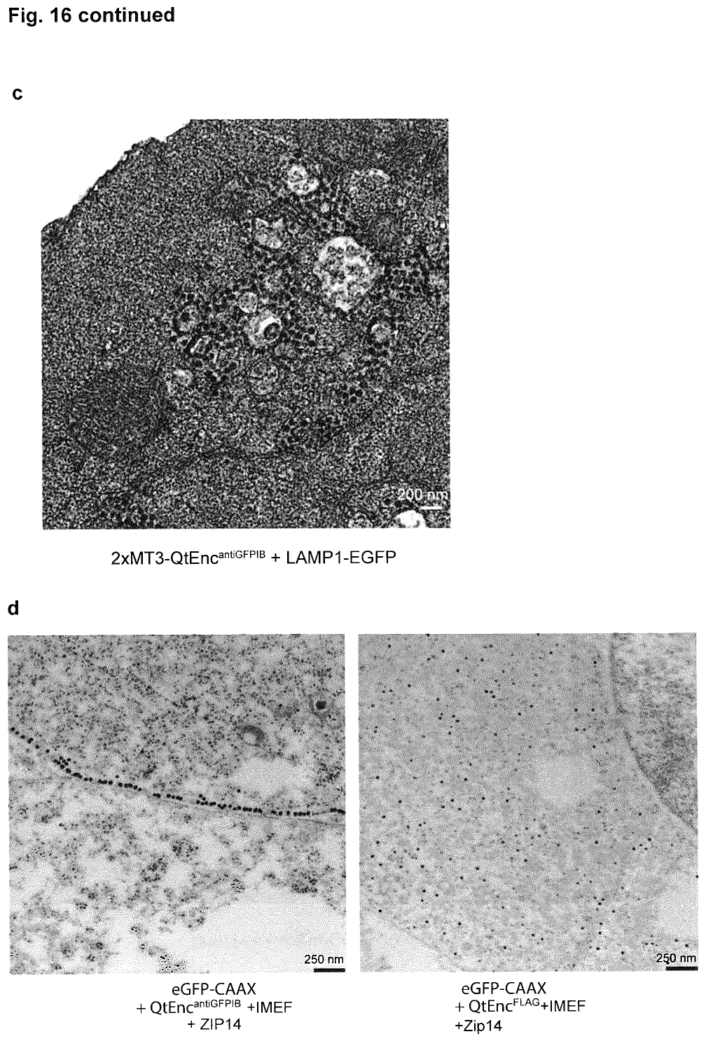

Such generated geometric patterns include vaults arrayed via SNAP25 (FIG. 11), APH or N-terminal IMEF, encapsulins bound to the membrane via farnesylation tags, or via membrane-localized GPF targets (FIG. 16), which can also be used to localize encapsulins to other organelles such as lysosome, multiple encapuslins organized via SasG or RNA as scaffolds (FIG. 17).

In one embodiment, the information on cellular states is information in relation to external stimuli, for example, environmental parameters such as pH, temperature, or, influx, or efflux of ions such as calcium, response to ions such as calcium, proteolytic activity, conformational changes, or receptor densities. In another embodiment, the information on cellular states can for example identify a normal, i.e., healthy cell from a diseased cell in a pathological state. In yet another embodiment, the information on cellular states can identify diseases such as neurological diseases or cancer.

The genetically controlled scaffold may be selected from the group consisting of an endogenous cellular scaffold, a de novo designed scaffold, one or more SasG elements (SEQ ID NO: 112), CsgA (SEQ ID NO: 112), RNA or DNA structures, or combinations thereof.

The genetically controlled scaffold interacts or binds to the genetically controlled structural elements and, therefore, organizes the structural element. In one embodiment, the genetically controlled structural element comprises a member of the encapsulin family, a vault, MS2 phage, Qbeta phage, AP205 phage, mono-to-multivalent hubs, a filament, a linker, or combinations or variants thereof. In a preferred embodiment, the genetically controlled structural element is modified at the C- or N-terminus, suitable modifications are, for example, myristoylation, palmitoylation, isoprenylation, or prenylation such as farnesylation and gernaylgeranylation, or glypation. In another preferred embodiment, the genetically controlled structural element is modified by C-terminal farnesylation. By said C-terminal farnesylation the genetically controlled structural element is targeted to the membranes of endogenous, i.e., cellular scaffolds.

In an embodiment, the genetically controlled scaffolds are endogenous cellular scaffolds, such as cellular membranes of organelles, i.e., the nucleus, endoplasmic reticulum, Golgi apparatus, mitochondria, plastids, lysosomes, the inner cell membrane, and vacuoles. In yet another embodiment, the endogenous cellular scaffold is the cytoskeleton, i.e, actin filaments and/or microtubuli.

In another embodiment, the genetically controlled scaffold is an exogenous scaffold, such as a de novo designed scaffold. In one embodiment, the de novo designed scaffold is selected from the group consisting of, for example, filaments such as DHF or designed RNA structures. In a preferred embodiment, the de novo designed scaffold is DHF or 13-01, a de novo designed icosahedron comprising 60 cognate SpyCatcher binding partners.

In one preferred embodiment, the targeted controlled structural element is an encapsulin. In a preferred embodiment, the encapsulin is QtEnc, MxEnc, or TmEnc. In an even more preferred embodiment, the genetically controlled structural element is an iron-mineralizing QtEnc C-terminally modified with a SpyTag, wherein said iron-mineralizing QtEnc is C-terminally modified with a SpyTag covalently clustered with 13-01. The iron-mineralized C-terminally modified QtEnc with a SpyTag that is covalently clustered with 13-01 leads to unbounded clusters. In said embodiment, the genetically controlled scaffold is geometrically unbounded. In a preferred embodiment, the genetically controlled scaffold arranges or organizes an encapsulin, even more preferably QtEnc, even more preferably a modified QtEnc comprising an antiGFP antibody and/or TmEnc with an anti-mCherry intrabody. In said embodiment, the genetically controlled scaffold is geometrically unbounded.

In yet another embodiment, the scaffold is a linear scaffold, such as a linear protein structure such as SasG. In even another embodiment, the scaffold is a concatenable linear protein. In a preferred embodiment, the linear scaffold is a bacterial surface protein, even more preferred SasG from S. aureus (SEQ ID NO: 207). In another embodiment, the SasG C-terminus and N-terminus of the genetically controlled structural element are fused to fluorescent proteins, wherein the C-terminus and N-terminus are fused to sfGFR and mCherry, respectively, or wherein the N-terminus and C-terminus are fused sfGFR and mCherry.

The spatial organization of the genetically controlled structural elements by the genetically controlled scaffold yields distinct patterns. In one embodiment, the genetically controlled scaffold yielding distinct patterns is SasG. In a preferred embodiment, the genetically controlled scaffold yielding patterns is concatenated SasG. In another embodiment, a pattern is formed by distinct pre-defined spacers and attachment points within SasG. In another embodiment, the SasG spaces and attachment points are fully controllable such that the resulting SasG pattern is a fully-genetically defined linear barcode, i.e., each genetic element on the DNA and RNA has its corresponding linear element on the protein level, whereby a geometrically defined pattern is formed. In yet another embodiment, the geometrical pattern is bounded. In one embodiment, the genetically controlled structural element is an encapsulin, or ferritin (iron-mineralizing QtEnc C-terminally modified with a SpyTag, wherein in said iron-mineralizing QtEnc is C-terminally modified with a SpyTag covalently clustered with SasG) and the genetically controlled scaffold is SasG. In another embodiment, a pattern is formed by SasG in a linear assembly, i.e., without one or more structural elements.

In a specifically preferred embodiment, the genetically controlled scaffold is SasG of variable lengths, wherein the SasG comprises and spatially organizes encapsulins as structural elements, wherein the encapsulins comprise and spatially organize a genetically controlled nanoscale contrast-generating unit comprising one or more MT3 domains or a series of different metallothionein species (SEQ ID NO: 169) as the metal interactor. In one embodiment, the genetically controlled scaffold comprises SasG having 2, 3, 4, 5, 6, 7, 8, 9 or 10 G5 domains connected by E domains, one encapsulin from Quasibacillius thermotolerans (Qt), Myxococcus xanthus (Mx), or Thermotoga maritima (Tm) as genetically controlled structural element, and one or more murine metallothionein-3 as genetically controlled nanoscopy contrast-generating unit. In another embodiment, the genetically controlled scaffold comprises SasG having 2, 3, 4, 5, 6, 7, 8, 9 or 10 G5 domains connected by E domains, an encapsulin selected from the group consisting of Quasibacillius thermotolerans (Qt), Myxococcus xanthus (Mx), or Thermotoga maritima (Tm) as genetically controlled structural element, and one or two murine metallothionein-3 as genetically controlled nanoscopy contrast-generating unit. In yet another embodiment, the genetically controlled scaffold comprises SasG having 2, 3, 4, 5, 6, 7, 8, 9 or 10 G5 domains connected by E domains, said SasG having N-terminally fused sfGFP and C-terminally fused mCherry, one encapsulin from Quasibacillius thermotolerans (Qt), Myxococcus xanthus (Mx), or Thermotoga maritima (Tm) as genetically controlled structural element, and one or two murine metallothionein-3 as genetically controlled nanoscopy contrast-generating unit. In even another embodiment, the genetically controlled scaffold comprises a SasG having 2, 3, 4, 5, 6, 7, 8, 9 or 10 G5 domains connected by E domains, said SasG having N-terminally fused sfGFP and C-terminally fused mCherry, one encapsulin selected from the group consisting of Quasibacillius thermotolerans (Qt) or Thermotoga maritima (Tm) as genetically controlled structural element, and one murine metallothionein-3 as genetically controlled nanoscopy contrast-generating unit.

In another embodiment, the genetically controlled scaffold comprises SasG having 2, 3, 4, 5, 6, 7, 8, or more G5 domains connected by E domains, said SasG having N-terminally fused sfGFP and C-terminally fused mCherry, one Quasibacillius thermotolerans (Qt) encapsulin as genetically controlled structural element having one murine metallothionein as genetically controlled nanoscopy contrast-generating unit, wherein said Qt encapsulin has one or more anti-GFP intrabody on its surface. In a further embodiment, the genetically controlled scaffold comprises SasG having 2, 3, 4, 5, 6, 7, 8, or more G5 domains connected by E domains, said SasG having N-terminally fused sfGFP and C-terminally fused mCherry, one Thermotoga maritima (Tm) encapsulin as genetically controlled structural element having one murine metallothionein as genetically controlled nanoscopy contrast-generating unit, wherein the Tm encapsulin has one or more anti-mCherry intrabodies on its surface.

In a further embodiment, the genetically controlled scaffold comprises SasG having 2, 3, 4, 5, 6, 7, 8, or more G5 domains connected by E domains, said SasG having N-terminally fused sfGFP and C-terminally fused mCherry, one Quasibacillius thermotolerans (Qt) encapsulin as genetically controlled structural element having one murine metallothionein as genetically controlled nanoscopy contrast-generating unit, wherein said Qt encapsulin has one or more anti-GFP intrabody on its surface, and one Thermotoga maritima (Tm) encapsulin as genetically controlled structural element having one murine metallothionein as genetically controlled nanoscopy contrast-generating unit, wherein the Tm encapsulin has one or more anti-mCherry intrabody on its surface. In one embodiment, the Qt encapsulin having one or more anti-GFP intrabody on its surface is linked to the matching anti-mCherry intrabody on the surface of the Tm encapsulin. In another embodiment, said Qt encapsulin having one or more anti-GFP intrabody on its surface results in an approximately 40 nm ring-shaped object, wherein the Tm encapsulin having one or more matching anti-mCherry intrabody on the surface results in an approximately 25 nm spherical object, wherein said spherical object surrounds said ring-shaped object.

In an embodiment, the genetically controlled scaffold is formed by amyloid curly fibrils such as CsgA from E. coli providing attachment points.

In another embodiment, the genetically controlled scaffold is designed from RNA origami structures that folds into rigid assemblies that can span tens of nanometers. In a preferred embodiment, said RNA origami structures contain aptamer loops such as PP7 or MS2 to which proteinaceous contrast-generating units can be attached. In another preferred embodiment, said RNA origami structures contain aptamers for exogenous fluorophores, such as Spinach. In another preferred embodiment, the aptamer structures are circularized via ligase RtcB via the Tornado system to stabilize them in mammalian cells.

In one embodiment, the genetically controlled scaffold comprises at least one attachment point. Said at least one attachment point is selected from the group consisting of nanobodies, frankenbodies, coiled-coil domains, isopeptide-forming partners such as SpyTag or SpyCatcher domains, SnoopTag/SnoopCatcher, DogTag/DogCatcher, bioconjugation tags such as SNAP/Clip-Tag domains, and/or Halo-Tag domains, split-inteins, calmodulin-binding peptides, phosphorylatable peptides, conformation-changing peptides such as troponins, as well as combinations thereof. In one embodiment, the genetically controlled scaffold comprises internal attachment points. Said internal attachment points are selected from the group consisting of nanobodies, frankenbodies, coiled-coil domains, isopeptide-forming partners such as SpyTag or SpyCatcher domains, SnoopTag/SnoopCatcher, DogTag/DogCatcher, bioconjugation tags such as SNAP/Clip-Tag domains, and/or Halo-Tag domains, split-inteins, calmodulin-binding peptides, phosphorylatable peptides, conformation-changing peptides such as troponins, as well as combinations thereof. In another embodiment, the genetically controlled scaffold comprises external attachment points. Said external attachment points are selected from the group consisting of nanobodies, frankenbodies, coiled-coil domains, isopeptide-forming partners such as SpyTag or SpyCatcher domains, SnoopTag/SnoopCatcher, DogTag/DogCatcher, bioconjugation tags for covalent protein labeling such as SNAP/Clip-Tag domains, and/or Halo-Tag domains, split-inteins, calmodulin-binding peptides, phosphorylatable peptides, conformation-changing peptides such as troponins, as well as combinations thereof. In yet another embodiment, the genetically controlled scaffold comprises internal attachment points and external attachment points. Said internal and external attachment points are selected from the group consisting of nanobodies, frankenbodies, coiled-coil domains, isopeptide-forming partners such as SpyTag or SpyCatcher domains, SnoopTag/SnoopCatcher, DogTag/DogCatcher, bioconjugation tags for covalent protein labeling such as SNAP/Clip-Tag domains, and/or Halo-Tag domains, split-inteins, calmodulin-binding peptides, phosphorylatable peptides, conformation-changing peptides such as troponins, as well as combinations thereof. In one embodiment, the genetically controlled scaffold interacts or binds to at least one external attachment point of a structural element.

In one embodiment, the genetically controlled structural elements comprise external attachment points. In a preferred embodiment, the external attachment points are monovalent. In one embodiment, the genetically controlled scaffold is SasG and the genetically controlled structural element is an encapsulin and comprises at least one attachment point, wherein the attachment point is selected from the group consisting of nanobodies, frankenbodies, coiled-coil domains, isopeptide-forming partners such as SpyTag or SpyCatcher domains, SnoopTag/SnoopCatcher, DogTag/DogCatcher, bioconjugation tags for covalent protein labeling such as SNAP/Clip-Tag domains, and/or Halo-Tag domains, split-inteins, calmodulin-binding peptides, phosphorylatable peptides, conformation-changing peptides such as troponins, as well as combinations thereof. In one preferred embodiment, the genetically controlled scaffold is SasG, and the genetically controlled structural element is an encapsulin and comprises at least one fluorescent molecule. In another embodiment, the genetically controlled scaffold is SasG, and the genetically controlled structural element is an encapsulin and comprises two fluorescent molecules such as sfGFP and mCherry. In another embodiment, the genetically controlled scaffold, the genetically controlled structural element, and the two fluorescent molecules are detectable by both electron microscopy and fluorescence microscopy techniques. In yet another embodiment, the genetically controlled scaffold, the genetically controlled structural element, and the two fluorescent molecules are compatible with both electron microscopy and fluorescence microscopy techniques. In one embodiment, SasG fusion proteins, such as sfGFP-SasG-mCherry to which structural elements can bind via bioorthogonal intrabodies to the fluorescent proteins. In a preferred embodiment, the SasG proteins form geometric patterns by connecting the structural elements QtEnc of about 40 nm in diameter with the structural elements TmEnc of about 20 nm diameter. The resulting pattern comprises QtEnc surrounded by TmEnc, wherein the low-contrast space between both structural elements is determined by the length of the rigid SasG linker. In another embodiment, the fusion proteins QtEnc^{antiGFPib} and TmEnc-LaM-4 are disclosed, also resulting in distinct patterns.

In a preferred embodiment, the SasG-organized patterns of multiple encapsulins as structural elements are modularly combined with the concentric barcodes generated inside the lumen of each structural element.

The genetically controlled scaffold may be of any combination of the above-listed scaffolds in any combination with any genetically controlled structural element and any genetically controlled nanoscopy contrast generating unit disclosed herein. The scaffold may comprise at least one structural element, wherein each structural element may comprise one or more contrast generating units comprising one or more of a metal-interacting polypeptide/protein, metal-binding peptide/protein, lipid-binding protein, RNA-binding protein, RNA or DNA molecule, polymerizing or synthesizing enzyme, or bioconjugation tag. This means, all of the above-listed contrast-providing genetically controlled nanoscopy contrast-generating units, can be combined with each other and can be spatially organized in any one or more of the above listed structural elements, wherein all such combined structural elements can be combined with each other and spatially organized by any of the disclosed scaffolds. The genetically controlled scaffolds, genetically controlled structural elements, and genetically controlled nanoscopy contrast-generating units disclosed herein are thus to be seen as modules, which may be combinable in form of modular combinatorics.

### Use of the Invention

In another aspect, the present invention relates to the use of a genetically controlled structural element, and/or a genetically controlled scaffold for nanoscopy detection, wherein nanoscopy detection comprises molecular mapping and/or geometric sensing. In certain embodiments, the nanoscopy detection further comprises geometric actuation.

Molecular mapping and/or geometric sensing is preferably performed in cells, organoids, tissue, or model organisms. In one embodiment, the cell is an animal cell selected from the group consisting of the genera Homo, Rattus, Mus, Sus, Bos, Danio, Canis, Felis, Equus, Salmo, Oncorhynchus, Gallus, Meleagris, Drosophila, or Caenorhabditis. In a preferred embodiment, the animal cell can be of the species Homo sapiens, Rattus norvegicus, Mus musculus, Sus scrofa, Bos taurus, Danio rerio, Danionella translucida, Salmo salar, Canis lupus, Felis catus, Equus caballus, Oncorhynchus mykiss, Gallus, Meleagris gallopavo, Drosophila melanogaster or Caenorhabditis elegans. The animal cell can be from a cow, pig, sheep, goat, bison, horse, donkey, mule, rabbit, chicken, duck, goose, turkey, or pigeon. In a particularly preferred embodiment, the animal cell is a mammalian cell and is selected from the group consisting of neuronal cells, glial cells, cardiomyocytes, glucose-responsive insulin-secreting pancreatic beta cells, hepatocytes, astrocytes, oligodendrocytes, chondrocytes, osteoblasts, retinal pigment epithelial cells, fibroblasts, keratinocytes, dendritic cells, hair follicle cells, renal duct epithelial cells, vascular endothelial cells, testicular progenitors, smooth and skeletal muscle cells, hematopoietic cells, cartilage cells or epidermal cells as well as insulin-secreting p-cells, embryonic stem cells, and mesenchymal cells. In an even more preferred embodiment, the mammalian cell is a human cell and is selected from the group consisting of neuronal cells, glial cells, cardiomyocytes, glucose-responsive insulin-secreting pancreatic beta cells, hepatocytes, astrocytes, oligodendrocytes, chondrocytes, osteoblasts, retinal pigment epithelial cells, fibroblasts, keratinocytes, dendritic cells, hair follicle cells, renal duct epithelial cells, vascular endothelial cells, testicular progenitors, smooth and skeletal muscle cells, hematopoietic cells, cartilage cells or epidermal cells as well as insulin-secreting p-cells, embryonic stem cells, and mesenchymal cells.

In another embodiment, the mapping and/or sensing is performed in organoids or tissues. In yet another embodiment, the mapping and/or sensing is performed in model organisms. In one embodiment, the model organism is, for example, C. elegans, Drosophila melanogaster, Danio rerio, Danionella translucida, Mus musculus, Taeniopygia guttata.

The genetically controlled structural element, and/or a genetically controlled scaffold may be used for nanoscopy detection to read-out multiplexed information on the promoter activity and gene expression state of cells. The genetically controlled structural element, and/or a genetically controlled scaffold may furthermore provide information about diffusion constants in different cells and tissues. The genetically controlled structural element, and/or a genetically controlled scaffold may furthermore be used as fiducial markers, throughout entire cells to improve alignment of tilt series, perform drift corrections, or serve as geometrically defined reference markers for image quality assessment, such as point-spread functions or contrast-to-noise-ratios.

The genetically controlled structural element, and/or a genetically controlled scaffold may be used for nanoscopy detection, wherein nanoscopy detection comprises molecular mapping. In this case, the molecular mapping provides information on the subcellular distribution of a molecule of interest within a cell or tissue via the binding of genetically controlled structural elements and/or genetically controlled scaffolds to a molecule of interest which thus determines the subcellular localization of the genetically controlled structural element and/or the genetically controlled scaffolds. In some embodiments, the nanoscopy detection is electron microscopy detection, such as scanning electron microscopy (SEM), transmission electron microscopy (TEM) and Focused Ion Beam Scanning Electron Microscopy (FIB-SEM). In one embodiment, no further incubations teps involving DAB and hydrogen peroxide or further exogenous substrates are required. In an alternative embodiment, further incubation steps involving DAB and hydrogen peroxide can be used, if for example, APEX2 is used as a cargo protein inside the lumen of the genetically controlled structural element such as an encapsulin.

In another embodiment, the information on the subcellular distribution of a molecule of interest within a cell or tissue is provided by the binding to a molecule of interest by the genetically controlled structural element comprising at least one genetically controlled nanoscopy contrast-generating unit, and/or the genetically controlled scaffolds. The molecule of interest may be localized in the lysosomes and endosomes, mitochondria, nucleus, plasma membrane, endoplasmatic reticulum, Golgi apparatus, and/or cytosol. In one embodiment, the genetically controlled structural element and/or the genetically controlled scaffold are targeted to one or more of the subcellular compartments. In another embodiment, the molecule of interest within a cell or a tissue is, for example, a protein, lipid, or carbohydrate. In a preferred embodiment, the molecule of interest is a functionally relevant epitope on organelles, a receptor or channel in a membrane.

In one embodiment, the genetically controlled structural element and/or the genetically controlled scaffold is targeted to an intracellular or extracellular protein target. In a preferred embodiment, the genetically controlled structural element is targeted to an intracellular protein target. In one embodiment, the genetically controlled structural element is targeted to the intracellular protein target by an intrabody. In a preferred embodiment, the genetically controlled structural element is an encapsulin, preferably QtEnc. In one embodiment, the intrabody is directly fused to the C-terminus of encapsulin as the genetically controlled structural element. In another preferred embodiment, the intracellular protein target is membrane localized EGFP (EGFP-CAAX) and the intrabody is an anti-GFP intrabody. In an equally preferred embodiment, the intrabody is covalently attached to the genetically controlled structural element by SpyTag-SpyCatcher chemistry, wherein the SpyTag is presented on the exterior of the genetically controlled structural element, and the intrabody is fused to a SpyCatcher domain. In a preferred embodiment, the genetically controlled structural element is an encapsulin, preferably QtEnc. In yet another equally preferred embodiment, a cognate coiled-coil pair is fused to the genetically controlled structural element and the respective cellular target. In a preferred embodiment, the genetically controlled structural element is an encapsulin, and the cellular target is an intracellular protein target. In one particularly preferred embodiment, the cognate coiled-coil pair is fused to the exterior of QtEnc; the cognate CC part P3 is N-terminally appended to the membrane-localized EGFP (EGFP-CAAX).

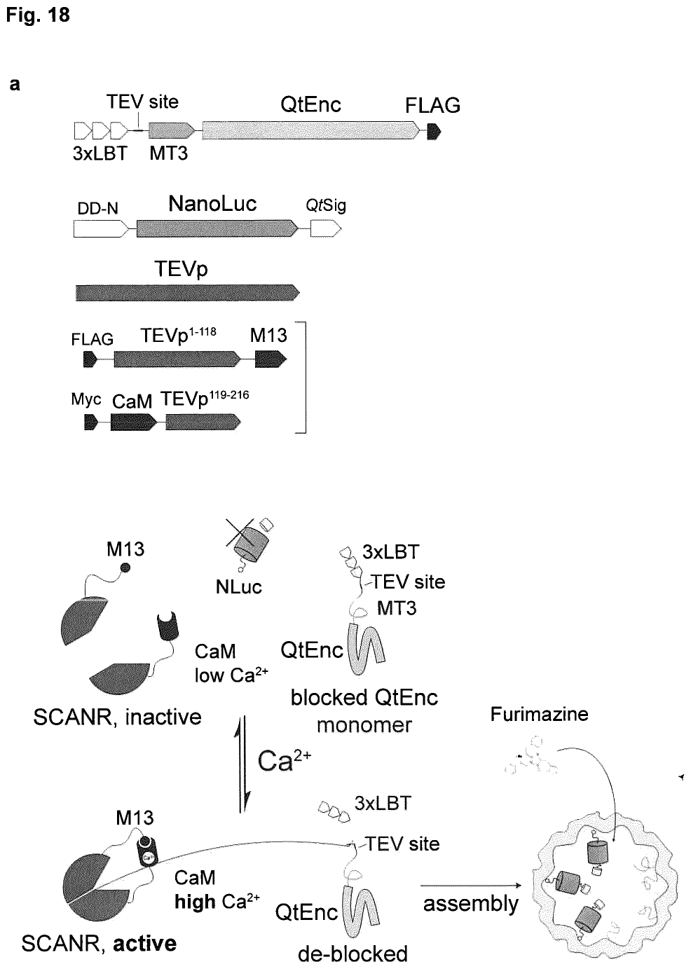

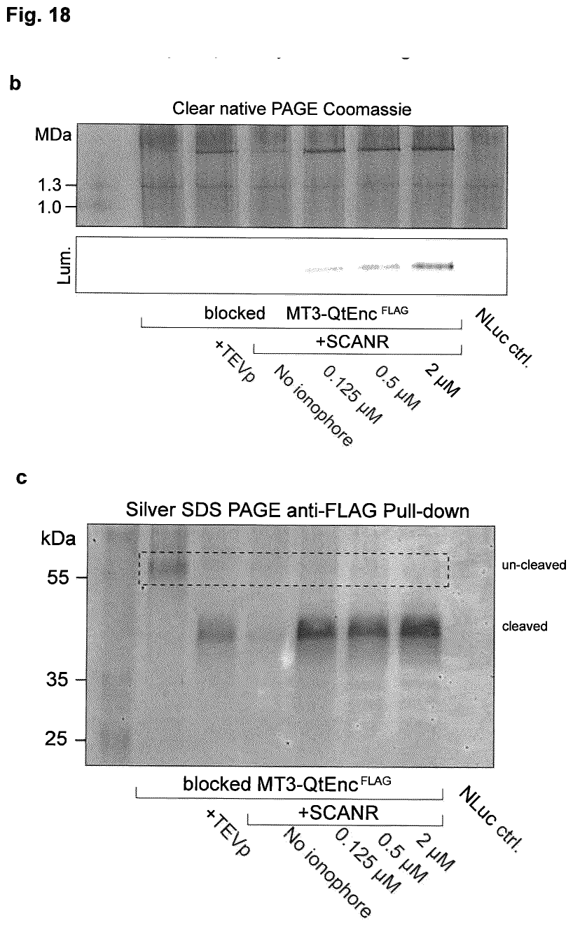

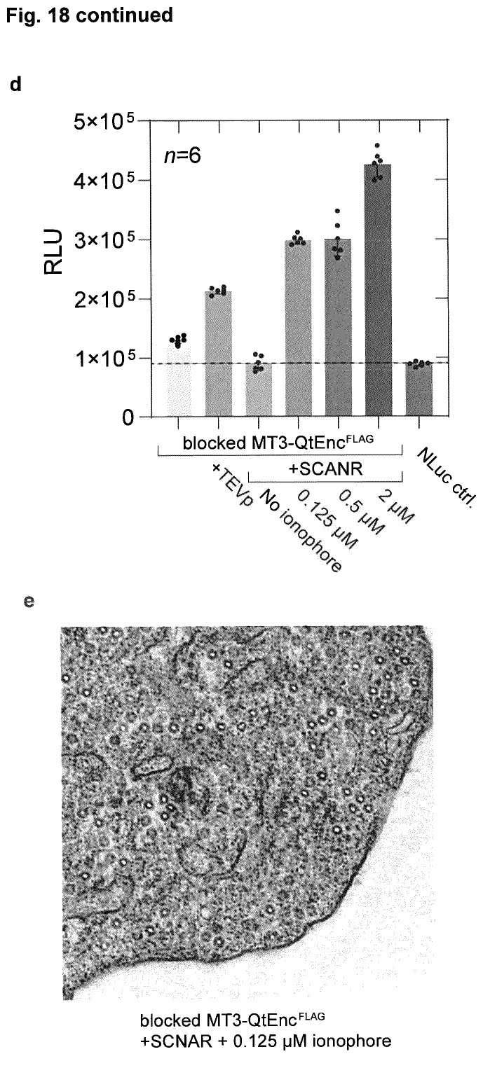

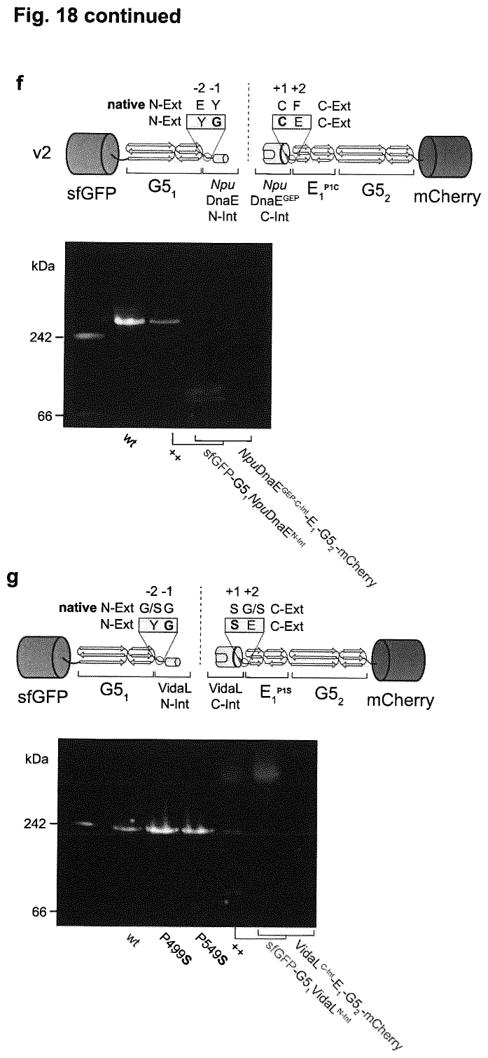

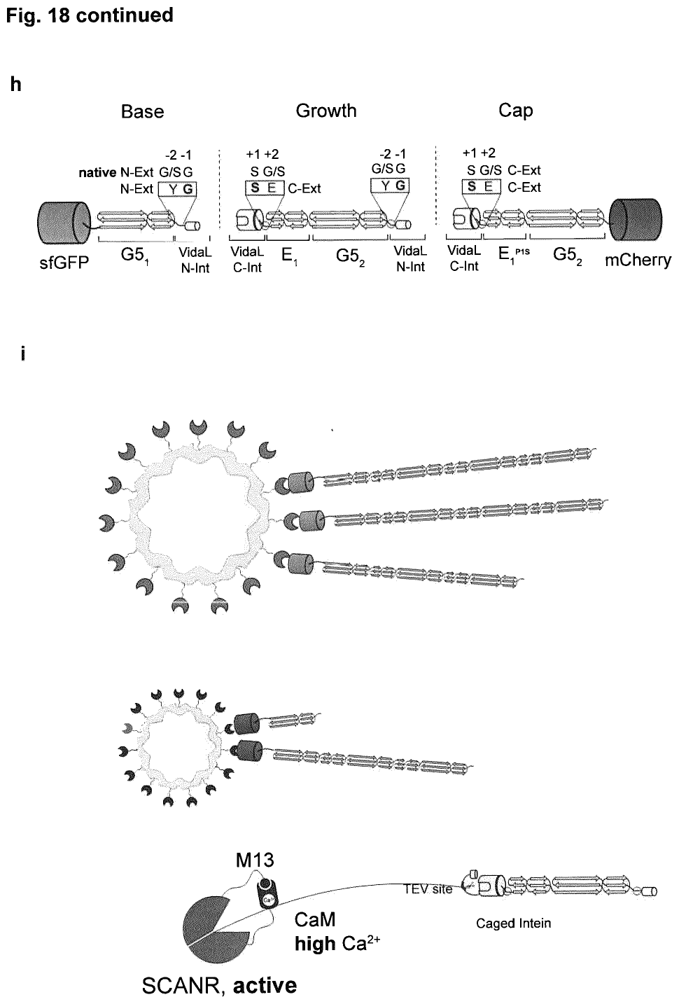

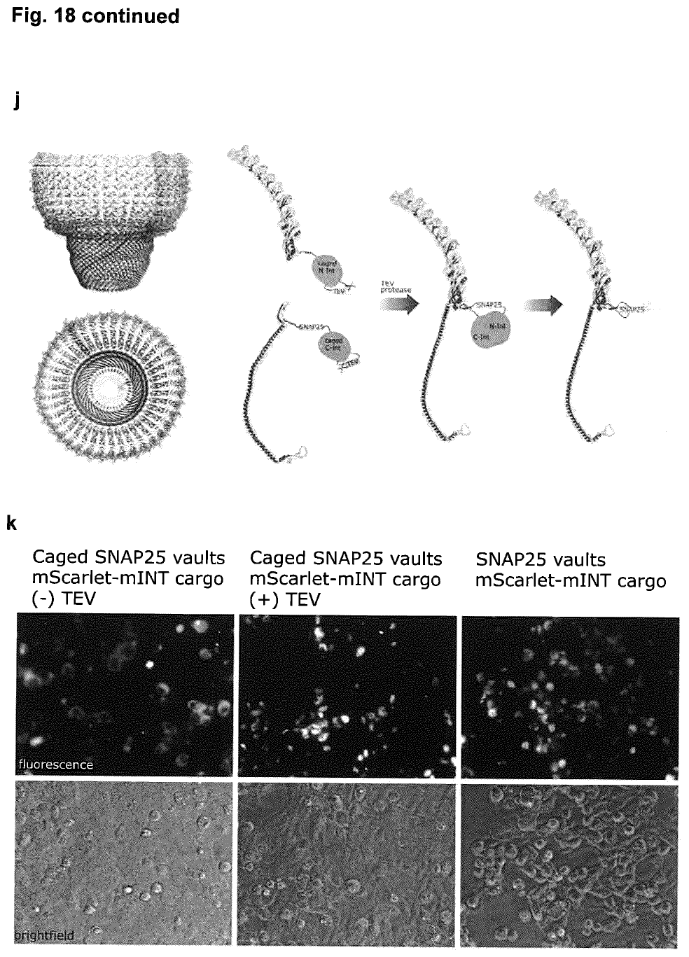

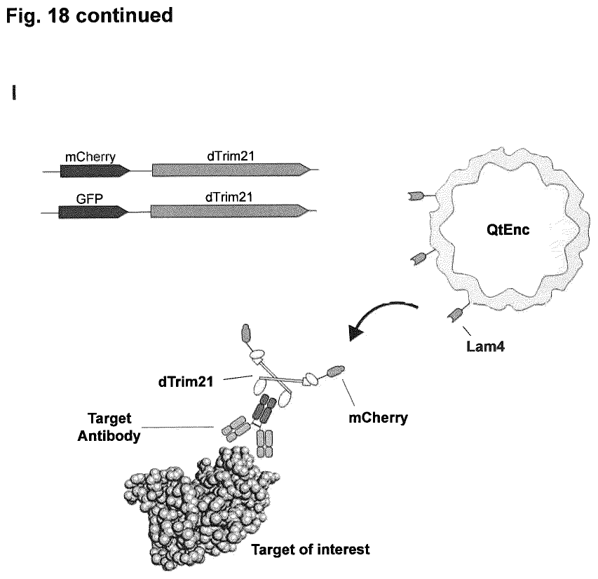

In another preferred embodiment, molecular mapping occurs via a heterobifunctional adapter made from inactivated Trim21 (dTrim21) fused to a fluorescent protein to which intrabodies fused to encapsulins as structural elements can bind (see, e.g., FIG. 18).

Alternatively, or in addition to the molecular mapping, the genetically controlled structural element, and/or a genetically controlled scaffold may be used for nanoscopy detection, wherein nanoscopy detection comprises geometric sensing. In this case, geometric sensing provides information on a specific cellular state, a cellular process, or the presence or absence of an analyte or environmental parameter of interest within a cell or tissue, or the response to an external stimulus. Such sensing is achieved in that the distinct shape generated by the genetically controlled structural element, and/or the geometric pattern generated by the genetically controlled scaffold changes in response to a pre-defined cellular state, a pre-defined cellular process, at least one analyte or at least one environmental parameter, or an external stimulus. Alternatively, a change in a distinct shape and/or distinct pattern is only generated if such analyte, environmental parameter, or external stimulus is present in sufficient strength, or only in response to specific cellular states or processes.

The change in shape occurs by a spatial rearrangement of the contrast-generating units on a structural element or via the rearrangement of the structural elements on a scaffold. This may occur by a conformational change in the scaffold, the structural element, or the contrast-generating unit.

A specific cellular state is, for instance, a specific constellation of promoter activities, the influx or efflux of ions such as calcium, response to ions such as calcium, proteolytic activity, conformational changes, or receptor densities, a response of a cell, a therapeutic response of the cell and activation of the cell. A specific cellular process is, for instance, protein expression and transport, or mitochondrial fusion or fission. An exemplary analyte is, for instance, calcium or cAMP. An external stimulus is, for instance, a depolarization or the application of electromagnetic or mechanical energy or magnetic gradients. In another embodiment, the at least one environmental parameter is for example, pH, temperature.

In one embodiment, shape change is brought about by an analyte-triggered assembly of a structural element via proteolytic unblocking of a sterically hindered precursor. In one embodiment, the structural elements are blocked, preferably with a triple LBT15 domain. In another embodiment, the blocking domain is cleaved in a calcium-dependent manner. In yet another embodiment, the blocking domain is cleaved when either TEVp or SCANR and ionophore are present. In one embodiment, the genetically controlled structural element assembles following calcium-dependent cleavage of the blocking domain in the presence of either TEVp or SCNAR and ionophore. In even another embodiment, the structural elements assemble upon proteolytic cleavage by a protease. In one embodiment, a degron destabilized NLuc cargo is co-expressed. In another embodiment, the co-expressed cargo serves as a read-out measure. In another preferred instantiation, proteolytic unmasking, which can be triggered by calcium, leads to the concatenation and thus stepwise growth of the scaffold SasG, capped off with distinct fluorescent proteins. In another preferred instantiation, proteolysis unblocks inteins to re-complement split vault monomers.

In another preferred embodiment, a change in the concentric barcode is obtained by the calcium-driven binding of calmodulin, fused to a contrast generating unit FABP, to calmodulin-binding peptides presented on the surface of encapsulins as structural elements.

In another preferred embodiment, the agglomeration state of structural elements such as encapsulin can be changed via calcium-mediated interactions of calmodulin and calmodulin-binding peptides.

In yet another preferred embodiment, a change in patterning can also be achieved by relocalization of structural elements to the membrane via calcium-dependent myristoyl-switch proteins.

In addition to the molecular mapping and geometric sensing, the genetically controlled structural element, and/or a genetically controlled scaffold may be used for nanoscopy detection, wherein nanoscopy detection further comprises geometric actuation. Geometric actuation alters a cellular state or process by a biomechanical and/or biochemical effect or via an external stimulus, ore the deposition of external energy, electromagnetic radiation, mechanical energy, or magnetic gradients.

An example for a biomechanical effect is clustering of a receptor via a multidentate structural element or scaffold, such that cellular signaling is modified. Electromagnetic radiation is, for instance, visible or near-infrared light. Mechanical energy is, for instance, mechanical waves covering the ultrasound frequency range.

Such genetically controlled structural element, and/or the genetically controlled scaffold preferably exerts a biomagnetic, biomechanical, and/or biochemical effect on a biological or non-biological structure.

In one embodiment, genetically controlled structural elements and scaffolds exert biomechanical forces by polyvalent interactions with their surfaces, such as clustering of endogenous receptors in model organisms, which can be monitored by nanoscopy.

In another embodiment, the genetically controlled structural element, and/or the genetically controlled scaffold exerts a biomagnetic effect on a biological or non-biological structure. In one preferred embodiment, encapsulins such as QtEnc are rendered magnetic by co-expression of the ferroxidase IMEF and optionally multicopper oxidase Mnx. The expressing cells can thus be magnetically sorted via Magnetic-activated cell sorting (MACS) and subsequently brought back in culture. In addition, or alternatively, the magnetic encapsulins can be bound to intracellular structures such as organelles to manipulate their subcellular localization via magnetic gradients.

In one embodiment, the biomagnetic effect causes specific (re)-arrangements or (re)-organization and for example, a physiological response. In another embodiment, the genetically controlled structural element, and/or the genetically controlled scaffold exerts a biomechanical effect on a biological or non-biological structure.

The geometric actuation can be combined with geometric sensing in that an analyte or parameter that causes a change in a geometric pattern for geometric sensing (e.g. an agglomeration) may then be the condition for effective geometric actuation (e.g. via magnetic gradient fields, which exert more forces on the agglomerated pattern than the monodisperse structural elements). Furthermore, geometric actuation can be monitored via molecular mapping and geometric sensing.

### Detection of the Inventive Patterns and Shapes

In another embodiment, the geometric pattern is defined by pre-determined shapes of the pre-determined nanoscale-sized genetically controlled structural elements comprising at least one genetically controlled nanoscopy contrast-generating unit.

In one embodiment, element-specific methods built into EM instruments such as for example, EDX, EELS, or nanoSIMS can detect the specific metal localized by the metal interactor an orthogonal dimension for multiplexing, in addition to the geometric shape and pattern. In one preferred embodiment, the pre-determined shape is, for instance, an encapsulin or a vault. In one embodiment, the concatenation of the genetically controlled nanoscopy contrast-generating unit to a genetically controlled structural element produces a distinct shape. In another embodiment, the distinct shape is a function of the 3D distribution of the genetically controlled nanoscopy contrast-generating units within the genetically controlled structural element. In other words, the geometrically distinct shape is generated by the interaction of the genetically controlled nanoscopy contrast-generating units and the genetically controlled structural element. In another preferred embodiment, the same shape or pattern can be detected by EM techniques such as TEM, SEM, FIB-SEM and fluorescence of optoacoustics nanoscopy, preferentially with super-resolution techniques.

### Nanobiomaterial

In one aspect, the invention relates to nanobiomaterial consisting of isolated genetically controlled structural elements and/or isolated genetically controlled scaffolds. This nanobiomaterial thus consists of the genetically controlled structural elements and/or genetically controlled scaffolds disclosed herein, which are all isolatable from their originating system, which is a genetically modified cell or a cell-free system.

“Genetically modified” means in this respect that its DNA has been altered by the deletion or addition of genetic material. A “cell-free system” refers to the in vitro synthesis of polypeptides in a reaction mix comprising biological extracts and/or defined reagents. The reaction mix will comprise a template for the production of the macromolecule, e.g. DNA, mRNA, etc.; monomers for the macromolecule to be synthesized, e.g. amino acids, nucleotides, etc.; and co-factors, enzymes and other reagents that are necessary for the synthesis, e.g. ribosomes, uncharged tRNAs, tRNAs charged with unnatural amino acids, polymerases, transcriptional factors, tRNA synthetases, etc.

In one embodiment, the nanobiomaterial is a genetically controlled structural element. In another embodiment, the nanobiomaterial is a genetically controlled structural element and a genetically controlled scaffold. In yet another embodiment, the nanobiomaterial is a genetically controlled scaffold.

Such nanobiomaterial can be used for any nanoscopy method such as TEM, SEM, SRLM, or optoacoustic superresolution microscopy as described herein, however in a two-step process which comprises the production of the nanobiomaterial, its isolation, and its subsequent use in a biological or non-biological system.

### Vectors

The invention further relates to one or more vectors comprising the nucleic acid encoding the genetically controlled nanoscopy contrast-generating unit, the genetically controlled structural element, and/or the genetically controlled scaffold.

A vector comprising a nucleic acid encoding a genetically controlled structural element such as encapsulin and optionally a genetically controlled nanoscopy contrast-generating unit. In some embodiments, the genetically controlled nanoscopy contrast-generating unit is already present in the cell which is transduced with the vector comprising the structural element. In another embodiment, the vector comprises the nucleic acid encoding a genetically controlled scaffold. Such vector may comprise the nucleic acid encoding the scaffold, the structural element, and the contrast-generating unit. In another embodiment, one of the genetically controlled nanoscopy contrast-generating unit, the genetically controlled structural element, and the genetically controlled scaffold is already present in the cell into with the vector is to be transduced.

The vector may be any vector suitable for the transduction of nucleic acids into cells. The encoded elements may be expressed under the control of a cell-specific promoter. The encoded element may be codon-optimized for cellular expression. Such vector may be a viral vector, a plasmid, an exosome, or any other known vector. A viral vector may be a AAV vector. Suitable vectors or constructs according to the invention are disclosed in Table 2 below.

In one aspect of the invention, the AAV vector comprises any combination of the genetically controlled scaffolds, genetically controlled structural elements, and genetically controlled nanoscopy contrast-generating units disclosed herein. In one embodiment, the AAV vector comprises a genetically controlled structural element, wherein said genetically controlled structural element is a Quasibacillius thermotolerans (Qt) encapsulin, further comprising a genetically controlled contrast-generating unit, wherein said genetically controlled contrast-generating unit is one or two murine metallothionein-3, wherein the genetically controlled structural element further comprises the fluorescent protein mScarlet-1. In a preferred embodiment, the AAV vector comprises a genetically controlled structural element, wherein said genetically controlled structural element is an encapsulin from Quasibacillius thermotolerans (Qt), further comprising a genetically controlled contrast-generating unit, wherein said genetically controlled contrast-generating unit is one murine metallothionein-3, wherein mScarlet-I is N-terminally fused, wherein a FLAG tag is C-terminally fused. In another preferred embodiment, the AAV vector comprises the genetically controlled structural element, wherein said genetically controlled structural element is a Quasibacillius thermotolerans (Qt) encapsulin, further comprising a genetically controlled contrast-generating unit, wherein said genetically controlled contrast-generating unit is two murine metallothionein-3, wherein mScarlet-I is N-terminally fused, wherein a FLAG tag is C-terminally fused. In another embodiment, m-Scarlet-I is fused to the two murine metallothionein-3 via P2A. In yet another embodiment, the AAV vector is a AAV5 vector and comprises a CaMKIIa promoter. In another preferred embodiment, the AAV vector comprises a genetically controlled structural element, wherein said genetically controlled structural element is a Quasibacillius thermotolerans (Qt) encapsulin, further comprising a genetically controlled contrast-generating unit, wherein said genetically controlled contrast-generating unit is two murine metallothionein-3, wherein mScarlet-I is N-terminally fused, wherein a FLAG tag is C-terminally fused. In another embodiment, m-Scarlet-I is fused to the two murine metallothionein-3 via P2A. In yet another embodiment, the AAV vector is a AAV5 vector and comprises a CaMKIIa promoter. In another embodiment, an AAV5 vector comprises the genetically controlled structural element, wherein said genetically controlled structural element is an encapsulin from Quasibacillius thermotolerans (Qt), further comprising a genetically controlled contrast-generating unit, wherein said genetically controlled contrast-generating unit is one murine metallothionein-3, wherein mScarlet-I is N-terminally fused to one murine metallothionein-3 via P2A, wherein a FLAG tag is C-terminally fused, wherein the AAV5 vector is under control of a CaMKIIa promoter, or put differently, latter vector is AAV5-CaMKIIa-mScarlet-I-P2A-2M-QtFLAG

In a further embodiment, the AAV vector is selected from the group consisting of AAV1, AAV2, AAV3, AAV4, AAV5, AAV6, AAV7, AAV8, AAV9. In a preferred embodiment, the AAV vector is AAV5. In another embodiment, the AAV vector is pseudtotyped. In another embodiment, the AAV1, AAV2, AAV3, AAV4, AAV5, AAV6, AAV7, AAV8, or AAV9 vector comprises a CAMKII promoter. In a preferred embodiment, the AAV5 vector comprises a CAMKII promoter.

In another aspect, the disclosed AAV vector is for use in medicine. In one embodiment, the disclosed AAV vector is for use in diagnostic imaging.

### EXAMPLES

### Example 1: Cell Expression and Specimen Preparation for Systematic Evaluation of Contrast-Generating Units for EM

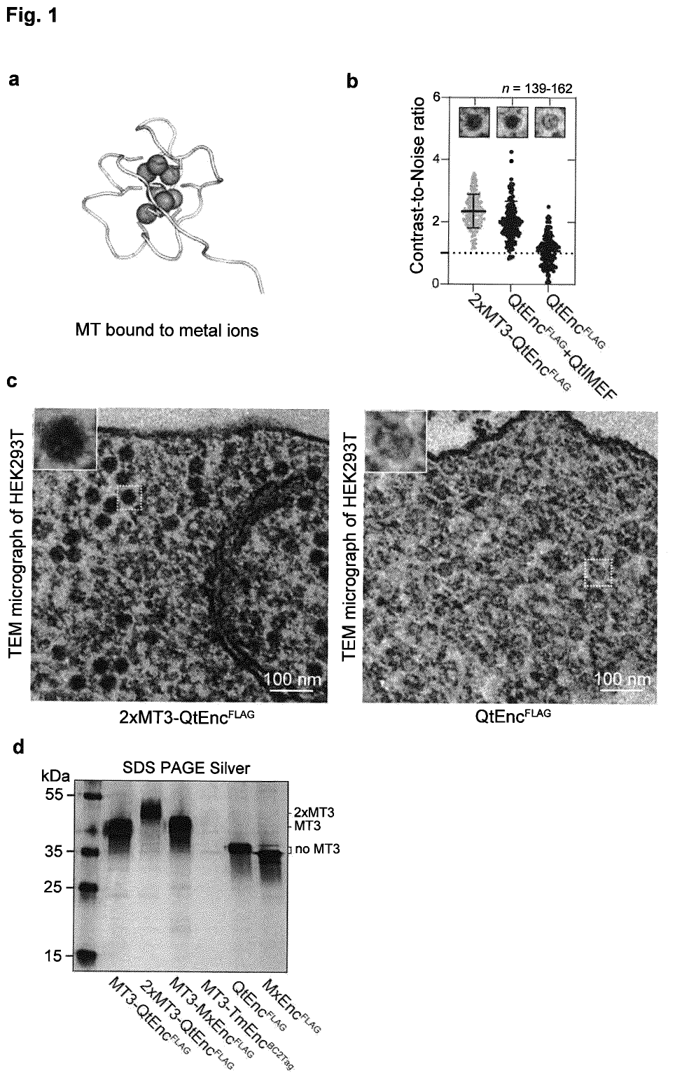

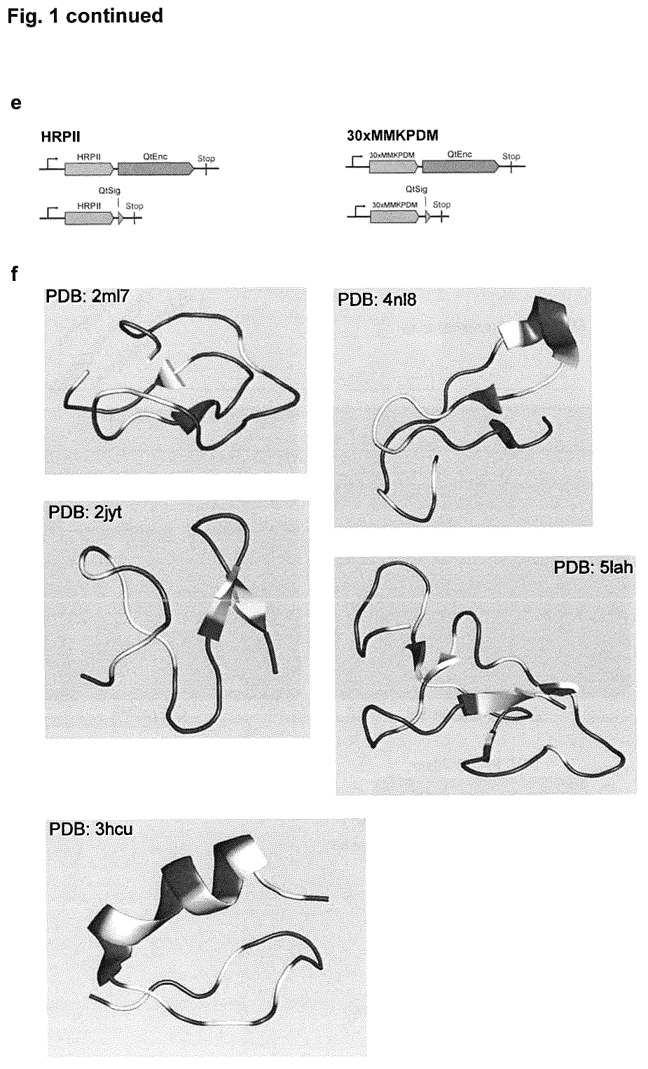

We generated a library of constructs that could be suitable for a genetically controlled contrast-generating unit based on their ability to spatially control metal-interactors, which may also serve as binders for exogenous fluorophores or chromophores. To this end, we adapted naturally occurring proteins and/or de novo designed proteins. For the search of proteins with high content of osmiophilic residues (shown in FIG. 1E, FIG. 1F) we used a Python script, which reads protein sequences of an input database such as PDB and calculates the number of osmiophilic residues (C H M W) divided by the length of the protein.

We used standard plasmid lipofection techniques in HEK293 cells to express a library of constructs coding for metal-interactors to screen for sufficient contrast in TEM after standard fixation, post-fixation, and staining protocols (FIGS. 1-6). All DNA constructs were either custom synthesized by IDT DNA Technologies or assembled from multiple fragments via HiFi assembly, traditional ligation cloning, or PCR-based mutagenesis methods and cloned into pcDNA 3.1 (+) Zeocin for mammalian expression.

Low passage number HEK293T cells (ECACC: 12022001, obtained via Sigma-Aldrich) were cultured in advanced DMEM with 10% FBS and penicillin-streptomycin at 100 μg/ml at 37° C. and 5% CO2. HEK293T cells were transfected using X-tremeGENE HP (Roche) transfection reagent according to the manufacturer's protocol (3:1 μl reagent:μg DNA). DNA amounts (ratio shell to cargos) were kept constant in all transient experiments to yield reproducible DNA-Lipoplex formation.

Expression was confirmed by native and SDS gel electrophoresis (12% BioRad TGX gels, run at 200 V for 40 min) (FIGS. 1, 2, 3, 5, 6). Constructs resulting in robust, non-toxic expression were then subjected to EM sample preparation using standard EM fixation, post-fixation, and staining protocols compatible with protocols also used for tissue from model organisms.

For EM sample preparation, the staining method was adapted from Deerinck et al., Microsc. Microanal. (2010). HEK293 cells were harvested 36 hours post-transfection, using accutase treatment, and pelleted by centrifugation. The pellets were then fixed with 2.5% Glutaraldehyde in 0.1 M sodium cacodylate buffer (pH 6.7-7.0) for 20 min. For Drosophila melanogaster brains, the fixation duration varied between 20 minutes and 24 hours. After removal of fixative, the material was post-fixed using the 1:1 mixture of 4% OsO₄ with 0.3 M CB, containing 3% potassium hexacyanoferrate (II) for 20 minutes on ice. The post-fixative solution was removed, and the material was washed twice with 0.1 M sodium cacodylate buffer for 2 minutes, followed by 2×5 min Milli-Q washing steps. Milli-Qwater was removed, and the material was optionally treated with 1% tannic acid (10 min, RT). Then, tannic acid was decanted, and the material was washed 5 times for 5 minutes using Milli-Q water. The material was then treated with 1% uranyl acetate solution (30 min, RT), with subsequent 5 times 5 minutes washing steps with Milli-Q water. Afterward, the material was subjected to treatment with 3% lead aspartate (15 min, RT). Again, the material was washed (5×5 min in Milli-Q water) before proceeding to epoxy embedding. The epoxy embedding process was conducted over 2 days. On the first day, the material was incubated on ice with 75% EtOH for 10 minutes, followed by incubation in 90% EtOH for 10 minutes and 100% EtOH for 30 minutes, then 100% EtOH for 30 min on ice. The EtOH solution was replaced with pure propylene oxide and incubated for 5 minutes under RT. Next, the solution was discarded, and the material was incubated in 1:1 mixture of EPON and Propylene oxide for 30 minutes under RT. Subsequently, the material was sequentially incubated at room temperature in 100% EPON for 30 minutes and again with fresh EPON solution overnight. On the next day, the old epoxy was removed, the material was briefly rinsed in fresh 100% epoxy and left in newly-poured 100% epoxy for 72 hours under 60° C. The Drosophila m. brain prior to final EPOXY curation was oriented such that several optical lobes were located parallel to the block's slicing plane. The resulting blocks were subjected to trimming and slicing. For cellular material, the trimming of excess EPOXY from the block's surface was done using an EM TRIM milling system (Leica Microsystems, Germany). Using an UltraCut E microtome (Reichert/Leica, Germany) the prepared blocks were prepared with a histo-knife (DIATOME, Switzerland) and then sequentially cut with an ultra-knife (DIATOME, Switzerland) at a slice thickness of 70 nm, verified by the slices' interference pattern. The slices were deposited either on the surface of a 200 mesh copper grid or the polished side of a silicon wafer.

TEM images were acquired on a Libra120 TEM (Carl Zeiss GmbH, Germany), equipped with a CCD camera (TRÖNDLE Restlichtverstärkersysteme, Germany) using ImageSP software (SYSPROG, Belarus). Before image acquisition, all grid-supported specimens were pre-irradiated with 120 kV beam voltage, 13 μA beam current, 200 μrad illumination angle without insertion of apertures. The actual image acquisition took place with the activated BIO-AIS condenser aperture system and 60 μm objective aperture under the same beam conditions as used for pre-irradiation, except for the illumination angle that was specified to be equal to 100 μrad. The target magnification for the presented images was selected as 8000× in the microscope control software, exposure timing was set equal to 1000 s. Selection of regions of interest for computing contrast-to-noise-ratios (CNR) was performed using ImageJ.

### Example 2: Complementation of Contrast-Generating Units with Light-Dependent Microscopy Methods

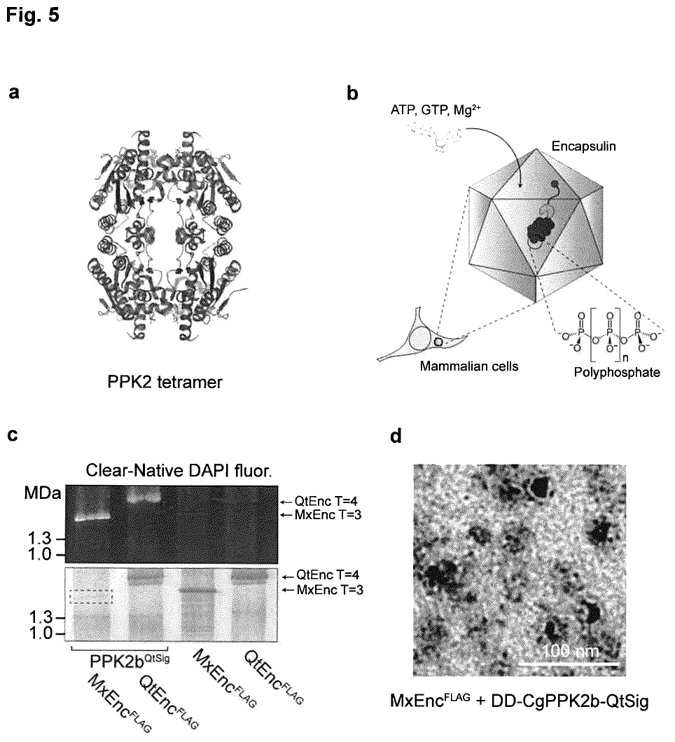

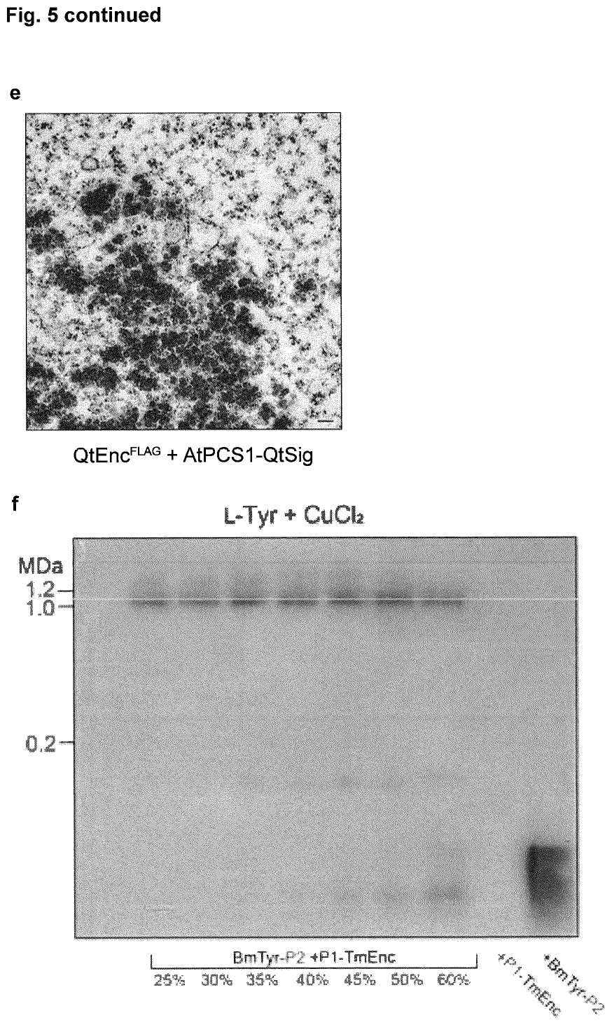

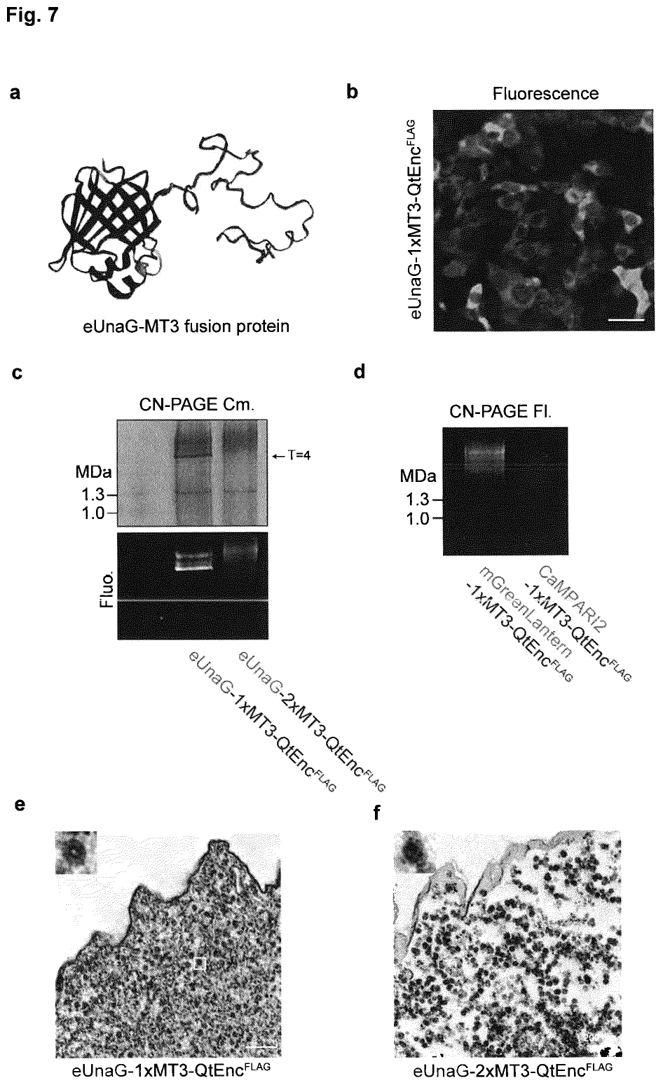

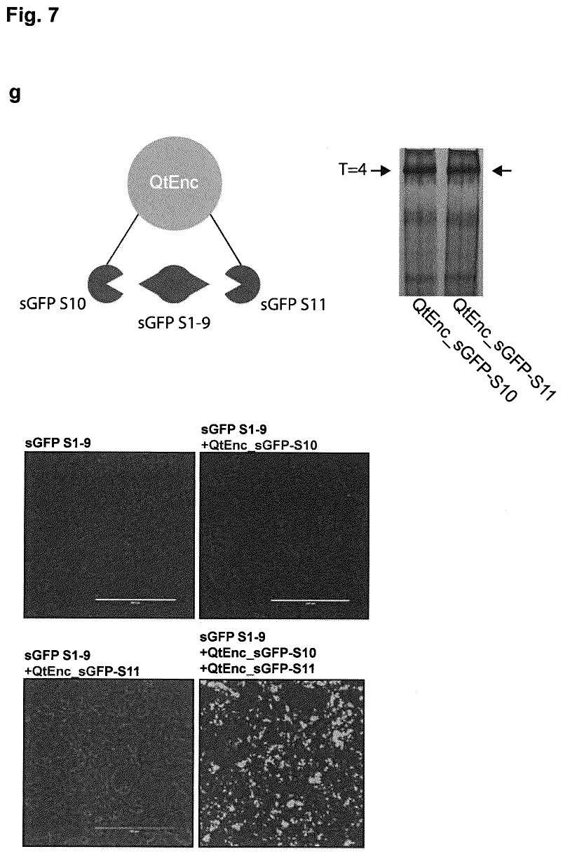

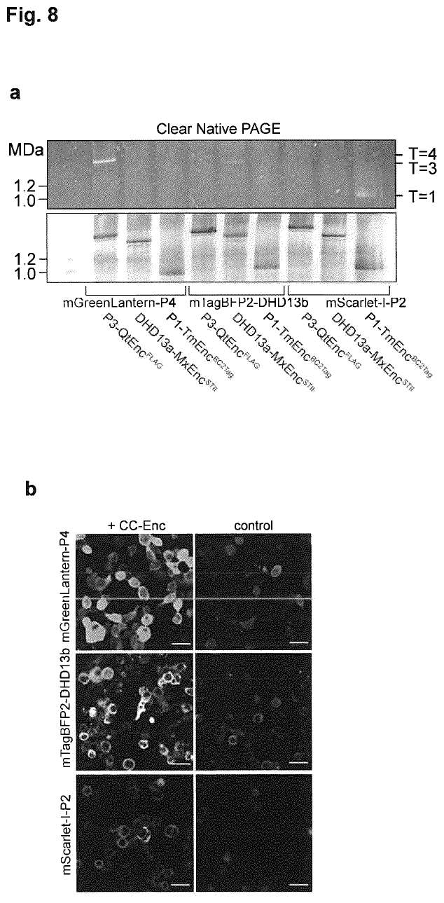

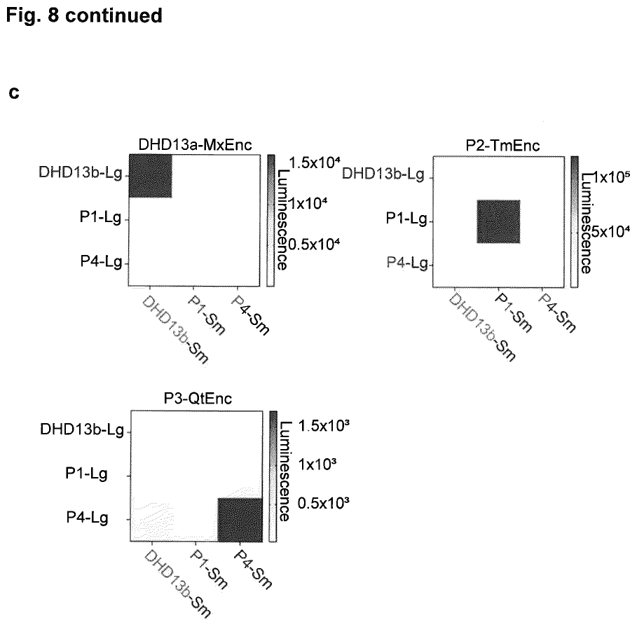

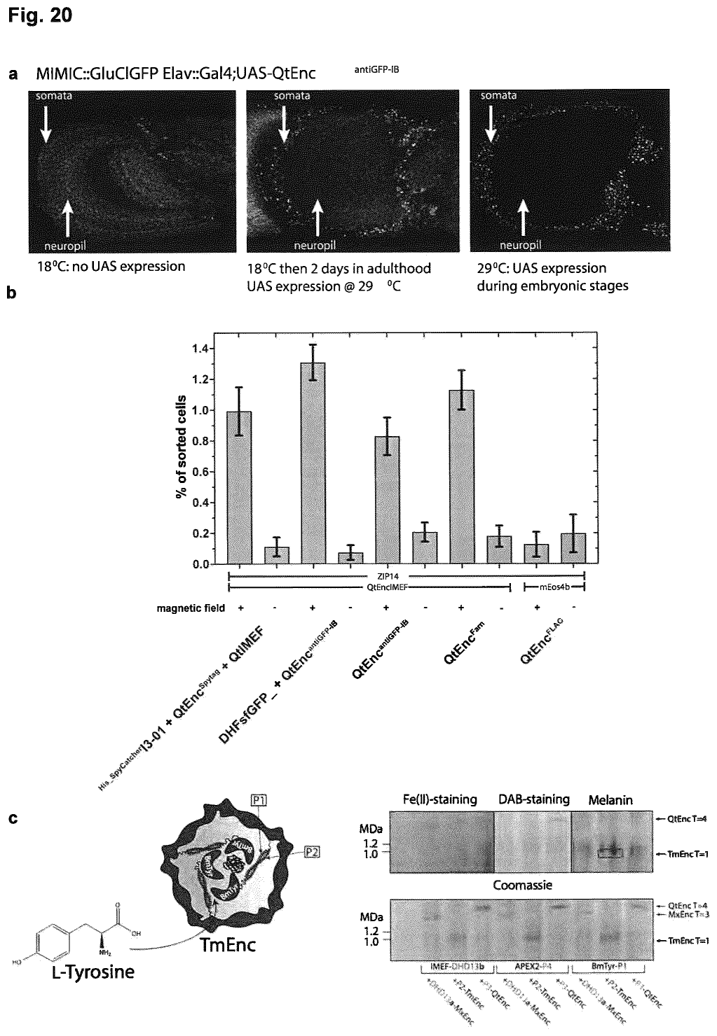

Constructs combing metal interactors and fluorophores were expressed in HEK293 cells as described in detail in Example 1.36 hours post-transfection, fluorescence microscopy was conducted on a confocal microscope (Leica SP5 using the 488 nm laser line, Leica Microsystems) or epifluorescence microscope (EVOS FL Auto Cell Imaging System (Thermo Fisher Scientific) (FIG. 7). Fluorescence on-gel analysis was performed by illuminating the CN-PAGE gel by UV light and capturing the image on a standard CCD camera (FIG. 5c after incubation with DAPI, FIG. 7c, FIG. 7d, FIG. 8a direct fluorescence). Multimodal detection, complementing EM contrast, was also extended to bioluminescence detection, via a luciferase targeted to the inside of the encapsulin via bioorthogonal coiled-coil adapters. Luminescence measurements were taken after the addition of fumirazine on a luminometer (Berthold Centro LB 960 (Berthold Technologies) at 0.1 s acquisition time.) (FIG. 8c). In a similarly modular fashion, the enzyme tyrosinase was encapsulated inside the TmEnc, and the formation of melanin was confirmed on a native gel by photoabsorbance, which is also the basis of the photoacoustic signal (FIG. 20c). Alternatively, fluorophores can also be attached via SNAP or HaloTags or can also be presented on the outer surface (e.g., via C-terminal eUnaG fusion) of structural elements such as encapsulins, which leaves the lumen of encapsulins unaltered for organizing contrast-generating units for EM. Since entire fluorescent proteins fused to the outward-facing N-terminus of encapsulins tend to disrupt encapsulin assembly, we decided to reconstitute the smaller fragments of tripartite split-GFPnon the surface as explained in FIGS. 7g-7i and verified assembly and fluorescence by BN-PAGE and fluorescent microscopy, respectively.

### Example 3: Geometric Multiplexing with Concentric Barcodes Based on Spherical Structural Elements

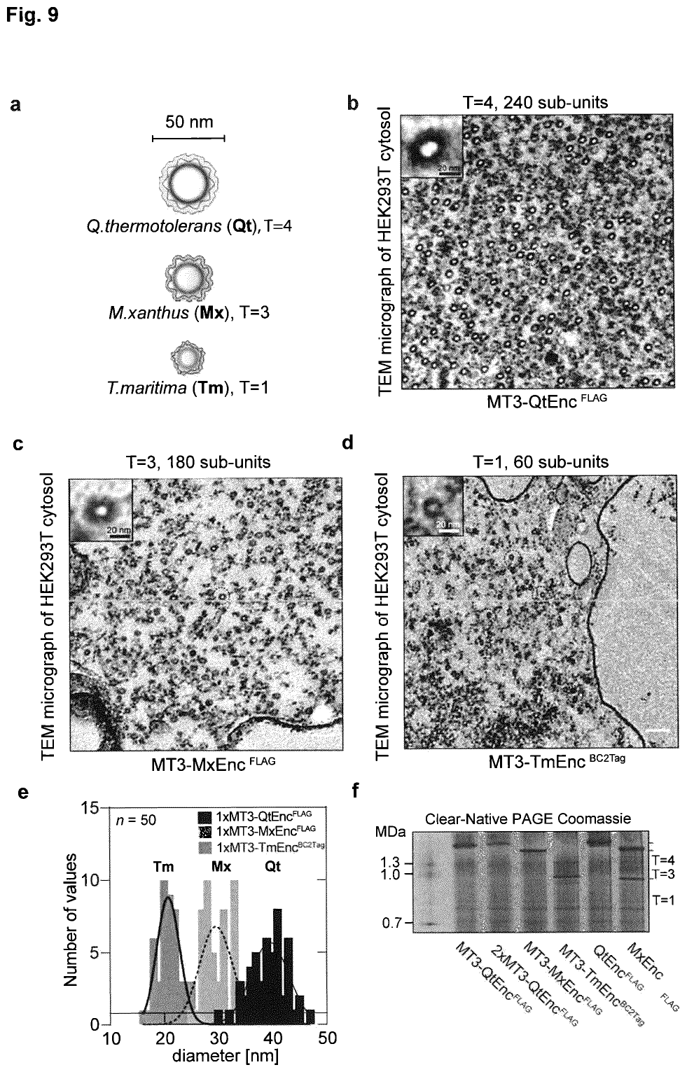

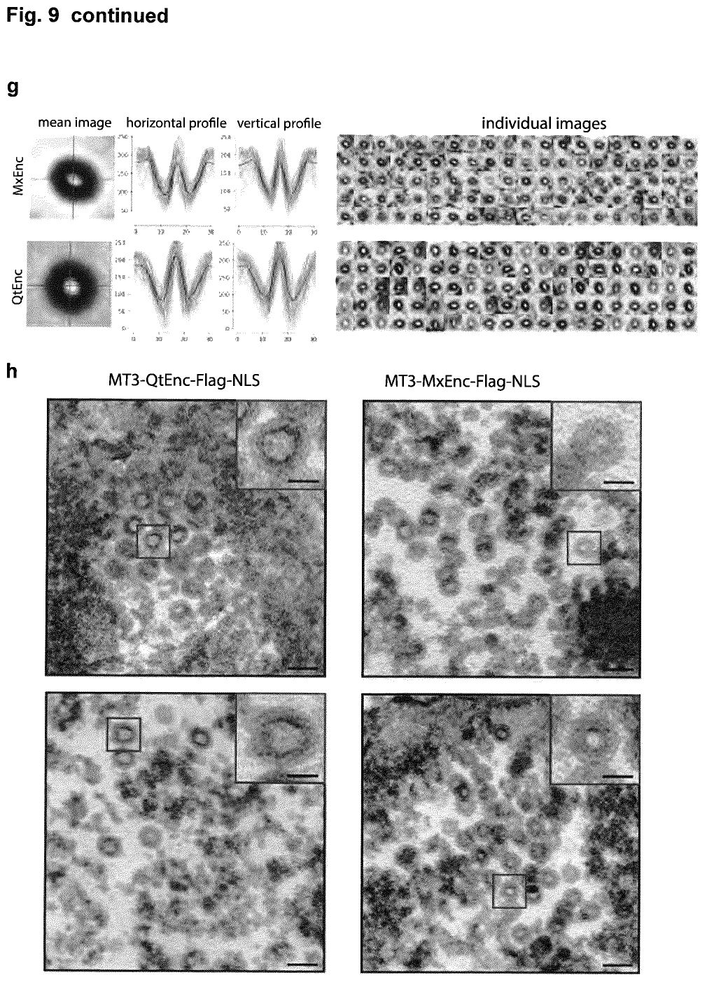

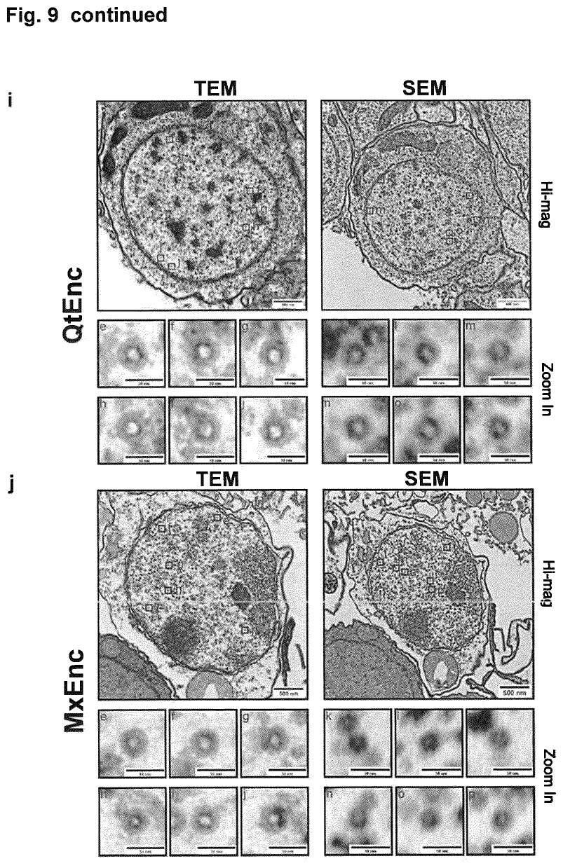

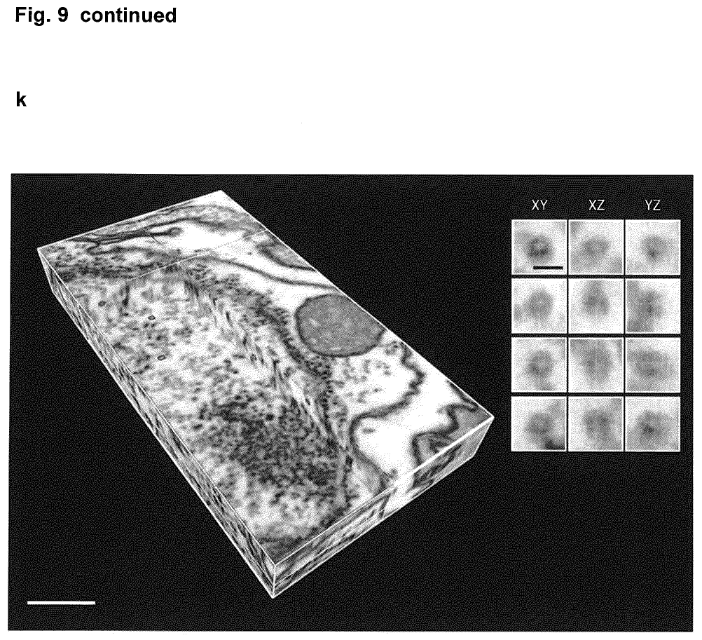

We genetically concatenated multiple contrast-generating units from metallothionein 3 (MT3) spherical structural elements based on variants of encapsulins and expressed these structural elements in HEK293 cells (FIGS. 9a-9h, see detailed protocol in Example 1), as well as in Drosophila neurons (FIGS. 9i-9k). To this end, a vector was generated for transgenesis by cloning the respective constructs into pJFRC7-20XUAS-IVS or pJFRC19-13XLexAop2-IVS and submitting those to a commercial Drosophila Embryo Injection Service for transgenesis. Sample preparation for TEM from HEK cells and freshly dissected Drosophila brains was performed as described in detail in Example 1. Histograms of the distribution of diameters were generated with the measurement tools implemented in ImageJ (FIG. 9e). Line profiles shown in FIG. 9f were computed by using ImageJ and custom-written python scripts.

We furthermore conducted comparative imaging via TEM and SEM of the identical nuclei of Drosophila neurons expressing MT3-QtEnc-Flag-NLS or MT3-MxEnc-Flag-NLS (FIGS. 9h-9j). To this end, ultrathin sections were captured on either TEM grids or on silica wafers for imaging via TEM or SEM, respectively. SEM images were acquired with an SEM Gemini 360 (Carl Zeiss GmbH, Germany), equipped with a Bio-BSD detector (Carl Zeiss GmbH, Germany) using ATLAS software (Carl Zeiss GmbH, Germany). Silicon wafers supporting the samples were glued to the stage with silver glue (PLANO GmbH, Germany). Image acquisition parameters were: 6.5 kV beam voltage, 2 nm nominal pixel size with a 30 μm objective aperture at a working distance of 3.7 mm. The stage was subjected to a bias voltage of 5 kV.

Furthermore, the different encapsulin: metallothionein combinations can be detected via focused ion beam (FIB)-SEM as shown in FIG. 9k). To this end, FIB-SEM image acquisition was performed with the SEM Crossbeam 550 (Carl Zeiss GmbH, Germany), equipped with the InLens, BSE, SESI detectors (Carl Zeiss GmbH, Germany) running ATLAS software. The metal-sputtered block with the specimen of interest was positioned on the stage with silver glue (PLANO GmbH, Germany) to ensure the proper adhesion and discharging of the specimen. A guiding pad was deposited in the proximity of the ROI to meet the requirements of image capture alignment during the three-dimensional volume recording. The following settings were used for image acquisition: SEM beam voltage 1.3 kV, a working distance of 5 mm, 4 nm nominal voxel size. The FIB Ga beam, accelerated by 30 kV voltage and holding a current of 700 μA, was used for three-dimensional slicing of the block's volume.

As shown in FIG. 10, by concatenating one or multiple metallothioneins, different concentric barcodes can be generated that provide rotational invariant contours also on non-isotropic imaging slices (i.e. in cross-sections or maximum intensity projections) defined by multiple rings of contrast in the lumen of encapsulins (line plots in FIG. 10d generated by ImageJ). Those concentric barcodes can be complemented by additional rings (FIG. 10e) using any of the contrast-generating units detailed in previous Examples and Figures, for instance, by FABPs targeted via an isopeptide bond formation. Shown in FIG. 10f) is the schematic of the genetic constructs for binding MT3 or FABP to the surface to the spherical QtEnc via SpyTag/SpyCatcher isopeptide formation (also compare FIG. 3b).

### Example 4: Geometric Multiplexing with Non-Spherical Structural Elements

Different modifications of the major vault protein were assembled from synthesized DNA and expressed in HEK293 cells, as detailed in Example 1. Fluorescent cargo proteins fused to the enzyme APEX2 were co-expressed and imaged via confocal fluorescence microscopy on an SP5 Imaging System, Confocal Microscope (Leica, Wetzlar, Germany). Histochemistry was performed using a standard protocol for APEX2, applying 3,3′-diaminobenzidine (DAB) and hydrogen peroxide (H₂O₂) (FIGS. 11a, 11b). TEM imaging was performed as described in detail in Example 1 (FIGS. 11c, 11d). Epifluorescence microscopy (EVOS FL Auto Cell Imaging system (Thermo Fisher Scientific) was performed to confirm the dual fluorescence labeling of the major vault protein by UnaG, fused to the internal HLH domain, and the red fluorescent protein mScarlet, targeted to the lumen via an mINT motif.

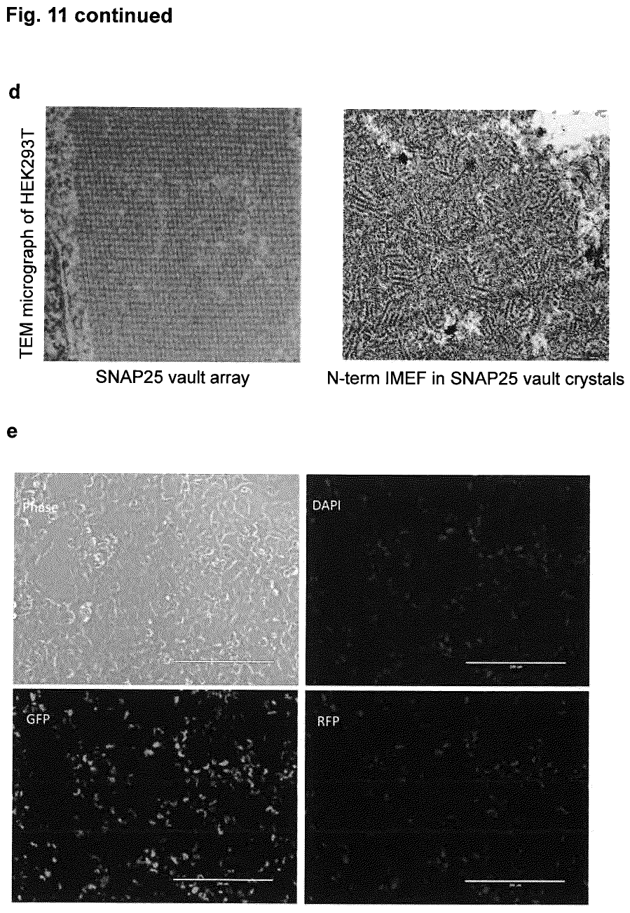

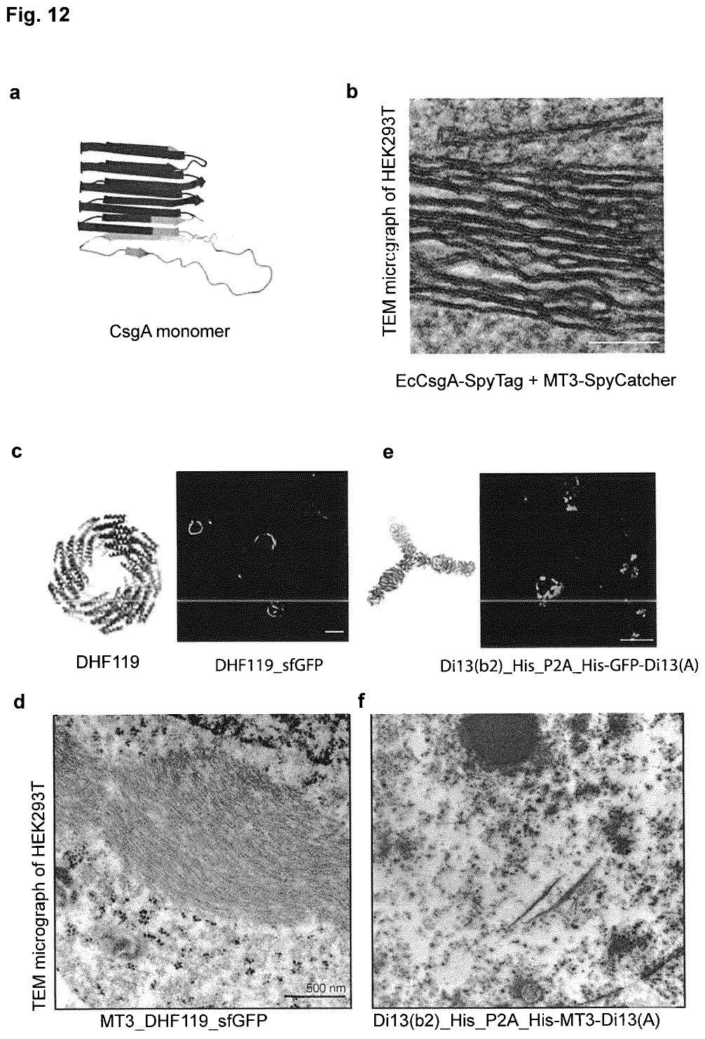

We furthermore generated non-spherical structural elements by fusing MT3 to auto-assembling Csga monomers (FIGS. 12a, 12b), fibril- or lattice-forming elements (FIGS. 12 c-12f), which were evaluated by TEM as described in detail in Example 1. In addition, structural elements can be formed by the concatenation of the nanoscopy contrast-generating units based on FABPs connected via minimal linkers (as detailed in FIG. 13).

### Example 5: Monovalent-to-Polyvalent Hubs as Structural Elements and Control of Valency on Structural Elements

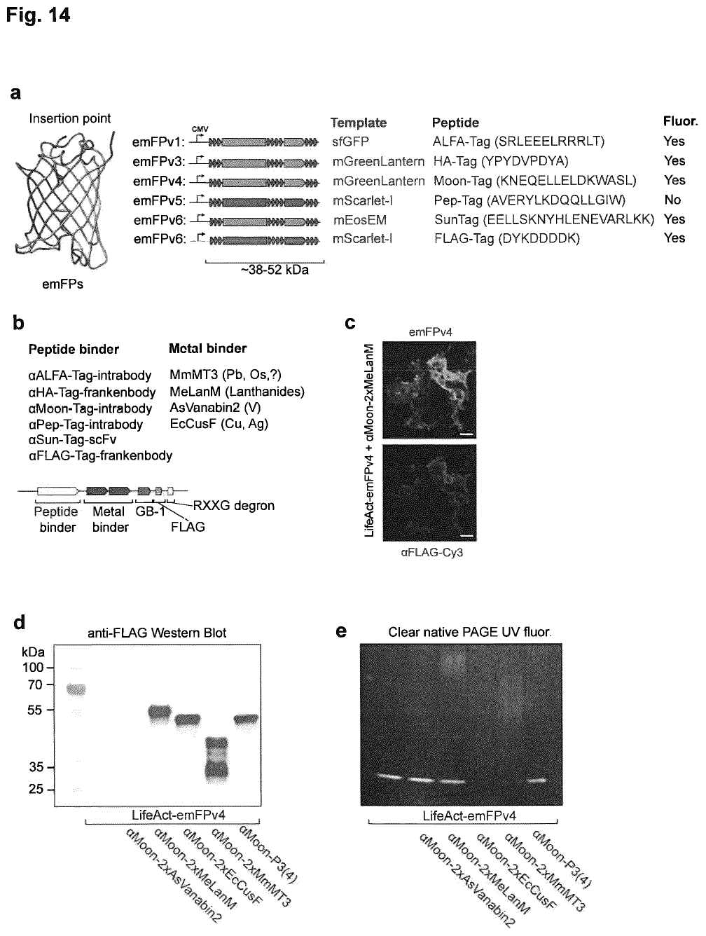

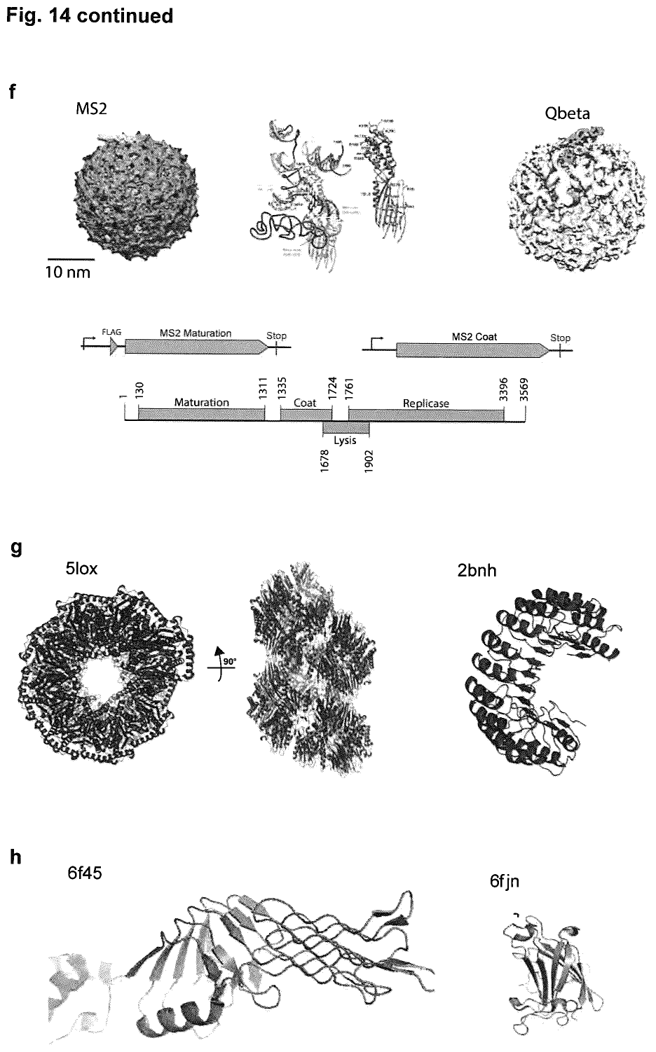

We constructed monovalent adapters to multiple contrast-generating units by constructing variants of spaghetti monster (emFP, FIGS. 14a-14e) that harbor several epitopes for intrabodies or frankenbodies, which can be fused to the respective nanoscopy contrast-generating units as detailed in FIG. 14b). In order to test, whether those variants are still fluorescent, we expressed them in HEK293 cells as described in Example 1 and imaged them on an EVOS FL Auto Cell Imaging system (Thermo Fisher Scientific) (FIGS. 14a, 14c). Furthermore, we evaluated via combined fluorescence imaging and immunohistochemistry whether the emFPs could be targeted to subcellular structures and still provide the binding sites for the multiple binders. FIG. 14c shows emFPv4 yielding green fluorescence via mGreenLantern that partially overlaps with Cy-3 fluorescence indicating binding of the antiMoon-Tag intrabody. We furthermore demonstrated the successful interaction on the monovalent-to-polyvalent hub by using CN-PAGE, which shows higher-molecular band species under UV illumination corresponding to bound anti-moon intrabody fused to different metal-interactors (FIG. 14i). As an alternative to the use of monovalent-to-polyvalent hubs, we evaluated programmable translational read-through to control the stoichiometry of attachment points on the surface of encapsulins to systematically reduce the valency of the surface-presented attachment points (which would otherwise only be determined by the icosahedral symmetry). To this end, we expressed the genetic constructs depicted in FIGS. 15a, 15b, such that variable amounts of the bioluminescent tag HiBit were surface-expressed on QtEnc. We subsequently conducted a HiBit assay (Promega) following the protocols suggested by the commercial system using a standard luminometer.

### Example 6: Geometric Multiplexing by Spatially Organizing Structural Elements on Cellular Scaffolds

Given the nanometer-scale resolution of electron microscopy and super-resolution microscopy techniques, multiplexed information can be readily encoded by modifying the subcellular localization of the structural elements detailed in previous figures via scaffolds expressed at distinct subcellular locations inside cells. We demonstrate several examples of this strategy by expressing in HEK293 cells—followed by TEM analysis—both as described in Example 1, either a membrane localization signal (farnesylation tag, FIG. 16a), a peptide from a cargo adaptor which binds to endogenous motor proteins (KinTag, FIG. 16b), or an anti-GFP intrabody targeted to lysosomal-localized GFP (FIG. 16c), or membrane-localized GFP (FIG. 16d, in the control condition on the right, a Flag epitope was expressed instead of the anti-GFP intrabody).

### Example 7: Geometric Multiplexing by Designer Scaffolds

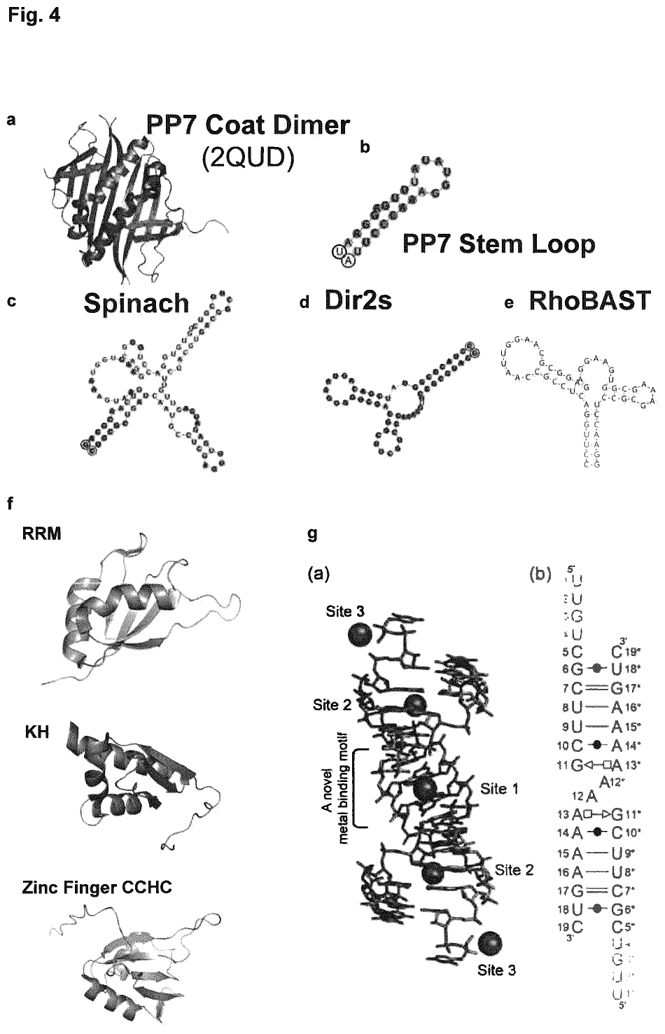

As an alternative to using endogenous scaffolds, we demonstrate the versatile application of the rigid hetero-bifunctional protein from S.aureus as an engineered scaffold, whose length can be systematically varied to present bio-orthogonal adapters at both ends (FIG. 17a). To demonstrate the precise linear extension of the designed SasG linker when expressed in mammalian cells, we expressed a version of variable length with the FRET pair sfGFP and mCherry on either end, expressed them in HEK293 cells (described in Example 1), and subjected the cell lysate to CN-PAGE under UV illumination. This analysis shows a FRET effect indicated by a yellow signal for only the direct fusion of sfGPF and mCherry. In distinction, the bands corresponding to the constructs with longer spacers between the fluorescent proteins give a green fluorescent signal indicating the absence of FRET and the isolated fluorescence from sfGFP (FIG. 17b). We furthermore validated the rigid hetero-bifunctional crosslinker SasG in combined fluorescence microscopy and TEM (FIG. 17c, d) based on the protocols described in detail in the previous examples. The results show distinct spatial patterns of two differently sized encapsulins (QtEnc and TmEnc), indicating that constraints from steric hindrance are sufficient to obtain distinct barcoding patterns from the combination of several structural elements and contrast-generating units (FIG. 17e, f). In addition to the use of a single SasG element, several of these linear connectors can be expressed to generate more complexly shaped structural elements such as triangles and other platonic shapes. By expressing 3 bio-orthogonal pairs of adapters chosen in this case from isopeptide-forming pairs and inteins, triangular shapes can be enforced upon co-expression of the respective components in HEK293 cells and using on-gel fluorescent analysis of the individual combinations of co-expressed components. The results also show a high-molecular species corresponding to the combination of all three elements (last lane in FIG. 17h). Alternatively, to proteinaceous scaffolds such as SasG, scaffolds can also be designed from RNA as shown in FIG. 17g, which may in parallel provide contrast based on the motifs detailed in FIG. 4.

For completion, in addition to the geometrically precise and bounded scaffolds, one can also use simpler clustering of structural elements such as encapsulins surface-presenting SpyTags with multivalent crosslinkers (in this case SpyCatcher mounted to the de novo cage 13-01 (FIG. 17i). The EM contrast, in this case, is provided by the ferroxidase IMEF, resulting in a complete filling of the encapsulin lumen. Alternatively, modular combinations with the concentric barcodes detailed in Example 3 can be used.

### Example 8: Molecular Mapping and Geometric Sensing

For mapping the subcellular distribution of biomolecules of interest, structural elements or scaffolds can be targeted to the biomolecule of interest via a range of attachment points whose valency can be controlled via mono-to-polyvalent hubs, which allow for the attachment of several contrast-generating units or structural elements to a bioorthogonal attachment point to the biomolecule of interest (FIG. 14) or via read-through control of the stoichiometries of the attachment points (FIG. 15). Targeting of the target biomolecule of interest can also be achieved via the intracellular delivery of exogenous antibodies (via electroporation or microinjection) that can be connected to a structural element or scaffold via a deactivated Trim21 molecule (dTrim21), which can be fused to an attachment point such as a fluorescent protein (FIG. 18l). This “sandwich” arrangement allows binding of a structural element and additional mapping capabilities via fluorescence microscopy).

To demonstrate that the geometrically distinct structural elements can be made responsive to cellular states, we expressed sterically hindered variants of encapsulins as structural elements containing the contrast-generating unit MT3 (genetic constructs shown in FIG. 18a) that allow for proteolytic unmasking by the tobacco etch virus-derived (TEV) bio-orthogonal TEV protease, of which a calcium-dependent version has been generated that is termed SCANR. After expression in HEK293T cells and inducing calcium influx by addition of increasing concentrations of the calcium ionophore (Calcimycin A23187, Sigma-Aldrich), a higher molecular weight band appears on the CN-PAGE (FIG. 18b), corresponding to the accumulation of a band corresponding to the proteolytically cleaved protein on SDS-PAGE (FIG. 18c). In addition, we co-expressed degron-destabilized luciferase (NanoLuc) such that we could use a luciferase signal as an indicator for the amount of NanoLuc that was encapsulated inside the lumen of the encapsulins (via the appropriate encapsulation signal, QtSig). Thus, we could use simple bioluminescent detection with the luciferase-substrate fumirazine to quantify the calcium-dependent proteolytically triggered encapsulin assembly via the amount of luciferase that was protected inside the encapsulin lumen to give a bioluminescent signal (FIG. 18d). The corresponding TEM image shows the expected ring-shaped MT3-QtEncFlag structural elements after calcium influx (FIG. 18e).

As an extension to geometric sensing via changing the shape obtained from structural elements, we also demonstrate how the geometric pattern formed by several structural elements can be altered by altering the geometry of the scaffold as a function of an analyte of interest. FIGS. 18f-18i show how SasG scaffolds can be concatenated via split-intein-mediated processes. These components thus realize a step-wise growing counter/timer mechanism, which can be triggered or stopped by using caged inteins, or modifying the concentration of unmasked Base or Cap modules (FIGS. 18h, 18i).

We furthermore show that the concept of the proteolytic unmasking of sterically-hindered precursors to structural elements can also be applied to non-spherical structural elements such as vaults. FIG. 18j shows a schematic demonstrating a split major vault protein that is modified by split-intein pairs blocked by masking peptide connected via a TEV-protease cleavage site (FIG. 18j). To show that vault auto-assembly occurs upon co-expression of TEV protease, we co-expressed the fluorescent protein mScarlet with an mINT targeting signal addressing it to the vault lumen and subjected the respective HEK293 cells to epifluorescence microscopy (EVOS FL Auto Cell Imaging system (Thermo Fisher Scientific)). The images show that a cytosolic accumulation of fluorescence signal occurs upon TEV co-expression, which is similar to the signal observed when non-sterically blocked vault monomers (right column) were expressed (FIG. 18k).

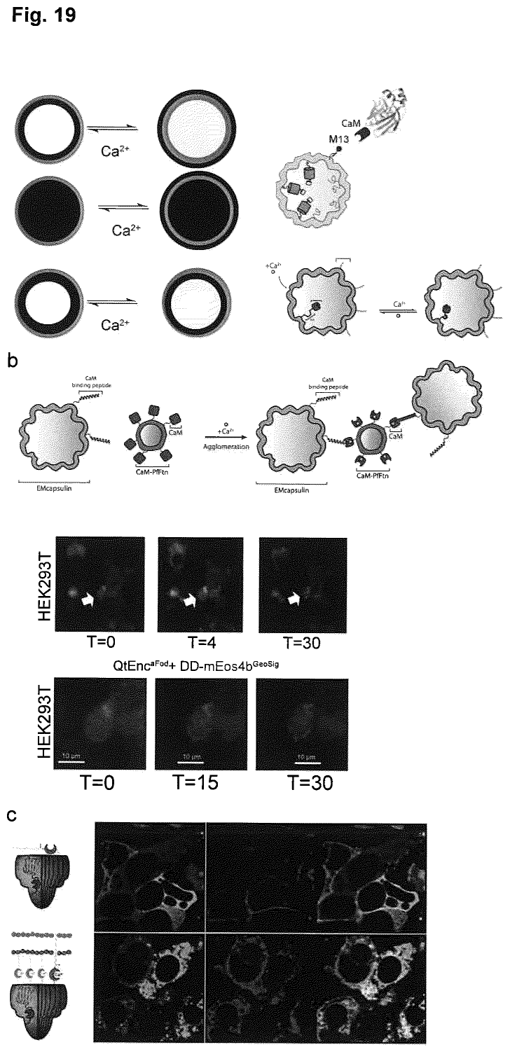

In addition to the mechanism of proteolytic unmasking of the building blocks of structural elements, concentric barcodes can be added to the outside of spherical structural elements via, for instance, the surface presentation of calmodulin-binding peptides such as M13 to which calmodulin (CaM) fused to contrast-generating units can bind (FIG. 19a) similar to the interaction of isopeptide-forming partners displayed in FIGS. 3b,3 c. We have furthermore co-expressed QtEnc surface-modified with the CaM-binding peptide aFod and a multi-dentate cross-linker constructed from a ferritin variant from Pyrococcus furiosus surface-modified with CaM (FIG. 19b) and the degron-destabilized fluorescent protein mEos4b. We subsequently conducted epifluorescence microscopy and observed the formation of fluorescent intracellular clusters after minutes of triggering calcium influx via the calcium ionophore A23187 (Sigma-Aldrich). These data thus demonstrate the agglomeration of multi-dentate structural elements as a reporter mechanism. The analogous mechanism can be established for phosphorylatable peptides and phosphopeptide-binding proteins such as SEQ ID NOs: 239, 206. Similarly, relocalization of structural elements, demonstrated on the example of vaults in FIG. 19c, can be demonstrated by expressing a variant of vaults modified with a myristoyl-switch motif adapted from recoverin, which relocalizes to the membrane upon binding to calcium. The confocal microscopy images show the formation of fluorescent clusters upon pharmacological triggering of calcium influx (30 μM A23187), derived from the co-expressed vault-packaged mEos4b.

### Example 9: Geometric Actuation

We have generated several Drosophila lines as described in Example 1 in which a multi-dentate QtEnc surface-modified with anti-GFP intrabodies (QtEncantiGFPIB) is expressed via a panneuronal promoter (Elav) in a standard temperature-dependent fashion via the Gal4:UAS system. When this line is crossed with a line expressing a GFP-tagged receptor GluCl in a transposon Minos-mediated integration cassette (MiMIC) (Venken et al., 2011), the intraneuronal localization of receptors can be controlled via biomechanical actuation through multi-dentate binding to the gradually expressed multi-dentate nanoscopy-visible structural element, which is detected by immunohistochemistry to the Flag-tag in FIG. 20a. Please note the temperature-dependent release (switch from 18° C. to 29° C., using the standard method to control the conventional GAL4-UAS system via a temperature-sensitive allele of GAL80) of the receptor to the neuropil of the respective Drosophila neurons.

In addition to the biomechanical manipulation of the subcellular distribution of biomolecules of interest, we demonstrate in FIG. 20b how cells can be magnetically sorted when they express encapsulins that are expressing the ferroxidase IMEF. The graph shows the selective retention of HEK293 cells expressing QtEnc and IMEF on commercially available columns (Miltenyi), demonstrating that the magnetic-field-dependent force is sufficient to retain entire cells on magnetic columns. Importantly, all examples here show biomagnetic encapsulins as structural elements, which are patterned by either de novo (the cage 13_01 or the filament DHF, FIG. 12d) or natural (farnesylation, FIG. 16a) scaffolds which can localize magnetic actuation in the cell while the precise patterns can be read out by nanoscopy, confirming the distinct geometry for the magnetically actuatable structures.

In addition to biomagnetic manipulation with EM-visible nanoscale patterns, we furthermore demonstrate in FIG. 20c that the strongly photoabsorbing and thus photoablatable polymer melanin, visible standard EM stains, can be formed inside the geometrically distinct structural element such as TmEnc. After the expression of the different genetic combinations of coiled-coil adapter pairs (e.g., P1/P2 for encapsulating Bacillus megaterium BmTyr-P1 to P2-TmEnc as specified in FIG. 20c, melanin formation could be demonstrated by color formation on CN-PAGE.

### Example 10: Fixation-Stable, Nanoscale EM Contrast

Our first objective was to generate variants of encapsulins, which produce robust EM contrast with nanoscale precision (EMcapsulins) using standard EM fixation and staining protocols, and without additional incubation steps necessary to allow diffusion of DAB and H₂O₂ or other exogenous substrates. EMcapsulins are one exemplary genetically controlled structural element according to the invention.

Since murine Metallothionein-3 (M) has been demonstrated to be a potent lead binder and acidic stretches of other metallothionein domains have been shown to interact with uranyl ions, we reasoned that M might not only work in specialized EM procedures but also provide localized EM contrast organized by encapsulins and standard staining protocols. As laid out in the aspects and embodiments of the invention below, the genetically controlled structural element (here: EMcapsulin) comprises at least one contrast-generating unit. According to the invention and this example, the EM capsulins comprises one or two murine metallothionein-3 (SEQ ID NO: 1, SEQ ID NO: 4), or a chimeric metallothionein sequence comprising three metallothionein, i.e. murine metallothionein-3 (MmT3), Synechococcus elongatus (SmtA) metallothionein, and Triticum aestivum Ec-1 metallothionein (TaEC1) (SEQ ID NO: 5).

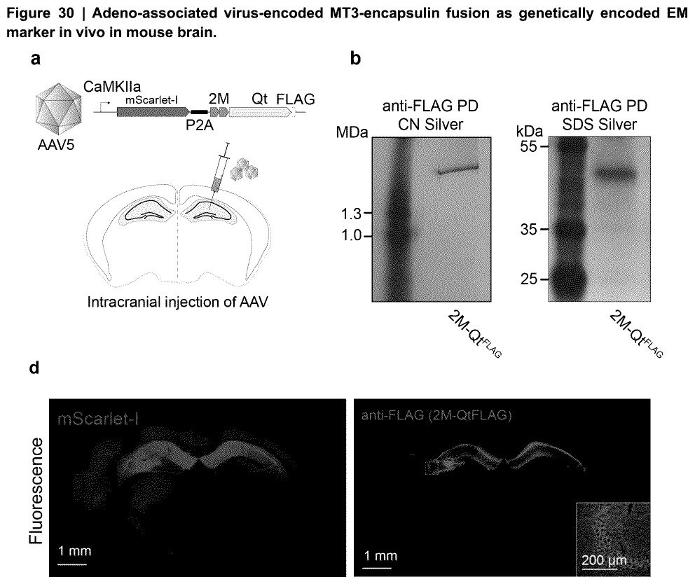

Given M's small size and flexibility, we thought it unlikely to disrupt encapsulin assembly. We thus generated direct fusions to the lumen-facing N-terminus of encapsulin monomers instead of encapsulating M as cargo protein to obtain better control over the stoichiometry and suppress background from non-encapsulated cargo (FIG. 30a).

When we expressed M as N-terminal fusions to encapsulins from Quasibacillus thermotolerans (Qt), Myxococcus xanthus (Mx), and Thermotoga maritima (Tm) in HEK293T, we could confirm by Clear Native (CN) PAGE that Qt^{FLAG} and Mx^{FLAG} variants showed the expected electrophoretic running behavior for T=4 and T=3 icosahedral assemblies under native conditions, irrespective of the N-terminal fusion of M (FIG. 30a). 1M-Tm^{BC2} showed similar electrophoretic mobility as the T=1 assembly of Mx^{FLAG}, which is known to occur when no cargo proteins are co-expressed.

Based on these promising biochemical data, we next expressed the 1M-Qt^{IIAG} fusion and wild-type encapsulin (QtFLAG) in HEK293T cells and subjected them to a standard EM sample preparation protocol, consisting of fixation with glutaraldehyde, post-fixation with OsO₄, followed by heavy metal staining with uranyl acetate and lead citrate, Epon embedding, microtome sectioning and imaging on TEM grids.

The resulting TEM micrographs showed annular contrast shapes with a brighter center spot outlined by a darker ring for 1M-Qt^{FLAG}.

### Example 11: Concentric EM Barcodes

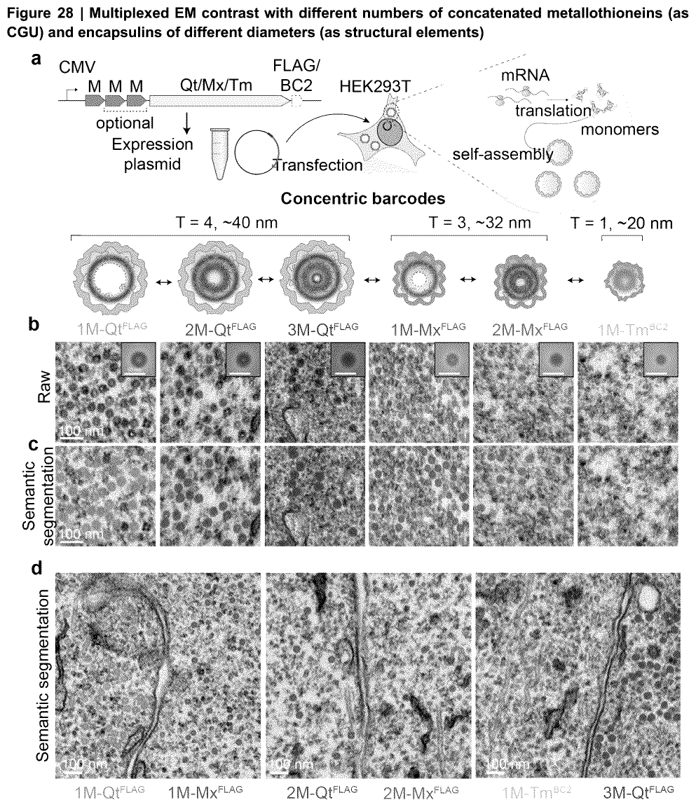

We next sought to evaluate whether concatenating multiple copies of M on the inner surface of EMcapsulins could add distinct layers of annular EM contrast (FIG. 28a). Indeed, in comparison to the TEM contrast of 1M-QtFLAG, we observed a broadening of the contrasted ring for 2M-QtFLAG and the vanishing of the bright spot in the center when a third metallothionein domain (3M-Qt^{FLAG}) was added (FIGS. 28a, 28b). Since a concatenation of 3 murine metallothionein sequences prevented EMcapsulin assembly, we constructed 3M from a chimeric sequence of three metallothionein domains from different species (MmMT3²⁰, SeSmtA²², and TaEC1²⁵).

In the case of Mx^{FLAG}, the outer diameter of the contrasted edges was expectedly smaller than that of QtFLAG such that a single M (1M-Mx^{FLAG}) resulted in a bright center similar in diameter to that seen for 2M-Qt^{FLAG}. Adding a second M to Mx^{FLAG} (2M-Mx^{FLAG}) abolished the bright central spot. Complementarily, 1M-Tm^{BC2} exhibited the smallest outer diameter without a prominent bright center (FIG. 30b). Thus, the modular combination of metal interactors and different-sized protein shells led to well-separated outer diameters for the three types of spherical protein shells and distinct radial profiles for all six classes of EMcapsulins.

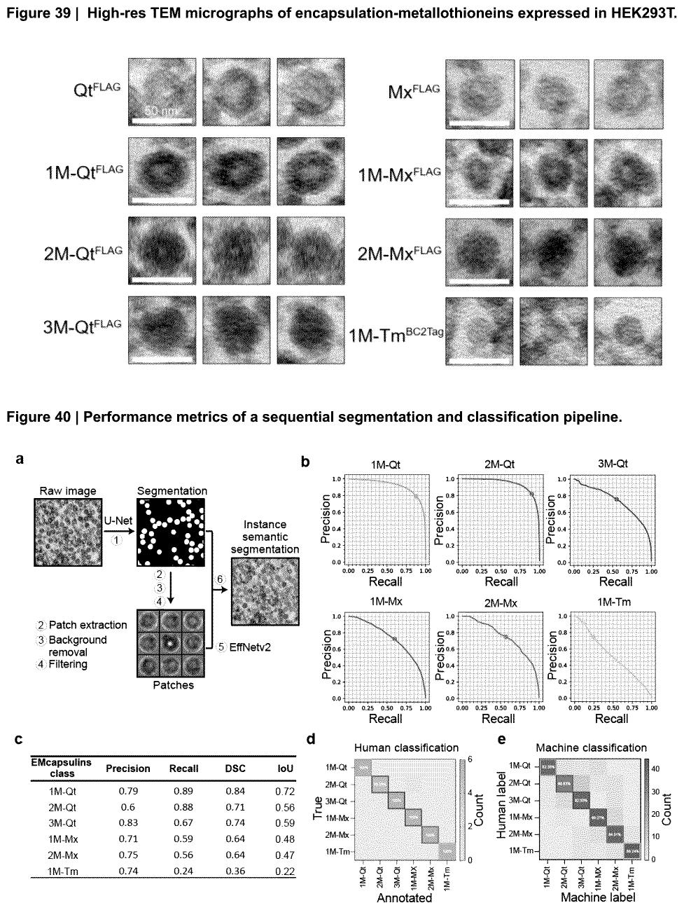

Higher TEM magnifications demonstrate that the layering of contrasted rings is in line with the location of M on the inner surface of the protein shell (FIG. 39).

### Example 12: Automatic Semantic Segmentation

To test how robustly the EMcapsulins can be identified and classified, we trained an end-to-end U-net for multi-class semantic segmentation of the 6 EMcapsulins classes on 250 TEM images obtained from all experimental categories reported here. Semantic segmentation results are shown as overlays in FIG. 28d, color-coded by class as defined in FIG. 28a. Segmentation metrics, such as the Dice similarity coefficients (DSC) per EMcapsulin class, are tabulated in Table 1, giving an average DSC score of 0.65 (validation set), 0.71 (test set 1) and 0.63 (test set 2).

We also report several object-detection metrics, such as an average recognition quality (harmonic mean of precision and recall) of 0.64, 0.70, and 0.61 for the validation set, test sets 1 and 2, respectively.

For comparison, we also implemented a sequential segmentation (U-net) followed by classification (EfficientNetV2-M) of the segmented image patches after background subtraction to ensure that the classification signal emerges from the EMcapsulins themselves (FIG. 40a). Segmentation and classification scores compared with human annotations are given in FIGS. 40 40b, 40d, 40e).

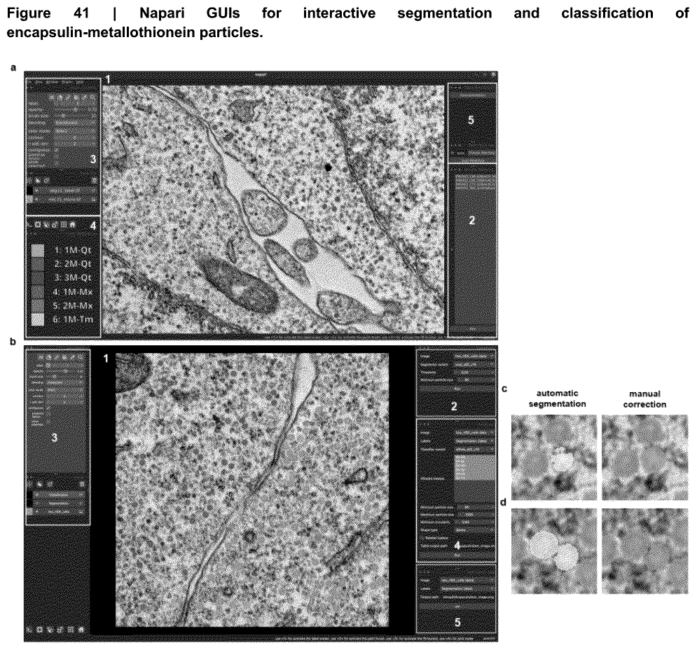

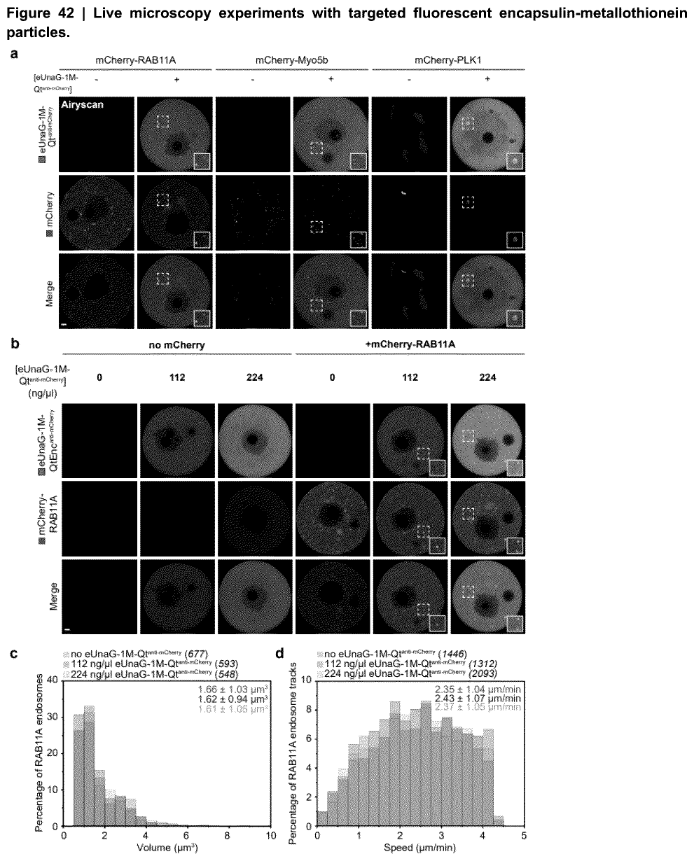

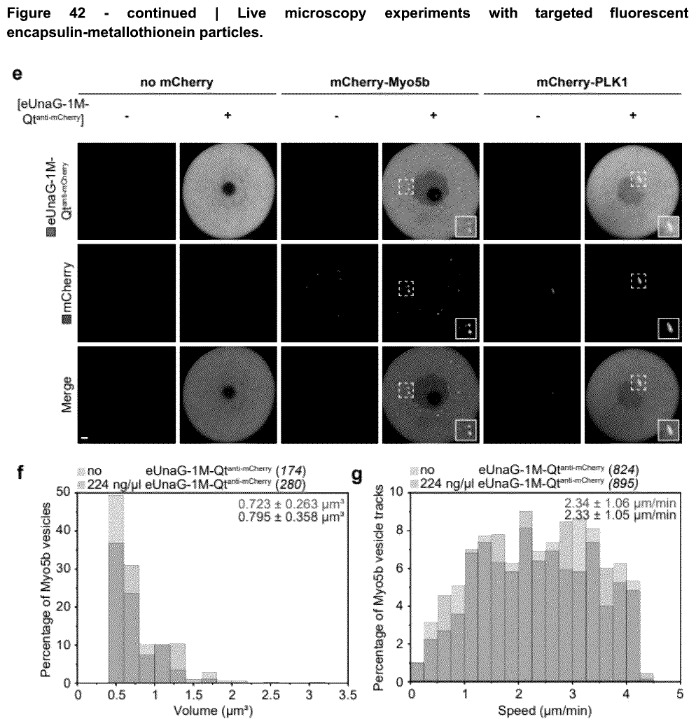

When we then expressed combinations of two EMcapsulin classes in separate HEK cells, pooled in the same sample for multiplexed detection, we found that the end-to-end network (FIG. 28d) produced slightly better semantic segmentation results than the sequential segmentation-classification pipeline (FIGS. 42e, 42f).

For optimal usability, we also generated a napari graphical user interface (GUI) that allows for interactive curation of semantic segmentations using the publicly available pre-trained models.

### Example 13: Programmable EMcapsulin Nanopatterns

We next aimed to further increase the encoding capacity of the multiplexed EM gene reporters by arranging multiple EMcapsulins into different patterns.

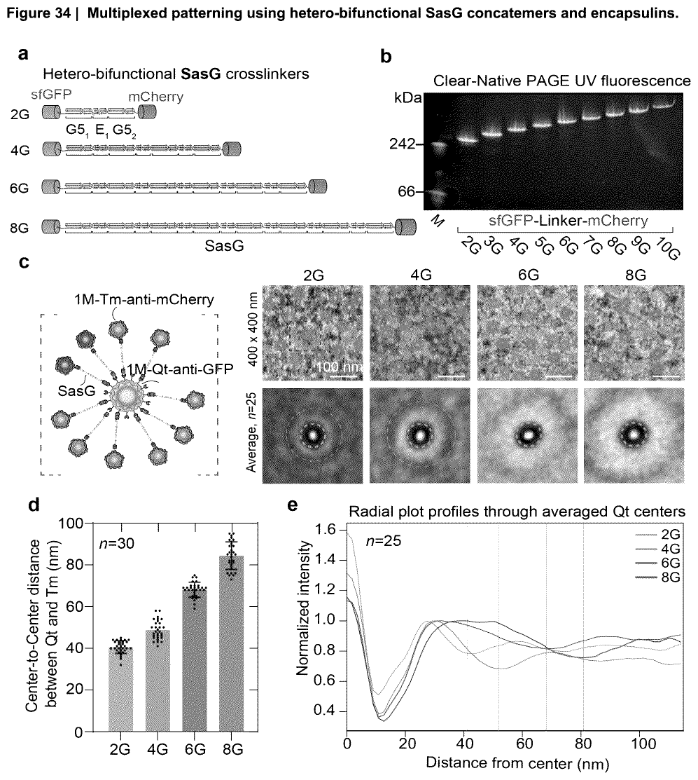

We thus generated a series of rigid heterobifunctional crosslinkers from the microbial filamentous protein SasG capped off with the fluorescent proteins superfolder GFP (sfGFP) and mCherry, for which bio-orthogonal intrabodies exist (FIG. 34a).

We obtained increasing linker lengths by concatenating G5 domains connected via E domains (2G-8G) that resulted in distinct protein species as shown by the sharp yellow fluorescent bands on CN-PAGE upon UV illumination (FIG. 34b). Only a direct fusion of the fluorescent proteins without SasG (sGFP-0G-mCherry) emitted at a red-shifted wavelength, indicating Forster resonance energy transfer (FRET).

When we then co-expressed the fluorescent linkers together with different EMcapsulins that were surface-modified with the matching intrabodies (1M-Qt^{anti-GFP} and 1M-Tm^{anti-mcherry}), we observed distinct EMcapsulin patterns in TEM, in which the annular 1M-Qt shapes were surrounded by 1M-Tm at distinct interparticle distances dependent on the linker length (FIGS. 34 c-34e).

### Example 14: Dual EM and Fluorescent Gene Reporters

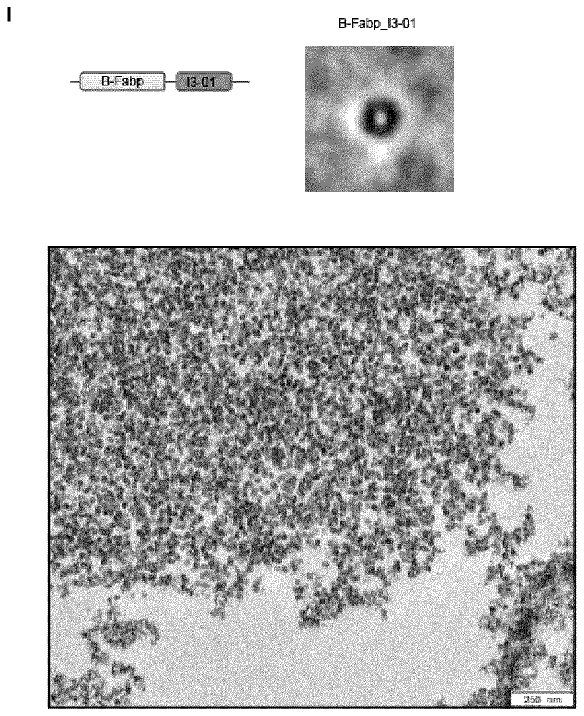

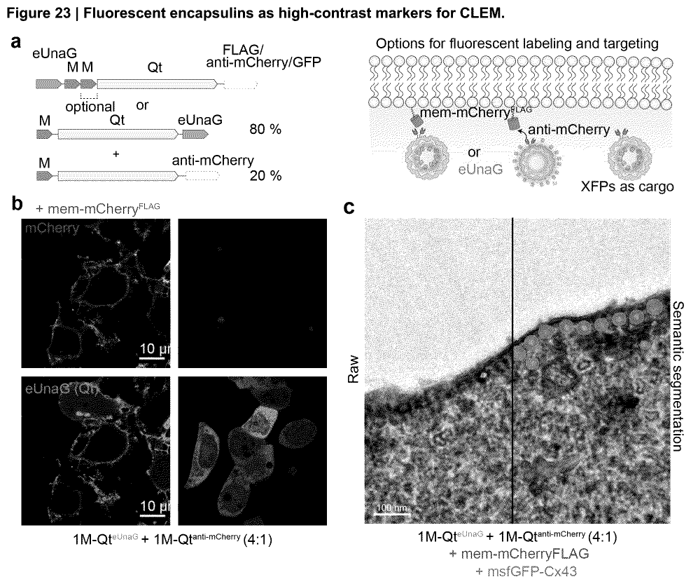

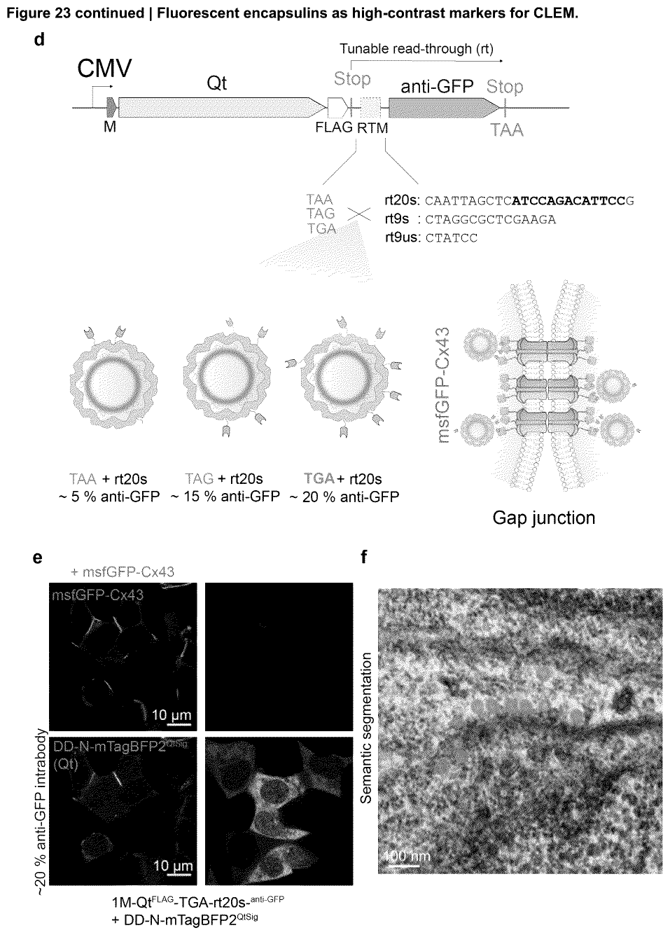

While direct fusions of fluorescent proteins of the GFP family tended to disrupt encapsulin assembly in our hands, we reasoned that the flexible M might function as a linker to a small fluorescent protein such as eUnaG to allow for proper assembly. The resulting dual-contrast EMcapsulins could be targeted to subcellular structures of interest for sequential analysis by fluorescent and electron microscopy (FIG. 23a).

Indeed, eUnaG-1M-Qt^{FLAG} migrated as a well-defined band on Clear-Native PAGE corresponding to an assembly with T=4 icosahedral symmetry. In comparison, a blurred band was detected for eUnaG-2M-Qt^{FLAG}, indicating more heterogeneous assemblies, possibly due to space limitations in the lumen of the nanospheres.

We thus investigated whether eUnaG is also tolerated on the outer surface of Qt, thus reserving the EMcapsulin lumen for variable copies of M (FIG. 23a). This variant indeed showed a sharp fluorescent band with decreased electrophoretic mobility as compared to eUnaG-1M-Qt^{FLAG} in agreement with the expected increase in the hydrodynamic diameter from adding proteins on the outer surface.

### Example 15: Labeling of Subcellular Targets

Next, we wanted to test how well the dual-mode EMcapsulins could be directed to intracellular locations of interest. We, therefore, installed anti-mCherry-intrabodies on the outer surface of the fluorescent EMcapsulin (1M-Qt^{eUnaG}) by co-expressing 1M-Qt^{anti-mcherry} in a ^{˜}4:1 ratio.

When we co-expressed membrane-targeted mCherry (mem-mCherry^{FLAG}), the fluorescent EMcapsulin co-localized to the membrane (FIG. 23b) (Manders' coefficient M1=0.870, Manders' M2=0.966, Costes P-value: 1.00), and the corresponding TEM images could resolve individual EMcapsulins lined up on the membrane (FIG. 23c).

To showcase the modularity of the labeling approaches, we tested the expression of SpyTag/SpyCatcher adapters or bioorthogonal coiled-coil pairs as targeting moieties and fluorescent proteins or APEX2 as cargo proteins.

Based on these promising results, we next chose connexins as a molecular target for fluorescent EMcapsulins. Connexins assemble into connexons forming gap-junctions that contribute to cell-to-cell communication in many biological systems, including in neuronal networks.

Connexins have also previously been fused to fluorescent proteins and modified with tetracysteine tags targeted by biarsenical fluorophores, including ReAsH. These were then used for photo-induced production of singlet oxygen to polymerize DAB, leading to electron-dense precipitates on the gap junctions, validated by immunogold labeling.

To gain control over the stoichiometry of our fully-genetic system from a single genetic construct, we developed a translational read-through (rt) system based on shortened variants of previously identified rt motifs (FIGS. 23 d-23f), in which the nascent amino-acid chain of an EMcapsulin is released at a leaky stop codon in the majority of cases. In contrast, the ribosome continues translating over the adjacent rt motif in a tunable fraction of cases to also translate the fused C-terminal intrabody. By combining three stop codons with three shortened rt motifs (rt20s, rt9s and rt9us), we created a small library yielding rt efficiencies between ^{˜}1% and ^{˜}20% (FIG. 3d).

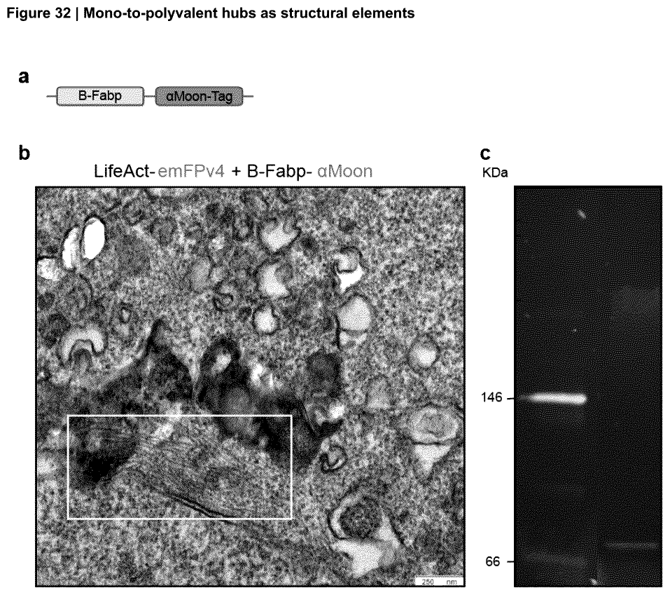

When co-expressing the rt cassette yielding ^{˜}20% translational read-through, i.e., ^{˜}50 intrabodies per EMcapsulin (1M-Qt^{FLAG}-TGA-rt20s-^{anti-GFP}) with msfGFP-Cx43, we found proper transport of Cx43 to the membrane (green channel) and adequate labeling by EMcapsulins (blue channel) on the membrane (FIG. 23e) (Manders' coefficient M1=0.989, Manders' coefficient M2=0.953, Costes P-value: 1.00), which was confirmed in the TEM micrographs from corresponding samples (FIGS. 23 f, 23g). The same rt cassette (TGA-rt20s) yielded similar connexin-labeling also for Cx43 with a C-terminally fused msfGFP (FIG. 32c), while a control condition without an rt cassette, i.e., an expression of 100% intrabodies per 1M-Qt (i.e., 240 copies on the surface) resulted in clustering of Cx43 in the ER and no cell surface signal as expected (FIG. 3d).

To further demonstrate the value of fluorescent EMcapsulins, we performed live-cell microscopy in mammalian oocytes co-expressing different mCherry-tagged targets with eUnaG-1M-Qt^{anti-mcherry} (FIG. 42). Green fluorescent EMcapsulin colocalized with mCherry-RAB11A on recycling endosomes (FIGS. 42b-42d) or with mCherry-Myo5b on cargo vesicles (FIGS. 42e-42g), in line with previously reported subcellular localizations. Automated segmentation and tracking analyses revealed no substantial difference in the volume and speed of labeled compartments in the absence or presence of EMcapsulins. In contrast, a homogenous background was observed in the absence of mCherry targets (FIGS. 42 b, 42e). In addition, we targeted mCherry-PLK1 to label relatively static acentriolar microtubule-organizing centers (FIG. 42e).

### Example 16: Multiplexed EMcapsulin Contrast in Drosophila Neurons

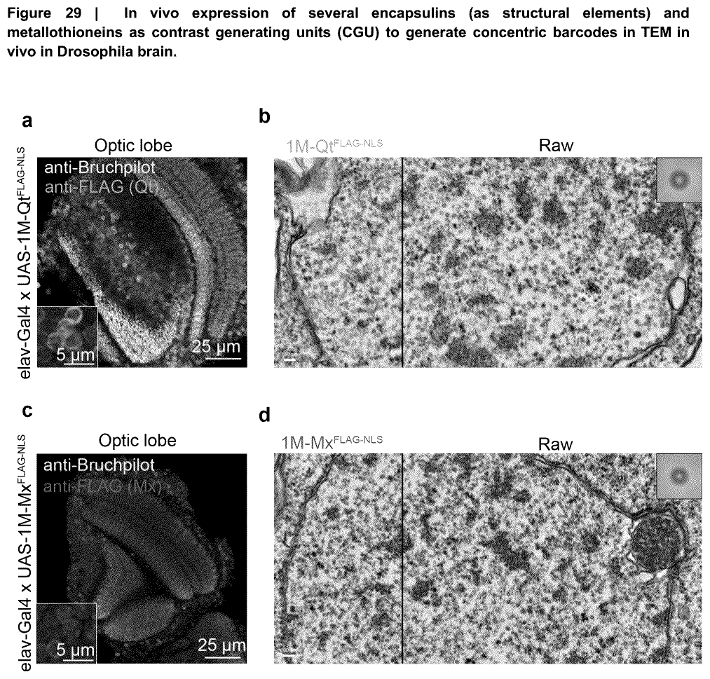

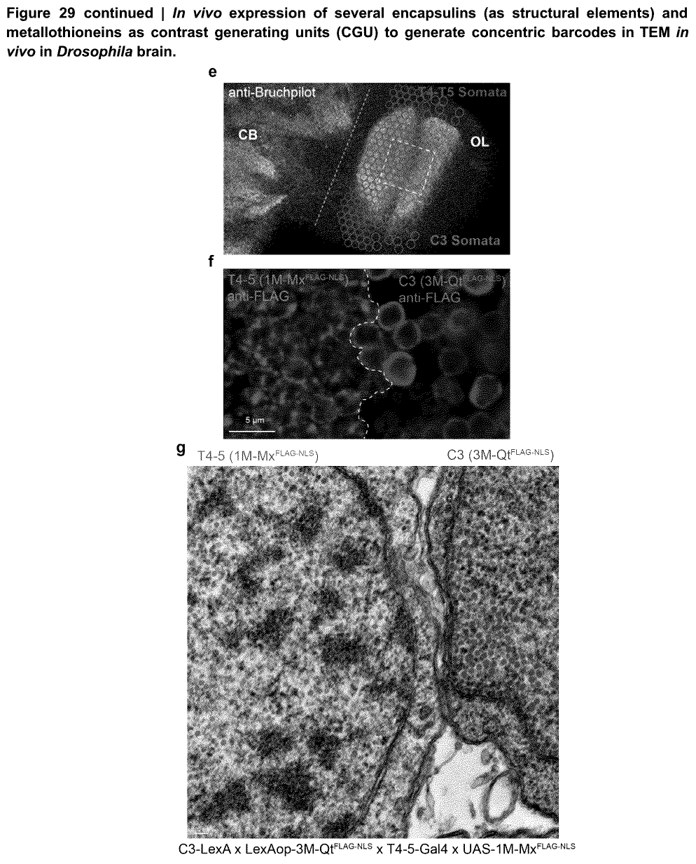

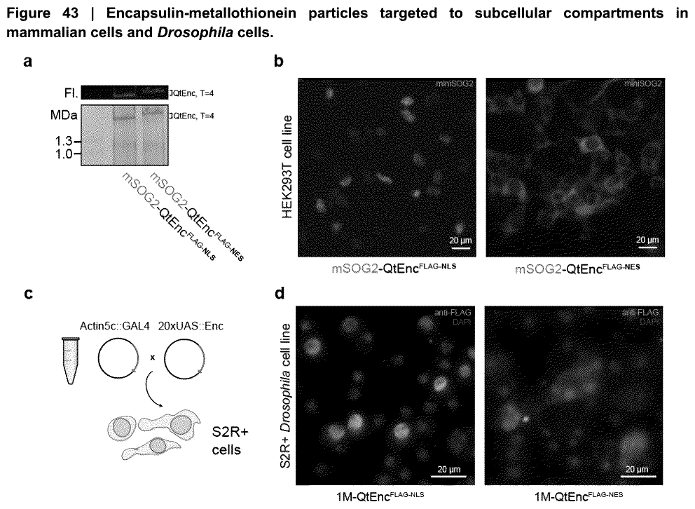

We next sought to assess whether the EMcapsulin contrast in cell culture would also transfer to in vivo applications in neurons. We thus generated transgenic Drosophila lines with pan-neuronal expression of 1M-Qt^{FLAG-NLS} and 1M-Mx^{FLAG-NLS} harboring a nuclear localization signal (NLS) (FIG. 29), whose functionality was confirmed beforehand in cell culture (FIG. 43) via immunohistochemical analyses (insets in FIGS. 29 a, 29c).

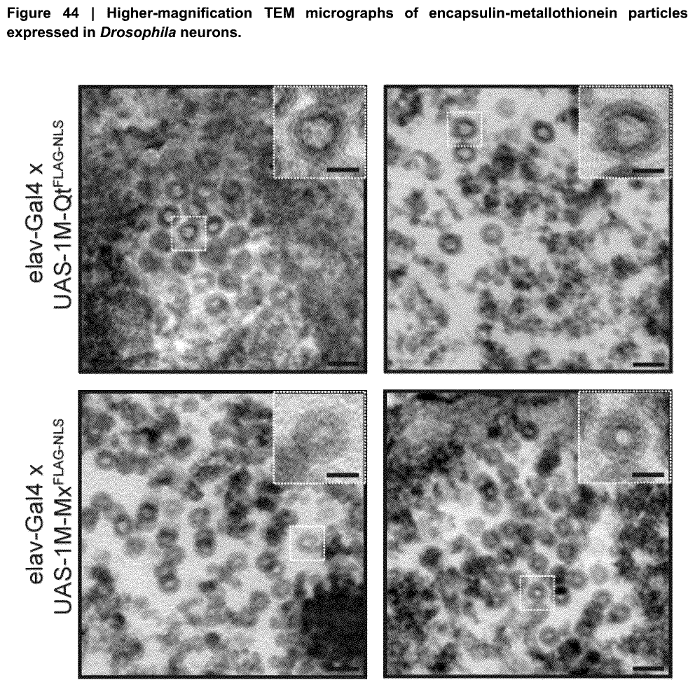

Higher TEM magnifications again showed increased contrast on the inner surface of the EMcapsulin protein shells, similar to what was observed in HEK cells (FIG. 44).

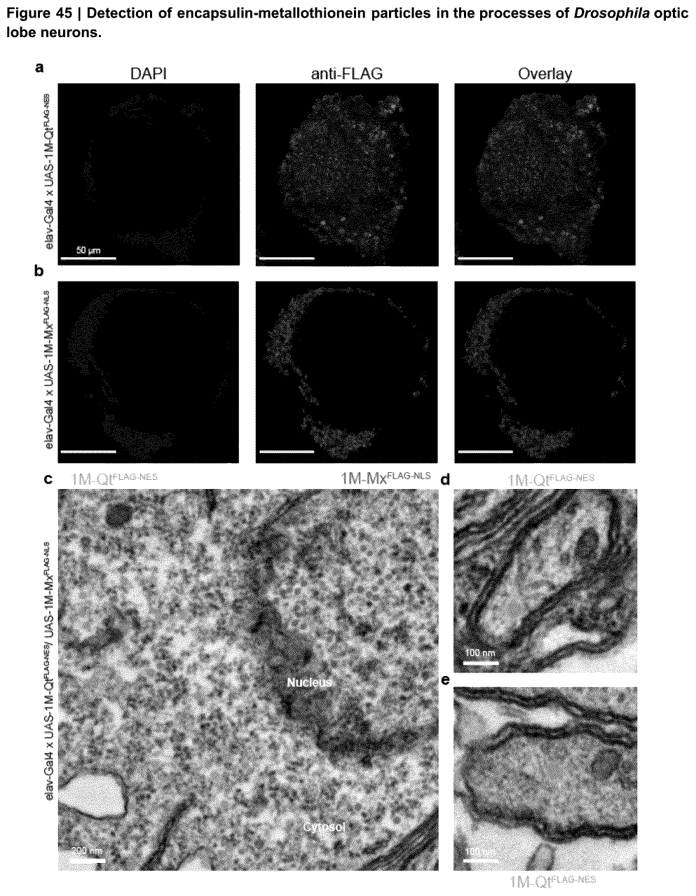

To demonstrate multiplexed EMcapsulin detection in different neuronal types in the same animal, we generated a transgenic Drosophila line expressing 3M-Qt^{FLAG-NLS} in C3 neurons and 1M-Mx^{FLAG}-^{NL}S in T4-5 neurons, whose somata are adjacent in the optic lobe, such that one can capture them in the same field-of-view in TEM (FIGS. 29e-29g). Furthermore, we have co-expressed 1M-Qt with a nuclear export signal (1M-Qt^{FLAG}-^{NES}) and 1M-Mx^{FLAG-NLS} in the same neuronal type and observed substantial expression of 1M-Qt^{NE}S-^{FLAG} in neuronal processes (FIG. 45).

### Example 17: EMcapsulin Contrast in Volume SEM

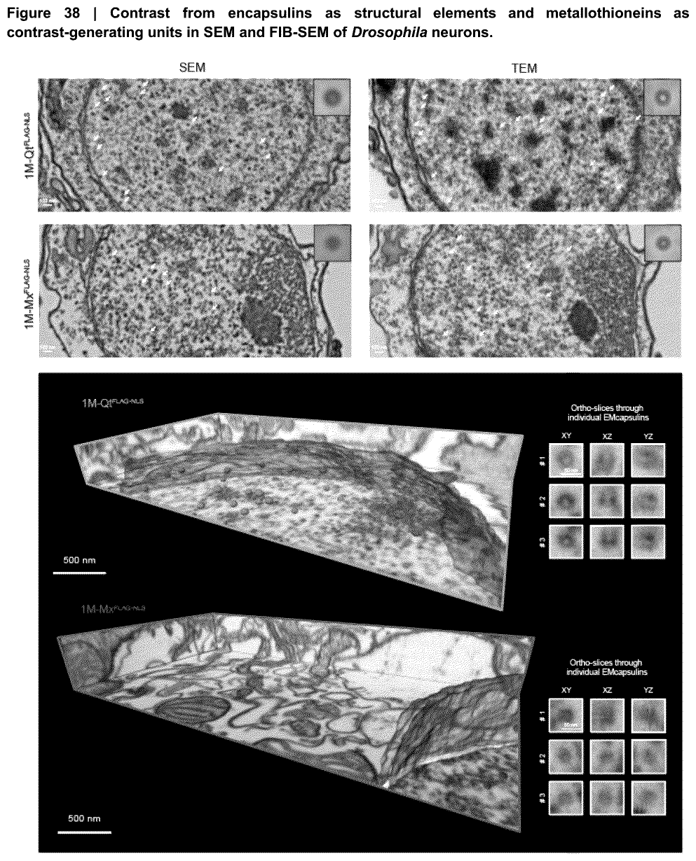

Since SEM is the other common mode for volume EM, we compared SEM and TEM contrast directly from adjacent ultramicrotome sections of the same Drosophila neurons. We found that similar image information can be obtained from EMcapsulins in TEM and SEM (FIG. 38). The line profiles through the EMcapsulins in TEM and SEM show similar outer diameters based on the contrast edges, whereas the lumen of both nanospheres appears brighter in TEM than in SEM.

We also tested whether the EMcapsulin contrast is compatible with FIB-SEM tomography, which enables fully automated volume imaging with isotropic nanometer resolution. We chose a voxel size of 4 nm, a resolution informative for connectome analyses and could readily discern 1M-Qt^{FLAG-NLS} (FIG. 38c) and 1M-Mx^{FLAG-NLS} (FIG. 38d).

### Example 18: EMcapsulin Expression in Mouse Brain

To assess EMcapsulin contrast in mammalian neurons, we co-expressed 2M-Qt^{FLAG} with m-Scarlet-I in mouse hippocampus via viral transduction. Native gel analysis verified EMcapsulin expression and assembly (FIG. 30b). Immunohistochemical fluorescence analysis confirmed EMcapsulin expression in CamKIIa-positive neurons in the hippocampus (FIG. 30c).

EM analysis revealed EMcapsulin expression in neuronal somata (FIG. 30d) and processes (FIGS. 30f-30g), although some membrane discontinuities were observed in some regions, occasionally precluding a clear delineation of cellular boundaries. The narrow size distribution and sphericity of the rigid proteinaceous EMcapsulin shells produced concentric contrast edges at the inner and outer circumferences of the annular cross-sections. This appearance differentiated the EMcapsulins from synaptic vesicles, which exhibited more variable sizes and non-circular luminal and external borders consistent with their flexible lipid membranes.

Jointly, these imaging data show robust detection of barcoded EMcapsulins in different cell types and EM modalities.

### Materials and Methods of Examples 10-18

### Molecular Biology

All DNA constructs were custom-synthesized by IDT DNA Technologies or assembled from multiple fragments via HiFi assembly, traditional ligation cloning (using EcoRI and NotI sites), or PCR-based mutagenesis methods, and cloned into pcDNA 3.1(+) Zeocin for mammalian expression. The constructs are presented in Table 2 below.

### Mammalian Cell Culture

Low passage number HEK293T cells (ECACC: 12022001, obtained via Sigma-Aldrich) were cultured in advanced DMEM with 10% FBS and penicillin-streptomycin at 100 μg/ml at 37° C. and 5% CO₂. HEK293T cells were transfected using X-tremeGENE HP (Roche) transfection reagent according to the manufacturer's protocol (3 μl reagent per μg of DNA).

### Fly Husbandry and Strains

Flies were raised in the facilities at the Max Planck Institute for Biological Intelligence at 25° C. and 60% humidity on standard cornmeal agar medium at 12 h light/dark cycle. Only female brains were analyzed. The following driver lines were used: elavC155-Gal4 (pan-neuronal expression, Bloomington Drosophila Stock Center (BDSC) 458), R35A03-LexA (expression in C3 neurons, BDSC 54706) and R42F06-Ga/4 (T4-T5 neurons expression, BDSC 41253). The UAS-EMcapsulin (1M-Qt^{FLAG-NLS} and 1M-Mx^{FLAG-NLS}) strains were generated as follows: The DNA cassette encoding the EMcapsulins was custom-synthesized and subsequently cloned into XhoI/XbaI sites of pJFRC7-20XUAS-IVS-mCD8::GFP (Addgene plasmid No. 26220), after removal of the mCD8::GFP cassette. The plasmids were injected into the attP2 landing site strain (Bloomington Drosophila Stock Center, no. 8622) for PhiC31 integrase-mediated transgenesis (BestGene Inc.). The LexAop-EMcapsulin (₃M-Qt^{FLAG}-^{NLS}) strain was generated as follows: The DNA cassette encoding the EMcapsulin was custom-synthesized and subsequently cloned pJFRC19-13XLexAop2-IVS-myr::GFP (Addgene plasmid No. 26224) using XhoI/XbaI after removal of the myr::GFP cassette. For the expression of the 3M-QtFLAG-NLs and 1M-MxFLAG-NLS EMcapsulins in C3 and T4-T5 neurons, respectively, we used both the Gal4/UAS and LexA/LexAop binary expression systems. The final genotype allowed for the simultaneous expression of LexAop-3M-Qt^{FLAG-NLS} by the C3-neuronal driver 35A03-LexA and the expression of UAS-1M-Mx^{FLAG-NLS} by the T4-T5-neuronal driver R42F06-Ga/4. In a separate experiment, we pan-neuronally (elavC155-Ga/4 driver) co-expressed UAS-1M-Qt^{FLAG-NES} and UAS-1M-Mx^{FLAG-NLS}.

Experiments with Mice

All in vivo experiments in mice were approved by the government of Upper Bavaria. Experiments were carried out in three male C57BL/6N, 3 months old mice. Animals were housed in individually ventilated cages in specific pathogen-free conditions and a 12 h light/dark cycle. Water and food were provided ad libitum. Surgical preparations. Mice were administered 0.1 mg/kg buprenorphine (Temgesic, Indivior UK) intraperitoneally 30 minutes before the start of the surgery. Isoflurane was used for inhalation anesthesia: 5% for induction and 1.5-2% for maintenance. Anesthesia depth was checked by corneal and toe pinch reflexes, and the surgery started once these reflexes were absent. Body temperature was maintained around 36.5° C. with a heating mat, and corneal hydration was ensured using eye ointment (Bepanthen, Bayer). Viral injections. Mice were positioned in a stereotaxic frame, and the skin was disinfected with Betadine (Braunoderm, Braun). 20 μL lidocaine 2% (Braun) were injected subcutaneously for additional local skin and periosteum anesthesia. A 10 mm long scalp incision was made, and the fascia was gently pushed to the side. The skull was cleaned and allowed a few minutes to dry. A 400 μm in diameter burr hole was drilled while avoiding overheating or damage to the meninges. A 33 gauge stainless steel injection cannula was inserted about 1500 μm below the surface of the cortex, and one μL of AAV5-CaMKIIa-mScarlet-I-P2A-2M-Qt^{FLAG} (^{˜}1×10¹² particles) solution was injected over a 10 min period using a syringe pump (PHD 22/2000, Harvard Apparatus). The cannula was held in position for 5 minutes after the injection to allow the viral solution to diffuse in the brain tissue and then slowly retracted. The incision was closed with tissue glue (Vetbond, 3M), and lidocaine 2% was applied to the skin to prevent postoperative pain. For postoperative analgesia, 5 mg/kg Meloxicam (Metacam 2 mg/ml, Boehringer Ingelheim) was injected subcutaneously, and the animals were kept on a heated mat until they woke up. 5 mg/kg Meloxicam was administered subcutaneously once a day for the two subsequent days to provide postoperative analgesia. Brain dissection. One month after the virus injection, mice were sacrificed with an overdose of Ketamine/Xylazine and perfused with PBS. The brains were dissected and stored in 4% PFA solution for further processing for immunohistochemical analysis or directly homogenized in MPER lysis buffer using a Dounce tissue homogenizer without fixation agent for pull-down experiments.

### Gel Electrophoresis

Blue native (BN) and Clear Native (CN) PAGE analyses were performed using the NativePAGE Novex Bis-Tris Gel System (Invitrogen) according to the protocol of the manufacturer.

For CN-PAGE, the cathode buffer contained 0.05% of the anionic detergent sodium deoxycholate. Briefly, cell lysate volumes containing 100-500 ng of nanocompartments were loaded onto pre-cast NativePAGE™ 3 to 12% gels and run at 150 V for 2 h at room temperature. If fluorescent protein assemblies were separated on CN-PAGE, the apparatus was shielded from light to avoid bleaching. CN-PAGE gels were illuminated on a standard UV table and documented using a conventional cell phone camera to detect fluorescently-labeled protein assemblies. To stain for the total protein content in the cell lysates, we performed a Coomassie staining on the BN/CN-PAGE gels. For on-gel APEX2-mediated DAB-polymer formation, gels were treated with DAB and hydrogen peroxide using the SIGMAFAST™ DAB Kit (Sigma Aldrich, D0426). SDS-PAGE was performed using a Bio-Rad Mini-PROTEAN cell and pre-cast 12% Bio-Rad TGX gels (40 min at 200 V). Protein bands from pull-down experiments were visualized using SilverQuest™ Silver Staining Kit (Thermo Fisher). For pull-downs of FLAG-tagged EMcapsulins, anti-FLAG® M2 Magnetic Beads (Sigma-Aldrich, M8823) were used according to the manufacturer's protocol and eluted using 3× FLAG® Peptide (Sigma Aldrich, F4799). BC2-tagged EMcapsulins were pulled down with Spot-Trap Magnetic Agarose beads (Chromotek, ‘etma’) according to the manufacturer's protocol and eluted in the native state using an alkaline elution buffer.

### Immunolabeling and Confocal Microscopy (Drosophila)

For immunolabeling, brains were dissected in cold PBS and fixed in 4% paraformaldehyde (containing 0.1% Triton X-100) at room temperature for 22 min. Afterward, the brains were washed three times with PBT (PBS containing 0.3% Triton X-100) and blocked with 10% normal goat serum in PBT at room temperature for 1 h. Brains were then incubated with primary antibodies diluted in PBT containing 5% normal goat serum for 24-48 h at 4° C.

After five wash steps with PBT, brains were incubated with secondary antibodies diluted in PBT containing 5% normal goat serum for 24-48 h at 4° C. Brains were subsequently washed five times with PBT and once with PBS before being mounted in SlowFade Gold Antifade Mountant (Thermo Fisher Scientific). Imaging was performed using a Leica SP8 laser scanning confocal microscope equipped with 488-, 561- and 633-nm lasers and a 63× objective. Image processing was performed with the ImageJ software package⁵². The following antibodies were used. Primary antibodies: rat anti-FLAG (1:200, Novus Biologicals, NBP-1-06712), rabbit anti-Sox102F (1:200⁵³), and mouse anti-Bruchpilot (1:20, Developmental Studies Hybridoma Bank (DSHB), AB2314866). Secondary antibodies (used at 1:400): Alexa Fluor 568-conjugated goat anti-mouse (Invitrogen, A11004), Alexa Fluor 568-conjugated goat anti-rabbit (Invitrogen, A-11011), and Alexa Fluor 647-conjugated goat anti-rat (Invitrogen, A21247). DAPI (1:1000; Invitrogen) was applied for 5 min at the end of the immunolabeling protocol to stain nuclei, followed by extensive washing with PBT. The identity of adjacent C3 and T4-5 neurons in the IHC shown in main FIG. 4f was established based on the co-staining against Sox102f.

### Mouse Brain IHC and Microscopy

Brain tissue was cut into 70 μm thick slices on a cryotome before incubation for 1 hour at room temperature in a 1% BSA and 0.2% Triton X-100 in PBS solution to ensure permeabilization/blocking. Next, tissue slices were incubated at room temperature for 2 hours with an anti-FLAG FITC (F4049, Sigma Aldrich) monoclonal antibody conjugate diluted to a 2 μg/ml. Slices were washed 3 times for 5 min in PBS and incubated with 10 mM DAPI. Again, tissue was washed 3 times for 5 minutes with PBS, and brain slices were mounted on microscopy slides with Aqua Poly Mount (Polysciences, Warrington, PA). Imaging was performed on a Leica SP5 (Leica Microsystems) to acquire the FITC signal corresponding to 2M-Qt^{FLAG} EMcapsulins and the signal from co-expressed mScarlet-I and DAPI.

### Fluorescence Microscopy on HEK Cells

Widefield fluorescence microscopy was performed on an EVOS fluorescence microscopy system (Invitrogen) equipped with filter cubes to image DAPI, GFP, and RFP. The GFP filter cube was used to image eUnaG. A Leica SP5 (Leica Microsystems) equipped with 405, 488, 561, and 633 nm laser lines were used for confocal microscopy imaging, shown in FIG. 3. Colocalization analysis was performed with the Coloc2 plugin (release 3.0.5) for ImageJ (v1.53u). The region of interest was set around the prominent cells to exclude background. A PSF of 3.0px was assumed as a conservative estimation for standard confocal microscopes. Background subtraction was performed in ImageJ on both channels with a 50px rolling ball subtraction without smoothing. Costes P-value was calculated with 100 randomizations.

### Live-Cell Microscopy on Mammalian Oocytes

Oocytes were isolated from the ovaries of 8- to 12-week-old FVB/N female mice. Fully grown oocytes of around 75 μm in diameter with a centered nucleus were arrested at prophase in homemade phenol red-free M2 supplemented with 250 μM dibutyryl cyclic AMP (dbcAMP) (Sigma-Aldrich) under paraffin oil (ACROS Organics) at 37° C. For eUnaG imaging, the medium was supplemented with 3 μm bilirubin (Sigma-Aldrich). eUnaG-1M-Qt^{anti-mcherry}, mCherry-Myo5b, mCherry-Plk1, and mCherry-RAB11A mRNAs were synthesized and quantified as previously described. Mouse oocytes were microinjected with 3.5 μl of mRNAs. eUnaG-1M-Qt^{anti-mcherry} mRNA was microinjected at a needle concentration of 112 or 224 ng/μl, mCherry-Myo5b mRNA at 152.1 ng/μl, mCherry-PLK1 mRNA at 111.1 ng/μl, and mCherry-RAB11A mRNA at 84.1 ng/μl. Oocytes were allowed to express the mRNAs for 3 hours before confocal or Airyscan imaging on LSM880 (Zeiss).

Automated segmentations of RAB11A-positive recycling endosomes and Myo5b-positive vesicles were performed using the Machine Learning Trainer function of Vision4D (Arivis). Segmented objects were tracked using the Tracking function. Specific parameters used were: Brownian Motion (Centroid) for motion type, 1 μm for max. distance, Center of Geometry for centroid, no fusions or divisions, none for continue tracks, 2 for max. time gap and none for weighting. The volumes and speeds of RAB11A-positive recycling endosomes and Myo5b-positive vesicles were then exported into Excel (Microsoft) and OriginPro (OriginLab).

### EM Sample Preparation

For EM sample preparation, the staining method was adapted from Briggman et al., Nature (2011) and used for both HEK cells and freshly dissected Drosophila brains. HEK293T cells were harvested with Accutase (Sigma-Aldrich) 36 hours post-transfection and pelleted by centrifugation. As an initial demonstration for multiplexed EMcapsulin detection across different cells, cell suspensions of HEK293T cells transiently expressing a single class were mixed and pelleted by centrifugation. Following the initial preparation, the material was fixed with 2.5% Glutaraldehyde (Electron Microscopy Sciences) in 0.1 M sodium cacodylate buffer (CB) (pH 6.7-7.0) for 20 min (for Drosophila brains, the fixation duration was varying between 20 minutes and 24 hours). After removal of the fixative, the material was postfixed using a 1:1 mixture of 4%0.0 (Electron Microscopy Sciences) with 0.3 M CB, containing 3% potassium hexacyanoferrate (II) (Sigma-Aldrich) for 20 minutes on ice. The post-fixative solution was removed, and the material was washed twice with 0.1 M sodium cacodylate buffer for 2 minutes, followed by 2×5 min ddH₂O washing steps. ddH₂O water was removed, and the material was stained using 1% tannic acid (Sigma-Aldrich) for the duration of 10 min, RT. Then, tannic acid was decanted, and the material was washed 5 times for 5 minutes using ddH₂O water. Subsequently, the material was treated with 1% uranyl acetate (Electron Microscopy Sciences) solution (30 min, RT), with successive ddH₂O 5×5 minutes washing. Afterward, the material was treated with 3% Lead aspartate (Sigma-Aldrich) for 15 min under RT. Again, the material was washed (5×5 min in ddH₂O water) before proceeding to epoxy embedding. The epoxy medium for the embedding process was prepared as follows: 61.5 g 2-Dodecenylsuccinic-acid anhydride (Serva) was mixed with 81.5 g of Methyl nadic anhydride (Serva) as well as with 130.5 g Glycidether 100 (Serva). The resulting mixture was stirred, and 3750 μL of 2,4,6-Tris(dimethylaminomethyl)phenol (Serva) was added to the mixture, stirred, and aliquoted for storage at −20° C. The epoxy embedding process was conducted over 2 days. Immediately after the last washing step, the material was incubated on ice with 75% EtOH for 10 minutes, followed by incubation in 90% EtOH for another 10 minutes. Then, the sample was left in absolute ethanol for 1 hour on ice with the solvent replacement after 30 minutes. The EtOH solution was replaced with pure propylene oxide and incubated for 5 minutes at RT. Next, the solution was discarded, and the material was incubated in 1:1 mixture of Epon and Propylene oxide (Electron Microscopy Sciences) for 30 minutes under RT. Subsequently, the material was incubated at room temperature in 100% Epon for 30 minutes and left in fresh Epon for another 12 hours. Epoxy was removed, and the material was briefly rinsed in fresh 100% epoxy and left in newly-poured 100% epoxy for 72 hours at 60° C. The Drosophila brain prior to final epoxy curation was oriented such that optical lobes were parallel to the block's slicing plane. The resulting blocks were subjected to trimming and slicing. For cellular material, the trimming of excess epoxy from the block's surface was done using an EM TRIM milling system (Leica Microsystems, Germany). Using an UltraCut E microtome (Reichert/Leica, Germany) the prepared blocks were prepared with a histo-knife (DIATOME, Switzerland) and then sequentially cut with an ultra-knife (DIATOME, Switzerland) at a slice thickness of 70 nm, verified by the slices' interference pattern. The slices were deposited either on the surface of a 200 mesh copper grid or the polished side of a silicon wafer.

### TEM

TEM images were acquired on a Libra120 TEM (Carl Zeiss GmbH, Germany), equipped with a CCD camera (TRÖNDLE Restlichtverstärker-Systeme, Germany) using ImageSP software (SYSPROG, Belarus). Before image acquisition, all grid-supported specimens were pre-irradiated at 120 kV beam voltage and 200 μrad illumination angle without apertures. The actual image acquisition took place with the activated BIO-AIS condenser aperture system and a 60 μm objective aperture. The same beam conditions used for pre-irradiation were also applied for imaging, except for an illumination angle of 100 μrad. The magnification for most of the TEM images was chosen such that a pixel size of 1.81 nm was achieved at an exposure time of 1000 milliseconds.

### SEM

SEM images were acquired using a Gemini 360 scanning electron microscope (Carl Zeiss GmbH, Germany), equipped with a sense-BSD detector (Carl Zeiss GmbH, Germany). Silicon wafers, supporting the samples were glued to the stage with silver glue (PLANO GmbH, Germany) and loaded on a sample holder. SEM image acquisition was performed with a beam voltage of 6.5 kV, 2 nm nominal pixel size, a 30 μm objective aperture, and a at a working distance of 3.7 mm. The stage was subjected to a bias voltage of 5 kV.

### FIB-SEM

FIB-SEM images were acquired on an SEM Crossbeam 550 (Carl Zeiss GmbH, Germany), equipped with InLens, BSE, and SESI detectors (Carl Zeiss GmbH, Germany) running ATLAS software. The block with the specimen of interest was firmly attached to the stage using silver glue (PLANO GmbH, Germany). The sample was coated with an electron-transparent carbon layer (approx. 5 nm thickness) using an external carbon evaporator device. The carbon-coated sample was loaded into the FIB-SEM chamber, and a platinum guiding pad was deposited on the identified ROI to aid the localization of the ROI after sputter coating.

The sample was retrieved and loaded into an external sputter coater, where an electron-opaque iridium layer (˜30 nm thickness) was deposited. Back in the FIB-SEM chamber, a 3D platinum pad with a thickness of 2 μm was placed onto the block in proximity to the region of interest. Subsequently, a tracking pattern was milled in the deposited pad to simplify image registration. Finally, the platinum pad and grooves were covered with the carbon layer (2 μm thickness) on top. The following settings were used for image acquisition: SEM beam voltage 1.3 kV, a working distance of 5 mm, 6 μs target dwell time, and 4 nm nominal voxel size using an InLens detector. The FIB Ga beam was accelerated by 30 kV voltage at a current of 700 μA. An image volume of 3696 nm (width)×1956 nm (height)×404 nm (milling length) was acquired in ˜70 minutes using ATLAS 3D software (Carl Zeiss GmbH, Germany). The acquired volume was pre-aligned by template matching to the surface landmarks and post-aligned with the Linear Stack Alignment using the SIFT algorithm implemented in ImageJ. Renderings of the acquired FIB-SEM data were computed either in Imaris (Imaris 9.8 Oxford Instruments PLC, Great Britain) or in Dragonfly software (Dragonfly 2021.3, Object Research Systems (ORS) Inc, USA).

### End-to-End Multi-Class Semantic Segmentation Network

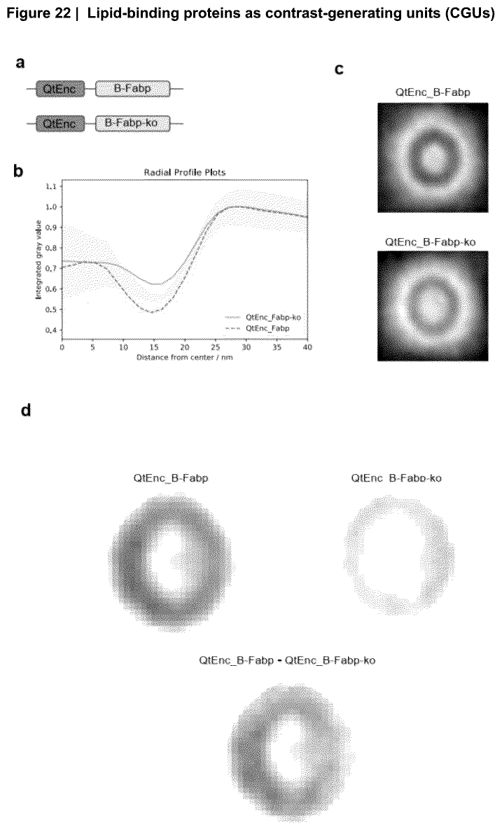

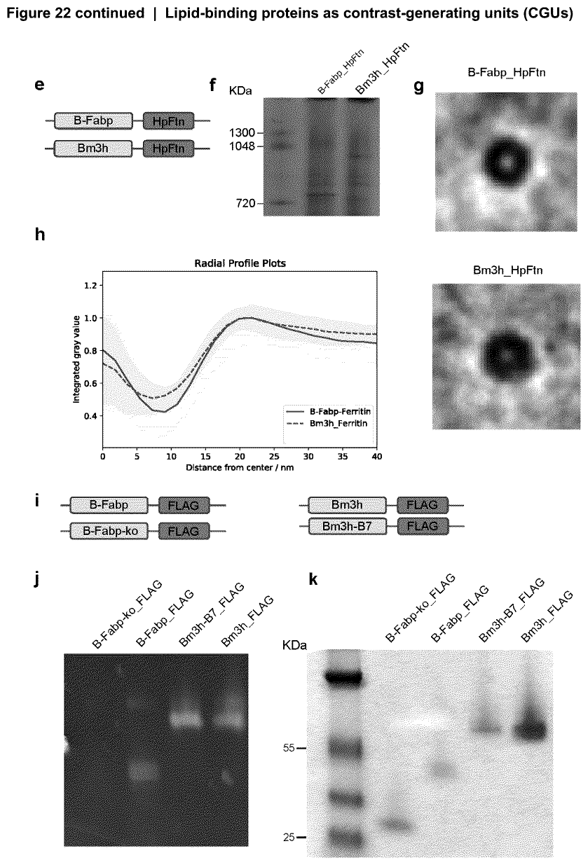

We employed a basic U-Net architecture inspired by Falk and colleagues. For our training runs, we used a dropout of 0.1 and mish activation function and otherwise used the defaults of the MONAI implementation (docs.monai.io/en/stable/networks.html #basicunet). This implementation represents a standard U-Net architecture with an encoder and decoder connected by skip connections and has been proven successful in other biomedical segmentation tasks. The network features 1 input and 8 output channels. Besides an output channel for each of the six EMcapsulin classes, we implemented a background channel and a channel for the EMcapsulin patterns consisting of cross-linked 1M-Qt and 1M-Tm (FIG. 22). A percentile-based normalization is applied for training and inference. Training. We used 250 TEM micrographs (pixel size of 1.81 nm) taken on a Libra120 TEM (Carl Zeiss GmbH, Germany). We trained the model for 3000 epochs with Ranger21 optimizer, an initial learning rate of 1e-2, and a batch size of 2. A batch consisted of 20 random crops sized 512×512 pixels. During training, we employed basic augmentation strategies, namely Gaussian noise, flips, and random affine transformations. An equally weighted sum of soft Dice and Binary Cross Entropy (BCE) inspired by Isensee et al. served as a loss function for our training runs. Inference. To derive segmentations, we combined test time augmentations (TTA), namely flips and Gaussian noise, with a sliding window inference (SWI). For the SWI, we used a batch size of 32 and an overlap of 0.5. We derive multi-class segmentation maps by computing argmax on the six class channels. Further, we provide the possibility to preserve network outputs for all eight channels enabling downstream analysis. Post-Processing. To refine the segmentation maps, we conducted conservative post-processing in a multi-step procedure and provided means to fine-tune each of the steps on an individual basis. Therefore, we first binarize the six-channel segmentation maps. Then we compute a connected component analysis on the binarized segmentation maps using cc3d. We then removed particles with less than 42 pixels because EMcapsulins are at least 20 nm in diameter (˜96 pixels area). We chose this threshold value as a compromise to remove ‘noise’ but maintain partially successful segmentations (e.g., half-rings on the borders of EMcapsulins). Next, we conducted conditional majority voting within each binary connected component. We thus assigned the class represented by the majority of pixels. This step was applied only if the structures were below a maximum of 500 pixels to refrain from modifying the class of touching objects and a circularity larger than 0.2. We also refrained from the majority voting in case of ties (several majority classes with the same number of pixels). Ultimately, we filled up holes in the multi-class segmentation maps for pixels that were completely surrounded by foreground pixels belonging to the same class. Datasets and annotation. An intra-image split into training (70% of the raw image) and validation set (30% of the raw image) was performed by randomly choosing respective ‘stripes’ in each TEM micrograph. Pixel-accuracy annotations were performed in ImageJ, yielding a total number of 35282 annotated particles in the training data set. Independently acquired TEM micrographs were annotated for the independent test set 1 (57 images, 10 images annotated for the subset “two EMcapsulins classes in adjacent HEK cells”) and test 2 (single EMcapsulin class, 26 images, 14 images annotated). Evaluation. Besides qualitative analysis, we relied on quantitative metrics for the comparison of our CNN models. Therefore, we report pixel-wise Dice similarity coefficients (DSC), sensitivity, and precision. Furthermore, we computed instance-level metrics based on an intersection over union (IOU) criterion of 0.5. We further report the three panoptic quality metrics (see Table 1).

We summed up the respective confusion matrices globally for the computation of both pixel and instance metrics across all microscopy slices.

We conducted this procedure to treat all EMcapsulins equally, irrespective of their occurrence, in a dense or sparse microscopy image. We approximated instances with a connected component analysis using cc3d since our annotations were not optimized for instance semantic segmentation.

This heuristic is not perfect, as closely located EMcapsulins touching each other can be merged into one instance. Therefore, factually correct network predictions might be classified as false positives resulting in overly pessimistic instance-level metric computations. Based on the above criteria, we select the checkpoint from epoch 2070, producing the lowest loss and, coincidentally, also the best volumetric DSC for training. We report results on the test set 1 in FIG. 28d, inter alia, showing multi-class segmentation maps as an overlay. Hardware. Computations were run on a rack server equipped with an AMD EPYC 7313 16-Core Processor in combination with NVIDIA RTX 8000 and A5000 GPUs using CUDA version 11.4 in conjunction with Pytorch 1.13.0 and MONAI version 1.0.

### Sequential Segmentation-Classification Pipeline.

The model was implemented in PyTorch using the elektronn3 neural network toolkit github.com/ELEKTONN/eletronn3) and trained and tested on NVIDIA A40 GPUs, hosted at the Max Planck Computing facility MPCDF in Garching, Germany. The same 250 TEM micrographs with intra-image splits were also used for training and validation of the segmentation-classification pipeline. The U-Net model for segmentation was enhanced by including an additional batch normalization layer after each convolution layer and trained for 160,000 steps using the AdamW optimizer and a batch size of 8. In order to mitigate the impact of the strong foreground-to-background class imbalance of the training data, the training objective was chosen to be the sum of a weighted dice loss function (and a weighted cross-entropy loss function (foreground pixels were weighted 5 times more than background pixels). The following augmentations were applied during training of the U-net segmentation model: cropping of 384×384 pixel patches from random regions of the source images, random flipping, shifting, scaling and rotation, additive Gaussian noise, random gamma correction, and random brightness and contrast changes.

The EfficientNetV2-M model for patch-based EMcapsulin classification was trained with random flipping, scaling, and rotation augmentations. It was trained for 120,000 steps using the AdamW optimizer with a batch size of 128. The dataset of patch images was rebalanced by undersampling overrepresented classes. In order to prevent the model from fitting onto potentially informative background information in the vicinity of the EMcapsulins, the background was locally masked from the patches by setting all pixels to 0 values that did not belong to the foreground segmentation mask produced by the U-Net. To filter out falsely merged neighboring EMcapsulin segmentation instances and irregularly shaped segmentation masks, the circularity, defined as 4*π*area/perimeter², of each connected-component instance was calculated. Segmentation instances below an area of 60 or above 2304 pixels, and instances touching the image borders were not considered for classification. Additionally, objects with a circularity below 0.8 were not classified. To construct the majority-vote-based confusion matrices, we sampled n particles from the test set and assigned them the most frequently occurring class, as determined by the EfficientNetV2-M.

A napari GUI was developed in Python to enable interactive segmentation, classification, and visualization of TEM images.

### SEQUENCES

The following sequences have been used in the preparation of the invention. It is clear to the skilled person that such sequences are exemplary sequences. Any functional fragment or variant of a sequence is comprised within the scope of the invention. Within the scope of the invention are also sequences having 80%, 81%, 82%, 83%, 84%, 85%, 86%, 87%, 88%, 89%, 90%, 91%, 92%, 93%, 94%, 95%, 96%, 97%, 98%, 99% sequence identity with the given sequences.

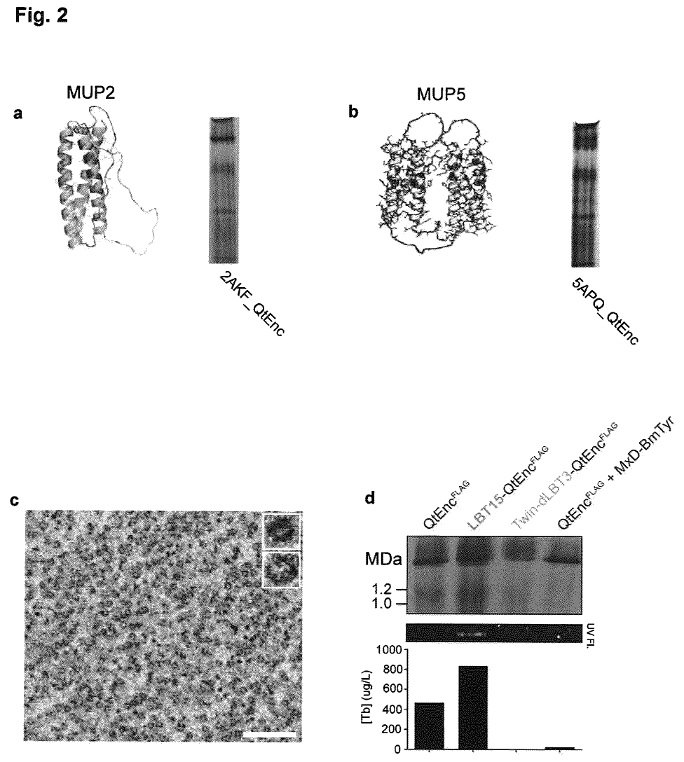

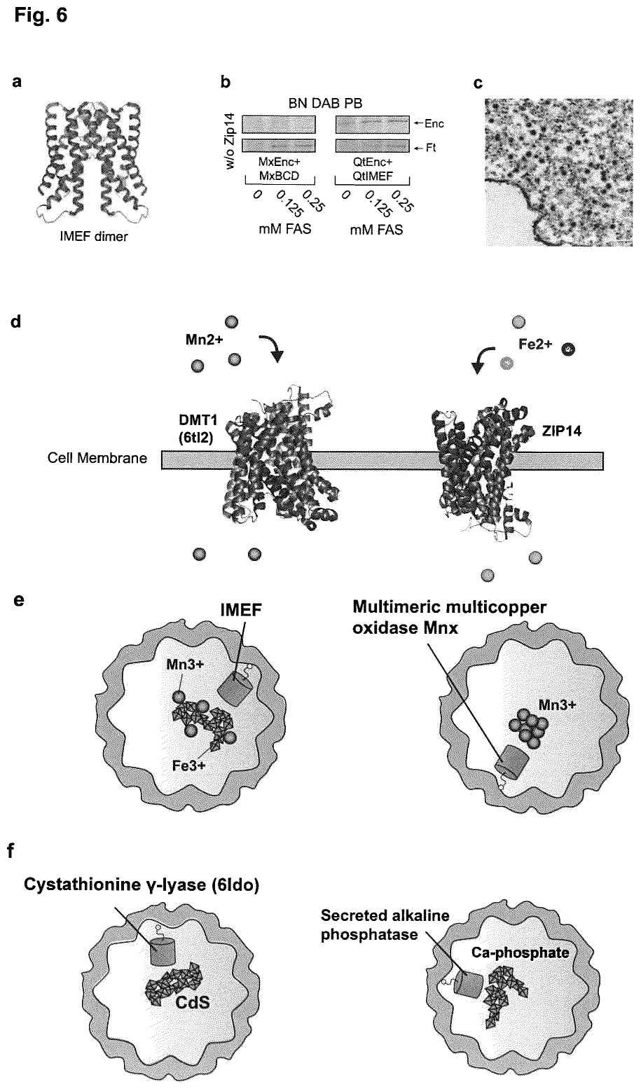

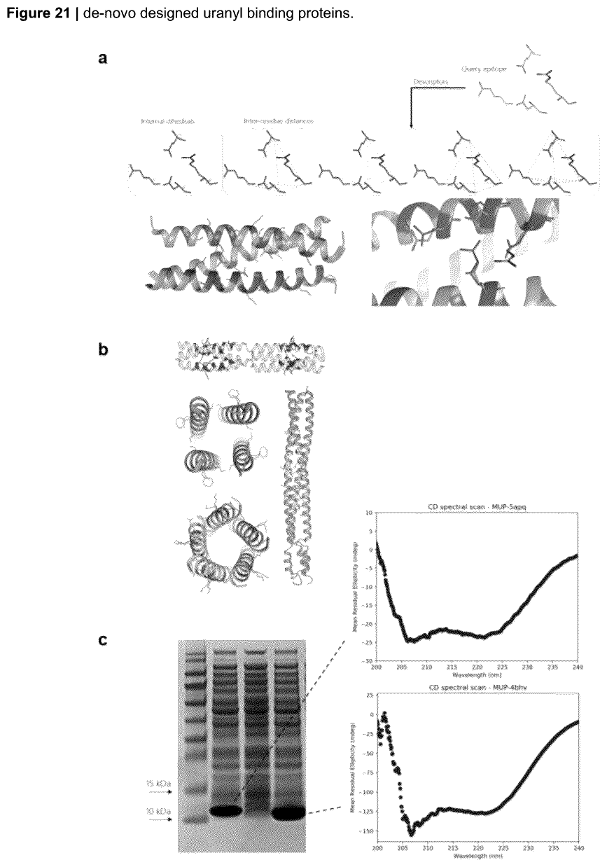

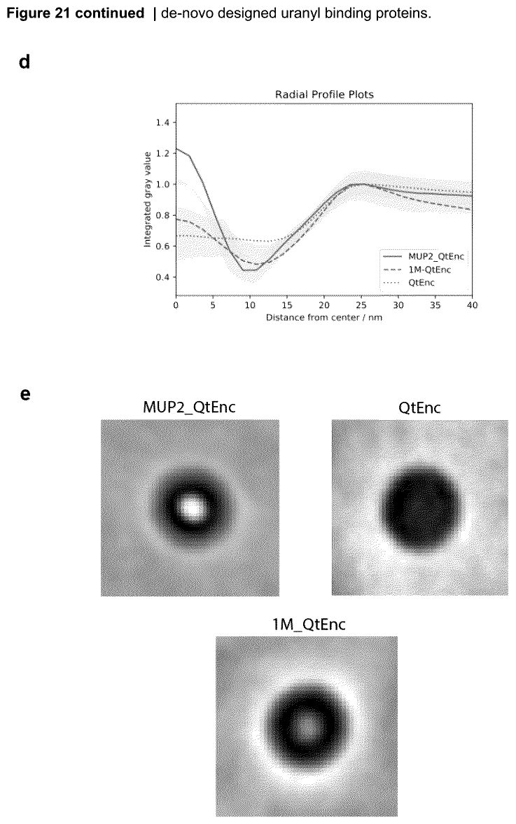

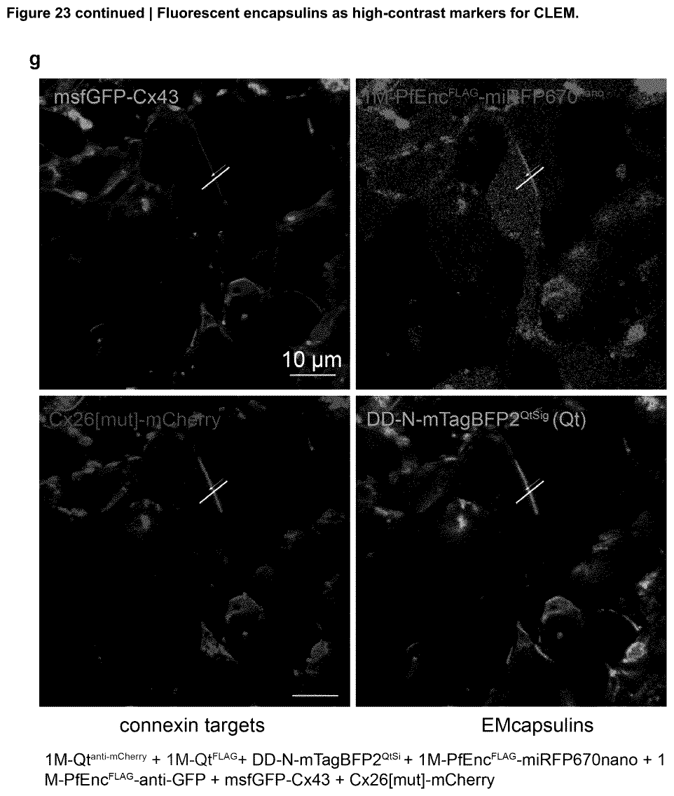

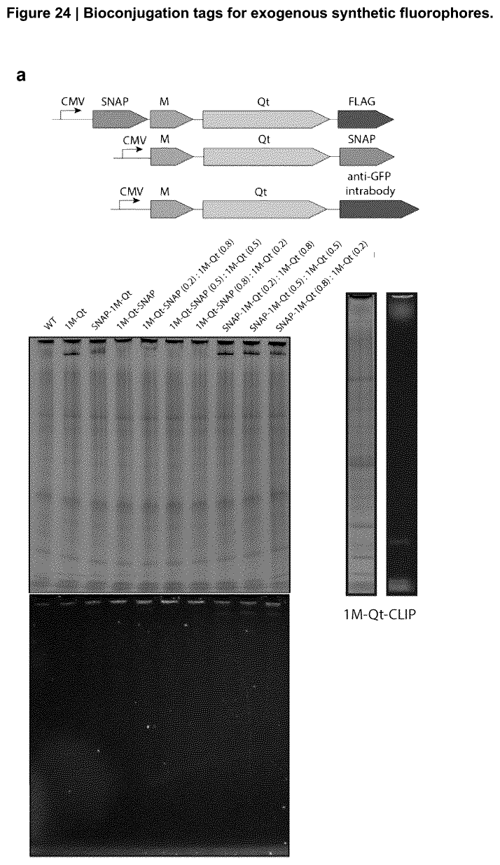

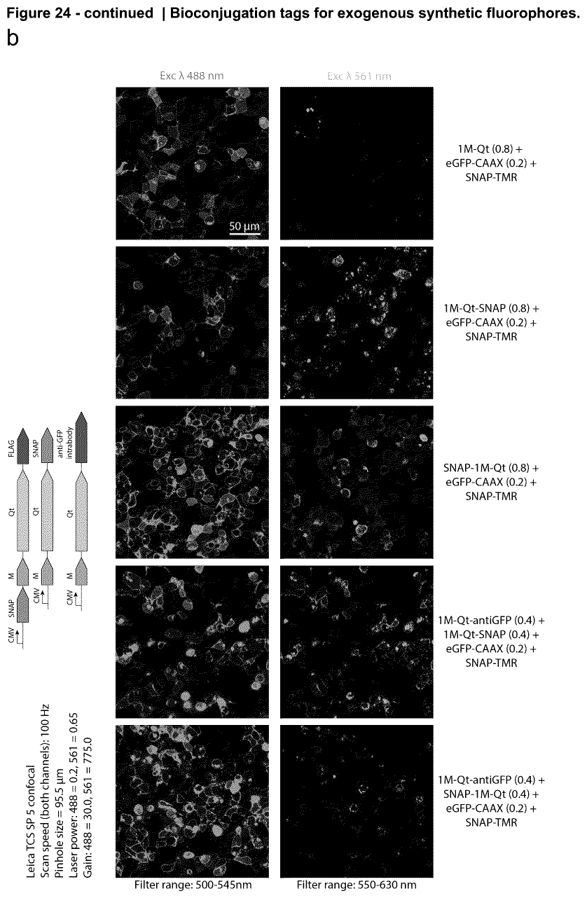

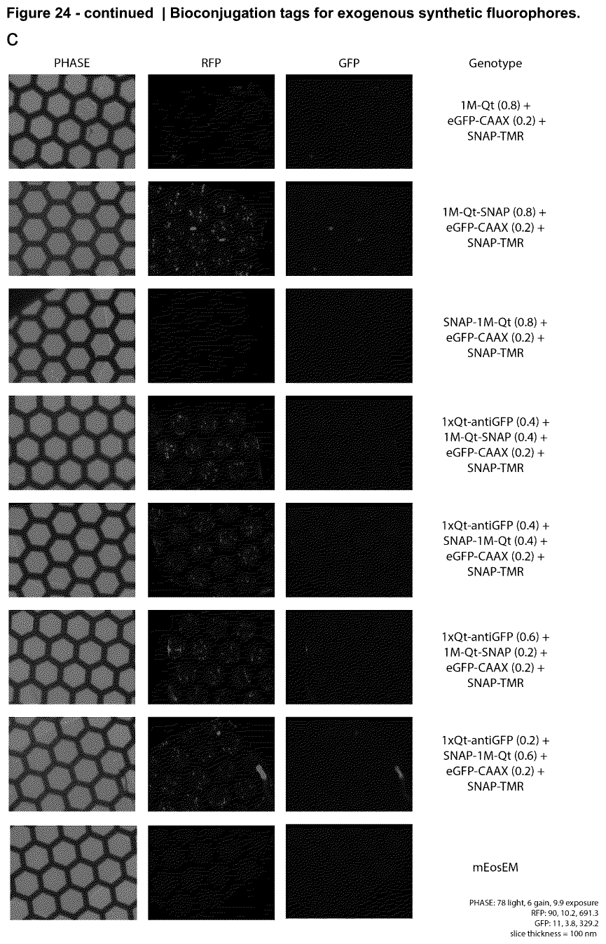

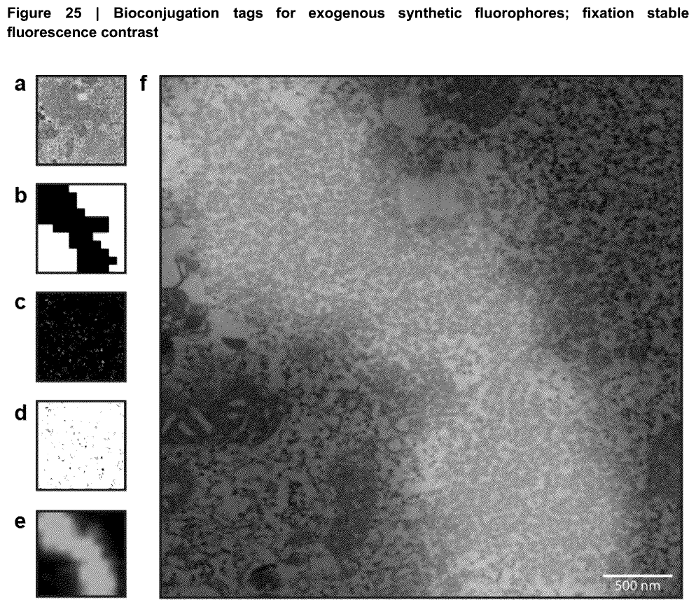

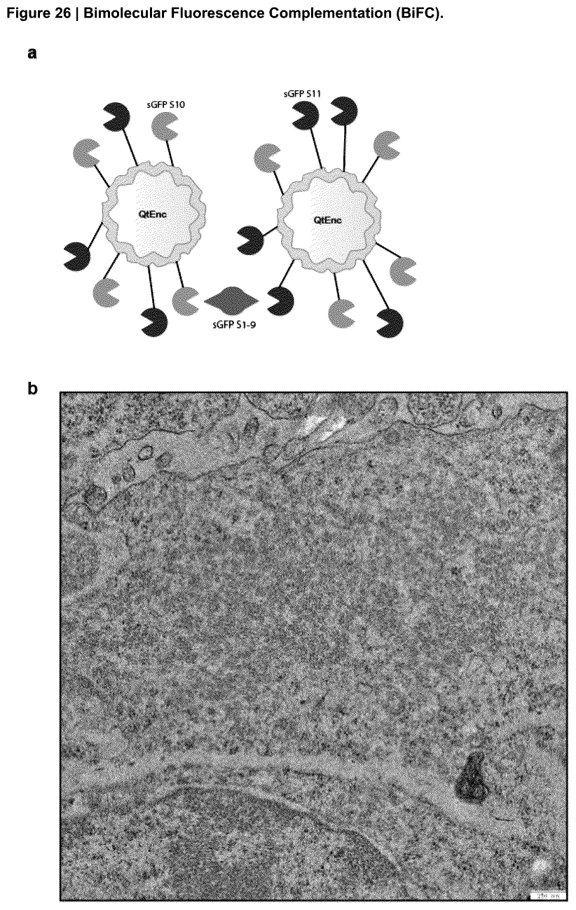

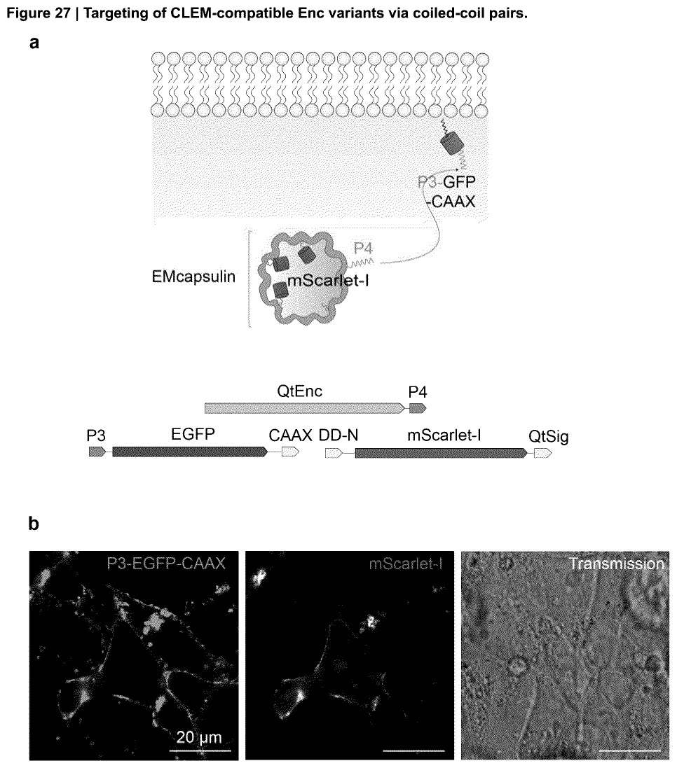

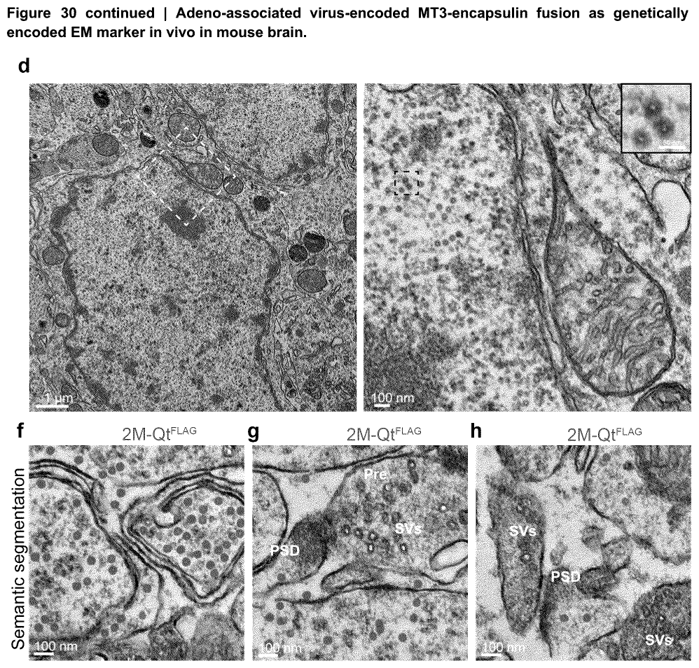

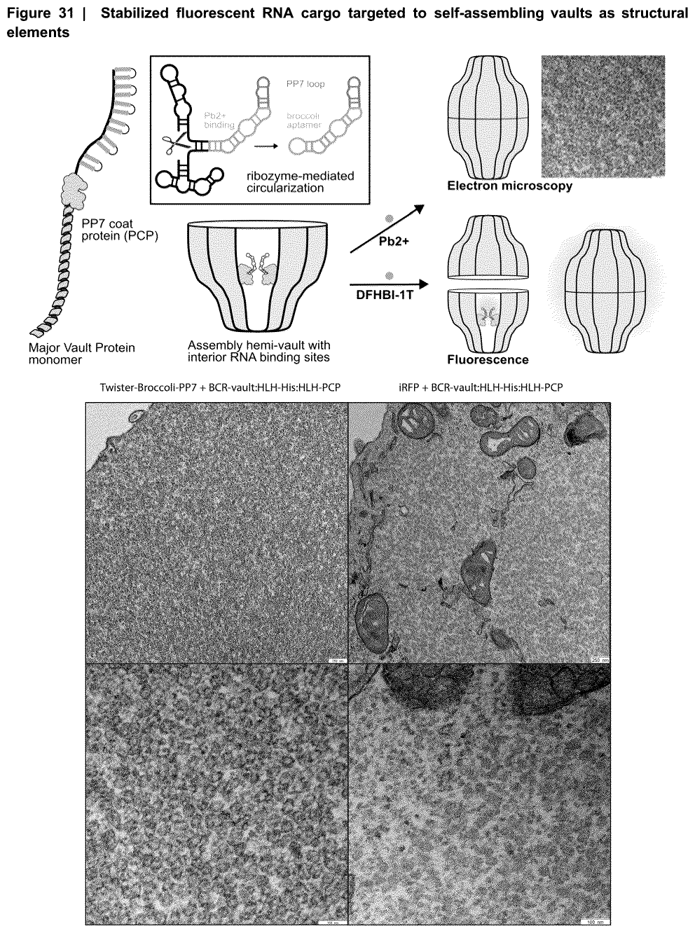

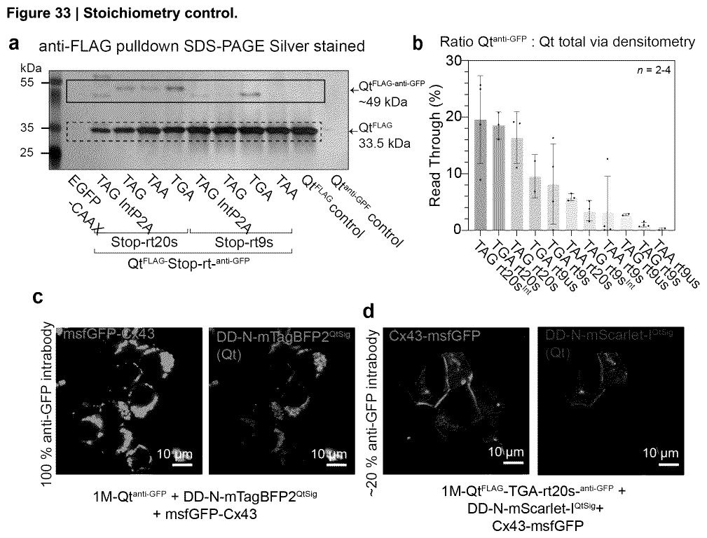

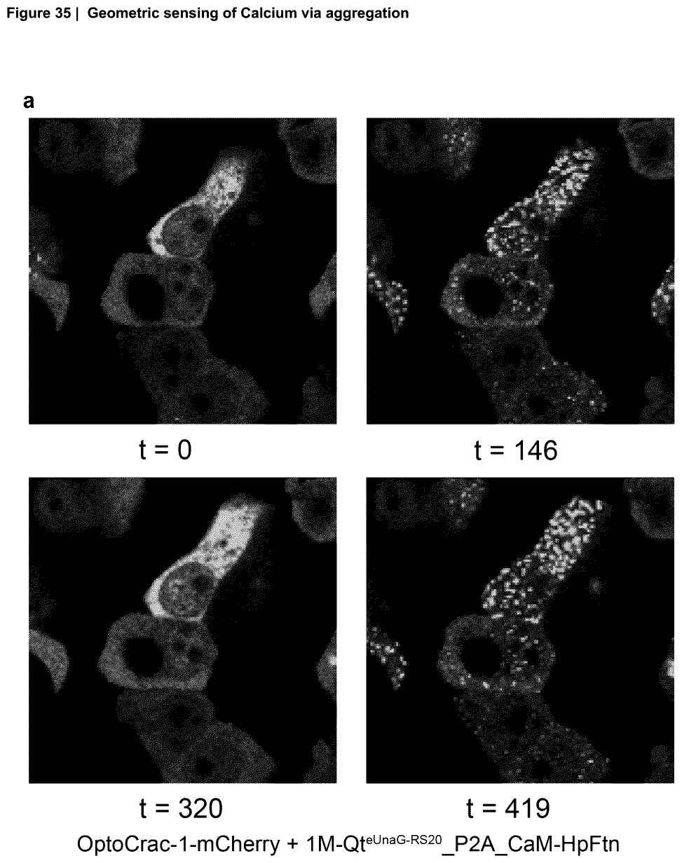

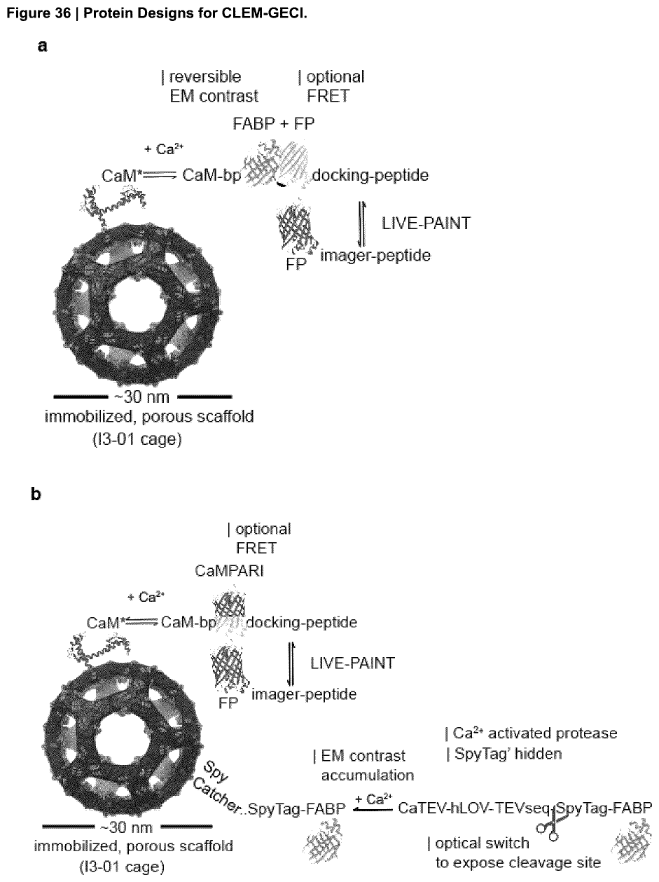

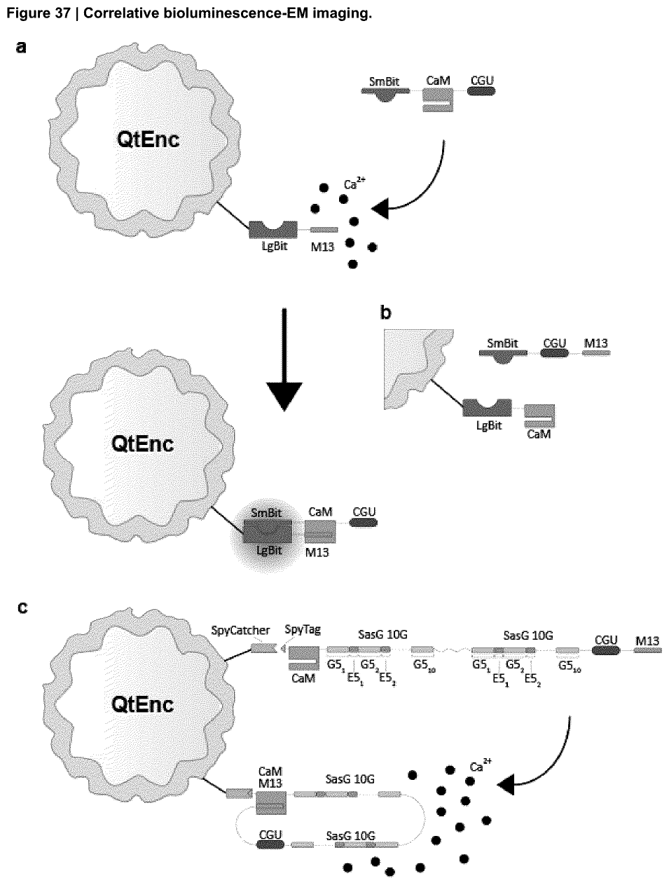

TABLE 2
Sequences used for this invention

| EQ | | | |
|---|---|---|---|
| ID NO | Name | Used in FIG. | Comment |
| 1 | MmMT3 (MT3) | FIG. 1d, FIGS. | Metallothionein 3 from Mus musculus |
| | | 9b, 9c, 9d, 9f, 9g, | |
| | | 9h, 9i, 9j, 9k | |
| 2 | His-6 MmMT3 (His- | FIG. 1 related | Metallothionein 3 from Mus musculus with an N- |
| | 6-1xMT3) | | terminal His6 tag for Co/Ni binding |
| 3 | His-9 MmMT3 | FIG. 1 related | Metallothionein 3 from Mus musculus with an N- |
| | | | terminal His9 tag for Co/Ni binding |
| 4 | 2xMmMT3 (2xMT3) | FIGS. 1a 1b, 1d | Tandem of Metallothionein 3 from Mus musculus |
| | | FIG. 9f | connecter via GGGSGGGSGGSA linker. |
| 5 | 3x chimeric MT | FIGS. 10b, 10d | Chimeric Metallothionein triplet consisting of MmMT3, |
| | (3xMT) | | Metallothionein SmtA from Synechococcus elongatus, |
| | | | and the Metallothionein EC1 from Tricium aestivum |
| | | | connected via short GS linkers. |
| 6 | 30xMMKPDM | FIG. 1 e | Methionine-rich repeat protein consisting of 30 |
| | repeats | | MMKPDM repeats. Major protein component of the |
| | | | hinge ligament of the pearl oyster Pinctada fucata |
| 7 | PDB: 2ml7 | FIG. 1 f | Cysteine Rich peptide from Panax ginseng containing |
| | | | many osmiophilic residues |
| 8 | PDB: 4nl8 | FIG. 1 f | Cysteine Rich peptide from Klebsiella pneumoniae |
| | | | containing many osmiophilic residues |
| 9 | PDB: 2jyt | FIG. 1 f | Cysteine Rich peptide from Homo sapiens containing |
| | | | many osmiophilic residues |
| 10 | PDB: 5lah | FIG. 1 f | Cysteine Rich peptide from Urticina eques containing |
| | | | many osmiophilic residues |
| 11 | PDB: 3hcu | FIG. 1 f | Cysteine Rich peptide from Homo sapiens containing |
| | | | many osmiophilic residues |
| 12 | MeLanM | FIGS. 14b, 14d, | Lanthanide-binding protein from Methylobacterium |
| | (Lanmodulin) | 14e | extorquens |
| 13 | Lanthanide binding | FIG. 2d | Synthethic lanthanide binding tag |
| | tag 15 (LBT15) | | |
| 14 | AssVanabin2 | FIGS. 14b, 14d, | Vanadmium-binding protein from Ascidia sydneiensis |
| | (Vanabin 2 | 14e | samea |
| 15 | EcCusF | FIGS. 14b, 14d, | Silver and Copper-binding protein CusF from Escherichia |
| | | 14e | coli |
| 16 | QtSig | FIG. 1 e, | Encapsulation signal for the Encapsulin system of |
| | | | Quasibacillus thermotolerans |
| 17 | PfHRPII-QtSig | FIG. 1 e | Histidine-rich protein II of Plasmodium falciparum fused |
| | | | to a QtSig via a GGGGSGGGGS linker. |
| 18 | eUnaG-MT3 | FIGS. 7a, 7b, 7c, | Fusion protein consisting of enhanced UnaG(V2L) from |
| | | 7d, 7e, 7f | Anguilla japonica and MmMT3 |
| 19 | SNAP-Tag | related to FIG. 7 | human O6-alkylguanine-DNA-alkyltransferase variant |
| | | | that forms covalent bonds with benzylguanine |
| | | | derivatives |
| 20 | Halo-Tag | related to FIG. 7 | modified bacterial dehalogenase that forms covalent |
| | | | bonds with chloroalkane derivatives |
| 21 | P2-MT3-QtEnc-FLAG | related to FIG. 7 | Fusion of a synthetic coiled-coil (CC) domain (P2), the |
| | | | CGU MmMT3, the QtEnc shell-forming monomer from |
| | | | the Encapsulin system of Q. thermotolerans and a FLAG- |
| | | | epitope |
| 22 | P1-mEosEM-hPEST | related to FIG. 7 | Fusion of synthetic coiled-coil (CC) domain P1 (cognate |
| | | | partner of P2), OsO4 post-fixation stable switchable |
| | | | fluorescent protein mEosEM and a human PEST |
| | | | degradation sequence |
| 23 | sGFP_S1-9 | FIG. 7g | Strand 1 to 9 of a Tripartite split superfolderGFP |
| 24 | QtEnc_sGFP-S1-9 | related to FIG. 7 | The shell forming monomer of the Ecapsulin system of |
| | | | Quasibacillus thermotolerans fused to superfolder GFP |
| | | | strands 1 to 9 |
| 25 | QtEnc_sGFP-S10 | FIG. 7 g | The shell forming monomer of the Ecapsulin system of |
| | | | Quasibacillus thermotolerans fused to superfolderGFP |
| | | | strand 10 |
| 26 | QtEnc_sGFP-S11 | FIG. 7 g | The shell forming monomer of the Ecapsulin system of |
| | | | Quasibacillus thermotolerans fused to superfolderGFP |
| | | | strand 11 |
| 27 | QtEnc_eUnaG | FIG. 7-related | The shell forming monomer of the Ecapsulin system of |
| | | | Quasibacillus thermotolerans fused to enhanced |
| | | | UnaG(V2L) from Anguilla japonica and MmMT3 |
| 28 | MxEnc_eUnaG | FIG. 7-related | The shell forming monomer of the Ecapsulin system of |
| | | | Myxococcus xanthus fused to enhanced |
| | | | UnaG(V2L) from Anguilla japonica and MmMT3 |
| 29 | BM3h (PDB:6H1O) | FIG. 3 d | Heme domain of BM3 a natural fusion protein |
| | | | constructed of cytochrome P450 and NADPH- |
| | | | cytochrome P450 reductase domains from Priestia |
| | | | megaterium |
| 30 | Liver human FABP | FIG. 3 a | Soluble protein binding fatty acids expressed in human |
| | | | liver |
| 31 | Intestinal human | FIG. 3-related | Soluble protein binding fatty acids expressed in human |
| | FABP | | intestine |
| 32 | Heart human FABP | FIG. 3-related | Soluble protein binding fatty acids expressed in human |
| | | | heart |
| 33 | Adipocyte human | FIG. 3-related | Soluble protein binding fatty acids expressed in human |
| | FABP | | adipocytes |
| 34 | Epidermal human | FIG. 3-related | Soluble protein binding fatty acid expressed in human |
| | FABP | | epidermis |
| 35 | Ileal human FABP | FIG. 3-related | Soluble protein binding fatty acid expressed in human |
| | | | ileum |
| 36 | Brain human FABP | FIG. 3 a | Soluble protein binding fatty acid expressed in human |
| | | | brain |
| 37 | Myelin human FABP | FIG. 3-related | Soluble protein binding fatty acid expressed in human |
| | PMP2 | | peripheral nerves |
| 38 | Testis human FABP | FIG. 3-related | Soluble protein binding fatty acid expressed in human |
| | | | testis |
| 39 | Retinal human FABP | FIG. 3-related | Soluble protein binding fatty acid expressed in human |
| | | | retina |
| 40 | Farn-Signal (Farn) | FIG. 16 a | 281-290 of rat H-Ras, C-terminal farnesylation signal |
| 41 | SNAP25-derived | FIGS. 1a, 1b, 1c, | Amino acids 85-120 of Rattus norvegicus SNAP25 |
| | palmitoylation signal | 1d, also relates to | |
| | (SNAP25) | FIG. 3 | |
| 42 | Fyn-Kinase derived | relates to FIG. 3 | N-terminal myristoylation signal of human Fyn-Kinase, |
| | N-terminal | | Amino acids 1-15 |
| | myristoylation signal | | |
| 43 | Microtubule- | relates to FIG. 3 | Microtubule-associated proteins 1A/1B light chain 3A |
| | associated proteins | | (PDB:MLP3A_MOUSE) |
| | 1A/1B light chain 3A | | |
| | (PDB:MLP3A_MOUSE) | | |
| 44 | MSP26 | relates to FIG. 3 | Engineered apo-lipoprotein A-1 (MSP1) flanked by DnaE |
| | | | split intein fragments C-intein (AA 103-137) on the N- |
| | | | term and N-Intein (AA 1-102) on the C-term (Homo |
| | | | sapiens, Nostoc punctiforme): |
| 45 | MUP2 | FIG. 2 a | Designed uranyl binding proteins with 6 binding sites for |
| | | | uranyl ions |
| 46 | MUP5 | FIG. 2 b | Designed uranyl binding proteins with 6 binding sites for |
| | | | uranyl ions |
| 47 | DD-CgPPK2b-QtSig | FIG. 5 a, b, c, d | Degron-tagged polyphosphate kinase 2b of |
| | | | Corynebacterium glutamicum C-terminanlly tagged with |
| | | | the encapsulation signal of Quasibacillus thermotolerans |
| 48 | CgPPK2b | FIGS. 5a, 5b, 5c, | Polyphopsphate kinase 2b of Corynebacterium |
| | | 5d | glutamicum |
| 49 | AtPCS1-QtSig | FIG. 5 e | Phytochelatin synthase 1 of with a |
| | | | C-terminal encapsulation signal |
| 50 | AtPCS2-QtSig | relates to FIG. 5 e | Phytochelatin synthase 2 of Arabidopsis thaliana with a |
| | | | C-terminal encapsulation signal |
| 51 | BmTyr | FIG. 5 f | B. megaterium tyrosinase |
| 52 | QtIMEF | FIG. 1 b, FIGS. | Iron-mineraizing ferritin-like cargo protein from the |
| | | 6a, 6b, 6c, 6d, | Encapsulin system of Q. thermotolerans |
| | | FIG. 16 a, FIG. | |
| | | 17 i, FIG. 20 b | |
| 53 | PP7 phage coat | FIG. 4-related | The coat protein of the single-stranded RNA |
| | protein (PCP) | | bacteriophage Pseudomonas phage |
| 54 | PP7 phage coat | FIG. 4 a | Tandem dimer of the coat protein of the single-stranded |
| | protein tandem | | RNA bacteriophage Pseudomonas phage binding the PP7 |
| | dimer (tdPCP) | | stem loop |
| 55 | Spinach Aptamer | FIG. 4 c | Synthetically derived RNA aptamer designed to be an |
| | | | RNA mimic of green fluorescent protein (GFP) |
| 56 | PP7 stem loop | FIG. 4 b | Small RNA sequence binding partner of the tandem PCP |
| | | | dimer from Pseudomonas phage |
| 57 | Dir2s Aptamer | FIG. 4 d | Synthetically derived RNA aptamer designed to be an |
| | | | RNA mimic of red fluorescent protein (RFP) |
| 58 | Metal-binding RNA- | FIG. 4-related | Small synthetic aptamer coordination metal ions in |
| | oligomer | | homodimeric form |
| 59 | RhoBAST Aptamer | FIG. 4 e | Synthetic aptamer that binds a fluorogenic rhodamine |
| | | | dye with fast association and dissociation kinetics |
| 60 | RRM domain | FIG. 4 f | RNA-binding domain that are known to bind single- |
| | | | stranded RNAs. |
| 61 | KH2 domain | FIG. 4 f | Protein domain that binds RNA, and can function in RNA |
| | | | recognition |
| 62 | Zinc Finger CCHC | FIG. 4 f | Zinc finger domain subspecies know to bind RNA |
| 63 | L7Ae | FIG. 4 f related | Multifunctional RNA-binding protein that recognizes the |
| | | | K-turn motif in ribosomal RNA |
| 64 | BIV-tat | FIG. 4 f related | BIV-Tat from the Bovine immunodeficiency virus binds to |
| | | | a hairpin structure at the 5′-end of all nascent viral |
| | | | mRNAs |
| 65 | QtEnc | FIGS. 1, 2, 3, 5, 6, | Shell forming monomer of the Encapsulin system of |
| | | 7, 8, 9, 15, 16, 17, | Q. thermotolerans |
| | | 18, 19, 20 (in their | |
| | | entirety) | |
| 66 | MxEnc | FIGS. 1, 5, 8, 9, | Shell forming monomer of the Encapsulin system of |
| | | 10 (in their | M. xanthus |
| | | entirety) | |
| 67 | TmEnc | FIGS. 1, 8, 9, 10 | Shell forming monomer from the encapsulin system of |
| | | (in their entirety) | T. maritima (AA 1-264) |
| 68 | P1 | FIG. 8 | Synthetic coiled-coil domain which pairs with P2 |
| 69 | P2 | FIG. 8 | Synthetic coiled-coil domain which pairs with P1 |
| 70 | P3 | FIG. 8 | Synthetic coiled-coil domain which pairs with P4 |
| 71 | P4 | FIG. 8 | Synthetic coiled-coil domain which pairs with P3 |
| 72 | DHD13a | FIG. 8 | Synthetic coiled-coil domain which pairs DHD13b |
| 73 | DHD13b | FIG. 8 | Synthetic coiled-coil domain which pairs DHD13a |
| 74 | RT9s | FIG. 15 | DNA sequence promoting ribosomal (translational) read- |
| | | | through after a STOP codon |
| 75 | K-turn motif | related to FIG. 4 | RNA motif binding to L7Ae |
| 76 | RT20s | FIG. 15 | DNA sequence promoting ribosomal (translational) read- |
| | | | through after a STOP codon |
| 77 | Major vault protein | FIG. 11 | Vault forming protein from Rattus norvegicus |
| | (RnMVP) | | |
| 78 | Major vault protein | FIG. 11 | Vault forming protein from Rattus norvegicus with |
| | with SNAP25- | | SNAP25-derived palmitoylation signal (AA 85-120) |
| | derived | | inserted in helix-loop-helix region |
| | palmitoylation signal | | |
| | (85-120) (RnMVP- | | |
| | SNAP25) | | |
| 79 | RnMVP-APH | FIG. 11 | Vault forming protein from Rattus norvegicus with |
| | | | antiparallel coiled-coil domain inserted into helix-loop- |
| | | | helix region |
| 80 | RnMVP-APH-short | FIG. 11 | Vault forming protein from Rattus norvegicus with a |
| | | | shortened antiparallel coiled-coil domain inserted into |
| | | | helix-loop-helix region |
| 81 | RnMVP-caged-Gp41- | FIG. 11 | TEV protease inducible Vault based on caged Gp41-1 |
| | 1-TEV v1 | | split inteins, Version 1 |
| 82 | RnMVP-caged-Gp41- | FIG. 11 | TEV protease inducible Vault based on caged Gp41-1 |
| | 1-TEV v2 | | split inteins, Version 2 |
| 83 | MT3-mINT | FIG. 11 | MmMT3 fused to endogenous vault cargo protein mINT |
| 84 | RnMVP_HLH-MT3 | FIG. 11 | MmMT3 inserted into rat Vault Helix-Loop-Helix motif |
| 85 | RnMVP_Nterm_BCR | FIG. 11 | Rat Vault with N-terminal anti-parallel coiled-coil |
| | HLH- | | domain stabilizing the holo-vault configuration and MT3 |
| | His6_HLH_MT3 | | and His6-tag in the HLH domain |
| 86 | DeNovoTIM15 | FIG. 14 related | Synthetic hyperstable de novo TIM barrel variant |
| 87 | Porcine | FIG. 14 g | Porcine Ribonuclease Inhibitor from Sus scrofa wich |
| | Ribonuclease | | exposes modifiable helices |
| | Inhibitor | | |
| 88 | Helical Assembly of | FIG. 14 g | Proteasome that forms a dodecameric complex |
| | the Anbu Complex | | including modifiable helices |
| | from Pseudomonas | | |
| | aeruginosa | | |
| 89 | gp37-gp38 adhesin | FIG. 14 h | Adhesin complex of the bacteriophage S16 long tail |
| | tip complex. Chain A | | fiber. The variable loops can be extended to display |
| | | | binding tags. |
| 90 | gp37-gp38 adhesin | FIG. 14 h | Adhesin complex of the bacteriophage S16 long tail |
| | tip complex. Chain B | | fiber. The variable loops can be extended to display |
| | | | binding tags. |
| 91 | Adenovirus species | FIG. 14h | Knob protein from human adenovirus 26. Its loop region |
| | 26 knob protein | | can be engineered to display binding tags. |
| 92 | emFPv1 | all in FIGS. 14a, | Engineered fluorescent protein based on sfGFP, with |
| | | 14b, 14c, 14d, 14e | ALFA-tags (SRLEEELRRRLT) inserted into loop region and |
| | | | at the termini. |
| 93 | emFPv3 | all in FIGS. 14a, | Engineered fluorescent protein based on |
| | | 14b, 14c, 14d, 14e | mGreenLantern, with HA-tags (YPYDVPDYA) inserted |
| | | | into loop region and at the termini. |
| 94 | emFPv4 | all in FIGS. 14a, | Engineered fluorescent protein based on |
| | | 14b, 14c, 14d, 14e | mGreenLantern, with Moon-Tags (KNEQELLELDKWASL) |
| | | | inserted into loop region and at the termini. |
| 95 | emFPv5 | all in FIGS. 14a, | Engineered fluorescent protein based on mScarlet-I with |
| | | 14b, 14c, 14d, 14e | PepTags (AVERYLKDQQLLGIW) inserted into loop region |
| | | | and at the termini. |
| 96 | emFPv6 | all in FIGS. 14a, | Engineered fluorescent protein based on mEosEM, with |
| | | 14b, 14c, 14d, 14e | MoonTags (EELLSKNYHLENEVARLKK) inserted into loop |
| | | | region and at the termini. |
| 97 | emFPv7 | all in FIGS. 14a, | Engineered fluorescent protein based on mScarlet-I, |
| | | 14b, 14c, 14d, 14e | with triple FLAG-tags (DYKDHDGDYKDHDIDYKDDDDK) |
| | | | inserted into loop region and at the termini. |
| 98 | 24xGCN_v4(SunTag)- | FIGS. 14a, 14b, | Engineered fluorescent protein based on emFPv4 with |
| | emFPv4- | 14c, 14d, 14e- | 24x SunTags on both termini. |
| | 24xGCN_v4(SunTag) | related | |
| 99 | CCC-Tag(10xP3)- | FIGS. 14a, 14b, | Engineered fluorescent protein based on emFPv4 with |
| | emFPv4-CCC- | 14c, 14d, 14e- | 10x P3 CCC tags on each terminus. |
| | Tag(10xP3) | related | |
| 100 | MS2 phage capsid | FIG. 14f | Coat protein of the RNA bacteriophage MS2 which binds |
| | protein | | a specific stem-loop structure in viral RNA to accomplish |
| | | | encapsidation of its genome. |
| 101 | MS2 phage | FIG. 14f | Maturation protein of the RNA bacteriophage MS2 |
| | Maturation protein | | required for the typical attachment of the phage to the |
| | A | | side of the bacterial pili. Binds to sequences located |
| | | | toward each end of the genome, hence circularizing it. |
| 102 | Qbeta phage capsid | FIG. 14f | Coat protein of the Escherichia virus Qbeta which binds |
| | protein | | a specific stem-loop structure in viral RNA to accomplish |
| | | | encapsidation of its genome. |
| 103 | Qbeta phage | FIG. 14f | Maturation protein of the Escherichia virus Qbeta |
| | Maturation protein | | required for the typical attachment of the phage to the |
| | A2 | | side of the bacterial pili; Binds to sequences located |
| | | | toward each end of the genome, hence circularizing it. |
| 104 | QtEnc-FLAG | FIGS. 9 h, 18 a | Capsid forming monomer from the encapsulin system of |
| | | | Q. thermotolerans tagged with C-termina FLAG epitope |
| 105 | SasG G51-G52 | Related to FIGS. | G51-E1-G52 domains of S. aureus protein SasG (amino |
| | | 10 d, 13 a, 17 a, 17 b, | acids 419-629) and mCherry |
| | | 17 c, 17 d, 17 e, 17 f, | |
| | | 17 g, 18 f, 18 g, | |
| | | 18 h, 20 a | |
| 106 | QtEnc- | FIGS. 16 d, 18 k, | Capsid forming monomer from the encapsulin system of |
| | antiGFPintrabody | 20 a, 20 b | Q. thermotolerans tagged with a C-terminal anti-GFP |
| | (QtEnc^{aGFPIB}) | | intrabody |
| 107 | MxEnc-FLAG | FIG. 1 | Capsid forming monomer from the encapsulin system of |
| | | | M. xanthus tagged with C-terminal FLAG epitope |
| 108 | TmEnc-BC2Tag | related to FIG. 9 | Capsid forming monomer from the encapsulin system of |
| | | | T. maritima (amino acids 1-264) tagged with C-termina |
| | | | BC2 epitope |
| 109 | P2-TmEnc- | FIGS. 17 c, 17 d | Capsid forming monomer from the encapsulin system of |
| | antimCherry | | T. maritima (amino acids 1-264) N-terminally fused to a |
| | intrabody (LaM-4) | | synthetic coiled-coil domain ‘P2’ (Lebar et al., 2020) and |
| | | | C-terminally fused to an anti-mCherry Intrabody (LaM-4) |
| 110 | MT3-DHF119-GFP | FIGS. 12 c, 12 d | Fusion protein consisting of MT3, a de novo designed |
| | | | fibril forming protein(Shen et al. 2018) and GFP |
| 111 | His6-SpyCatcher-13- | FIG. 17 i | Fusion protein consisting of hexa-Histidine Tag, a |
| | 01-MmMT3 | | SpyCatcher domain (Zakeri et al. 2012), a de novo |
| | | | designed, single component icosahedron(Hsia et al. |
| | | | 2016) and an MT3 domain |
| 112 | CsgA-SpyTag003 | FIGS. 12 a, 12 b | Fusion protein of E. coli CsgA where the secretion |
| | | | peptide and N22 were deleted (amino acids 36-144) and |
| | | | SpyTag peptide |
| 113 | QtEnc-KinTag | FIG. 16 b | Fusion protein of capsid forming monomer of the |
| | | | Encapsulin system of Q. thermotolerans and a synthetic |
| | | | peptide interacting with motor proteins (KinTag(Cross et |
| | | | al. 2021)) |
| 114 | sfGFP-SasG(G51- | FIGS. 17 a, 17 c | Fusion protein consisting of super-folder GFP, the G51- |
| | G52)-mCherry | | E1-G52 domains of S. aureus protein SasG (amino acids |
| | | | 419-629) and mCherry |
| 115 | Gp41- | FIG. 17 g | Fusion protein consisting of Gp41-1 C-intein, superfolder |
| | 1_sfGFP_SasG_G51- | | GFP, SasG (comprising 4 G5 domains yielding GEGEGEG), |
| | G54_SpyCatcher003 | | and engineered SpyCatcher003 |
| 116 | SnoopTag_mTurquoise_{—} | FIG. 17 g | Fusion protein consisting of a SnoopTag, mTurquoise2, |
| | SasG_G51- | | SasG (comprising 4 G5 domains yielding GEGEGEG) and |
| | G54_Gp41-1-N-Int | | the N-intein split fragment of Gp41-1 |
| 117 | SpyTag003_mCherry_{—} | FIG. 17 g | Fusion protein consisting of SpyTag003, the red |
| | SasG_G51- | | fluorescent protein mCherry, SasG (comprising 4 G5 |
| | G54_SnoopCatcher | | domains yielding GEGEGEG) and a SnoopCatcher domain |
| 118 | sfGFP_SasG_G_Npu | FIG. 18 f, 18 g | Fusion protein consisting of superfolder GFP, the G51 |
| | DnaE_N-Int_V2 | | domain of SasG and the Npu DnaE N-intein split part |
| 119 | NpuDnaE_GEP_C- | FIG. 18 h | Fusion protein consisting of Npu DnaE GEP C-Intein split |
| | Int_SasG_EG_mCherry_{—} | | fragment, SasG EG domains, where the proline residue |
| | V2 | | in the G51-E junction was mutated to cysteine to serve |
| | | | as nucleophile in the splicing reaction, and mCherry |
| 120 | NpuDnaE_GEP_C- | FIGS. 18 g, 18 h | Fusion protein consisting of Npu DnaE GEP C-Intein split |
| | Int_SasG_EG_Npu_{—} | | fragment, SasG EG domains and the Npu DnaE N-intein |
| | DnaE_N-Int_V2 | | split part on its C-terminus. This yields a self-growing |
| | | | fragment which can be capped off with |
| | | | NpuDnaE_GEP_C-Int_SasG_EG_mCherry_V2 |
| 121 | sfGFP_SasG_G_Vida | FIG. 18 g | Fusion protein consisting of superfolder GFP, the G51 |
| | L-N-Int | | domain of SasG and the VidaL N-Intein split part |
| 122 | VidaL-C- | FIG. 18 h | Fusion protein consisting of VidaL C-Intein split part, |
| | Int_SasG_EG_{—} | | SasG EG domains and mCherry |
| | mCherry | | |
| 123 | VidaL-C- | FIGS. 17 a, 18 g, | Fusion protein consisting of VidaL C-Intein split part, |
| | Int_SasG EG _VidaL- | 18 h | SasG EG domains and VidaL N-Intein split part. This |
| | N-Int | | building block is a self-growing unit and can be capped |
| | | | off with VidaL-C-Int_SasG_EG_mCherry |
| 124 | Tobacco etch virus | related to FIGS. | Tobacco etch virus protease |
| | protease (TEVp) | 18 b, 18 j | |
| 125 | plum pox virus | related to FIG. | plum pox virus protease, alternative to TEVp |
| | protease (PPVp) | 18 (in its entirety) | |
| 126 | soybean mosaic | related to FIG. | soybean mosaic virus protease, alternative to TEVp |
| | virus protease | 18 (in its entirety) | |
| | (SbMVp) | | |
| 127 | sunflower mild | related to FIG. | sunflower mild mosaic virus protease, alternative to |
| | mosaic virus | 18 (in its entirety) | TEVp |
| | protease (SuMMVp) | | |
| 128 | 3xLBT15-TEVsite- | FIG. 18 a | Fusion protein consisting of a triple engineered |
| | MT3-QtEnc-FLAG | | Lanthanide binding domain (LBT15) (Sculimbrene and |
| | | | Imperiali 2006), a TEV cleavage site, the capsid forming |
| | | | monomer of the Encapsulin system of Q. thermotolerans |
| | | | and a FLAG epitope |
| 129 | FLAG-N-TEV-1-118- | FIG. 18 a | Fusion protein consisting of a FLAG epitope, the N- |
| | M13 | | terminal fragment of the Tobacco etch virus protease |
| | | | (TEV) (amino acids 1-118 and a C-terminal M13 |
| | | | calmodulin-binding peptide |
| 130 | Myc-Calmodulin- | FIG. 18 a | Fusion protein consisting of a Myc epitope, Calmodulin |
| | TEV-119-219 | | and the C-terminal split fragment of Tobacco etch virus |
| | | | protease (amino acids 119-219, S153N(Sanchez and Ting |
| | | | 2020)) |
| 131 | MxEnc-RS20 | FIG. 19 | Fusion of Encapsulin forming monomer of the |
| | | | Encapsulin system M. xanthus and the Calmodulin |
| | | | binding peptide RS20 |
| 132 | QtEnc-RS20 | FIG. 19 | Fusion of Encapsulin forming monomer of the |
| | | | Encapsulin system Q. thermotolerans and the Calmodulin |
| | | | binding peptide RS20 |
| 133 | TmEnc-RS20 | FIG. 19 | Fusion of Encapsulin forming monomer of the |
| | | | Encapsulin system T. maritima and the Calmodulin |
| | | | binding peptide RS20 |
| 134 | CaM-HpFtn | FIG. 19 b | Fusion protein of human calmodulin and H. pylori ferritin |
| | | | (AA 5-167, N19Q) |
| 135 | CaM-MT3 | Related to FIG. | Fusion of human Calmodulin and MmMT3 which will be |
| | | 19 b | recruited to the scaffold (Enc) to form a contrast halo |
| | | | around the element when elevated Ca²⁺ levels are |
| | | | present |
| 136 | MT3-TnCmin-QtSig | FIG. 19 a | Fusion protein of MT3, an engineered minimal Troponin |
| | | | C domain of Opsanus tau and the Encapsulin signal of |
| | | | the Encapsulin system of Q. thermotolerans |
| 137 | MT3-TnCmin-QtEnc- | FIG. 19 a | Calcium-responsive contractile linker to MT3. |
| | FLAG | | |
| 138 | mCherry-dTrim21 | FIG. 18 l | Fusion of E3 ubiquitin-protein ligase TRIM21, with |
| | | | inactivating mutations I79R and S80A, and mCherry |
| | | | fluorescence protein |
| 139 | sfGFP-dTrim21 | FIG. 18 l | Fusion of E3 ubiquitin-protein ligase TRIM21, with |
| | | | inactivating mutations I79R and S80A, and superfolder |
| | | | GFP |
| 140 | pcDNA 3.1 (+) | FIG. 4 g | mammalian expression vector |
| | Zeocin | | |
| 141 | pIRES | FIG. 4 g | internal ribosome entry site |
| 142 | QtEnc^{FLAG} | FIG. 1 b | Shell forming monomer of the Encapsulin system of |
| | | | Quasibacillus thermotolerans with a C-terminally |
| | | | appended FLAG epitope connected via a short GSG |
| | | | linker. |
| 143 | 2xMT3-QtEnc^{FLAG} | FIGS. 1 b, 4 g | A tandem of the Mus musculus Metallothionein 3 |
| | | | connected via GGGGSGGGGS linker fused to the shell- |
| | | | forming monomer of the Encapsulin system of |
| | | | Quasibacillus thermotolerans with a C-terminally |
| | | | appended FLAG epitope connected via a short GSG |
| | | | linker. |
| 144 | MT3-TmEnc^{BC2-Tag} | FIGS. 1 d, 9 b | Fusion of Mus musculus Metallothionein 3 fused to the |
| | | | shell forming monomer of the Encapsulin system of |
| | | | Thermotoga maritima fused to a C-terminal BC2-Tag |
| 145 | LBT15-QtEnc^{FLAG} | FIG. 2 d | Synthetic Lanthanide-bindering tag 15 fused to the shell |
| | | | forming monomer of the Encapsulin system of |
| | | | Quasibacillus thermotolerans fused to a C-terminal FLAG |
| | | | tag- |
| 146 | Twin-dLBT3- | FIG. 2 d | Engineered tandem Lanthanide-binding tag fused to the |
| | QtEnc^{FLAG} | | shell forming monomer of the Encapsulin system of |
| | | | Quasibacillus thermotolerans fused to a C-terminal FLAG |
| | | | tag |
| 147 | QtEnc-SpyTag(003) | FIGS. 3 a, 3 e | Capsid forming monomer from the encapsulin system of |
| | | | Q. thermotolerans tagged with C-terminal SpyTag003 |
| 148 | MUP2-QtEnc | FIG. 2 c | Designed Uranyl-binding protein MUP2 fused to the |
| | | | shell forming monomer of the Encapsulin system of |
| | | | Quasibacillus thermotolerans |
| 149 | MUP5-QtEnc | Related to | Designed Uranyl-binding protein MUP5 fused to the |
| | | FIG. 2 a | shell forming monomer of the Encapsulin system of |
| | | | Quasibacillus thermotolerans |
| 150 | B-FABP- | FIG. 3 b | Engineered SpyCatcher 003 domain fused to fatty acid |
| | SpyCatcher003 | | binding protein FABP |
| 151 | BM3h- | FIG. 3 e | Heme binding domain BM3h fused to engineered |
| | SpyCatcher003 | | SpyCatcher 003 domain |
| 152 | MS2 stem loop | FIG. 4 a | RNA motif for MCP |
| 153 | 2xGGGGS linker | FIG. 5 a | flexible linker |
| 154 | DD-N | FIG. 5 a | Engineered FKBP-derived degron |
| 155 | eUnaG-MT3- | FIG. 7 b | Fusion of engineered ultrasmall fluorescent protein |
| | QtEnc^{FLAG} | | eUnaG followed by MT3, QtEnc and a FLAG-tag |
| 156 | eUnaG-2xMT3- | FIG. 7 c | Fusion of engineered ultrasmall fluorescent protein |
| | QtEnc^{FLAG} | | eUnaG followed by 2xMT3, QtEnc and a FLAG-tag |
| 157 | mGreenLantern- | FIG. 7 d | Fusion of the engineered green fluorescent protein |
| | MT3-QtEnc^{FLAG} | | mGreenLantern, MT3, QtEnc and a FLAG epitope |
| 158 | CAMPARI2-MT3- | FIG. 7 d | Fusion of the engineered fluorescent Calcium integrator |
| | QtEnc^{FLAG} | | CAMPARI2, MT3, QtEnc and a FLAG tag |
| 159 | P2-TmEnc^{BC2-Tag} | FIGS. 5 f, 8 a, | Synthetic coiled-coil domain P2 (partner of P1) fused to |
| | | 20 c | the TmEnc (AA 1-264) with a C-terminal BC2-Tag |
| 160 | DHD13a- | FIG. 8 a | Synthetic coiled-coil domain DHD13a (partner of |
| | MxEnc^{StrepTagII} | | DHD13b) fused to MxEnc with a StrepTagII |
| 161 | P3-QtEnc^{FLAG} | FIG. 8 a | Synthetic coiled-coil domain P3 (partner of P4) fused to |
| | | | QtEnc with a FLAG-Tag |
| 162 | DD-N-mScarlet-I-P1 | FIG. 8 a | Fusion of DD-N degron with mScarlet-I and synthetic |
| | | | coiled-coil domain P1 (partner of P2) |
| 163 | DD-N-mTaBFP2- | FIG. 8 a | Fusion of DD-N degron with mTagBFP2 and synthetic |
| | DHD13b | | coiled-coil domain DHD13b (partner of DHD13a) |
| 164 | DD-N- | FIG. 8 a | Fusion of DD-N degron with mGreenLantern and |
| | mGreenLantern-P4 | | synthetic coiled-coil domain P4 (partner of P3) |
| 165 | MT3-QtEnc^{FLAG} | FIG. 9 b | Fusion of Mus musculus Metallothionein 3, the shell |
| | | | forming monomer of the Encapsulin system of |
| | | | Quasibacillus thermotolerans and a C-terminal FLAG-Tag |
| 166 | MT3-MxEnc^{FLAG} | FIG. 9 c | Fusion of Mus musculus Metallothionein 3, the shell |
| | | | forming monomer of the Encapsulin system of |
| | | | Myxococcus xanthus and a C-terminal FLAG-Tag |
| 167 | MT3-MxEnc-FLAG- | FIGS. 9 h, 9 j | Fusion of Mus musculus Metallothionein 3, the shell |
| | cMyc-NLS | | forming monomer of the Encapsulin system of |
| | | | Myxococcus xanthus and a C-terminal FLAG-Tag followed |
| | | | by a cMyc-derived nuclear localization signal (NLS) |
| 168 | MT3-QtEnc-FLAG- | FIG. 9 i | Fusion of Mus musculus Metallothionein 3, the shell |
| | cMyc-NLS | | forming monomer of the Encapsulin system of |
| | | | Quasibacillus thermotolerans and a C-terminal FLAG-Tag |
| | | | followed by a cMyc-derived nuclear localization signal |
| | | | (NLS) |
| 169 | 3xchimericMT | FIG. 10 a | Fusion of 3xMT with QtEnc and a C-terminal FLAG-Tag. |
| | (3xMT)-QtEnc^{FLAG} | | |
| 170 | mINT | FIG. 11 a | attachment signal to vault |
| 171 | MT3-SpyCatcher003 | FIGS. 10 f, 12 b | MT3 fused to SpyCatcher003 |
| 172 | antiALFATag- | FIGS. 14 a, 14 b | Fusion of antiALFATag intrabody with an MT3 tandem, |
| | intrabody-2xMT3- | | GB1 domain, a FLAG-Tag and C-terminal RXXG degron. |
| | GB1-FLAG-RXXG | | |
| 173 | antiHA- | FIGS. 14 a, 14 b | Fusion of antiHA frankenbody with a MeLanM tandem, |
| | frankenbody- | | GB1 domain, a FLAG-Tag and C-terminal RXXG degron. |
| | 2xMeLanM-GB1- | | |
| | FLAG-RXXG | | |
| 174 | antiMoonTag- | FIGS. 14 a, 14 b | Fusion of antiMoonTag intrabody with a AssVanabin2 |
| | intrabody- | | tandem, GB1 domain, a FLAG-Tag and C-terminal RXXG |
| | 2xAssVanabin2-GB- | | degron. |
| | 1-FLAG-RXXG | | |
| 175 | antiMoonTag- | FIGS. 14 a, 14 b | Fusion of antiMoonTag intrabody with a MeLanM |
| | intrabody- | | tandem, GB1 domain, a FLAG-Tag, and C-terminal RXXG |
| | 2xMeLanM-GB-1- | | degron. |
| | FLAG-RXXG | | |
| 176 | antiMoonTag- | FIGS. 14 a, 14 b | Fusion of antiMoonTag intrabody with a 4xP3 CCC-Tag, |
| | intrabody-4x(P3)- | | GB1 domain, a FLAG-Tag, and C-terminal RXXG degron. |
| | GB-1-FLAG-RXXG | | |
| 177 | antiMoonTag- | FIGS. 14 a, 14 b | Fusion of antiMoonTag intrabody with an MT3 tandem, |
| | intrabody-2xMT3- | | GB1 domain, a FLAG-Tag, and C-terminal RXXG degron. |
| | GB-1-FLAG-RXXG | | |
| 178 | antiMoonTag- | FIGS. 14 a, 14 b | Fusion of antiMoonTag intrabody with an EcCusF |
| | intrabody-2xEcCusF- | | tandem, GB1 domain, a FLAG-Tag, and C-terminal RXXG |
| | GB-1-FLAG-RXXG | | degron. |
| 179 | LifeAct-emFPv4 | FIGS. 14 b, 14 d | Fusion of LifeAct-peptide to emFPv4 |
| 180 | QtEnc^{FLAG-}(TGA) | FIG. 15 b | DNA sequence encoding QtEnc-FLAG followed by a TGA |
| | RT20s- | | stop codon, followed by the read-through promoting |
| | antiGFPintrabody- | | sequence “RT20s” followed by an anti-GFPintrabody |
| | HiBit | | with a C-terminal HiBit peptide. |
| 181 | QtEnc^{FLAG-}(TGA) | FIG. 15 b | DNA sequence encoding QtEnc-FLAG followed by a TGA |
| | RT20s-Ssp DnaE | | stop codon, followed by a read-through promoting |
| | (N159A)QtEnc- | | sequence “RT20s”, followed by an Ssp DnaE intein |
| | antiGFPintrabody- | | mutant (N159A) which exhibits hyperfast N-terminal |
| | HiBit | | cleavage, followed by a P2A peptide followed by a |
| | | | second full-length QtEnc with a C-terminal antiGFP- |
| | | | intrabody and a HiBit peptide. |
| 182 | P2A peptide | FIG. 15 b | |
| 183 | 2xMT3-QtEnc- | FIG. 16 c | Tandem MT3 fused to QtEnc which has a C-terminal anti |
| | antiGFPintrabody | | GFP intrabody |
| 184 | sfGFP | Related to FIG. | |
| | | 12 c, 12 d , 17 a, | |
| | | 17 b, 17 c, 17 d, 17 e, | |
| | | 17 f, 17 g, 18 g, 18 k | |
| 185 | mCherry | FIG. 1 17 a, 17 b, | |
| | | 17 c, 17 d, 17 e, | |
| | | 17 f, 17 g, 18 g, 18 k | |
| 186 | antiGFP-intrabody | FIGS. 17 a, 18 l | |
| 187 | antimCherry | FIG. s17 a, 18 l | |
| | intrabody (LaM-4) | | |
| 188 | sfGFP-SasG-G51- | FIG. 17 a | SasG capped off with two fluorscent proteins |
| | G54-mCherry | | |
| 189 | MT3-QtEnc- | FIG. 17 c | MT3 fused to QtEnc with a C-terminal anti GFP intrabody |
| | antiGFPintrabody | | |
| 190 | BmTyr-P2 | FIG. 20 c | Fusion of BmTyr and synthetic coiled-coil domain P2 |
| | | | (partner of P1) |
| 191 | B-FABP-sfGFP | FIG. 10 e | Fusion of Brain FABP and superfolder GFP |
| 192 | BM3h-sfGFP | FIG. 10 e | Fusion of BM3h and superfolder GFP |
| 193 | cpFABP | FIG. 13 b | Circularly permuted FABP, Nterm and Cterm are both |
| | | | alpha helices |
| 194 | Intein C-6xB-FABP- | FIG. 13 a | Fusion of six B-FABP flanked by DnaE split intein |
| | Intein N | | fragments C-intein (AA 103-137) on the N-term and N- |
| | | | Intein (AA 1-102) |
| 195 | Intein C-3x(B-ABP- | FIG. 13 a | Fusion of three repeats of B-FABP fused to MmMT3 |
| | MmMT3)-Intein N | | flanked by DnaE split intein fragments C-intein (AA 103- |
| | | | 137) on the N-term and N-Intein (AA 1-102) |
| 196 | Intein C-2xB-FABP- | FIG. 13 c | Fusion of three repeats of 2x B-FABP intermixed by |
| | SpyTag003-2xB- | | SpyTag003 and Spycatcher003 and flanked by DnaE split |
| | FABP- | | intein fragments C-intein (AA 103-137) on the N-term |
| | Spycatcher003-2xB- | | and N-Intein (AA 1-102) |
| | FABP-Intein N | | |
| 197 | QtEnc-M13 | FIG. 19 a | Fusion of QtEncapsulin and M13 a synthetic peptide |
| | | | binding Calmodulin |
| 198 | B-FABP-CaM | FIG. 19 a | Fusion of Brain FABP and Calmodulin |
| 199 | AG4 | related to FIG. 2 | Synthetic Silver mineralizing peptide 4 |
| 200 | dCas13 | related to FIG. 4 | Catalytically inactive Cas13b is from Prevotella sp as an |
| | | | RNA-binding protein |
| 201 | Pumilio homology | related to FIG. 4 | RNA-binding protein Uniprot: Q14671 |
| | domain | | |
| 202 | eUnaG | Related to FIGs. | Enhanced Ultra small fluorescent protein from eel |
| | | 7 a, 7 b, 7 c, 7 e, | |
| | | 7 f, 11 e, | |
| 203 | mGreenLantern | FIG. 7 d | Improved green fluorescent protein based on GFP |
| 204 | CAMPARI 2 | FIG. 7 d | Engineered fluorescent Calcium integrator |
| 205 | mRFP | | Monomeric red fluorescent protein |
| 206 | FHA2 | related to FIG. | phostphopetptide binding Forkhead-associated domain |
| | | 19b | |
| 207 | AP205 Phage | FIGS. 10 b, 10d, 14 f | Coat protein |
| 208 | mEos4b:APEX2:mINT | FIGS. 11a, 11b | Fusion of mEos4b together with APEX2 to a C-terminal |
| | | | minimal interaction domain (mINT) capable to interact |
| | | | with the interior of the vault complex. |
| 209 | MmuGap43- | FIGS. 11a, 11b | Fusion of mouse Gap43 palmitoylation signal to the N- |
| | palmi:MVP | | terminus of the major vault protein (MVP). |
| 210 | SNAPtag-QtEnc^{FLAG} | FIG. 8 b | N-terminal fusion of SNAP-tag connected via GS linker to |
| | | | QtEnc with a C-terminal FLAG epitope |
| 211 | Halotag-QtEnc^{FLAG} | FIG. 8 b | N-terminal fusion of Halo-tag connected via GS linker to |
| | | | QtEnc with a C-terminal FLAG epitope |
| 212 | MT3-PfEnc^{FLAG} | FIG. 9 d | Fusion of MT3 to AA 110-346 of Pyrococcus furiosus |
| | | | Encapsulin monomer with a C-terminal FLAG epitope. |
| 213 | PfFt | FIG. 19 | Ferritin from Pyrococcus furiosus |
| 214 | mEos4b | Related to FIGS. | [symbol] www.fpbase.org/protein/meos4b/ |
| | | 11 a, 11 b | |
| 215 | mEosEM | Related to FIG. 7 | [symbol] www.fpbase.org/protein/meosem/ |
| 216 | iRFP713 | | [symbol] www.fpbase.org/protein/irfp713/ |
| 217 | TeAPCα | | [symbol] www.fpbase.org/protein/teapc/ |
| 218 | DMT1 | FIG. 6 d | Human divalent metal transporter 1 |
| 219 | Zip14 | FIG. 6 d | Human divalent metal transporter Zip14 |
| 220 | Cystathionine | FIG. 6 e | Fusion of Cystathionine gamma-lyase with QtSig |
| | gamma-lyase-QtSig | | |
| 221 | Secreted alkaline | FIG. 6 e | Fusion of SEAP, a secreted form of Human embryonic |
| | phosphatase-QtSig | | alkaline phosphatase, and QtSig |
| 222 | MnxG-QtSig | FIG. 6 e | Fusion of MnxG, component of the Bacillus sp. |
| | | | Multimeric multicopper oxidase Mnx, and QtSig |
| 223 | MnxE | FIG. 6 e | Component of the Bacillus sp. Multimeric multicopper |
| | | | oxidase Mnx |
| 224 | MnxF | FIG. 6 e | Component of the Bacillus sp. Multimeric multicopper |
| | | | oxidase Mnx |
| 225 | SUP | alternative to | Designed uranyl binder with femtomolar affinity |
| | | MUP2 and MUP5 | |
| | | in FIG. 2 | |
| 226 | P3-MT3-QtEnc | related to FIG. 8 | Coiled-coil adapter to recruit an encapsulated cargo, |
| | | | such as the fluorescent protein mEosEM fused to P4 to |
| | | | an encapsulin already harboring an MT3 for dual EM and |
| | | | fluorescence detection. |
| 227 | Mt3-QtEnc-eUnaG | alternative option | Fusion of the fluorescent protein eUnaG to the outer |
| | | to fluorescent | surface of encapsulin |
| | | protein | |
| | | complementation | |
| | | shown in FIG. 7 | |
| 228 | MT3-sfGFP | alternative option | CGU for attaching to the outer surface of encapsulin via |
| | | to fluorescent | anti-GFP antibody |
| | | protein | |
| | | complementation | |
| | | shown in FIG. 7 | |
| 229 | QtEnc-B-FABP | FIG. 10, | Fusion of QtEnc and Brain FABP, embodiment of |
| | | particularly 10e, | concentric barcodes added to the outer surface of |
| | | 10f | encapsulins |
| 230 | QtEnc-Bm3h | FIG. 10, | Fusion of QtEnc and Bm3h, embodiment of concentric |
| | | particularly 10e, | barcodes added to the outer surface of encapsulins |
| | | 10f | |
| 231 | QtEnc-MT3 | FIG. 10 e | Fusion of QtEnc and MT3, embodiment of concentric |
| | | | barcodes added to the outer surface of encapsulins |
| 232 | B-FABP-sfGFP | e.g. FIG. 10e | Fusion of Brain FABP and superfolder GFP, for |
| | | | attachment via anti-GFP intrabody attachment points, |
| | | | e.g. to the outer surface of Encapsulins. |
| 233 | Bm3h-sfGFP | e.g. FIG. 10e | Fusion of Bm3h and superfolder GFP, or attachment via |
| | | | anti-GFP intrabody attachment points, e.g. to the outer |
| | | | surface of Encapsulins. |
| 234 | MT3-Lam4 | related to FIG. | Fusion of MT3 and Lam4, for attachment to mCherry, |
| | | 17a | e.g. on SasG |
| 235 | B-FABP-Lam4 | related to FIG. | Fusion of Brain FABP and Lam4, for attachment to |
| | | 17a | mCherry, e.g. on SasG |
| 236 | Bm3h-Lam4 | related to FIG. | Fusion of Bm3h and Lam4, for attachment to mCherry, |
| | | 17a | e.g. on SasG |
| 237 | GGSG linker | FIG. 13 | General linker used for protein fusions, including direct |
| | | | fusions of CGUs to achieve the nanometer sizes of a |
| | | | structural element |
| 238 | Recoverin | FIG. 19c | myristoyl switch motif for calcium-dependent re- |
| | | | localization a structural element such as vaults to the |
| | | | membrane |
| 239 | synthide-2-FHA2 | related to FIG. | synthide-2 binding for FHA2 |
| | | 19b | |
| 240 | MT3_QtEnc_MT3_e | FIG. 35 | Fusion of murine MT3, Q. thermotolerans encapsulin |
| | UnaG_RS20 | | forming shell protein, murine MT3, enhanced fluorescent |
| | | | protein eUnaG (Anguilla japonica) and the synthetic |
| | | | Calmodulin-binding peptide RS20 |
| 241 | MT3_QtEnc_{—} | FIG. 35 | Fusion of murine MT3, Q. thermotolerans encapsulin |
| | eUnaG_RS20 | | forming shell protein, enhanced fluorescent protein |
| | | | eUnaG (Anguilla japonica) and the synthetic Calmodulin- |
| | | | binding peptide RS20 |
| 242 | QtEnc_eUnaG_RS20 | FIG. 35 | Fusion of Q. thermotolerans encapsulin forming shell |
| | | | protein, enhanced fluorescent protein eUnaG (Anguilla |
| | | | japonica) and the synthetic Calmodulin-binding peptide |
| | | | RS20 |
| 243 | MT3_QtEnc_{—} | FIG. 35 | Fusion of murine MT3, Q. thermotolerans encapsulin |
| | eUnaG_RS20_P2A | | forming shell protein, enhanced fluorescent protein |
| | CaM-HpFtn | | eUnaG (Anguilla japonica) and the synthetic Calmodulin- |
| | | | binding peptide RS20, followed by a P2A peptide |
| | | | (Tporcine teschovirus-1), followed by CaM-HpFtn (Seq ID |
| | | | 134) |
| 244 | MT3_QtEnc_MT3_e | FIG. 35 | Fusion of murine MT3, Q. thermotolerans encapsulin |
| | UnaG_RS20_P2A_{—} | | forming shell protein, murine MT3, enhanced fluorescent |
| | CaM-HpFtn | | proteine eUnaG (Anguilla japonica) and the synthetic |
| | | | Calmodulin-binding peptide RS20, followed by a P2A |
| | | | peptide (porcine teschovirus-1), followed by CaM-HpFtn |
| | | | (Seq ID 134) |
| 245 | QtIMEF_P2A_QtEnc_{—} | FIG. 35 | Q. thermotolerans iron-mineralizing cargo protein IMEF |
| | eUnaG_RS20_P2A_{—} | | followed by P2A (porcine teschovirus-1), followed by |
| | CaM- | | Q. thermotolerans shell forming monomer fused to |
| | HpFtn_P2A_MmZip14 | | eUnaG, followed by synthetic Calmodulin binding |
| | | | peptide RS20, followed by P2A peptide (porcine |
| | | | teschovirus-1), followed by murine divalent metal uptake |
| | | | pump Zip14 |
| 246 | 1M-PfEnc- | FIG. 23 | Fusion of murine MT3, shell forming portion of the |
| | miRFP670nano | | encapsulin system of Pyrococcus furiosus and the |
| | | | monomeric far-red fluorescent protein mRFP670nano |
| | | | (Nostoc punctiforme) |
| 247 | 1M-PfEnc-FLAG- | FIG. 23 | Fusion of murine MT3, shell forming portion of the |
| | antiGFP | | encapsulin system of Pyrococcus furiosus, a FLAG epitope |
| | | | and an anti-GFP nanobody (intrabody) (Seq ID 186) |
| 248 | B-Fabp-ko | FIG. 22 | Mutant of Brain FABP inhibiting lipid binding |
| 249 | Bm3h-B7 | FIG. 22 | Mutant of Bm3h binding dopamine |
| 250 | QtEnc_B-Fabp-ko | FIG. 22 | Fusion of QtEnc and mutant Brain FABP |
| 251 | B-Fabp_HpFtn | FIG. 22 | Fusion of Brain FABP to H. pylori ferritin (AA 5-167, |
| | | | N19Q) |
| 252 | B-Fabp-ko_HpFtn | FIG. 22 | Fusion of Brain FABP mutant to H. pylori ferritin (AA 5- |
| | | | 167, N19Q) |
| 253 | Bm3h-HpFtn | FIG. 22 | Fusion of Bm3h to H. pylori ferritin (AA 5-167, N19Q) |
| 254 | Bm3h-B7-HpFtn | FIG. 22 | Fusion of Bm3h-B7 to H. pylori ferritin (AA 5-167, N19Q) |
| 255 | BFabp-FLAG | FIG. 22 | Fusion of Brain FABP and the FLAG epitope |
| 256 | BFabp-ko-FLAG | FIG. 22 | Fusion of the mutant Brain FABP and the FLAG epitope |
| 257 | Bm3h-FLAG | FIG. 22 | Fusion of Bm3h and the FLAG epitope |
| 258 | Bm3h-B7-FLAG | FIG. 22 | Fusion of Bm3h-B7 and the FLAG epitope |
| 259 | BFabp-αMoon-Tag | FIG. 32 | Fusion of B-Fabp to αMoon-Tag for targeting the EmFPs |
| | | | proteins |
| 260 | 1M_QtEnc_SNAP- | FIGS. 24, 25, 26 (n | Fusion to bioconjugation tags for preferred use with |
| | Tag | their entirety) | fixation stable fluorophores (such as SNAP-cell TMR star) |
| | | | for CLEM |
| 261 | 1M_QtEnc_Clip-TAG | FIGS. 24, 25, 26 (n | Fusion to bioconjugation tags for preferred use with |
| | | their entirety) | fixation stable fluorophores for CLEM |
| 262 | dcAvidin_GpAG83A | FIGS. 24, 25, 26 (n | In addition to SNAP/CLIP tag, also biotinylated ligands |
| | mCherry | their entirety) | could be semi-genetically appended using engineered |
| | | | Avidin variants |
| 263 | scdAvidin_GpAG83A_{—} | FIGS. 24, 25, 26 (n | In addition to SNAP/CLIP tag, also biotinylated ligands |
| | mCherry | their entirety) | could be semi-genetically appended using engineered |
| | | | Avidin variants |
| 264 | Mito-mEosEM | FIGS. 24, 25, 26 (n | mEosEM as fully-genetic fixation-stable fluorophore |
| | | their entirety) | |
| 265 | QtSig_mEosEM | FIGS. 24, 25, 26 (n | fixation-stable fluorophore with cargo targeting |
| | | their entirety) | sequence for encapsulation into QtSig. |
| 266 | SNAP- | FIGS. 24, 25, 26 (n | Combination of N-terminal SNATP tag with M for CLEM |
| | Tag_1M_QtEnc | their entirety) | with fixation-stable fluororphres such as TMR star |
| 267 | des_4bhv | FIG. 21 | Oligomeric designed uranyl binding protein |
| 268 | des_7bau | FIG. 21 | Oligomeric designed uranyl binding protein |
| 269 | des_2akf_3bs | FIG. 21 | Oligomeric designed uranyl binding protein |
| 270 | des_2akf_6bs | FIG. 21 | Oligomeric designed uranyl binding protein |
| 271 | des_5apq_6bs | FIG. 21 | Oligomeric designed uranyl binding protein |
| 272 | QtEnc_LgBit_M13 | FIG. 37 | Qt_Encapsulin as scaffold fused to the Large Bit of split |
| | | | nanoluc and the M13 peptide binding Calmodulin (Cam) |
| | | | in the presence of calcium |
| 273 | SmBit_B-Fabp_CaM | FIG. 37 | Small Bit of split Nanoluciferase fused to B-Fabp (as CGU) |
| | | | and Calmodulin |
| 274 | SmBit_CaM_B-Fabp | FIG. 37 | Small Bit of split Nanoluciferase fused to Calmodulin and |
| | | | B-Fabp (as CGU) |
| 275 | QtEnc_LgBit_CaM | FIG. 37 | Fusions of QtEnc to Large Bit of split Nanoluciferase and |
| | | | Calmodulin |
| 276 | SpyTag003_CaM_Sas | FIG. 37 | Spytag003, used for targeting to the structural element, |
| | G10_linker_SasG10 | | is fused to Calmodulin, Binding the M13 peptide present |
| | B-Fabp_M13 | | at the end of the construct only in presence of calcium |
| | | | ions. B-Fabp as a contrasting generating unit is also fused |
| | | | before the M13 Peptide. Separating Calmodulin and B- |
| | | | Fabp_M13 are two rigid protein filaments, SasG G10 |
| | | | linked by a flexible linker permitting rotational freedom. |
| 277 | QtEnc_CaM | FIG. 37 | QtEnc fusion to calmodulin for correlative calcium |
| | | | indicator imaging |
| 278 | M13_B- | FIG. 36 | M13 synthetic peptide binding Cam in the presence of |
| | FABP_TagRFP_{—} | | calcium ions is fused to B-Fabp as a CGU for EM contrast, |
| | Synzip17 | | TagRFP a red fluorescent protein as the FRET acceptor |
| | | | and the Synzip17 peptide binding the Synzip18 |
| 279 | TagGFP2_Synzip18 | FIG. 36 | TagGFP2 as the FRET donor fused to the antiparallel |
| | | | Synzip18 binding to Synzip 17 |
| 280 | CaTEV_hLOV_TEVseq_{—} | FIG. 36 | scFLARE system containing Calcium activated protease |
| | SpyTag003_B-FABP | | TEV (CaTEV), the light-dependent hLOVE domain and the |
| | | | cleavable TEV site (TEVseq), fused to Spytag003 and B- |
| | | | FABP as a CGU |
| 281 | Skipped sequence | | |
| 282 | Skipped sequence | | |
| 283 | Skipped sequence | | |
| 284 | Nterm-BCR_{—} | FIG. 31 | Engineered variant of Major Vault Protein (MVP) with |
| | RnMVP_HLHₒᵤₜₜₑᵣᵢₒᵣ- | | antiparallel coiled coils (Nterm-BCR) for closed Vault |
| | His6_HLHᵢₙₜₑᵣᵢₒᵣ- | | conformation, PCP for binding of PP7 aptamers in Vaults |
| | PCP | | interior, His Tag for Vaults purification |
| 285 | RnMVP_HLHₒᵤₜₜₑᵣᵢₒᵣ- | FIG. 31 | Engineered variant of Major Vault Protein (MVP), PCP for |
| | His6_HLHᵢₙₜₑᵣᵢₒᵣ-PCP | | binding of PP7 aptamers in Vaults interior, His Tag for |
| | | | Vaults purification |
| 286 | RnMVP_HLHₒᵤₜₜₑᵣᵢₒᵣ- | FIG. 31 | Engineered variant of Major Vault Protein (MVP), PCP for |
| | SNAP25- | | binding of PP7 aptamers in Vaults interior, SNAP Tag for |
| | palmitoylation- | | intracellular crystallization |
| | site_HLHᵢₙₜₑᵣᵢₒᵣ-PCP | | |
| 287 | MiniSOG_QtEnc- | FIG. 43 | Fluorescent encapsulins with nuclear export signal |
| | FLAG_NES | | |
| 288 | MiniSOG_QtEnc- | FIG. 43 | Fluorescent encapsulins with nuclear localization signal |
| | FLAG_cMycNLS | | |
| 289 | 1xMmMT3_QtEnc_{—} | FIG. 43 | Nuclear export signal to obtain preferentially cytosolic |
| | FLAG_NES | | contrast |
| 290 | mCherry-RAB11A | FIG. 42 | Fluorescent tag on RAB11A for live-microscopy tracking |
| | | | experiments |
| 291 | mCherry-PLK1 | FIG. 42 | Fluorescent tag on RAB11A for live-microscopy tracking |
| | | | experiments |
| 292 | mCherry-Myo5b | FIG. 42 | Fluorescent tag on RAB11A for live-microscopy tracking |
| | | | experiments |
| 293 | pcDNA_Zeo_1M- | No reference to | |
| | QtFLAG | figures is provided- | |
| 294 | pcDNA_Zeo_2M- | No reference to | |
| | QtFLAG | figures is provided- | |
| 295 | pcDNA_Zeo_3M- | No reference to | |
| | QtFLAG | figures is provided- | |
| 296 | pcDNA_Zeo_1M- | No reference to | |
| | MxFLAG | figures is provided- | |
| 297 | pcDNA_Zeo_2M- | No reference to | |
| | MxFLAG | figures is provided- | |
| 298 | pcDNA_Zeo_1M- | No reference to | |
| | TmBC2Tag | figures is provided- | |
| 299 | pcDNA_Zeo_1M- | No reference to | |
| | Tm_anti-mCherry | figures is provided- | |
| 300 | pcDNA_Zeo_1M- | No reference to | |
| | Qt_anti-GFP | figures is provided- | |
| 301 | pcDNA_sfGFP-2G- | No reference to | |
| | mCherry | figures is provided- | |
| 302 | pcDNA_sfGFP-3G- | No reference to | |
| | mCherry | figures is provided- | |
| 303 | pcDNA_sfGFP-4G- | No reference to | |
| | mCherry | figures is provided- | |
| 304 | pcDNA_sfGFP-5G- | No reference to | |
| | mCherry | figures is provided- | |
| 305 | pcDNA_sfGFP-6G- | No reference to | |
| | mCherry | figures is provided- | |
| 306 | pcDNA_sfGFP-7G- | No reference to | |
| | mCherry | figures is provided- | |
| 307 | pcDNA_sfGFP-8G- | No reference to | |
| | mCherry | figures is provided- | |
| 308 | pcDNA_sfGFP-9G- | No reference to | |
| | mCherry | figures is provided- | |
| 309 | pcDNA_sfGFP-10G- | No reference to | |
| | mCherry | figures is provided- | |
| 310 | pcDNA_1M_QtFLAG_{—} | No reference to | |
| | TGA_rt20s_antiGFP | figures is provided- | |
| 311 | EGFP-N1-msfGFP- | No reference to | |
| | Cx43, Addgene | figures is provided- | |
| | 69024 | | |
| 312 | EGFP-N1-Cx43- | No reference to | |
| | msfGFP, Addgene | figures is provided- | |
| | 69007 | | |
| 313 | pcDNA_HA-DD-N- | No reference to | |
| | mTagBFP2_QtSig | figures is provided- | |
| 314 | pcDNA_HA-DD-N- | No reference to | |
| | mScarlet-I_QtSig | figures is provided- | |
| 315 | pcDNA-1M-Qt- | No reference to | |
| | eUnaG | figures is provided- | |
| 316 | pcDNA-eUnaG-1M- | No reference to | |
| | Qt-anti-mCherry | figures is provided- | |
| 317 | pcDNA-eUnaG-1M- | No reference to | |
| | Qt-anti-GFP | figures is provided- | |
| 318 | pcDNA-eUnaG-1M- | No reference to | |
| | QtFLAG | figures is provided- | |
| 319 | pcDNA-eUnaG-2M- | No reference to | |
| | Qt-anti-mCherry | figures is provided- | |
| 320 | pcDNA-eUnaG-2M- | No reference to | |
| | Qt-anti-GFP | figures is provided- | |
| 321 | pcDNA-eUnaG-2M- | No reference to | |
| | QtFLAG | figures is provided- | |
| 322 | pJFRC7-20XUAS- | No reference to | |
| | IVS_1M_QtFLAG-NLS | figures is provided- | |
| 323 | pJFRC7-20XUAS- | No reference to | |
| | IVS_1M_MxFLAG- | figures is provided- | |
| | NLS | | |
| 324 | pJFRC19- | No reference to | |
| | 13XLexAop2-IVS- | figures is provided- | |
| | 3M_QtFLAG-NLS | | |
| 325 | pAAV_CaMKlla_{—} | No reference to | |
| | mScarlet- | figures is provided- | |
| | I_P2A_2M_QtFLAG | | |
| 326 | pcDNA_Zeo_QtFLAG | No reference to | |
| | | figures is provided- | |
| 327 | pcDNA_Zeo_MxFLAG | No reference to | |
| | | figures is provided- | |
| 328 | pcDNA_Zeo_TmBC2Tag | No reference to | |
| | | figures is provided- | |
| 329 | pcDNA-mCherryFLAG | No reference to | |
| | | figures is provided- | |
| 330 | pcDNA-1M- | No reference to | |
| | QtEnc_anti-mCherry | figures is provided- | |
| 331 | pcDNA-1M- | No reference to | |
| | QtEnc_StrepTagII | figures is provided- | |
| 332 | pcDNA_sfGFP-OG- | No reference to | |
| | mCherry | figures is provided- | |
| 333 | pcDNA_Zeo_DD- | No reference to | |
| | N_APEX2_EncTag_GA | figures is provided- | |
| 334 | pcDNA_Zeo_QtEnc_SpyTag | No reference to | |
| | | figures is provided- | |
| 335 | pcDNA_Zeo_antiGFP- | No reference to | |
| | SpyCatcherFLAG | figures is provided- | |
| 336 | pcDNA_Zeo_QtEnc_P4 | No reference to | |
| | | figures is provided- | |
| 337 | pCMV_EGFP_CAAX | No reference to | |
| | | figures is provided- | |
| 338 | pCMV_P3_EGFP_CAAX | No reference to | |
| | | figures is provided- | |
| 339 | pcDNA- | No reference to | |
| | QtFLAG_TAG_rt9s_{—} | figures is provided- | |
| | IntP2A_Qt- | | |
| | antiGFP_HiBit | | |
| 340 | pcDNA- | No reference to | |
| | QtFLAG_TAG_rt9s_{—} | figures is provided- | |
| | antiGFP_HiBit | | |
| 341 | pcDNA- | No reference to | |
| | QtFLAG_TAA_rt9s_{—} | figures is provided- | |
| | antiGFP_HiBit | | |
| 342 | pcDNA- | No reference to | |
| | QtFLAG_TGA_rt9s_{—} | figures is provided- | |
| | antiGFP_HiBit | | |
| 343 | pcDNA- | No reference to | |
| | QtFLAG_TAG_rt9us_{—} | figures is provided- | |
| | antiGFP_HiBit | | |
| 344 | pcDNA- | No reference to | |
| | QtFLAG_TAA_rt9us_{—} | figures is provided- | |
| | antiGFP_HiBit | | |
| 345 | pcDNA- | No reference to | |
| | QtFLAG_TGA_rt9us_{—} | figures is provided- | |
| | antiGFP_HiBit | | |
| 346 | mCherry-Rab11a, | No reference to | |
| | Addgene 55124 | figures is provided- | |
| 347 | mCherry-PLK1, | No reference to | |
| | Addgene 55119 | figures is provided- | |
| 348 | pcDNA_Zeo_MiniSOG_{—} | No reference to | |
| | QtEnc-FLAG_NES | figures is provided- | |
| 349 | pcDNA_Zeo_MiniSOG_{—} | No reference to | |
| | QtEnc- | figures is provided- | |
| | FLAG_cMycNLS | | |
| 350 | pJFRC7-20XUAS- | No reference to | |
| | IVS_1xMmMT3_QtEnc_{—} | figures is provided- | |
| | FLAG_NES | | |
| 351 | pU6_U6 + 27_5′-3′- | No reference to | |
| | Tornado_Broccoli- | figures is provided- | |
| | PP7_3′- | | |
| | tevopreQ1_6xT | | |
| 352 | pU6_U6 + 27_5′-3′- | No reference to | |
| | Tornado_Broccoli(miniCTE- | figures is provided- | |
| | PP7)_3′-tevopreQ1_6xT | | |
| 353 | pU6_U6 + 27_5′-3′- | No reference to | |
| | Tornado_Broccoli(miniCTE(PP7))_{—} | figures is provided- | |
| | 3'-tevopreQ1_6xT | | |
| 354 | Nterm- | No reference to | |
| | BCR_RnMVP_HLHₒᵤₜₜₑᵣᵢₒᵣ- | figures is provided- | |
| | His6_HLHᵢₙₜₑᵣᵢₒᵣ- | | |
| | PCP | | |
| 355 | RnMVP_HLHₒᵤₜₜₑᵣᵢₒᵣ- | No reference to | |
| | His6_HLHᵢₙₜₑᵣᵢₒᵣ-PCP | figures is provided- | |
| 356 | RnMVP_HLHₒᵤₜₜₑᵣᵢₒᵣ- | No reference to | |
| | SNAP25- | figures is provided- | |
| | palmitoylation- | | |
| | site_HLHᵢₙₜₑᵣᵢₒᵣ-PCP | | |
| 357 | QtIMEF_P2A_QtEnc_{—} | FIG. 35 | Encapsulins co-clustering with Ferritin (HpFtn) can also |
| | eUnaG_{—} | | be induced by far-red light (MagRed system). When |
| | Aff6V18deltaN_{—} | | encapsulins are iron-loaded, this light-triggered |
| | P2A_DrBphP_HpFtn | | clustering can be used to temporally modulate signals in |
| | | | magnetic resonance imaging to improve SNR. |
| 358 | QtIMEF_P2A_QtEnc_{—} | FIG. 35 | Encapsulins co-clustering with Ferritin (HpFtn) can also |
| | pMagFast2_P2A_{—} | | be induced by blue-light (pMag/nMag system). When |
| | nMagHigh1_HpFtn | | encapsulins are iron-loaded, this light-triggered |
| | | | clustering can be used to temporally modulate signals in |
| | | | magnetic resonance imaging to improve SNR. |
| 359 | antiGFPintrabody_{—} | Related to 7, 8 , 23, | Relate to Encapsulin targeting. These constructs allow for |
| | TEVsite_FKBP12 | 24, 25, 26, 27, 28, | blue-light induced targeting of Encapsulins to GFP-tagged |
| | | 29, 37 | sub-cellular structures. |
| 360 | MT3-QtEnc-FRB | Related to 7, 8 , 23, | Relate to Encapsulin targeting. These constructs allow for |
| | | 24, 25, 26, 27, 28, | blue-light induced targeting of Encapsulins to GFP-tagged |
| | | 29, 37 | sub-cellular structures. |
| 361 | MT3-QtEnc- | Related to 7, 8 , 23, | Relate to Encapsulin targeting. These constructs allow for |
| | nMagHigh1 | 24, 25, 26, 27, 28, | rapamycin-induced targeting of Encapsulins, in this case |
| | | 29, 37 | to membrane localized EGFP (via CAAX signal) |
| 362 | pMagFast2_EGPF_{—} | Related to 7, 8 , 23, | Relate to Encapsulin targeting. These constructs allow for |
| | CAAX | 24, 25, 26, 27, 28, | rapamycin-induced targeting of Encapsulins, in this case |
| | | 29, 37 | to membrane localized EGFP (via CAAX signal) |
| 363 | pU6_U6 + 27_5′-3′- | FIG. 31 | Twister-stabliized RNA aptamer (PP7) for CLEM contrast |
| | Tornado_Broccoli- | | targetable to structural elements containing PCP, |
| | PP7_3′- | | tevopreQ1 for 3′ RNA stabilization. |
| | tevopreQ1_6xT | | |
| 364 | pU6_U6 + 27_5′-3′- | FIG. 31 | Twister-stabilized RNA aptamer (PP7) for CLEM contrast |
| | Tornado_{—} | | targetable to structural elements containing PCP, |
| | Broccoli(miniCTE-PP7)_{—} | | miniCTE for nuclear export of RNA components, |
| | 3′-tevopreQ1_6xT | | tevopreQ1 for 3′ RNA stabilization. |
| 365 | pU6_U6 + 27_5′--3′- | FIG. 31 | Twister-stabilized RNA aptamer (PP7) for CLEM contrast |
| | Tornado_{—} | | targetable to structural elements containing PCP,Spinach |
| | Broccoli(Spinach-PP7))_{—} | | aptamer for Pb2+ binding, tevopreQ1 for 3′ RNA |
| | 3′-tevopreQ1_6xT | | stabilization. |
| No | rt9us (sequence: | FIG. 33 | DNA sequence promoting ribosomal (translational) read- |
| ID | CTATCC) | | through after a STOP codon |

1. A genetically controlled nanoscopy contrast-generating unit for providing enhanced resolution of a molecular target comprising a metal interactor,
wherein the metal interactor is compatible with nanoscopy fixation protocols, nanoscopy post-fixation protocols, and nanoscopy metal staining protocols,
wherein the metal interactor is a molecule to which a metal ion binds to or reacts with, and
wherein the metal interactor is not a ferroxidase or an enzymatic product from an exogenous substrate.

2. The genetically controlled nanoscopy contrast-generating unit according to claim 1, wherein the metal interactor comprises one or more of the following elements:
a metal-interacting peptide/protein,
a metal-binding peptide/protein,
a lipid-binding protein,
an RNA-binding protein,
a DNA or RNA molecule,
a polymerizing or synthesizing enzyme,
a biomineralizing enzyme,
a bioconjugation tag,
or a combination thereof.

3. The genetically controlled nanoscopy contrast-generating unit according to claim 1, wherein the metal interactor comprises one or more of the following elements:
a metallothionein,
osmiophilic amino acids,
a lead binder,
a lanthanide binder,
a uranyl-binder,
a vanadium binder,
a copper and silver-binder,
a cobalt or nickel binder,
a fatty-acid-binding protein,
a lipid-binding domain of a cytochrome P450,
an RNA aptamer-binder:RNA aptamer pair,
a programmable RNA binding protein,
a polymerizing kinase,
a phytochelatin synthase, or
a tyrosinase.

4. The genetically controlled nanoscopy contrast-generating unit according to claim 1,
wherein the genetically controlled nanoscopy contrast-generating unit further comprises a fluorophore or a chromophore, wherein
(i) the fluorophore is a fluorescent, a co-factor in a fluorescent protein, or an exogenous fluorophore, or
(ii) the chromophore is a chromoprotein, a co-factor in a chromoprotein, or an exogenous chromophore, and
wherein the exogenous fluorophore or the exogenous chromophore binds directly to the metal interactor.

5. The genetically controlled nanoscopy contrast-generating unit according to claim 1, wherein the metal interactor comprises
(i) at least one metallothionein, at least one fatty-acid binding protein, at least one lipid-binding domain, or at least one RNA molecule, and
ii) a fluorophore, wherein the fluorophore is a fluorescent protein.

6. (canceled)

7. A genetically controlled structural element for nanoscopic detection,
wherein the genetically controlled structural element spatially organizes the genetically controlled nanoscopy contrast-generating unit according to claim 1,
wherein the genetically controlled structural element has a pre-determined shape and a pre-determined nanoscale size, and
wherein the genetically controlled structural element generates a shape that can be differentiated at nanoscale resolution.

8. The genetically controlled structural element according to claim 7 further comprising a binder or at least one attachment point for labeling a subcellular target of interest selected from a member of the encapsulin family, a vault, an MS2 phage, a Qbeta phage, an AP205 phage, an engineered mono-to-polyvalent hub, a filament, a linker, or combinations thereof.

9. The genetically controlled structural element according to claim 8,
wherein said at least one attachment point is selected from the group consisting of nanobodies, frankenbodies, coiled-coil domains, isopeptide-forming partners, bioconjugation-tags, split-inteins, calmodulin-binding peptides, phosphorylatable peptides, conformation-changing peptides including troponins, and combinations thereof, and
wherein the genetically controlled nanoscopy contrast-generating unit interacts with or binds to the at least one attachment point.

10. The genetically controlled structural element according to claim 7,
wherein the genetically controlled structural element is an encapsulin,
wherein the genetically controlled contrast-generating unit is selected from the group of one or more metallothionein MT3, one or more different metallothionein species, one or more fatty acid-binding proteins (FABPs), and one or more of the lipid-binding domain of the cytochrome P450, or combinations thereof,
wherein one to three copies of the MT3 or one to three different metallothionein species are bound to the N-terminus of the encapsulin,
wherein one or more MT3s, FABPs, or BM3hs are bound to the surface of the encapsulin, and
wherein the spatial organization is configured to generate the distinct shape of a barcode, wherein the barcode is a concentric barcode differentiable at nanoscale resolution.

11. A genetically controlled scaffold for organizing structural elements for nanoscopic detection,
wherein the genetically controlled scaffold comprises and spatially organizes the genetically controlled structural elements according to claim 7, and
wherein the spatial organization generates distinct geometric patterns that are differentiated at nanoscale resolution.

12. The genetically controlled scaffold according to claim 11, wherein the genetically controlled scaffold is selected from the group consisting of an endogenous cellular scaffold, a designed scaffold, a CsgA element, a SasG element, an RNA or DNA structure, and combinations thereof.

13. The genetically controlled scaffold according to claim 11, comprising
a SasG of variable length,
at least one encapsulin structural element, and
at least one contrast generating unit MT3, or a series of different metallothionein species,
wherein the at least one encapsulin is connected and spatially organized via SasG, and
wherein the at least one encapsulin comprises the at least one contrast generating unit MT3, or a series of different metallothionein species contrast generating units.

14. A nanoscopy method for nanoscopic detection comprising:
(a) providing the genetically controlled nanoscopy contrast-generating unit according to claim 1, and/or
(b) providing the genetically controlled structural element according to claim 7, and/or
(c) the genetically controlled scaffold according to claim 11,
wherein the nanoscopy detection comprises molecular mapping and/or geometric sensing.

15. The method according to claim 14,
wherein the nanoscopy detection is molecular mapping, and
wherein the molecular mapping provides information on the subcellular distribution of a molecule of interest within a cell or tissue via the binding of the genetically controlled structural element and/or genetically controlled scaffold to the molecule, which thus determines the subcellular localization of the genetically controlled structural element and/or the genetically controlled scaffold.

16. The method according to claim 14,
wherein the nanoscopy detection is geometric sensing, and
wherein the geometric sensing provides information on a specific cellular state, a cellular process, or the presence or absence of an analyte or environmental parameter of interest within a cell or tissue, or the response to an external stimulus, and
wherein the shape generated by the genetically controlled structural element and/or the geometric pattern generated by the genetically controlled scaffold changes in response to a pre-defined cellular state, a pre-defined cellular process, at least one analyte, at least one environmental parameter, or an external stimulus.

17. The method according to claim 14,
wherein the nanoscopy detection comprising mapping and/or the geometrical sensing further comprises geometric actuation,
wherein the geometric actuation alters a cellular state or process by a biomechanical, and/or biochemical effect or via an external stimulus, or the deposition of external energy, electromagnetic radiation, mechanical energy, or magnetic gradients.

18. The method according to claim 14,
wherein the contrast generating unit, structural element and/or scaffold is compatible with intact cell systems, preferably with mammalian cells and organoids or biomedical model organisms.

19-133. (canceled)

134. The genetically controlled structural element according to claim 7, wherein the generated shape is a barcode encoding information for a cellular state using geometric sensing.

135. The genetically controlled structural element according to claim 10, wherein the one or more metallothioneins, the lipid binder, the RNA aptamer, and the one or more fluorescent proteins of the genetically controlled nanoscopy contrast-generating unit are bound to the surface of the encapsulin genetically controlled structural element.


SEQUENCE LISTING (365 sequences)

SEQ ID NO: 1 - protein, 68 aa, Mus musculus
MDPETCPCPTGGSCTCSDKCKCKGCKCTNCKKSCCSCCPAGCEKCAKDCVCKGEEGAKAEAEKCSCCQ

SEQ ID NO: 2 - protein, 74 aa, Mus musculus
HHHHHHMDPETCPCPTGGSCTCSDKCKCKGCKCTNCKKSCCSCCPAGCEKCAKDCVCKGEEGAKAEAEKCSCCQ

SEQ ID NO: 3 - protein, 77 aa, Mus musculus
HHHHHHHHHMDPETCPCPTGGSCTCSDKCKCKGCKCTNCKKSCCSCCPAGCEKCAKDCVCKGEEGAKAEAEKCSCCQ

SEQ ID NO: 4 - protein, 147 aa, Mus musculus
MDPETCPCPTGGSCTCSDKCKCKGCKCTNCKKSCCSCCPAGCEKCAKDCVCKGEEGAKAEAEKCSCCQGGGSGGGSGGSADPETCPCPTGGSCTCSDKCKCKGCKCTNCKKSCCSCCPAGCEKCAKDCVCKGEEGAKAEAEKCSCCQ

SEQ ID NO: 5 - protein, 214 aa, synthetic construct
  REGION 1..214: note = cimeric metallothionein
MDPETCPCPTGGSCTCSDKCKCKGCKCTNCKKSCCSCCPAGCEKCAKDCVCKGEEGAKAEAEKCSCCQGGTGGSTSTTLVKCACEPCLCNVDPSKAIDRNGLYYCSEACADGHTGGSKGCGHTGCNCHGSAGGCDDKCGCAVPCPGGTGCRCTSARSGAAAGEHTTCGCGEHCGCNPCACGREGTPSGRANRRANCSCGAACNCASCGSATAPG

SEQ ID NO: 6 - protein, 300 aa, Pinctada fucata
MKFLAAVAFVAVLSHSTVYSYGPRNRGKMMPMMPPAKKMMNNDMMKMMKPDDAMMKPDKQNQMKPDDMKQMMKMMMDKMMMMDKMMMMDMMMKMMKPDMMMKPDMMMKPDMMMKPDMMMKPDMMMKPDMMMKPDMMMKPDMMMKPDMMMKPDMMMKPDMMMKPDMMMKPDMMMKPDMMMKPDMMMKPDMMMKPDMMMKPDMMMKPDMMMKPDMMMKPDMMMKPDMMMKPDMMMKPDMMMKPDMMMKPDMMMKPDMMMKPDMMMKPDMMMKPDMMMKPDMGMMKPDMGMMMHPTPYNLYQG

SEQ ID NO: 7 - protein, 31 aa, Panax ginseng
CKSGGAWCGFDPHGCCGNCGCLVGFCYGTGC

SEQ ID NO: 8 - protein, 28 aa, Klebsiella pneumoniae
ECPRCDSYYTLHQAQHHLRCHHCDSQRP

SEQ ID NO: 9 - protein, 26 aa, Homo sapiens
EAVCCSDHQHCCPQGYTCVAEGQCQK

SEQ ID NO: 10 - protein, 45 aa, Utricina eques
CYPGQPGCGHCSRPNYCEGARCESGFHDCGSDHWCDASGDRCCCA

SEQ ID NO: 11 - protein, 27 aa, Homo sapiens
LMVKCPNEGCLHKMELRHLEDHQAHCE

SEQ ID NO: 12 - protein, 111 aa, Methylobacterium extorquens
PTTTTKVDIAAFDPDKDGTIDLKEALAAGSAAFDKLDPDKDGTLDAKELKGRVSEADLKKLDPDNDGTLDKKEYLAAVEAQFKAANPDNDGTIDARELASPAGSALVNLIR

SEQ ID NO: 13 - protein, 20 aa, synthetic construct
  REGION 1..20: note = Synthethic lanthanide binding tag
FIDTNNDGWIEGDELLLEEG

SEQ ID NO: 14 - protein, 119 aa, Ascidia sydneiensis samea
SKVIFALVLVVVLVACINATYVEFEEAYAPVDCKGQCTTPCEPLTACKEKCAESCETSADKKTCRRNCKKADCEPQDKVCDACRMKCHKACRAANCASECPKHEHKSDTCRACMKTNCK

SEQ ID NO: 15 - protein, 88 aa, Escherichia coli
NEHHHETMSEAQPQVISATGEVKGIDLESKKITIHHDPIAAVNWPEMTMRFTITPQTKMSEIKTGDKVAFNFVQQGNLSLLQDIKVSQ

SEQ ID NO: 16 - protein, 10 aa, synthetic construct
  REGION 1..10: note = Encapsulation signal for the Encapsulin system of Quasibacillus thermotolerans
KGFTVGSLIQ

SEQ ID NO: 17 - protein, 298 aa, Plasmodium falciparum
FNNNLCSKNAKGLNLNKRLLHETQAHVDDAHHAHHVADAHHAHHVADAHHAHHVADAHHAHHAADAHHAHHAADAHHAHHAADAHHAHHAHHAADAHHAHHAADAHHAHHAADAHHAHHAADAHHAHHAADAHHAHHASDAHHAHHAAYAHHAHHASDAHHAADAHHAAYAHHAHHAADAHHAADAHHATDAHHAADAHHAADAHHAADAHHATDAHHAHHAADAHHATDAHHAHHAADAHHAAAHHATDAHHAAAHHATDAHHAAAHHEAATHCLRHGGGGSGGGGSKGFTVGSLIQ

SEQ ID NO: 18 - protein, 212 aa, synthetic construct
  REGION 1..212: note = Fusion protein consisting of enhanced UnaG(V2L) from Anguilla japonica and MmMT3
MLEKFVGTWKIADSHNFGEYLKAIGAPKELSDGGDATTPTLYISQKDGDKMSVKIENGPPTFLDTQVKFKLGEEFDEFPSDRRKGVKSVVNLVGEKLVYVQKWDGKETTYVREIKDGKLVVTLTMGDVVAVRSYRRATEGGTGGSDPETCPCPTGGSCTCSDKCKCKGCKCTNCKKSCCSCCPAGCEKCAKDCVCKGEEGAKAEAEKCSCCQ

SEQ ID NO: 19 - protein, 182 aa, Homo sapiens
MDKDCEMKRTTLDSPLGKLELSGCEQGLHRIIFLGKGTSAADAVEVPAPAAVLGGPEPLMQATAWLNAYFHQPEAIEEFPVPALHHPVFQQESFTRQVLWKLLKVVKFGEVISYSHLAALAGNPAATAAVKTALSGNPVPILIPCHRVVQGDLDVGGYEGGLAVKEWLLAHEGHRLGKPGLG

SEQ ID NO: 20 - protein, 297 aa, synthetic construct
  REGION 1..297: note = modified bacterial dehalogenase that forms covalent bonds with chloroalkane derivatives
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 21 - protein, 404 aa, synthetic construct
  REGION 1..404: note = Fusion of a synthetic coiled-coil domain (P2), the CGU MmMT3, the QtEnc shell-forming monomer from the Encapsulin system of Q.thermotolerans and a FLAG-epitope
MSPEDKIAQLKEKNAALKEKNQQLKEKIQALKYGGGTGGSDPETCPCPTGGSCTCSDKCKCKGCKCTNCKKSCCSCCPAGCEKCAKDCVCKGEEGAKAEAEKCSCCQGSGPGNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGSGDYKDDDDK

SEQ ID NO: 22 - protein, 333 aa, synthetic construct
  REGION 1..333: note = Fusion of synthetic coiled-coil domain P1 (cognate partner of P2), OsO4 post-fixation stable switchable fluorescent protein mEosEM and a human PEST degradation sequence
MVSAIKPDMRIKLRMEGNVNGHHFVIDGEGTGKPYEGKQTMDLEVKEGGPLPFAFDILTTAFHYGNRVFVKYPDNIQDYFKQSFPKGYSWERSMTFEDGGICNARNDITMEGDTFYNKVRFYGTNFPANGPVMQKKTLKWEPSTEKMYVRDGVLTGDIEMALLLEGGAHYRCDFRTTYKAKEKGVKLPGAHFVDHAIEILSHDKDYNKVKLYEHAVAHSGLPDNARRGGGGSGGGGSGPGTGSPEDEIQALEEENAQLEQENAALEEEIAQLEYGSGSETPGTSESATPESAISHGFPPEVEEQAAGTLPMSCAQESGMDRHPAACASARINV

SEQ ID NO: 23 - protein, 195 aa, synthetic construct
  REGION 1..195: note = Strand 1 to 9 of a Tripartite split superfolderGFP
MRGEELFTGVVPILIELDGDVNGHKFFVRGEGEGDATIGKLSLKFICTTGKLPVPWPTLVTTLTYGVQCFSRYPDHMKRHDFFKSAMPEGYVQERTIYFKDDGTYKTRAEVKFEGDTLVNRIELKGIDFKEDGNILGHKLEYNFNSHKVYITADKQNNGIKANFTIRHNVEDGSVQLADHYQQNTPIGDGPVLLP

SEQ ID NO: 24 - protein, 487 aa, synthetic construct
  REGION 1..487: note = The shell forming monomer of the Ecapsulin system of Quasibacillus thermotolerans fused to superfolder GFP strands 1 to 9
MNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGGGGSGPGGSGMRGEELFTGVVPILIELDGDVNGHKFFVRGEGEGDATIGKLSLKFICTTGKLPVPWPTLVTTLTYGVQCFSRYPDHMKRHDFFKSAMPEGYVQERTIYFKDDGTYKTRAEVKFEGDTLVNRIELKGIDFKEDGNILGHKLEYNFNSHKVYITADKQNNGIKANFTIRHNVEDGSVQLADHYQQNTPIGDGPVLL

SEQ ID NO: 25 - protein, 313 aa, synthetic construct
  REGION 1..313: note = The shell forming monomer of the Ecapsulin system of Quasibacillus thermotolerans fused to superfolderGFP strand 10
MNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGGGGSGPGGSGTMDLPDNHYLSTQTILLKDL

SEQ ID NO: 26 - protein, 315 aa, synthetic construct
  REGION 1..315: note = The shell forming monomer of the Ecapsulin system of Quasibacillus thermotolerans fused to superfolderGFP strand 11
MNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGGGGSGPGGSGSEKRDHMVLLEYVTAAGITDAS

SEQ ID NO: 27 - protein, 432 aa, synthetic construct
  REGION 1..432: note = The shell forming monomer of the Ecapsulin system of Quasibacillus thermotolerans fused to enhanced UnaG(V2L) from Anguilla japonica and MmMT3
MNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGGGGSGPGGSGMLEKFVGTWKIADSHNFGEYLKAIGAPKELSDGGDATTPTLYISQKDGDKMSVKIENGPPTFLDTQVKFKLGEEFDEFPSDRRKGVKSVVNLVGEKLVYVQKWDGKETTYVREIKDGKLVVTLTMGDVVAVRSYRRATE

SEQ ID NO: 28 - protein, 437 aa, synthetic construct
  REGION 1..437: note = The shell forming monomer of the Ecapsulin system of Myxococcus xanthus fused to enhanced UnaG(V2L) from Anguilla japonica and MmMT3
MADFLGHAENPLREEEWARLNETVIQVARRSLVGRRILDIYGPLGAGVQTVPYDEFQGVSPGAVDIVGEQETAMVFTDARKFKTIPIIYKDFLLHWRDIEAARTHNMPLDVSAAAGAAALCAQQEDELIFYGDARLGYEGLMTANGRLTVPLGDWTSPGGGFQAIVEATRKLNEQGHFGPYAVVLSPRLYSQLHRIYEKTGVLEIETIRQLASDGVYQSNRLRGESGVVVSTGRENMDLAVSMDMVAAYLGASRMNHPFRVLEALLLRIKHPDAICTLEGAGATERRGGGGSGPGGSGMLEKFVGTWKIADSHNFGEYLKAIGAPKELSDGGDATTPTLYISQKDGDKMSVKIENGPPTFLDTQVKFKLGEEFDEFPSDRRKGVKSVVNLVGEKLVYVQKWDGKETTYVREIKDGKLVVTLTMGDVVAVRSYRRATE

SEQ ID NO: 29 - protein, 456 aa, synthetic construct
  REGION 1..456: note = Heme domain of BM3 a natural fusion protein constructed of cytochrome P450 and NADPH-cytochrome P450 reductase domains from Priestia megaterium
MTIKEMPQPKTFGELKNLPLLNTDKPVQALMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPL

SEQ ID NO: 30 - protein, 127 aa, Homo sapiens
MSFSGKYQLQSQENFEAFMKAIGLPEELIQKGKDIKGVSEIVQNGKHFKFTITAGSKVIQNEFTVGEECELETMTGEKVKTVVQLEGDNKLVTTFKNIKSVTELNGDIITNTMTLGDIVFKRISKRI

SEQ ID NO: 31 - protein, 132 aa, Homo sapiens
MAFDSTWKVDRSENYDKFMEKMGVNIVKRKLAAHDNLKLTITQEGNKFTVKESSAFRNIEVVFELGVTFNYNLADGTELRGTWSLEGNKLIGKFKRTDNGNELNTVREIIGDELVQTYVYEGVEAKRIFKKD

SEQ ID NO: 32 - protein, 133 aa, Homo sapiens [sequence omitted]

SEQ ID NO: 33 - protein, 132 aa, Homo sapiens
MCDAFVGTWKLVSSENFDDYMKEVGVGFATRKVAGMAKPNMIISVNGDVITIKSESTFKNTEISFILGQEFDEVTADDRKVKSTITLDGGVLVHVQKWDGKSTTIKRKREDDKLVVECVMKGVTSTRVYERA

SEQ ID NO: 34 - protein, 135 aa, Homo sapiens
MATVQQLEGRWRLVDSKGFDEYMKELGVGIALRKMGAMAKPDCIITCDGKNLTIKTESTLKTTQFSCTLGEKFEETTADGRKTQTVCNFTDGALVQHQEWDGKESTITRKLKDGKLVVECVMNNVTCTRIYEKVE

SEQ ID NO: 35 - protein, 128 aa, Homo sapiens
MAFTGKFEMESEKNYDEFMKLLGISSDVIEKARNFKIVTEVQQDGQDFTWSQHYSGGHTMTNKFTVGKESNIQTMGGKTFKATVQMEGGKLVVNFPNYHQTSEIVGDKLVEVSTIGGVTYERVSKRLA

SEQ ID NO: 36 - protein, 132 aa, Homo sapiens
MVEAFCATWKLTNSQNFDEYMKALGVGFATRQVGNVTKPTVIISQEGDKVVIRTLSTFKNTEISFQLGEEFDETTADDRNCKSVVSLDGDKLVHIQKWDGKETNFVREIKDGKMVMTLTFGDVVAVRHYEKA

SEQ ID NO: 37 - protein, 132 aa, Homo sapiens
MSNKFLGTWKLVSSENFDDYMKALGVGLATRKLGNLAKPTVIISKKGDIITIRTESTFKNTEISFKLGQEFEETTADNRKTKSIVTLQRGSLNQVQRWDGKETTIKRKLVNGKMVAECKMKGVVCTRIYEKV

SEQ ID NO: 38 - protein, 132 aa, Homo sapiens
MVEPFLGTWKLVSSENFEDYMKELGVNFAARNMAGLVKPTVTISVDGKMMTIRTESSFQDTKISFKLGEEFDETTADNRKVKSTITLENGSMIHVQKWLGKETTIKRKIVDEKMVVECKMNNIVSTRIYEKV

SEQ ID NO: 39 - protein, 140 aa, Homo sapiens
MIDQLQGTWKSISCENSEDYMKELGIGRASRKLGRLAKPTVTISTDGDVITIKTKSIFKNNEISFKLGEEFEEITPGGHKTKSKVTLDKESLIQVQDWDGKETTITRKLVDGKMVVESTVNSVICTRTYEKVSSNSVSNS

SEQ ID NO: 40 - protein, 10 aa, Rattus rattus
GCMSCKCVLS

SEQ ID NO: 41 - protein, 36 aa, Rattus norvegicus
CGLCVCPCNKLKSSDAYKKAWGNNQDGVVASQPARV

SEQ ID NO: 42 - protein, 15 aa, Homo sapiens
MGCVQCKDKEATKLT

SEQ ID NO: 43 - protein, 121 aa, Mus musculus
MPSDRPFKQRRSFADRCKEVQQIRDQHPSKIPVIIERYKGEKQLPVLDKTKFLVPDHVNMSELVKIIRRRLQLNPTQAFFLLVNQHSMVSVSTPIADIYEQEKDEDGFLYMVYASQETFGF

SEQ ID NO: 44 - protein, 338 aa, synthetic construct
  REGION 1..338: note = Consisting of engineered apo-lipoprotein A-1 (MSP1) flanked by DnaE split intein fragments C-intein (AA 103-137) on the N-term and N-Intein (AA 1-102) on the C-term) (Homo sapiens, Nostoc punctiforme):
MIKIATRKYLGKQNVYDIGVGEPHNFALKNGFIASNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 45 - protein, 139 aa, synthetic construct
  REGION 1..139: note = Designed uranyl binding proteins with 6 binding sites for uranyl ions
MVSRLEERVRDLEAEVDKLRERLDRLEETVQAKGGGGSGGGGSGGGGSGGGGSGGVSRLERVRDLEAEVDKLRERLDRLEETVQAKGGGGSGGGGSGGGGSGGGGSGGVSRLEERVRDLAEVDKLRERLDRLEETVQAK

SEQ ID NO: 46 - protein, 220 aa, synthetic construct
  REGION 1..220: note = Designed uranyl binding proteins with 6 binding sites for uranyl ions
MQLEDKVEELLSKVYHLENEVDRLRKLIENKGGGGSGGGGSQLEDKVEELESEVDHLRNEVAKKLVGGGGSGGGGSQLEDKVEELLSKVYHLENEVDRLRKLIENKGGGGSGGGGSQLEDKVEELESEVDHLRNEVARLKKLVGGGGSGGGGSQLEDKVEELLSKVYHLENEVDRLRKLIENKGGGGSGGGGSQLEDKVEELESEVDHLRNEVARLKKLV

SEQ ID NO: 47 - protein, 435 aa, synthetic construct
  REGION 1..435: note = Degron-tagged polyphopsphate kinase 2b of Corynebacterium glutamicum C-terminanlly tagged with the encapsulation signal of Quasibacillus thermotolerans
GVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKPEGSGVGKLPIMAETNENDLPVIDLAQIEGYVVDDSDEDDPVLLRPDGTPIETWREDFPYEERVTREDYEKVKRSLQIELLKWQNWTKETGQRHIILFEGRDAAGKGGTIKRFNEHLNPRGARTVALEKPSPRESTSWYFQRYIQHFPAAGEIVFFDRSWYNRSGVERVMGFCTESQHAEFLREVPMLENMILGSGISLTKFWFSVTRKEQRTRFAIRQVDPVRQWKLSPMDLASLDRWDDYTRAKEEQFRYTDTDESPWITIKSNDKKRARINAMRYVLSKFDYTDKDYELVGEPDPKVVLRGRDQIGDGGGGSGGGGSKGFTVGSLIQ

SEQ ID NO: 48 - protein, 305 aa, Corynebacterium glutamicum
VGKLPIMAETNENDLPVIDLAQIEGYVVDDSDEDDPVLLRPDGTPIETWREDFPYEERVTREDYEKVKRSLQIELLKWQNWTKETGQRHIILFEGRDAAGKGGTIKRFNEHLNPRGARTVALEKPSPRESTSWYFQRYIQHFPAAGEIVFFDRSWYNRSGVERVMGFCTESQHAEFLREVPMLENMILGSGISLTKFWFSVTRKEQRTRFAIRQVDPVRQWKLSPMDLASLDRWDDYTRAKEEQFRYTDTDESPWITIKSNDKKRARINAMRYVLSKFDYTDKDYELVGEPDPKVVLRGRDQIGD

SEQ ID NO: 49 - protein, 469 aa, synthetic construct
  REGION 1..469: note = Phytochelatin synthase 1 of Arabidopsis thaliana with a C-terminal encapsulation signal
MEGFFRLISYFQTQSEPAYCGLASLSVVLNALSIDPGRKWKGPWRWFDESMLDCCEPLEVVKEKGISFGKVVCLAHCSGAKVEAFRTSQSTIDDFRKFVVKCTSSENCHMISTYHRGVFKQTGTGHFSPIGGYNAERDMALILDVARFKYPPHWVPLKLLWEAMDSIDQSTGKRRGFMLISRPHREPGLLYTLSCKDESWIEIAKYLKEDVPRLVSSQHVDSVEKIISVVFKSLPSNFNQFIRWVAEIRITEDSNQNLSAEEKSRLKLKQLVLKEVHETELFKHINKFLSTVGYEDSLTYAAAKACCQGAEILSGSPSKEFCCRETCVKCIKGPDDSEGTVVTGVVVRDGNEQKVDLLVPSTQTECECGPEATYPAGNDVFTALLLALPPQTWSGIKDQALMHEMKQLISMASLPTLLQEEVLHLRRQLQLLKRCQENKEEDDLAAPAYGGGGSGGGGSKGFTVGSLIQ

SEQ ID NO: 50 - protein, 472 aa, synthetic construct
  REGION 1..472: note = Phytochelatin synthase 2 of Arabidopsis thaliana with a C-terminal encapsulation signal
MSMASLYRRSLSPPAIDFASFEGKQIFNEALQKGTMEGFFGLISYFQTQSEPAFCGLASLSMVLNSLSIDPGRKWKGPWRWFDESMLECCEPLEIVKDKGISFGKVVCLAHSSGAKVEAFRTNQSTIDDFRKYVVKCSTSDNCHMISTYHRQVLKQTGTGHFSPIGGYNAERDMALILDVARFKYPPHWVPLKLLWDAMDSIDQSTGRRRGFMLISRPHREPGLLYTLSCKDESWISIAKYLKEDVPRLVSSQHVDTIERILYVVFKSLPANFNQFIKWMAEIRRTEDVNQNLSSEEKSRLKLKQELLKQVQETKLFKHVDKFLSSVYEDNLPYVAAKVYCDGDEILSGYESDESCCKETCVKCIKGLGEEKVTVVAYPSGNDVFTALLLALPPQTWSGIKDQSLLQEMKQLISMVSHPTLLQQEVLHLRRQLEMLKRCQENKEDEELSAPAGGGGSGGGGSKGFTVGSLIQ

SEQ ID NO: 51 - protein, 296 aa, Bacillus megaterium
SNKYRVRKNVLHLTDTEKRDFVRTVLILKEKGIYDRYIAWHGAAGKFHTPPGSDRNAAHMSSAFLPWHREYLLRFERDLQSINPEVTLPYWEWETDAQMQDPSQSQIWSADFMGGNGNPIKDFIVDTGPFAAGRWTTIDEQGNPSGGLKRNFGATKEAPTLPTRDDVLNALKITQYDTPPWDMTSQNSFRNQLEGFINGPQLHNRVHRWVGGQMGVVPTAPNDPVFFLHHANVDRIWAVWQIIHRNQNYQPMKNGPFGQNFRDPMYPWNTTPEDVMNHRKLGYVYDIELRKSKRSS

SEQ ID NO: 52 - protein, 192 aa, Quasibacillus thermotolerans
MKEELDAFHQIFTTTKEAIERFMAMLTPVIENAEDDHERLYYHHIYEEEEQRLSRLDVLIPLIEKFQDETDEGLFSPSNNAFNRLLQELNLEKFGLHNFIEHVDLALFSFTDEERQTLLKELRKDAYEGYQYVKEKLAEINARFDHDYADPHAHHDEHRDHLADMPSAGSSHEEVQPVAHKKKGFTVGSLIQ

SEQ ID NO: 53 - protein, 128 aa, Bacteriophage Pseudomonas phage
MSKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVATSQVEDLVVNLVPLGR

SEQ ID NO: 54 - protein, 255 aa, synthetic construct
  REGION 1..255: note = Tandem dimer of The coat protein of the single-stranded RNA bacteriophage Pseudomonas phage binding the PP7 stem loop
MLASKTIVLSVGEATRTLTEIQSTADRQIFEEKVGPLVGRLRLTASLRQNGAKTAYRVNLKLDQADVVDSGLPKVRYTQVWSHDVTIVANSTEASRKSLYDLTKSLVATSQVEDLVVNLVPLGRADPLASKTIVLSVGEATRTLTEIQSTADRQIFEEKVGPLVGRLRLTASLRQNGAKTAYRVNLKLDQADVVDSGLPKVRYTQVWSHDVTIVANSTEASRKSLYDLTKSLVATSQVEDLVVNLVPLGRPPVAT

SEQ ID NO: 55 - RNA, 98 nt, synthetic construct
  misc_feature 1..98: note = Synthetically derived RNA aptamer designed to be an RNA mimic of green fluorescent protein (GFP)
  source 1..98: mol_type = other RNA
gacgcaactgaatgaaatggtgaaggacgggtccaggtgtggctgcttcggcagtgcagcttgttgagtagagtgtgagctccgtaactagtcgcgtc

SEQ ID NO: 56 - RNA, 25 nt, synthetic construct
  misc_feature 1..25: note = Small RNA sequence binding partner of the tandem PCP dimer from Pseudomonas phage
  source 1..25: mol_type = other RNA
ggagcagacgatatggcgtcgctcc

SEQ ID NO: 57 - RNA, 57 nt, synthetic construct
  misc_feature 1..57: note = Synthetically derived RNA aptamer designed to be an RNA mimic of red fluorescent protein (RFP)
  source 1..57: mol_type = other RNA
ggatgcgccttgaaaagcctgcttcggcagctggtgaatgacagctatggcgcatcc

SEQ ID NO: 58 - RNA, 19 nt, synthetic construct
  misc_feature 1..19: note = Small synthetic aptamer coordination metal ions in homodimeric form
  source 1..19: mol_type = other RNA
ttgtcgcttcgaaaaagtc

SEQ ID NO: 59 - RNA, 55 nt, synthetic construct
  misc_feature 1..55: note = Synthetic aptamer that binds a fluorogenic rhodamine dye with fast association and dissociation kinetics
  source 1..55: mol_type = other RNA
ccttggactccgccaattggaacgcggagaggaagtggcgaaagcgcctccaagg

SEQ ID NO: 60 - protein, 85 aa, synthetic construct
  REGION 1..85: note = RNA-binding domain that are known to bind single-stranded RNAs. It was found in many eukaryotic proteins
GSMATTKRVLYVGGLAEEVDDKVLHAAFIPFGDITDIQIPLDYETEKHRGFAFVEFELAEDAAAAIDNMNESELFGRTIRVNLAK

SEQ ID NO: 61 - protein, 78 aa, synthetic construct
  REGION 1..78: note = Protein domain that was first identified in the human binds RNA, and can function in RNA recognition
MSPNLPGQTTVQVRVPYRVVGLVVGPKGATIKRIQQQTHTYIVTPSRDKEPVFEVTGMPENVDRAREEIEMHIAMRTG

SEQ ID NO: 62 - protein, 87 aa, synthetic construct
  REGION 1..87: note = Zinc finger domain subspecies know to bind RNA
MSGGLAPSKSTVYVSNLPFSLTNNDLYRIFSKYGKVVKVTIMKDKDTRKSKGVAFILFLDKDSAQNCTRAINNKQLFGRVIKASIAI

SEQ ID NO: 63 - protein, 127 aa, synthetic construct
  REGION 1..127: note = Multifunctional RNA-binding protein that recognizes the K-turn motif in ribosomal RNA
MSKASYVKFEVPQDLADKVLEAVRKAKESGKIKKGTNETTKAVERGQAKLVIIAEDVQPEEIVAHLPLLCDEKKIPYVYVSSKKALGEACGLQVATASAAILEPGEAKDLVDEIIKRVNEIKGKTSS

SEQ ID NO: 64 - protein, 17 aa, synthetic construct
  REGION 1..17: note = BIV-Tat from the Bovine immunodeficiency virus binds to a hairpin structure at the 5'-end of all nascent viral mRNAs
SGPRPRGTRGKGRRIRR

SEQ ID NO: 65 - protein, 281 aa, Quasibacillus thermotolerans
NKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEE

SEQ ID NO: 66 - protein, 287 aa, Myxococcus xanthus
MADFLGHAENPLREEEWARLNETVIQVARRSLVGRRILDIYGPLGAGVQTVPYDEFQGVSPGAVDIVGEQETAMVFTDARKFKTIPIIYKDFLLHWRDIEAARTHNMPLDVSAAAGAAALCAQQEDELIFYGDARLGYEGLMTANGRLTVPLGDWTSPGGGFQAIVEATRKLNEQGHFGPYAVVLSPRLYSQLHRIYEKTGVLEIETIRQLASDGVYQSNRLRGESGVVVSTGRENMDLAVSMDMVAAYLGASRMNHPFRVLEALLLRIKHPDAICTLEGAGATERR

SEQ ID NO: 67 - protein, 264 aa, Thermotoga maritima
MEFLKRSFAPLTEKQWQEIDNRAREIFKTQLYGRKFVDVEGPYGWEYAAHPLGEVEVLSDENEVVKWGLRKSLPLIELRATFTLDLWELDNLERGKPNVDLSSLEETVRKVAEFEDEVIFRGCEKSGVKGLLSFEERKIECGSTPKDLLEAIVRALSIFSKDGIEGPYTLVINTDRWINFLKEEAGHYPLEKRVEECLRGGKIITTPRIEDALVVSERGGDFKLILGQDLSIGYEDREKDAVRLFITETFTFQVVNPEALILLK

SEQ ID NO: 68 - protein, 33 aa, synthetic construct
  REGION 1..33: note = Synthetic coiled-coil domain which pairs with P2
SPEDEIQALEEENAQLEQENAALEEEIAQLEYG

SEQ ID NO: 69 - protein, 33 aa, synthetic construct
  REGION 1..33: note = Synthetic coiled-coil domain which pairs with P1
SPEDKIAQLKEKNAALKEKNQQLKEKIQALKYG

SEQ ID NO: 70 - protein, 33 aa, synthetic construct
  REGION 1..33: note = Synthetic coiled-coil domain which pairs with P4
SPEDEIQQLEEEIAQLEQKNAALKEKNQALKYG

SEQ ID NO: 71 - protein, 33 aa, synthetic construct
  REGION 1..33: note = Synthetic coiled-coil domain which pairs with P3
SPEDKIAQLKQKIQALKQENQQLEEENAALEYG

SEQ ID NO: 72 - protein, 78 aa, synthetic construct
  REGION 1..78: note = Synthetic coiled-coil domain which pairs DHD13b
MGTKEDILERQRKIIERAQEIHRRQQEILEELERIIRKPGSSEEAMKRMLKLLEESLRLLKELLELSEESAQLLYEQR

SEQ ID NO: 73 - protein, 83 aa, synthetic construct
  REGION 1..83: note = Synthetic coiled-coil domain which pairs DHD13a
GTEKRLLEEAERAHREQKEIIKKAQELHRRLEEIVRQSGSSEEAKKEAKKILEEIRELSKRSLELLREILYLSQEQKGSLVPR

SEQ ID NO: 74 - DNA, 16 bp, synthetic construct
  misc_feature 1..16: note = engineered DNA sequence promoting ribosomal(translational) read-through after a STOP codon
  source 1..16: mol_type = other DNA
ctaggcgctcgaagat

SEQ ID NO: 75 - RNA, 15 nt, synthetic construct
  misc_feature 1..15: note = engineered RNA motif binding to L7Ae
  source 1..15: mol_type = other RNA
cctgattggtgaggg

SEQ ID NO: 76 - DNA, 24 bp, synthetic construct
  misc_feature 1..24: note = engineered DNA sequence promoting ribosomal(translational) read-through after a STOP codon
  source 1..24: mol_type = other DNA
caattagctcatccagacattccg

SEQ ID NO: 77 - protein, 861 aa, Rattus norvegicus
MATEEAIIRIPPYHYIHVLDQNSNVSRVEVGPKTYIRQDNERVLFAPVRMVTVPPRHYCIVANPVSRDTQSSVLFDITGQVRLRHADQEIRLAQDPFPLYPGEVLEKDITPLQVVLPNTALHLKALLDFEDKNGDKVMAGDEWLFEGPGTYIPQKEVEVVEIIQATVIKQNQALRLRARKECFDREGKGRVTGEEWLVRSVGAYLPAVFEEVLDLVDAVILTEKTALHLRALQNFRDLRGVLHRTGEEWLVTVQDTEAHVPDVYEEVLGVVPITTLGPRHYCVILDPMGPDGKNQLGQKRVVKGEKSFFLQPGERLERGIQDVYVLSEQQGLLLKALQPLEEGESEEKVSHQAGDCWLIRGPLEYVPSAKVEVVEERQAIPLDQNEGIYVQDVKTGKVRAVIGSTYMLTQDEVLWEKELPSGVEELLNLGHDPLADRGQKGTAKPLQPSAPRNKTRVVSYRVPHNAAVQVYDYRAKRARVVFGPELVTLDPEEQFTVLSLSAGRPKRPHARRALCLLLGPDFFTDVITIETADHARLQLQLAYNWHFELKNRNDPAEAAKLFSVPDFVGDACKAIASRVRGAVASVTFDDFHKNSARIIRMAVFGFEMSEDTGPDGTLLPKARDQAVFPQNGLVVSSVDVQSVEPVDQRTRDALQRSVQLAIEITTNSQEAAAKHEAQRLEQEARGRLERQKILDQSEAEKARKELLELEAMSMAVESTGNAKAEAESRAEAARIEGEGSVLQAKLKAQALAIETEAELERVKKVREMELIYARAQLELEVSKAQQLANVEAKKFKEMTEALGPGTIRDLAVAGPEMQVKLLQSLGLKSTLITDGSSPINLFSTAFGLLGLGSDGQPPAQK

SEQ ID NO: 78 - protein, 978 aa, synthetic construct
  REGION 1..978: note = Vault forming protein from Rattus norvegicus with SNAP25-derived palmitoylation signal (AA 85-120) inserted in helix-loop-helix region
MATEEAIIRIPPYHYIHVLDQNSNVSRVEVGPKTYIRQDNERVLFAPVRMVTVPPRHYCIVANPVSRDTQSSVLFDITGQVRLRHADQEIRLAQDPFPLYPGEVLEKDITPLQVVLPNTALHLKALLDFEDKNGDKVMAGDEWLFEGPGTYIPQKEVEVVEIIQATVIKQNQALRLRARKECFDREGKGRVTGEEWLVRSVGAYLPAVFEEVLDLVDAVILTEKTALHLRALQNFRDLRGVLHRTGEEWLVTVQDTEAHVPDVYEEVLGVVPITTLGPRHYCVILDPMGPDGKNQLGQKRVVKGEKSFFLQPGERLERGIQDVYVLSEQQGLLLKALQPLEEGESEEKVSHQAGDCWLIRGPLEYVPSAKVEVVEERQAIPLDQNEGIYVQDVKTGKVRAVIGSTYMLTQDEVLWEKELPSGVEELLNLGHDPLADRGQKGTAKPLQPSAPRNKTRVVSYRVPHNAAVQVYDYRAKRARVVFGPELVTLDPEEQFTVLSLSAGRPKRPHARRALCLLLGPDFFTDVITIETADHARLQLQLAYNWHFELKNRNDPAEAAKLFSVPDFVGDACKAIASRVRGAVASVTFDDFHKNSARIIRMAVFGFEGSPEDELAANEEELQQNEQKLAQIKQKLQAIKYGGGGSGGGGSGGGGCGLCVCPCNKLKSSDAYKKAWGNNQDGVVASQPARVGGGGSGGGGSGGGGSPEDEIQQLEEEIAQLEQKNAALKEKNQALKYGKARDQAVFPQNGLVVSSVDVQSVEPVDQRTRDALQRSVQLAIEITTNSQEAAAKHEAQRLEQEARGRLERQKILDQSEAEKARKELLELEAMSMAVESTGNAKAEAESRAEAARIEGEGSVLQAKLKAQALAIETEAELERVKKVREMELIYARAQLELEVSKAQQLANVEAKKFKEMTEALGPGTIRDLAVAGPEMQVKLLQSLGLKSTLITDGSSPINLFSTAFGLLGLGSDGQPPAQK

SEQ ID NO: 79 - protein, 996 aa, synthetic construct
  REGION 1..996: note = Vault forming protein from Rattus norvegicus with aniparallel coiled-coil domain inserted into helix-loop-helix region
MATEEAIIRIPPYHYIHVLDQNSNVSRVEVGPKTYIRQDNERVLFAPVRMVTVPPRHYCIVANPVSRDTQSSVLFDITGQVRLRHADQEIRLAQDPFPLYPGEVLEKDITPLQVVLPNTALHLKALLDFEDKNGDKVMAGDEWLFEGPGTYIPQKEVEVVEIIQATVIKQNQALRLRARKECFDREGKGRVTGEEWLVRSVGAYLPAVFEEVLDLVDAVILTEKTALHLRALQNFRDLRGVLHRTGEEWLVTVQDTEAHVPDVYEEVLGVVPITTLGPRHYCVILDPMGPDGKNQLGQKRVVKGEKSFFLQPGERLERGIQDVYVLSEQQGLLLKALQPLEEGESEEKVSHQAGDCWLIRGPLEYVPSAKVEVVEERQAIPLDQNEGIYVQDVKTGKVRAVIGSTYMLTQDEVLWEKELPSGVEELLNLGHDPLADRGQKGTAKPLQPSAPRNKTRVVSYRVPHNAAVQVYDYRAKRARVVFGPELVTLDPEEQFTVLSLSAGRPKRPHARRALCLLLGPDFFTDVITIETADHARLQLQLAYNWHFELKNRNDPAEAAKLFSVPDFVGDACKAIASRVRGAVASVTFDDFHKNSARIIRMAVFGFEGSPEDELAANEEELQQNEQKLAQIKQKLQAIKYGGGGSGGGGSGGGGSGGGGKQLEKELKQLEKELQAIEKQLAQLQWKAQARKKKLAQLKKKLQAGGGGSGGGGSGGGGSGGGGSPEDEIQQLEEEIAQLEQKNAALKEKNQALKYGKARDQAVFPQNGLVVSSVDVQSVEPVDQRTRDALQRSVQLAIEITTNSQEAAAKHEAQRLEQEARGRLERQKILDQSEAEKARKELLELEAMSMAVESTGNAKAEAESRAEAARIEGEGSVLQAKLKAQALAIETEAELERVKKVREMELIYARAQLELEVSKAQQLANVEAKKFKEMTEALGPGTIRDLAVAGPEMQVKLLQSLGLKSTLITDGSSPINLFSTAFGLLGLGSDGQPPAQK

SEQ ID NO: 80 - protein, 978 aa, synthetic construct
  REGION 1..978: note = Vault forming protein from Rattus norvegicus with a shortened aniparallel coiled-coil domain inserted into helix-loop-helix region
MATEEAIIRIPPYHYIHVLDQNSNVSRVEVGPKTYIRQDNERVLFAPVRMVTVPPRHYCIVANPVSRDTQSSVLFDITGQVRLRHADQEIRLAQDPFPLYPGEVLEKDITPLQVVLPNTALHLKALLDFEDKNGDKVMAGDEWLFEGPGTYIPQKEVEVVEIIQATVIKQNQALRLRARKECFDREGKGRVTGEEWLVRSVGAYLPAVFEEVLDLVDAVILTEKTALHLRALQNFRDLRGVLHRTGEEWLVTVQDTEAHVPDVYEEVLGVVPITTLGPRHYCVILDPMGPDGKNQLGQKRVVKGEKSFFLQPGERLERGIQDVYVLSEQQGLLLKALQPLEEGESEEKVSHQAGDCWLIRGPLEYVPSAKVEVVEERQAIPLDQNEGIYVQDVKTGKVRAVIGSTYMLTQDEVLWEKELPSGVEELLNLGHDPLADRGQKGTAKPLQPSAPRNKTRVVSYRVPHNAAVQVYDYRAKRARVVFGPELVTLDPEEQFTVLSLSAGRPKRPHARRALCLLLGPDFFTDVITIETADHARLQLQLAYNWHFELKNRNDPAEAAKLFSVPDFVGDACKAIASRVRGAVASVTFDDFHKNSARIIRMAVFGFEGSPEDELAANEEELQQNEQKLAQIKQKLQAIKYGGGGSGGGGSGGGGKELKQLEKELQAIEKQLAQLQWKAQARKKKLAQLKKGGGGSGGGGSGGGGSPEDEIQQLEEEIAQLEQKNAALKEKNQALKYGKARDQAVFPQNGLVVSSVDVQSVEPVDQRTRDALQRSVQLAIEITTNSQEAAAKHEAQRLEQEARGRLERQKILDQSEAEKARKELLELEAMSMAVESTGNAKAEAESRAEAARIEGEGSVLQAKLKAQALAIETEAELERVKKVREMELIYARAQLELEVSKAQQLANVEAKKFKEMTEALGPGTIRDLAVAGPEMQVKLLQSLGLKSTLITDGSSPINLFSTAFGLLGLGSDGQPPAQK

SEQ ID NO: 81 - protein, 1255 aa, synthetic construct
  REGION 1..1255: note = TEV protease inducible Vault based on caged Gp41-1 split inteins, Version 1
MATEEAIIRIPPYHYIHVLDQNSNVSRVEVGPKTYIRQDNERVLFAPVRMVTVPPRHYCIVANPVSRDTQSSVLFDITGQVRLRHADQEIRLAQDPFPLYPGEVLEKDITPLQVVLPNTALHLKALLDFEDKNGDKVMAGDEWLFEGPGTYIPQKEVEVVEIIQATVIKQNQALRLRARKECFDREGKGRVTGEEWLVRSVGAYLPAVFEEVLDLVDAVILTEKTALHLRALQNFRDLRGVLHRTGEEWLVTVQDTEAHVPDVYEEVLGVVPITTLGPRHYCVILDPMGPDGKNQLGQKRVVKGEKSFFLQPGERLERGIQDVYVLSEQQGLLLKALQPLEEGESEEKVSHQAGDCWLIRGPLEYVPSAKVEVVEERQAIPLDQNEGIYVQDVKTGKVRAVIGSTYMLTQDEVLWEKELPSGVEELLNLGHDPLADRGQKGTAKPLQPSAPRNKTRVVSYRVPHNAAVQVYDYRAKRARVVFGPELVTLDPEEQFTVLSLSAGRPKRPHARRALCLLLGPDFFTDVITIETADHARLQLQLAYNWHFELKNRNDPAEAAKLFSVPDFVGDACKAIASRVRGAVASVTFDDFHKNSARIIRMAVFGFEGSPEDELAANEEELQQNEQKLAQIKQKLQAIKYGGGGSGGGGSGGGGCLDLKTQVQTPQGMKEISNIQVGDLVLSNTGYNEVLNVFPKSKKKSYKITLEDGKEIICSEEHLFPTQTGEMNISGGLKEGMCLYVKEGSGGENLYFQSAMGSHGAAAGSGGMMLKKILKIEELDERELIDIEVSGNHLFYANDGSGATNFSLLKQAGDVEENPGPGSSSKKKSYKITLEDGKEIICSEEHLFPTQTGEMNISGGLKEGMCLYVKEGSGGENLYFQSAMGSHGAAAGSGGMMLKKILKIEELDERELIDIEVSGNHLFYANDILTHNCGLCVCPCNKLKSSDAYKKAWGNNQDGVVASQPARVGGGGSGGGGSGGGGSPEDEIQQLEEEIAQLEQKNAALKEKNQALKYGKARDQAVFPQNGLVVSSVDVQSVEPVDQRTRDALQRSVQLAIEITTNSQEAAAKHEAQRLEQEARGRLERQKILDQSEAEKARKELLELEAMSMAVESTGNAKAEAESRAEAARIEGEGSVLQAKLKAQALAIETEAELERVKKVREMELIYARAQLELEVSKAQQLANVEAKKFKEMTEALGPGTIRDLAVAGPEMQVKLLQSLGLKSTLITDGSSPINLFSTAFGLLGLGSDGQPPAQK

SEQ ID NO: 82 - protein, 1255 aa, synthetic construct
  REGION 1..1255: note = TEV protease inducible Vault based on caged Gp41-1 split inteins, Version 2
MATEEAIIRIPPYHYIHVLDQNSNVSRVEVGPKTYIRQDNERVLFAPVRMVTVPPRHYCIVANPVSRDTQSSVLFDITGQVRLRHADQEIRLAQDPFPLYPGEVLEKDITPLQVVLPNTALHLKALLDFEDKNGDKVMAGDEWLFEGPGTYIPQKEVEVVEIIQATVIKQNQALRLRARKECFDREGKGRVTGEEWLVRSVGAYLPAVFEEVLDLVDAVILTEKTALHLRALQNFRDLRGVLHRTGEEWLVTVQDTEAHVPDVYEEVLGVVPITTLGPRHYCVILDPMGPDGKNQLGQKRVVKGEKSFFLQPGERLERGIQDVYVLSEQQGLLLKALQPLEEGESEEKVSHQAGDCWLIRGPLEYVPSAKVEVVEERQAIPLDQNEGIYVQDVKTGKVRAVIGSTYMLTQDEVLWEKELPSGVEELLNLGHDPLADRGQKGTAKPLQPSAPRNKTRVVSYRVPHNAAVQVYDYRAKRARVVFGPELVTLDPEEQFTVLSLSAGRPKRPHARRALCLLLGPDFFTDVITIETADHARLQLQLAYNWHFELKNRNDPAEAAKLFSVPDFVGDACKAIASRVRGAVASVTFDDFHKNSARIIRMAVFGFEGSPEDELAANEEELQQNEQKLAQIKQKLQAIKYGGGGSGGGGSGGGGCGLCVCPCNKLKCLDLKTQVQTPQGMKEISNIQVGDLVLSNTGYNEVLNVFPKSKKKSYKITLEDGKEIICSEEHLFPTQTGEMNISGGLKEGMCLYVKEGSGGENLYFQSAMGSHGAAAGSGGMMLKKILKIEELDERELIDIEVSGNHLFYANDGSGATNFSLLKQAGDVEENPGPGSSSKKKSYKITLEDGKEIICSEEHLFPTQTGEMNISGGLKEGMCLYVKEGSGGENLYFQSAMGSHGAAAGSGGMMLKKILKIEELDERELIDIEVSGNHLFYANDILTHNSSDAYKKAWGNNQDGVVASQPARVGGGGSGGGGSGGGGSPEDEIQQLEEEIAQLEQKNAALKEKNQALKYGKARDQAVFPQNGLVVSSVDVQSVEPVDQRTRDALQRSVQLAIEITTNSQEAAAKHEAQRLEQEARGRLERQKILDQSEAEKARKELLELEAMSMAVESTGNAKAEAESRAEAARIEGEGSVLQAKLKAQALAIETEAELERVKKVREMELIYARAQLELEVSKAQQLANVEAKKFKEMTEALGPGTIRDLAVAGPEMQVKLLQSLGLKSTLITDGSSPINLFSTAFGLLGLGSDGQPPAQK

SEQ ID NO: 83 - protein, 241 aa, synthetic construct
  REGION 1..241: note = MmMT3 fused to endogenous vault cargo protein mINT
MDPETCPCPTGGSCTCSDKCKCKGCKCTNCKKSCCSCCPAGCEKCAKDCVCKGEEGAKAEAEKCSCCQGGGSGGGGSGGGDDEGRQSPVSWTSLFALQTEDGFWKLTPELGLILKLNVNALLNSLEEKGIRSLGTKGRERLLDLIATLLVLQFLYTKLEQEGMAAKSLIKMDDVLISRNIPWAFENIKKAREWARRTEGQYPSICQRLELGKDWESATKQLLGIQHQGNTSLHRILHYSQG

SEQ ID NO: 84 - protein, 996 aa, synthetic construct
  REGION 1..996: note = MmMT3 inserted into rat Vault Helix-Loop-Helix motif
MATEEAIIRIPPYHYIHVLDQNSNVSRVEVGPKTYIRQDNERVLFAPVRMVTVPPRHYCIVANPVSRDTQSSVLFDITGQVRLRHADQEIRLAQDPFPLYPGEVLEKDITPLQVVLPNTALHLKALLDFEDKNGDKVMAGDEWLFEGPGTYIPQKEVEVVEIIQATVIKQNQALRLRARKECFDREGKGRVTGEEWLVRSVGAYLPAVFEEVLDLVDAVILTEKTALHLRALQNFRDLRGVLHRTGEEWLVTVQDTEAHVPDVYEEVLGVVPITTLGPRHYCVILDPMGPDGKNQLGQKRVVKGEKSFFLQPGERLERGIQDVYVLSEQQGLLLKALQPLEEGESEEKVSHQAGDCWLIRGPLEYVPSAKVEVVEERQAIPLDQNEGIYVQDVKTGKVRAVIGSTYMLTQDEVLWEKELPSGVEELLNLGGGGSPEDKLQAIKYELAQNEEELAQIEEKLAANKEGGSGGGGSGGDPETCPCPTGGSCTCSDKCKCKGCKCTNCKKSCCSCCPAGCEKCAKDCVCKGEEGAKAEAEKCSCCQGGSGGGGSGGPEDENAALEEKIAQLKQKNAALKEEIQALEYSAPRNKTRVVSYRVPHNAAVQVYDYRAKRARVVFGPELVTLDPEEQFTVLSLSAGRPKRPHARRALCLLLGPDFFTDVITIETADHARLQLQLAYNWHFELKNRNDPAEAAKLFSVPDFVGDACKAIASRVRGAVASVTFDDFHKNSARIIRMAVFGFEMSEDTGPDGTLLPKARDQAVFPQNGLVVSSVDVQSVEPVDQRTRDALQRSVQLAIEITTNSQEAAAKHEAQRLEQEARGRLERQKILDQSEAEKARKELLELEAMSMAVESTGNAKAEAESRAEAARIEGEGSVLQAKLKAQALAIETEAELERVKKVREMELIYARAQLELEVSKAQQLANVEAKKFKEMTEALGPGTIRDLAVAGPEMQVKLLQSLGLKSTLITDGSSPINLFSTAFGLLGLGSDGQPPAQK

SEQ ID NO: 85 - protein, 1125 aa, synthetic construct
  REGION 1..1125: note = Rat Vault with N-terminal anti-parallel coiled-coil domain stabilizing the holo-vault configuration and MT3 and His6-tag in the HLH domain
MADIEQELERLKASIRRLEQEVNQERARMAALQTLLAKGGSGGATEEAIIRIPPYHYIHVLDQNSNVSRVEVGPKTYIRQDNERVLFAPVRMVTVPPRHYCIVANPVSRDTQSSVLFDITGQVRLRHADQEIRLAQDPFPLYPGEVLEKDITPLQVVLPNTALHLKALLDFEDKNGDKVMAGDEWLFEGPGTYIPQKEVEVVEIIQATVIKQNQALRLRARKECFDREGKGRVTGEEWLVRSVGAYLPAVFEEVLDLVDAVILTEKTALHLRALQNFRDLRGVLHRTGEEWLVTVQDTEAHVPDVYEEVLGVVPITTLGPRHYCVILDPMGPDGKNQLGQKRVVKGEKSFFLQPGERLERGIQDVYVLSEQQGLLLKALQPLEEGESEEKVSHQAGDCWLIRGPLEYVPSAKVEVVEERQAIPLDQNEGIYVQDVKTGKVRAVIGSTYMLTQDEVLWEKELPSGVEELLNLGGGGSPEDKLQAIKYELAQNEEELAQIEEKLAANKEGGSGGGGSGGDPETCPCPTGGSCTCSDKCKCKGCKCTNCKKSCCSCCPAGCEKCAKDCVCKGEEGAKAEAEKCSCCQGGSGGGGSGGPEDENAALEEKIAQLKQKNAALKEEIQALEYSAPRNKTRVVSYRVPHNAAVQVYDYRAKRARVVFGPELVTLDPEEQFTVLSLSAGRPKRPHARRALCLLLGPDFFTDVITIETADHARLQLQLAYNWHFELKNRNDPAEAAKLFSVPDFVGDACKAIASRVRGAVASVTFDDFHKNSARIIRMAVFGFEGSPEDELAANEEELQQNEQKLAQIKQKLQAIKYGGGGSGGGGSGGGGHHHHHHGGGGSGGGGSGGGGSPEDEIQQLEEEIAQLEQKNAALKEKNQALKYGKARDQAVFPQNGLVVSSVDVQSVEPVDQRTRDALQRSVQLAIEITTNSQEAAAKHEAQRLEQEARGRLERQKILDQSEAEKARKELLELEAMSMAVESTGNAKAEAESRAEAARIEGEGSVLQAKLKAQALAIETEAELERVKKVREMELIYARAQLELEVSKAQQLANVEAKKFKEMTEALGPGTIRDLAVAGPEMQVKLLQSLGLKSTLITDGSSPINLFSTAFGLLGLGSDGQPPAQK

SEQ ID NO: 86 - protein, 187 aa, synthetic construct
  REGION 1..187: note = Synthetic Hyperstable de novo TIM barrel varian
MDILIVNATDVDEMLKQVEILRRLGAKQIAVVSDDWRILQEALKKGGDILIVNATDVDEMLKQVEILRRLGAKQIAVVSDDWRILQEALKKGGDILIVNATDVDEMLKQVEILRRLGAKQIAVVSDDWRILQEALKKGGDILIVNATDVDEMLKQVEILRRLGAKQIAVVSDDWRILQEALKKGGLE

SEQ ID NO: 87 - protein, 456 aa, Sus scrofa
MNLDIHCEQLSDARWTELLPLLQQYEVVRLDDCGLTEEHCKDIGSALRANPSLTELCLRTNELGDAGVHLVLQGLQSPTCKIQKLSLQNCSLTEAGCGVLPSTLRSLPTLRELHLSDNPLGDAGLRLLCEGLLDPQCHLEKLQLEYCRLTAASCEPLASVLRATRALKELTVSNNDIGEAGARVLGQGLADSACQLETLRLENCGLTPANCKDLCGIVASQASLRELDLGSNGLGDAGIAELCPGLLSPASRLKTLWLWECDITASGCRDLCRVLQAKETLKELSLAGNKLGDEGARLLCESLLQPGCQLESLWVKSCSLTAACCQHVSLMLTQNKHLLELQLSSNKLGDSGIQELCQALSQPGTTLRVLCLGDCEVTNSGCSSLASLLLANRSLRELDLSNNCVGDPGVLQLLGSLEQPGCALEQLVLYDTYWTEEVEDRLQALEGSKPGLRVIS

SEQ ID NO: 88 - protein, 242 aa, Pseudomonas aeruginosa
TYCVAMHLADGLVFASDSRTNAGIDHIATFRKLFTFGTPGERLLVVQTAGNLATSQSVINLLQQRIRRDGASLLNVPSVYDATALVAETTREVMARDSGNLAGNTDLSCSFMVGGQIAGGPPALYSIYPQGNFIQATPDTPFLQLGESKYGKPILDRNLTFDTPLEQALRCALVSFDSTIRSNLSVGMPLDLLVYHRDSLILPEGYRVTEDDAYFSAIRRQWSAGLHDMLERLPSPPSAYNI

SEQ ID NO: 89 - protein, 249 aa, Bacteriophage S16
MAVQGPWVGSSYVAETGQNWASLAANELRVTERPFWISSFIGRSKEEIWEWTGENHSFNKDWLIGELRNRGGTPVVINIRAHQVSYTPGAPLFEFPGDLPNAYITLNIYADIYGRGGTGGVAYLGGNPGGDCIHNWIGNRLRINNQGWICGGGGGGGGFRVGHTEAGGGGGRPLGAGGVSSLNLNGDNATLGAPGRGYQLGNDYAGNGGDVGNPGSASSAEMGGGAAGRAVVGTSPQWINVGNIAGSWL

SEQ ID NO: 90 - protein, 204 aa, Bacteriophage S16
MGSSHHHHHHSQDPENLYFQGALGSASIAIGDNDTGLRWGGDGIVQIVANNAIVGGWNSTDIFTEAGKHITSNGNLNQWGGGAIYCRDLNVSSDRRIKKDIKAFENPVDILSTIGGYTYLIEKGFNEDGSQAYEESAGLIAQEVEAVLPRLVKISNDGTKDVKRLNYNGITALNTAAINVHTKEINELKKQLKELKDIVKFLTK

SEQ ID NO: 91 - protein, 184 aa, Human adenovirus 26 [sequence omitted]

SEQ ID NO: 92 - protein, 393 aa, synthetic construct
  REGION 1..393: note = Engineered fluorescent protein based on sfGFP, with ALFA-tags (SRLEEELRRRLT) inserted into loop region and at the termini
MGSRLEEELRRRLTQQQSRLEEELRRRLTQQQSRLEEELRRRLTGQVSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEGGSRLEEELRRRLTQQQSRLEEELRRRLTGGQQGGSRLEEELRRRLTQQQSRLEEELRRRLTGGDGSVQLADHYQQNTPIGDGPVLLPDNHYLSTQSVLSKDPNEKRDHMVLLEFVTAAGITHGMDELYKGQGSRLEEELRRRLTQQQSRLEEELRRRLTQQQSRLEEELRRRLT

SEQ ID NO: 93 - protein, 350 aa, synthetic construct
  REGION 1..350: note = Engineered fluorescent protein based on mGreenLantern, with HA-tags (YPYDVPDYA) inserted into loop region and at the termini
MGYPYDVPDYAGYPYDVPDYAGYPYDVPDYAGGVSKGEELFTGVVPILVELDGDVNGHKFSVRGEGEGDATNGKLTLKFICTTGKLPVPWPTLVTTLGYGVACFARYPDHMKQHDFFKSAMPEGYVQERTISFKDDGTYKTRAEVKFEGDTLVNRIVLKGIDFKEDGNILGHKLEYNFNSHKVYITADKQKNGIKANFKTRHNVEGGYPYDVPDYAGGYPYDVPDYAGGGGYPYDVPDYAGGYPYDVPDYAGGDGGVQLADHYQQNTPIGDGPVLLPDNHYLSHQSKLSKDPNEKRDHMVLKERVTAAGITHDMDELYKGGYPYDVPDYAGYPYDVPDYAGYPYDVPDYA

SEQ ID NO: 94 - protein, 369 aa, synthetic construct
  REGION 1..369: note = Engineered fluorescent protein based on mGreenLantern, with Moon-Tags(KNEQELLELDKWASL) inserted into loop region and at the termini
MVSKGEELFTGVVPILVELDGDVNGHKFSVRGEGEGDATNGKLTLKFICTTGKLPVPWPTLVTTLGYGVACFARYPDHMKQHDFFKSAMPEGYVQERTISFKDDGTYKTRAEVKFEGDTLVNRIVLKGIDFKEDGNILGHKLEYNFNSHKVYITADKQKNGIKANFKTRHNVEGGKNEQELLELDKWASLQQQKNEQELLELDKWASLGGQQGGKNEQELLELDKWASLQQQKNEQELLELDKWASLGGDGGVQLADHYQQNTPIGDGPVLLPDNHYLSHQSKLSKDPNEKRDHMVLKERVTAAGITHDMDELYKGQGKNEQELLELDKWASLQQQKNEQELLELDKWASLQQQKNEQELLELDKWASL

SEQ ID NO: 95 - protein, 422 aa, synthetic construct
  REGION 1..422: note = Engineered fluorescent protein based on mScarlet-I,with PepTags (AVERYLKDQQLLGIW) inserted into loop region and at the termini
MGTGSAVERYLKDQQLLGIWQQQAVERYLKDQQLLGIWQQQAVERYLKDQQLLGIWGQVSKGEAVIKEFMRFKVHMEGSMNGHEFEIEGEGEGRPYEGTQTAKLKVTKGGPLPFSWDILSPQFMYGSRAFIKHPADIPDYYKQSFPEGFKWERVMNFEDGGAVTVTQDTSLEDGTLIYKVKLRGTNFPPDGPVMQKKTMGWEASTERLYPEDGVLKGDIKMALRLKGGAVERYLKDQQLLGIWQQQAVERYLKDQQLLGIWGGQQGGAVERYLKDQQLLGIWQQQAVERYLKDQQLLGIWGGDGGRYLADFKTTYKAKKPVQMPGAYNVDRKLDITSHNEDYTVVEQYERSEGRHSTGGMDELYKGQGAVERYLKDQQLLGIWQQQAVERYLKDQQLLGIWQQQAVERYLKDQQLLGIWGPG

SEQ ID NO: 96 - protein, 457 aa, synthetic construct
  REGION 1..457: note = Engineered fluorescent protein based on mEosEM, with MoonTags (EELLSKNYHLENEVARLKK) inserted inserted into loop region and at the termini
MGTGSEELLSKNYHLENEVARLKKQQQEELLSKNYHLENEVARLKKQQQEELLSKNYHLENEVARLKKGQVSAIKPDMRIKLRMEGNVNGHHFVIDGEGTGKPYEGKQTMDLEVKEGGPLPFAFDILTTAFHYGNRVFVKYPDNIQDYFKQSFPKGYSWERSMTFEDGGICNARNDITMEGDTFYNKVRFYGTNFPANGPVMQKKTLKWEPSTEKMYVRDGVLTGDIEMALLLEGGEELLSKNYHLENEVARLKKQQQEELLSKNYHLENEVARLKKGGQQGGEELLSKNYHLENEVARLKKQQQEELLSKNYHLENEVARLKKGGGGAHYRCDFRTTYKAKEKGVKLPGAHFVDHAIEILSHDKDYNKVKLYEHAVAHSGLPDNARRGQGEELLSKNYHLENEVARLKKQQQEELLSKNYHLENEVARLKKQQQEELLSKNYHLENEVARLKKGPG

SEQ ID NO: 97 - protein, 339 aa, synthetic construct
  REGION 1..339: note = Engineered fluorescent protein based on mScarlet-I, with triple FLAG-tags (DYKDHDGDYKDHDIDYKDDDDK) inserted into loop region and at the termini
MGTGSDYKDHDGDYKDHDIDYKDDDDKGQVSKGEAVIKEFMRFKVHMEGSMNGHEFEIEGEGEGRPYEGTQTAKLKVTKGGPLPFSWDILSPQFMYGSRAFIKHPADIPDYYKQSFPEGFKWERVMNFEDGGAVTVTQDTSLEDGTLIYKVKLRGTNFPPDGPVMQKKTMGWEASTERLYPEDGVLKGDIKMALRLKGGDYKDHDGDYKDHDIDYKDDDDKGGGDYKDHDGDYKDHDIDYKDDDDKGGDGGRYLADFKTTYKAKKPVQMPGAYNVDRKLDITSHNEDYTVVEQYERSEGRHSTGGMDELYKGQGDYKDHDGDYKDHDIDYKDDDDKGPG

SEQ ID NO: 98 - protein, 1511 aa, synthetic construct
  REGION 1..1511: note = Engineered fluorescent protein based on emFPv4 with 24x SunTags on both termini
MEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKDYHLENEVARLKKGSGSGEELLSKNYHLENEVARLKKVSKGEELFTGVVPILVELDGDVNGHKFSVRGEGEGDATNGKLTLKFICTTGKLPVPWPTLVTTLGYGVACFARYPDHMKQHDFFKSAMPEGYVQERTISFKDDGTYKTRAEVKFEGDTLVNRIVLKGIDFKEDGNILGHKLEYNFNSHKVYITADKQKNGIKANFKTRHNVEGGKNEQELLELDKWASLQQQKNEQELLELDKWASLGGQQGGKNEQELLELDKWASLQQQKNEQELLELDKWASLGGDGGVQLADHYQQNTPIGDGPVLLPDNHYLSHQSKLSKDPNEKRDHMVLKERVTAAGITHDMDELYKGQGKNEQELLELDKWASLQQQKNEQELLELDKWASLQQQKNEQELLELDKWASLEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKDYHLENEVARLKKGSGSGEELLSKNYHLENEVARLKK

SEQ ID NO: 99 - protein, 1167 aa, synthetic construct
  REGION 1..1167: note = Engineered fluorescent protein based on emFPv4 with 10x P3 CCC tags on each terminus
MSPEDEIQQLEEEIAQLEQKNAALKEKNQALKYGLKSGPGSGSPEDEIQQLEEEIAQLEQKNAALKEKNQALKYGTGSGPGSGSPEDEIQQLEEEIAQLEQKNAALKEKNQALKYGASSGPGSGSPEDEIQQLEEEIAQLEQKNAALKEKNQALKYGLESGPGSGSPEDEIQQLEEEIAQLEQKNAALKEKNQALKYGSGPGSGSPEDEIQQLEEEIAQLEQKNAALKEKNQALKYLESGPGSGSPEDEIQQLEEEIAQLEQKNAALKEKNQALKYGASSGPGSGSPEDEIQQLEEEIAQLEQKNAALKEKNQALKYGTGSGPGSGSPEDEIQQLEEEIAQLEQKNAALKEKNQALKYGLKSGPGSGSPEDEIQQLEEEIAQLEQKNAALKEKNQALKYGVSKGEELFTGVVPILVELDGDVNGHKFSVRGEGEGDATNGKLTLKFICTTGKLPVPWPTLVTTLGYGVACFARYPDHMKQHDFFKSAMPEGYVQERTISFKDDGTYKTRAEVKFEGDTLVNRIVLKGIDFKEDGNILGHKLEYNFNSHKVYITADKQKNGIKANFKTRHNVEGGKNEQELLELDKWASLQQQKNEQELLELDKWASLGGQQGGKNEQELLELDKWASLQQQKNEQELLELDKWASLGGDGGVQLADHYQQNTPIGDGPVLLPDNHYLSHQSKLSKDPNEKRDHMVLKERVTAAGITHDMDELYKGQGKNEQELLELDKWASLQQQKNEQELLELDKWASLQQQKNEQELLELDKWASLSPEDEIQQLEEEIAQLEQKNAALKEKNQALKYGLKSGPGSGSPEDEIQQLEEEIAQLEQKNAALKEKNQALKYGTGSGPGSGSPEDEIQQLEEEIAQLEQKNAALKEKNQALKYGASSGPGSGSPEDEIQQLEEEIAQLEQKNAALKEKNQALKYGLESGPGSGSPEDEIQQLEEEIAQLEQKNAALKEKNQALKYGSGPGSGSPEDEIQQLEEEIAQLEQKNAALKEKNQALKYLESGPGSGSPEDEIQQLEEEIAQLEQKNAALKEKNQALKYGASSGPGSGSPEDEIQQLEEEIAQLEQKNAALKEKNQALKYGTGSGPGSGSPEDEIQQLEEEIAQLEQKNAALKEKNQALKYGLKSGPGSGSPEDEIQQLEEEIAQLEQKNAALKEKNQALKYG

SEQ ID NO: 100 - protein, 130 aa, Bacteriophage MS2
MASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 101 - protein, 393 aa, Bacteriophage MS2
MRAFSTLDRENETFVPSVRVYADGETEDNSFSLKYRSNWTPGRFNSTGAKTKQWHYPSPYSRGALSVTSIDQGAYKRSGSSWGRPYEEKAGFGFSLDARSCYSLFPVSQNLTYIEVPQNVANRASTEVLQKVTQGNFNLGVALAEARSTASQLATQTIALVKAYTAARRGNWRQALRYLALNEDRKFRSKHVAGRWLELQFGWLPLMSDIQGAYEMLTKVHLQEFLPMRAVRQVGTNIKLDGRLSYPAANFQTTCNISRRIVIWFYINDARLAWLSSLGILNPLGIVWEKVPFSFVVDWLLPVGNMLEGLTAPVGCSYMSGTVTDVITGESIISVDAPYGWTVERQGTAKAQISAMHRGVQSVWPTTGAYVKSPFSMVHTLDALALIRQRLSR

SEQ ID NO: 102 - protein, 133 aa, Bacteriophage Q-beta
MAKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAY

SEQ ID NO: 103 - protein, 420 aa, Bacteriophage Q-beta
MPKLPRGLRFGADNEILNDFQELWFPDLFIESSDTHPWYTLKGRVLNAHLDDRLPNVGGRQVRRTPHRVTVPIASSGLRPVTTVQYDPAALSFLLNARVDWDFGNGDSANLVINDFLFRTFAPKEFDFSNSLVPRYTQAFSAFNAKYGTMIGEGLETIKYLGLLLRRLREGYRAVKRGDLRALRRVIQSYHNGKWKPATAGNLWLEFRYGLMPLFYDIRDVMLDWQNRHDKIQRLLRFSVGHGEDYVVEFDNLYPAVAYFKLKGEITLERRHRHGISYANREGYAVFDNGSLRPVSDWKELATAFINPHEVAWELTPYSFVVDWFLNVGDILAQQGQLYHNIDIVDGFDRRDIRLKSFTIKGERNGRPVNVSASLSAVDLFYSRLHTSNLPFATLDLDTTFSSFKHVLDSIFLLTQRVKR

SEQ ID NO: 104 - protein, 294 aa, synthetic construct
  REGION 1..294: note = Capsid forming monomer from the encapsulin system of Q.thermotolerans tagged with C-termina FLAG epitope
MMNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGSGDYKDDDDK

SEQ ID NO: 105 - protein, 209 aa, synthetic construct
  REGION 1..209: note = Fusion of G51-E1-G52 domains of S.aureus protein SasG (amino acids 419-629) and mCherry
APKTITELEKKVEEIPFKKERKFNPDLAPGTEKVTREGQKGEKTITTPTLKNPLTGVIISKGEPKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGP

SEQ ID NO: 106 - protein, 407 aa, synthetic construct
  REGION 1..407: note = Capsid forming monomer from the encapsulin system of Q.thermotolerans tagged with a C-terminal anti-GFP intrabody
MNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGGGGSGPGGSGVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSS

SEQ ID NO: 107 - protein, 298 aa, synthetic construct
  REGION 1..298: note = Capsid forming monomer from the encapsulin system of M.xanthus tagged with C-terminal FLAG epitope
MPDFLGHAENPLREEEWARLNETVIQVARRSLVGRRILDIYGPLGAGVQTVPYDEFQGVSPGAVDIVGEQETAMVFTDARKFKTIPIIYKDFLLHWRDIEAARTHNMPLDVSAAAGAAALCAQQEDELIFYGDARLGYEGLMTANGRLTVPLGDWTSPGGGFQAIVEATRKLNEQGHFGPYAVVLSPRLYSQLHRIYEKTGVLEIETIRQLASDGVYQSNRLRGESGVVVSTGRENMDLAVSMDMVAAYLGASRMNHPFRVLEALLLRIKHPDAICTLEGAGATERRGSGDYKDDDDK

SEQ ID NO: 108 - protein, 283 aa, synthetic construct
  REGION 1..283: note = Capsid forming monomer from the encapsulin system of T.maritima (amino acids 1-264) tagged with C-termina BC2 epitope
MEFLKRSFAPLTEKQWQEIDNRAREIFKTQLYGRKFVDVEGPYGWEYAAHPLGEVEVLSDENEVVKWGLRKSLPLIELRATFTLDLWELDNLERGKPNVDLSSLEETVRKVAEFEDEVIFRGCEKSGVKGLLSFEERKIECGSTPKDLLEAIVRALSIFSKDGIEGPYTLVINTDRWINFLKEEAGHYPLEKRVEECLRGGKIITTPRIEDALVVSERGGDFKLILGQDLSIGYEDREKDAVRLFITETFTFQVVNPEALILLKGGGSGPGPDRKAAVSHWQQ

SEQ ID NO: 109 - protein, 439 aa, synthetic construct
  REGION 1..439: note = Capsid forming monomer from the encapsulin system of T.maritima (amino acids 1-264) N-terminally fused to a synthetic coiled-coil domain 'P2' and C-terminally fused to an anti-mCherry Intrabody (LaM-4)
MSPEDKIAQLKEKNAALKEKNQQLKEKIQALKYGGGGGSGPGEFLKRSFAPLTEKQWQEIDNRAREIFKTQLYGRKFVDVEGPYGWEYAAHPLGEVEVLSDENEVVKWGLRKSLPLIELRATFTLDLWELDNLERGKPNVDLSSLEETVRKVAEFEDEVIFRGCEKSGVKGLLSFEERKIECGSTPKDLLEAIVRALSIFSKDGIEGPYTLVINTDRWINFLKEEAGHYPLEKRVEECLRGGKIITTPRIEDALVVSERGGDFKLILGQDLSIGYEDREKDAVRLFITETFTFQVVNPEALILLKGSGSGSETTGAQVQLVESGGSLVQPGGSLRLSCAASGRFAESSSMGWFRQAPGKEREFVAAISWSGGATNYADSAKGRFTLSRDNTKNTVYLQMNSLKPDDTAVYYCAANLGNYISSNQRLYGYWGQGTQVTVS

SEQ ID NO: 110 - protein, 551 aa, synthetic construct
  REGION 1..551: note = Fusion protein consisting of MT3, a de novo designed fibril forming protein and GFP
MDPNCSCATDGSCSCAGSCKCKQCKCTSCKKSCCSCCPVGCAKCSQGCICKEASDKCSCCAGSGGSGGSGGMGPEDELKRVEKLVKEAEALLIVAKIKGSKRDLEKALRTAEEAAREAVKVLVQALLEGDPEVALRAVELVVRVAELLLRIAKESGSREALLRALIVAEEAAKLAKMVLELAEKQGDPEVALRAVELVVRVAELLLRIAKESGSEEALERALRVAEEAARLAKRVLELAEEQGDPLVAKMAVELVKRVAELLERIARESGSEEAKERAERVREEARELQERVKELREREGSGSGSGSGSKGEELFTGVVPILVELDGDVNGHKFSVRGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGSGSLE

SEQ ID NO: 111 - protein, 420 aa, synthetic construct
  REGION 1..420: note = Fusion protein consisting of hexa-Histidine Tag, a SpyCatcher domain, a de novo designed, single component icosahedron and an MT3 domain
MRGSHHHHHHENLYFQGAVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHIGGSGGSGGEKAAKAEEAARKMEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEGSGSGMDPNCSCATDGSCSCAGSCKCKQCKCTSCKKSCCSCCPVGCAKCSQGCICKEASDKCSCCA

SEQ ID NO: 112 - protein, 129 aa, synthetic construct
  REGION 1..129: note = Fusion protein of E.coli CsgA where the secretion peptide and N22 were deleted (amino acids 36-144) and SpyTag peptide
MSELNIYQYGGGNSALALQTDARNSDLTITQHGGGNGADVGQGSDDSSIDLTQRGFGNSATLDQWNGKNSEMTVKQFGGGNGAAVDQTASNSSVNVTQVGFGNNATAHQYGSGGSGAHIVMVDAYKPTK

SEQ ID NO: 113 - protein, 304 aa, synthetic construct
  REGION 1..304: note = Fusion protein of capsid forming monomer of the Encapsulin system of Q.thermotolerans and a synthetic peptide interacting with motor proteins
MNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGSGSAGTVFTTEDIYEWDDSAI

SEQ ID NO: 114 - protein, 697 aa, synthetic construct
  REGION 1..697: note = Fusion protein consisting of super-folder GFP, the G51-E1-G52 domains of S.aureus protein SasG (amino acids 419-629) and mCherry
MSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGGTGGSAPKTITELEKKVEEIPFKKERKFNPDLAPGTEKVTREGQKGEKTITTPTLKNPLTGVIISKGEPKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETGSGATGSVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 115 - protein, 876 aa, synthetic construct
  REGION 1..876: note = Fusion protein consisting of Gp41-1 C-intein, superfolder GFP, SasG (comprising 4 G5 domains yielding GEGEGEG), and engineered SpyCatcher003
MMLKKILKIEELDERELIDIEVSGNHLFYANDILTHNSSSGTGSGGSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGGTGGSAPKTITELEKKVEEIPFKKERKFNPDLAPGTEKVTREGQKGEKTITTPTLKNPLTGVIISKGEPKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETGSGATGSVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHT

SEQ ID NO: 116 - protein, 829 aa, synthetic construct
  REGION 1..829: note = Fusion protein consisting of a SnoopTag, mTurquoise2, SasG (comprising 4 G5 domains yielding GEGEGEG) and the N-intein split fragment of Gp41-1
MGKLGDIEFIKVNKSGGGSGVSKGEELFTGVVPILVELDGDVNGHKFSVSGEGEGDATYGKLTLKFICTTGKLPVPWPTLVTTLSWGVQCFARYPDHMKQHDFFKSAMPEGYVQERTIFFKDDGNYKTRAEVKFEGDTLVNRIELKGIDFKEDGNILGHKLEYNYFSDNVYITADKQKNGIKANFKIRHNIEDGGVQLADHYQQNTPIGDGPVLLPDNHYLSTQSKLSKDPNEKRDHMVLLEFVTAAGITLGMDELYKGGTGGSAPKTITELEKKVEEIPFKKERKFNPDLAPGTEKVTREGQKGEKTITTPTLKNPLTGVIISKGEPKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETGSGATGSSGYCLDLKTQVQTPQGMKEISNIQVGDLVLSNTGYNEVLNVFPKSKKKSYKITLEDGKEIICSEEHLFPTQTGEMNISGGLKEGMCLYVKE

SEQ ID NO: 117 - protein, 858 aa, synthetic construct
  REGION 1..858: note = Fusion protein consisting of SpyTag003, the red fluorescent protein mCherry, SasG (comprising 4 G5 domains yielding GEGEGEG) and a SnoopCatcher domain
MGRGVPHIVMVDAYKRYKSGGGSGVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKGGTGGSAPKTITELEKKVEEIPFKKERKFNPDLAPGTEKVTREGQKGEKTITTPTLKNPLTGVIISKGEPKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETGSGATGSLEKGSHMKPLRGAVFSLQKQHPDYPDIYGAIDQNGTYQNVRTGEDGKLTFKNLSDGKYRLFENSEPAGYKPVQNKPIVAFQIVNGEVRDVTSIVPQDIPATYEFTNGKHYITNEPIPPK

SEQ ID NO: 118 - protein, 426 aa, synthetic construct
  REGION 1..426: note = Fusion protein consisting of superfolder GFP, the G51 domain of SasG and the Npu DnaE N-intein split part
MSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGGTGGSAPKTITELEKKVEEIPFKKERKFNPDLAPGTEKVTREGQKGEKTITTPTLKNPLTGVIISKGEPKEEITKDPINELTEYGCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 119 - protein, 409 aa, synthetic construct
  REGION 1..409: note = Fusion protein consisting of Npu DnaE GEP C-Intein split fragment, SasG EG domains, where the proline residue in the G51-E junction was mutated to Cysteine to serve as nucleophile in the splicing reaction, and mCherry
MIKIATRKYLGKQNVYDIGVGEPHNFALKNGFIASNCETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETGSGATGSVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 120 - protein, 266 aa, synthetic construct
  REGION 1..266: note = Fusion protein consisting of Npu DnaE GEP C-Intein split fragment, SasG EG domains and the Npu DnaE N-intein split part on its C-terminus. This yields a self-growing fragment which can be capped off with NpuDnaE_GEP_C-Int_SasG_EG_mCherry_V2
MIKIATRKYLGKQNVYDIGVGEPHNFALKNGFIASNCETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 121 - protein, 340 aa, synthetic construct
  REGION 1..340: note = Fusion protein consisting of superfolder GFP, the G51 domain of SasG and the VidaL N-Intein split part
MSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGGTGGSAPKTITELEKKVEEIPFKKERKFNPDLAPGTEKVTREGQKGEKTITTPTLKNPLTGVIISKGEPKEEITKDPINELTEYGCLPKEAVVQIRLTKKG

SEQ ID NO: 122 - protein, 496 aa, synthetic construct
  REGION 1..496: note = Fusion protein consisting of VidaL C-Intein split part, SasG EG domains and mCherry
MIEEKKVTVQELRELYLSGEYTIEIDTPDGYQTIGKWFDKGVLSMVRVATATYETVCAFNHMIQLADNTWVQACELDVGVDIQTAAGIQPVMLVEDTSDAECYDFEVMHPNHRYYGDGIVSHNSETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETGSGATGSVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 123 - protein, 267 aa, synthetic construct
  REGION 1..267: note = Fusion protein consisting of VidaL C-Intein split part, SasG EG domains and VidaL N-Intein split part. This building block is a self-growing unit and can be capped off with VidaL-C-Int_SasG_EG_mCherry
MIEEKKVTVQELRELYLSGEYTIEIDTPDGYQTIGKWFDKGVLSMVRVATATYETVCAFNHMIQLADNTWVQACELDVGVDIQTAAGIQPVMLVEDTSDAECYDFEVMHPNHRYYGDGIVSHNSETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGCLPKEAVVQIRLTKKG

SEQ ID NO: 124 - protein, 255 aa, Tobacco etch virus
MEQKLISEEDLGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMSKPEEPFQPVKEATQLMSELVYSQYP

SEQ ID NO: 125 - protein, 244 aa, Plum pox virus
MSKSLFRGLRDYNPIASSICQLNNSSGARQSEMFGLGFGGLIVTNQHLFKRNDGELTIRSHHGEFVVKDTKTLKLLPCKGRDIVIIRLPKDFPPFPKRLQFRTPTTEDRVCLIGSNFQTKSISSTMSETSATYPVDNSHFWKHWISTKDGHCGLPIVSTRDGSILGLHSLANSTNTQNFYAAFPDNFETTYLSNQDNDNWIKQWRYNPDEVCWGSLQLKRDIPQSPFTICKLLTDLDGEFVYTQ

SEQ ID NO: 126 - protein, 244 aa, Soybean mosaic virus
MSKSVYKGLRDYSGISTLICQLTNSSDGHKETMFGVGYGSFIITNGHLFRRNNGMLTVKTWHGEFVIHNTTQLKIHFIQGRDVILIRMPKDFPPFGKRNLFRQPKREERVCMVGTNFQEKSLRATVSESSMILPEGKGSFWIHWITTQDGFCGLPLVSVNDGHIVGIHGLTSNDSEKNFFVPLTDGFEKEYLENADNLSWDKHWFWEPSKIAWGSLNLVEEQPKEEFKISKLVSDLFGNTVTVQ

SEQ ID NO: 127 - protein, 242 aa, sunflower mild mosaic virus
MGVSLSRGVRDYNAISSMVCRVTNDSGSSSTTMYGIGYGCYIITNKHLFRENNGRLLITSHHGEYICKNSASLKLSLVPGRDMLLIRLPKDCPPFPSKLKFREPTSEEKAVLVVTNFQEKHLSSMVSESSCVVQREDSPIWRHWISTKDGHCGAPIVSIRDGYIIGSHCGENPMTSNFFTSIPKDFQNLLNGKEANEWVSGWKYNIDAVCWGGLSVVNDAPSEPFITAKVVSALDTEGIKVQ

SEQ ID NO: 128 - protein, 458 aa, synthetic construct
  REGION 1..458: note = Fusion protein consisting of a triple engineered Lanthanide binding domain (LBT15), a TEV cleavage site, the capsid forming monomer of the Encapsulin system of Q.thermotolerans and a FLAG epitope
MFIDTNNDGWIEGDELLLEEGGSGGGGSFIDTNNDGWIEGDELLLEEGGSGGGGSFIDTNNDGWIEGDELLLEEGGSGGGGSENLYFQSGSGPGMDPETCPCPTGGSCTCSDKCKCKGCKCTNCKKSCCSCCPAGCEKCAKDCVCKGEEGAKAEAEKCSCCQGSGGNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGSGDYKDDDDK

SEQ ID NO: 129 - protein, 150 aa, synthetic construct
  REGION 1..150: note = Fusion protein consisting of a FLAG epitope, the N-terminal fragment of the Tobacco etch virus protease (TEV) (amino acids 1-118 and a C-terminal M13 calmodulin-binding peptide
MDYKDDDDKTGGESLFKGPRDYNPISSTICHLTNESDGHTTSLYGIGFGPFIITNKHLFRRNNGTLVVQSLHGVFKVKNTTTLQQHLIDGRDMIIIRMPKDFPPFPQKLKFREPQREERICLVTTNFQTSAGRRKWNKGHAVRAIGRLSS

SEQ ID NO: 130 - protein, 266 aa, synthetic construct
  REGION 1..266: note = Fusion protein consisting of a Myc epitope, Calmodulin and the C-terminal split fragment of Tobacco etch virus protease (amino acids 119-219, S153N
MASMQKLISEEDLTGDQLTEEQIAEFKEAFSLFDKDGDGTITTKELGTVMRSLGQNPTEAELQDMINEVDADGDGTIDFPEFLTMMARKMKDTDSEEEIREAFRVFDKDGNGYISAAELRHVMTNLGEKLTDEEVDEMIREADIDGDGQVNYEEFVQMMTAKSAGKSMSSMVSDTSCTFPSGDGIFWKHWIQTKDGQCGNPLVSTRDGFIVGIHSASNFTNTNNYFTSVPKNFMELLTNQEAQQWVSGWRLNADSVLWGGHKVFMV

SEQ ID NO: 131 - protein, 315 aa, synthetic construct
  REGION 1..315: note = Fusion of Encapsulin forming monomer of the Encapsulin system M.xanthus and the Calmodulin binding peptide RS20
MADFLGHAENPLREEEWARLNETVIQVARRSLVGRRILDIYGPLGAGVQTVPYDEFQGVSPGAVDIVGEQETAMVFTDARKFKTIPIIYKDFLLHWRDIEAARTHNMPLDVSAAAGAAALCAQQEDELIFYGDARLGYEGLMTANGRLTVPLGDWTSPGGGFQAIVEATRKLNEQGHFGPYAVVLSPRLYSQLHRIYEKTGVLEIETIRQLASDGVYQSNRLRGESGVVVSTGRENMDLAVSMDMVAAYLGASRMNHPFRVLEALLLRIKHPDAICTLEGAGATERRGSSEGGSGRRKWQKTGHAVRAIGRLSSS

SEQ ID NO: 132 - protein, 305 aa, synthetic construct
  REGION 1..305: note = Fusion of Encapsulin forming monomer of the Encapsulin system Q.thermotolerans and the Calmodulin binding peptide RS20
MNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGSGRRKWQKTGHAVRAIGRLSSS

SEQ ID NO: 133 - protein, 287 aa, synthetic construct
  REGION 1..287: note = Fusion of Encapsulin forming monomer of the Encapsulin system T.maritima and the Calmodulin binding peptide RS20
MEFLKRSFAPLTEKQWQEIDNRAREIFKTQLYGRKFVDVEGPYGWEYAAHPLGEVEVLSDENEVVKWGLRKSLPLIELRATFTLDLWELDNLERGKPNVDLSSLEETVRKVAEFEDEVIFRGCEKSGVKGLLSFEERKIECGSTPKDLLEAIVRALSIFSKDGIEGPYTLVINTDRWINFLKEEAGHYPLEKRVEECLRGGKIITTPRIEDALVVSERGGDFKLILGQDLSIGYEDREKDAVRLFITETFTFQVVNPEALILLKGSGRRKWQKTGHAVRAIGRLSSS

SEQ ID NO: 134 - protein, 315 aa, synthetic construct
  REGION 1..315: note = Fusion protein of human calmodulin and H.pylori ferritin (AA 5-167, N19Q)
MADQLTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAKSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 135 - protein, 235 aa, synthetic construct
  REGION 1..235: note = Fusion of human Calmodulin and MmMT3 which will be recruited to the scaffold (Enc) to form a contrast halo around the element when elevated Ca2+ levels are present
MDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGSGMDPETCPCPTGGSCTCSDKCKCKGCKCTNCKKSCCSCCPAGCEKCAKDCVCKGEEGAKAEAEKCSCCQ

SEQ ID NO: 136 - protein, 170 aa, synthetic construct
  REGION 1..170: note = Fusion protein of MT3, an engineered minimal Troponin C domain of Opsanus tau and the Encapsulin signal of the Encapsulin system of Q.thermotolerans
MDPETCPCPTGGSCTCSDKCKCKGCKCTNCKKSCCSCCPAGCEKCAKDCVCKGEEGAKAEAEKCSCCQTGRMQVADASEEELSECFRIFDFDGNGFIDREEFGDIIRLTGEQLTDEDVDEIFGDSDTDKNGRIDFDEFLKMVENVQPIYPGGGGSGGGGSKGFTVGSLIQ

SEQ ID NO: 137 - protein, 443 aa, synthetic construct
  REGION 1..443: note = Fusion protein MT3-TnCmin-QtEnc-FLAG
MDPETCPCPTGGSCTCSDKCKCKGCKCTNCKKSCCSCCPAGCEKCAKDCVCKGEEGAKAEAEKCSCCQTGRMQVADASEEELSECFRIFDFDGNGFIDREEFGDIIRLTGEQLTDEDVDEIFGDSDTDKNGRIDFDEFLKMVENVQPIYPGGGGSGGGGSVMNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEE

SEQ ID NO: 138 - protein, 723 aa, synthetic construct
  REGION 1..723: note = Fusion of E3 ubiquitin-protein ligase TRIM21, with inactivating mutations I79R and S80A, and mCherry fluorescence protein
TGSGATGSVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKGGGSGMASAARLTMMWEEVTCPICLDPFVEPVSIECGHSFCQECISQVGKGGGSVCPVCRQRFLLKNLRPNRQLANMVNNLKERAQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRCHSSALELLQEVIIVLERSESWNLKDLDITSPELRSVCHVPGLKKMLRTCAVHITLDPDTANPWLILSEDRRQVRLGDTQQSIPGNEERFDSYPMVLGAQHFHSGKHYWEVDVTGKEAWDLGVCRDSVRRKGHFLLSSKSGFWTIWLWNKQKYEAGTYPQTPLHLQVPPCQVGIFLDYEAGMVSFYNITDHGSLIYSFSECAFTGPLRPFFSPGFNDGGKNTAPLTLCPLNIGSQGSTDY

SEQ ID NO: 139 - protein, 806 aa, synthetic construct
  REGION 1..806: note = Fusion of E3 ubiquitin-protein ligase TRIM21, with inactivating mutations I79R and S80A, and superfolder GFP
MSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGGTGGSAPKTITELEKKVEEIPFKKERKFNPDLAPGTEKVTREGQKGEKTITTPTLKNPLTGVIISKGEPKEEITKDPINELTEYGPEGGGSGMASAARLTMMWEEVTCPICLDPFVEPVSIECGHSFCQECISQVGKGGGSVCPVCRQRFLLKNLRPNRQLANMVNNLKERAQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRCHSSALELLQEVIIVLERSESWNLKDLDITSPELRSVCHVPGLKKMLRTCAVHITLDPDTANPWLILSEDRRQVRLGDTQQSIPGNEERFDSYPMVLGAQHFHSGKHYWEVDVTGKEAWDLGVCRDSVRRKGHFLLSSKSGFWTIWLWNKQKYEAGTYPQTPLHLQVPPCQVGIFLDYEAGMVSFYNITDHGSLIYSFSECAFTGPLRPFFSPGFNDGGKNTAPLTLCPLNIGSQGSTDY

SEQ ID NO: 140 - DNA, 4955 bp, synthetic construct
  misc_feature 1..4955: note = expression vector pcDNA 3.1 (+) Zeocin containing elements from different organisms
  source 1..4955: mol_type = other DNA
aatggatgagctgtacaagtgataagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagc

SEQ ID NO: 141 - DNA, 6098 bp, synthetic construct
  misc_feature 1..6098: note = pIRES
  source 1..6098: mol_type = other DNA
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattcacgcgtcgagcatgcatctagggcggccaattccgcccctctcccccccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgggatcctctagagtcgacccgggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcga

SEQ ID NO: 142 - protein, 293 aa, synthetic construct
  REGION 1..293: note = Shell forming monomer of the Encapsulin system of Quasibacillus thermotolerans with a C-terminally appended FLAG epitope connected via a short GSG linker.
MNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGSGDYKDDDDK

SEQ ID NO: 143 - protein, 444 aa, synthetic construct
  REGION 1..444: note = A tandem of the Mus musculus Metallothionein 3 connected via GGGGSGGGGS linker fused to the shell forming monomer of the Encapsulin system of Quasibacillus thermotolerans with a C-terminally appended FLAG epitope connected via a short GSG
MDPETCPCPTGGSCTCSDKCKCKGCKCTNCKKSCCSCCPAGCEKCAKDCVCKGEEGAKAEAEKCSCCQGGGSGGGSGGSADPETCPCPTGGSCTCSDKCKCKGCKCTNCKKSCCSCCPAGCEKCAKDCVCKGEEGAKAEAEKCSCCQGSGPGNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGSGDYKDDDDK

SEQ ID NO: 144 - protein, 355 aa, synthetic construct
  REGION 1..355: note = Fusion of Mus musculus Metallothionein 3 fused to the shell forming monomer of the Encapsulin system of Thermotoga maritima fused to a C-terminal BC2-Tag
MDPETCPCPTGGSCTCSDKCKCKGCKCTNCKKSCCSCCPAGCEKCAKDCVCKGEEGAKAEAEKCSCCQGSGPGEFLKRSFAPLTEKQWQEIDNRAREIFKTQLYGRKFVDVEGPYGWEYAAHPLGEVEVLSDENEVVKWGLRKSLPLIELRATFTLDLWELDNLERGKPNVDLSSLEETVRKVAEFEDEVIFRGCEKSGVKGLLSFEERKIECGSTPKDLLEAIVRALSIFSKDGIEGPYTLVINTDRWINFLKEEAGHYPLEKRVEECLRGGKIITTPRIEDALVVSERGGDFKLILGQDLSIGYEDREKDAVRLFITETFTFQVVNPEALILLKGGGSGPGPDRKAAVSHWQQ

SEQ ID NO: 145 - protein, 318 aa, synthetic construct
  REGION 1..318: note = Synthetic Lanthanide-bindering tag 15 fused to the shell forming monomer of the Encapsulin system of Quasibacillus thermotolerans fused to a C-terminal FLAG tag-
MFIDTNNDGWIEGDELLLEEGGSGPGNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGSGDYKDDDDK

SEQ ID NO: 146 - protein, 380 aa, synthetic construct
  REGION 1..380: note = Engineered tandem Lanthanide-binding tag fused to the shell forming monomer of the Encapsulin system of Quasibacillus thermotolerans fused to a C-terminal FLAG tag
MGPGYIDTNNDGWIEGDELYIDTNNDGWIEGDELLAGGGSGGGSGGSAGPGYIDTNNDGWIEGDELYIDTNNDGWIEGDELLAGSGPGNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGSGDYKDDDDK

SEQ ID NO: 147 - protein, 304 aa, synthetic construct
  REGION 1..304: note = Capsid forming monomer from the encapsulin system of Q.thermotolerans tagged with C-terminal SpyTag003
MNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGGGGSGRGVPHIVMVDAYKRYK

SEQ ID NO: 148 - protein, 434 aa, synthetic construct
  REGION 1..434: note = Designed Uranyl-binding protein MUP2 fused to the shell forming monomer of the Encapsulin system of Quasibacillus thermotolerans
MVSRLEERVRDLEAEVDKLRERLDRLEETVQAKGGGGSGGGGSGGGGSGGGGSGGVSRLEERVRDLEAEVDKLRERLDRLEETVQAKGGGGSGGGGSGGGGSGGGGSGGVSRLEERVRDLEAEVDKLRERLDRLEETVQAKPPPPRPPPPRTGNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEE

SEQ ID NO: 149 - protein, 515 aa, synthetic construct
  REGION 1..515: note = Designed Uranyl-binding protein MUP5 fused to the shell forming monomer of the Encapsulin system of Quasibacillus thermotolerans
MQLEDKVEELLSKVYHLENEVDRLRKLIENKGGGGSGGGGSQLEDKVEELESEVDHLRNEVARLKKLVGGGGSGGGGSQLEDKVEELLSKVYHLENEVDRLRKLIENKGGGGSGGGGSQLEDKVEELESEVDHLRNEVARLKKLVGGGGSGGGGSQLEDKVEELLSKVYHLENEVDRLRKLIENKGGGGSGGGGSQLEDKVEELESEVDHLRNEVARLKKLVPPPPRPPPPRTGNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEE

SEQ ID NO: 150 - protein, 257 aa, synthetic construct
  REGION 1..257: note = Engineered SpyCatcher 003 domain fused to fatty acid binding protein FABP
MVEAFCATWKLTNSQNFDEYMKALGVGFATRQVGNVTKPTVIISQEGDKVVIRTLSTFKNTEISFQLGEEFDETTADDRNCKSVVSLDGDKLVHIQKWDGKETNFVREIKDGKMVMTLTFGDVVAVRHYEKAPPPPRTGPPPPRVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHT

SEQ ID NO: 151 - protein, 581 aa, synthetic construct
  REGION 1..581: note = Heme binding domain BM3h fused to engineered SpyCatcher 003 domain
MTIKEMPQPKTFGELKNLPLLNTDKPVQALMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLPPPPRTGPPPPRVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHT

SEQ ID NO: 152 - RNA, 21 nt, Bacteriophage MS2
  source 1..21: mol_type = unassigned RNA
aaacatgaggattacccatgt

SEQ ID NO: 153 - protein, 10 aa, synthetic construct
  REGION 1..10: note = 2xGGGGS linker
GGGGSGGGGS

SEQ ID NO: 154 - protein, 107 aa, synthetic construct
  REGION 1..107: note = Engineered FKBP-derived degron
GVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKPE

SEQ ID NO: 155 - protein, 509 aa, synthetic construct
  REGION 1..509: note = Fusion of engineered ultrasmall fluorescent protein eUnaG followed by MT3, QtEnc and a FLAG-tag
MLEKFVGTWKIADSHNFGEYLKAIGAPKELSDGGDATTPTLYISQKDGDKMSVKIENGPPTFLDTQVKFKLGEEFDEFPSDRRKGVKSVVNLVGEKLVYVQKWDGKETTYVREIKDGKLVVTLTMGDVVAVRSYRRATEGGTGGSDPETCPCPTGGSCTCSDKCKCKGCKCTNCKKSCCSCCPAGCEKCAKDCVCKGEEGAKAEAEKCSCCQGSGPGNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGSGDYKDDDDK

SEQ ID NO: 156 - protein, 588 aa, synthetic construct
  REGION 1..588: note = Fusion of engineered ultrasmall fluorescent protein eUnaG followed by 2xMT3, QtEnc and a FLAG-tag
MLEKFVGTWKIADSHNFGEYLKAIGAPKELSDGGDATTPTLYISQKDGDKMSVKIENGPPTFLDTQVKFKLGEEFDEFPSDRRKGVKSVVNLVGEKLVYVQKWDGKETTYVREIKDGKLVVTLTMGDVVAVRSYRRATEGGTGGSDPETCPCPTGGSCTCSDKCKCKGCKCTNCKKSCCSCCPAGCEKCAKDCVCKGEEGAKAEAEKCSCCQGGGSGGGSGGSADPETCPCPTGGSCTCSDKCKCKGCKCTNCKKSCCSCCPAGCEKCAKDCVCKGEEGAKAEAEKCSCCQGSGPGNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGSGDYKDDDDK

SEQ ID NO: 157 - protein, 609 aa, synthetic construct
  REGION 1..609: note = Fusion of the engineered green fluorescent protein mGreenLantern, MT3, QtEnc and a FLAG epitope
MVSKGEELFTGVVPILVELDGDVNGHKFSVRGEGEGDATNGKLTLKFICTTGKLPVPWPTLVTTLGYGVACFARYPDHMKQHDFFKSAMPEGYVQERTISFKDDGTYKTRAEVKFEGDTLVNRIVLKGIDFKEDGNILGHKLEYNFNSHKVYITADKQKNGIKANFKTRHNVEDGGVQLADHYQQNTPIGDGPVLLPDNHYLSHQSKLSKDPNEKRDHMVLKERVTAAGITHDMDELYKGGTGGSDPETCPCPTGGSCTCSDKCKCKGCKCTNCKKSCCSCCPAGCEKCAKDCVCKGEEGAKAEAEKCSCCQGSGPGNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGSGDYKDDDDK

SEQ ID NO: 158 - protein, 778 aa, synthetic construct
  REGION 1..778: note = Fusion of the engineered fluorescent Calcium integrator CaMPARI2, MT3, QtEnc and a FLAG tag
MGSDQLTEEQIAEFKEAFSLFDKDGDGTITTKELGTVMRSLGQNPTEAELQDMINEVDADGDGTIDFPEFLTMMARKMKDTDSEEEIREAFRVFDKDGNGYISAAELRHVMTNLGEKLTDEEVDEMIREADIDGDGQVNYEEFVVMMTAKLECEKIYVRDGVLTGDIHMILLLEGNAHYRCDFRTTYKAKEKGVKLPGVHYVDHTIEILSHDKDYNKVKIYEYAVAHSGLPDNARRGGTGGSMVSAIKPDMKIKLRMEGNVNGHHFVIDGDGTGKPYEGKQTMDLEVKEGGPLPFAFDILTTAFHYGNRVFVKYPDNIQDYFKQSFPKGYSWERSMTFEDGGICYARSDITMEGDTFYNKVRFYGTNFPANGPVMQKKTLKWMPSWTRSSRRKFNKTGHALRAIGRLSGGTGGSDPETCPCPTGGSCTCSDKCKCKGCKCTNCKKSCCSCCPAGCEKCAKDCVCKGEEGAKAEAEKCSCCQGSGPGNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGSGDYKDDDDK

SEQ ID NO: 159 - protein, 324 aa, synthetic construct
  REGION 1..324: note = Synthetic coiled-coil domain P2 (partner of P1) fused to the TmEnc (AA 1-264) with a C-terminal BC2-Tag
MSPEDKIAQLKEKNAALKEKNQQLKEKIQALKYGGGGGSGPGEFLKRSFAPLTEKQWQEIDNRAREIFKTQLYGRKFVDVEGPYGWEYAAHPLGEVEVLSDENEVVKWGLRKSLPLIELRATFTLDLWELDNLERGKPNVDLSSLEETVRKVAEFEDEVIFRGCEKSGVKGLLSFEERKIECGSTPKDLLEAIVRALSIFSKDGIEGPYTLVINTDRWINFLKEEAGHYPLEKRVEECLRGGKIITTPRIEDALVVSERGGDFKLILGQDLSIGYEDREKDAVRLFITETFTFQVVNPEALILLKGGGSGPGPDRKAAVSHWQQ

SEQ ID NO: 160 - protein, 385 aa, synthetic construct
  REGION 1..385: note = Synthetic coiled-coil domain DHD13a (partner of DHD13b) fused to MxEnc with a StrepTagII
MGTKEDILERQRKIIERAQEIHRRQQEILEELERIIRKPGSSEEAMKRMLKLLEESLRLLKELLELSEESAQLLYEQRGGGGSGPGPDFLGHAENPLREEEWARLNETVIQVARRSLVGRRILDIYGPLGAGVQTVPYDEFQGVSPGAVDIVGEQETAMVFTDARKFKTIPIIYKDFLLHWRDIEAARTHNMPLDVSAAAGAAALCAQQEDELIFYGDARLGYEGLMTANGRLTVPLGDWTSPGGGFQAIVEATRKLNEQGHFGPYAVVLSPRLYSQLHRIYEKTGVLEIETIRQLASDGVYQSNRLRGESGVVVSTGRENMDLAVSMDMVAAYLGASRMNHPFRVLEALLLRIKHPDAICTLEGAGATERRGSGSAWSHPQFEK

SEQ ID NO: 161 - protein, 334 aa, synthetic construct
  REGION 1..334: note = Synthetic coiled-coil domain P3 (partner of P4) fused to QtEnc with a FLAG-Tag
MSPEDEIQQLEEEIAQLEQKNAALKEKNQALKYGGGGGSGPGNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGSGDYKDDDDK

SEQ ID NO: 162 - protein, 389 aa, synthetic construct
  REGION 1..389: note = Fusion of DD-N degron with mScarlet-I and synthetic coiled-coil domain P1 (partner of P2)
GVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKPEGSGVSKGEAVIKEFMRFKVHMEGSMNGHEFEIEGEGEGRPYEGTQTAKLKVTKGGPLPFSWDILSPQFMYGSRAFIKHPADIPDYYKQSFPEGFKWERVMNFEDGGAVTVTQDTSLEDGTLIYKVKLRGTNFPPDGPVMQKKTMGWEASTERLYPEDGVLKGDIKMALRLKDGGRYLADFKTTYKAKKPVQMPGAYNVDRKLDITSHNEDYTVVEQYERSEGRHSTGGMDELYKGGGGSGGGGSGPGTGSPEDEIQALEEENAQLEQENAALEEEIAQLEYG

SEQ ID NO: 163 - protein, 444 aa, synthetic construct
  REGION 1..444: note = Fusion of DD-N degron with mTagBFP2 and synthetic coiled-coil domain DHD13b (partner of DHD13a)
GVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKPEGSGVSKGEELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANAKTTYRSKKPAKNLKMPGVYYVDYRLERIKEANNETYVEQHEVAVARYCDLPSKLGHKLNGGGGSGGGGSGPGTGGTEKRLLEEAERAHREQKEIIKKAQELHRRLEEIVRQSGSSEEAKKEAKKILEEIRELSKRSLELLREILYLSQEQKGSLVPR

SEQ ID NO: 164 - protein, 396 aa, synthetic construct
  REGION 1..396: note = Fusion of DD-N degron with mGreenLantern and synthetic coiled-coil domain P4 (partner of P3)
GVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKPEGSGVSKGEELFTGVVPILVELDGDVNGHKFSVRGEGEGDATNGKLTLKFICTTGKLPVPWPTLVTTLGYGVACFARYPDHMKQHDFFKSAMPEGYVQERTISFKDDGTYKTRAEVKFEGDTLVNRIVLKGIDFKEDGNILGHKLEYNFNSHKVYITADKQKNGIKANFKTRHNVEDGGVQLADHYQQNTPIGDGPVLLPDNHYLSHQSKLSKDPNEKRDHMVLKERVTAAGITHDMDELYKGGGGSGGGGSGPGTGSPEDKIAQLKQKIQALKQENQQLEEENAALEYG

SEQ ID NO: 165 - protein, 365 aa, synthetic construct
  REGION 1..365: note = Fusion of Mus musculus Metallothionein 3, the shell forming monomer of the Encapsulin system of Quasibacillus thermotolerans and a C-terminal FLAG-Tag
MDPETCPCPTGGSCTCSDKCKCKGCKCTNCKKSCCSCCPAGCEKCAKDCVCKGEEGAKAEAEKCSCCQGSGPGNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGSGDYKDDDDK

SEQ ID NO: 166 - protein, 370 aa, synthetic construct
  REGION 1..370: note = Fusion of Mus musculus Metallothionein 3, the shell forming monomer of the Encapsulin system of Myxococcus xanthus and a C-terminal FLAG-Tag
MDPETCPCPTGGSCTCSDKCKCKGCKCTNCKKSCCSCCPAGCEKCAKDCVCKGEEGAKAEAEKCSCCQGSGPGPDFLGHAENPLREEEWARLNETVIQVARRSLVGRRILDIYGPLGAGVQTVPYDEFQGVSPGAVDIVGEQETAMVFTDARKFKTIPIIYKDFLLHWRDIEAARTHNMPLDVSAAAGAAALCAQQEDELIFYGDARLGYEGLMTANGRLTVPLGDWTSPGGGFQAIVEATRKLNEQGHFGPYAVVLSPRLYSQLHRIYEKTGVLEIETIRQLASDGVYQSNRLRGESGVVVSTGRENMDLAVSMDMVAAYLGASRMNHPFRVLEALLLRIKHPDAICTLEGAGATERRGSGDYKDDDDK

SEQ ID NO: 167 - protein, 381 aa, synthetic construct
  REGION 1..381: note = Fusion of Mus musculus Metallothionein 3, the shell forming monomer of the Encapsulin system of Myxococcus xanthus and a C-terminal FLAG-Tag followed by a cMyc-derived nuclear localization signal (NLS)
MDPETCPCPTGGSCTCSDKCKCKGCKCTNCKKSCCSCCPAGCEKCAKDCVCKGEEGAKAEAEKCSCCQGSGPGPDFLGHAENPLREEEWARLNETVIQVARRSLVGRRILDIYGPLGAGVQTVPYDEFQGVSPGAVDIVGEQETAMVFTDARKFKTIPIIYKDFLLHWRDIEAARTHNMPLDVSAAAGAAALCAQQEDELIFYGDARLGYEGLMTANGRLTVPLGDWTSPGGGFQAIVEATRKLNEQGHFGPYAVVLSPRLYSQLHRIYEKTGVLEIETIRQLASDGVYQSNRLRGESGVVVSTGRENMDLAVSMDMVAAYLGASRMNHPFRVLEALLLRIKHPDAICTLEGAGATERRGSGDYKDDDDKGGPAAKRVKLD

SEQ ID NO: 168 - protein, 376 aa, synthetic construct
  REGION 1..376: note = Fusion of Mus musculus Metallothionein 3, the shell forming monomer of the Encapsulin system of Quasibacillus thermotolerans and a C-terminal FLAG-Tag followed by a cMyc-derived nuclear localization signal (NLS)
MDPETCPCPTGGSCTCSDKCKCKGCKCTNCKKSCCSCCPAGCEKCAKDCVCKGEEGAKAEAEKCSCCQGSGPGNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGSGDYKDDDDKGGPAAKRVKLD

SEQ ID NO: 169 - protein, 420 aa, synthetic construct
  REGION 1..420: note = Fusion of 3xMT with QtEnc and a C-terminal FLAG-Tag.
MDPETCPCPTGGSCTCSDKCKCKGCKCTNCKKSCCSCCPAGCEKCAKDCVCKGEEGAKAEAEKCSCCQGGTGGSTSTTLVKCACEPCLCNVDPSKAIDRNGLYYCSEACADGHTGGSKGCGHTGCNCHGSAGGCDDKCGCAVPCPGGTGCRCTSARSGAAAGEHTTCGCGEHCGCNPCACGREGTPSGRANRRANCSCGAACNCASCGSATAPGGGGSGGGSGGSANKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRV

SEQ ID NO: 170 - protein, 162 aa, Rattus norvegicus
GDDEGRQSPVSWTSLFALQTEDGFWKLTPELGLILKLNVNALLNSLEEKGIRSLGTKGRERLLDLIATLLVLQFLYTKLEQEGMAAKSLIKMDDVLISRNIPWAFENIKKAREWARRTEGQYPSICQRLELGKDWESATKQLLGIQHQGNTSLHRILHYSQG

SEQ ID NO: 171 - protein, 193 aa, synthetic construct
  REGION 1..193: note = MT3 fused to SpyCatcher003
MDPETCPCPTGGSCTCSDKCKCKGCKCTNCKKSCCSCCPAGCEKCAKDCVCKGEEGAKAEAEKCSCCQPPPPRTGPPPPRVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHT

SEQ ID NO: 172 - protein, 359 aa, synthetic construct
  REGION 1..359: note = Fusion of antiALFATag intrabody with a MT3 tandem, GB1 domain, a FLAG-Tag and C-terminal RXXG degron.
MEVQLQESGGGLVQPGGSLRLSCTASGVTISALNAMAMGWYRQAPGERRVMVAAVSERGNAMYRESVQGRFTVTRDFTNKMVSLQMDNLKPEDTAVYYCHVLEDRVDSFHDYWGQGTQVTVSSGAPGFSSISASGGTGGSDPETCPCPTGGSCTCSDKCKCKGCKCTNCKKSCCSCCPAGCEKCAKDCVCKGEEGAKAEAEKCSCCQGGGSGGGSGGSADPETCPCPTGGSCTCSDKCKCKGCKCTNCKKSCCSCCPAGCEKCAKDCVCKGEEGAKAEAEKCSCCQGSGPGYKLILNGKTLKGETTTEAVDAATAEKVFKQYANDNGVDGEWTYDDATKTFTVTEGDYKDDDDKGRRVG

SEQ ID NO: 173 - protein, 560 aa, synthetic construct
  REGION 1..560: note = Fusion of antiHA frankenbody with a MeLanM tandem, GB1 domain, a FLAG-Tag and C-terminal RXXG degron.
MAEVKLVESGGGLVKPGGSLKLSCAASGFTFSSYGMSWVRQTPEKRLEWVATISRGGSYTYYPDSVKGRFTISRDNAKNTLYLQMSSLRSEDTAIYYCARRETYDEKGFAYWGQGTTLTVSSGGGGSGGGGSGGGGSDIVLTQSPASLTVSLGQRATISCKSSQSLLNSGNQKNYLTWYQQKPGQPPKLLIYWASTRESGIPARFSGSGSGTDFTLNIHPVEEEDAATYYCQNDNSHPLTFGAGTKLEIGGTGGSPTTTTKVDIAAFDPDKDGTIDLKEALAAGSAAFDKLDPDKDGTLDAKELKGRVSEADLKKLDPDNDGTLDKKEYLAAVEAQFKAANPDNDGTIDARELASPAGSALVNLIRGGGGSGGGGSPTTTTKVDIAAFDPDKDGTIDLKEALAAGSAAFDKLDPDKDGTLDAKELKGRVSEADLKKLDPDNDGTLDKKEYLAAVEAQFKAANPDNDGTIDARELASPAGSALVNLIRGSGPGYKLILNGKTLKGETTTEAVDAATAEKVFKQYANDNGVDGEWTYDDATKTFTVTEGDYKDDDDKGRRVG

SEQ ID NO: 174 - protein, 450 aa, synthetic construct
  REGION 1..450: note = Fusion of antiMoonTag intrabody with a AssVanabin2 tandem, GB1 domain, a FLAG-Tag and C-terminal RXXG degron.
MEVQLVESGGGLVQPGGSLRLSCAASGSISSVDVMSWYRQAPGKQRELVAFITDRGRTNYKVSVKGRFTISRDNSKNMVYLQMNSLKPEDTADYLCRAESRTSWSSPSPLDVWGRGTQVTVSSGGTGGSSKVIFALVLVVVLVACINATYVEFEEAYAPVDCKGQCTTPCEPLTACKEKCAESCETSADKKTCRRNCKKADCEPQDKVCDACRMKCHKACRAANCASECPKHEHKSDTCRACMKTNCKGGGGSGGGGSSKVIFALVLVVVLVACINATYVEFEEAYAPVDCKGQCTTPCEPLTACKEKCAESCETSADKKTCRRNCKKADCEPQDKVCDACRMKCHKACRAANCASECPKHEHKSDTCRACMKTNCKGSGPGYKLILNGKTLKGETTTEAVDAATAEKVFKQYANDNGVDGEWTYDDATKTFTVTEGDYKDDDDKGRRVG

SEQ ID NO: 175 - protein, 434 aa, synthetic construct
  REGION 1..434: note = Fusion of antiMoonTag intrabody with a MeLanM tandem, GB1 domain, a FLAG-Tag and C-terminal RXXG degron.
MEVQLVESGGGLVQPGGSLRLSCAASGSISSVDVMSWYRQAPGKQRELVAFITDRGRTNYKVSVKGRFTISRDNSKNMVYLQMNSLKPEDTADYLCRAESRTSWSSPSPLDVWGRGTQVTVSSGGTGGSPTTTTKVDIAAFDPDKDGTIDLKEALAAGSAAFDKLDPDKDGTLDAKELKGRVSEADLKKLDPDNDGTLDKKEYLAAVEAQFKAANPDNDGTIDARELASPAGSALVNLIRGGGGSGGGGSPTTTTKVDIAAFDPDKDGTIDLKEALAAGSAAFDKLDPDKDGTLDAKELKGRVSEADLKKLDPDNDGTLDKKEYLAAVEAQFKAANPDNDGTIDARELASPAGSALVNLIRGSGPGYKLILNGKTLKGETTTEAVDAATAEKVFKQYANDNGVDGEWTYDDATKTFTVTEGDYKDDDDKGRRVG

SEQ ID NO: 176 - protein, 358 aa, synthetic construct
  REGION 1..358: note = Fusion of antiMoonTag intrabody with a 4xP3 CCC-Tag, GB1 domain, a FLAG-Tag and C-terminal RXXG degron.
MEVQLVESGGGLVQPGGSLRLSCAASGSISSVDVMSWYRQAPGKQRELVAFITDRGRTNYKVSVKGRFTISRDNSKNMVYLQMNSLKPEDTADYLCRAESRTSWSSPSPLDVWGRGTQVTVSSGGTGGSSPEDEIQQLEEEIAQLEQKNAALKEKNQALKYGLKSGPGSGSPEDEIQQLEEEIAQLEQKNAALKEKNQALKYGTGSGPGSGSPEDEIQQLEEEIAQLEQKNAALKEKNQALKYGLKSGPGSGSPEDEIQQLEEEIAQLEQKNAALKEKNQALKYGGSGPGYKLILNGKTLKGETTTEAVDAATAEKVFKQYANDNGVDGEWTYDDATKTFTVTEGDYKDDDDKGRRVG

SEQ ID NO: 177 - protein, 348 aa, synthetic construct
  REGION 1..348: note = Fusion of antiMoonTag intrabody with a MT3 tandem, GB1 domain, a FLAG-Tag and C-terminal RXXG degron.
MEVQLVESGGGLVQPGGSLRLSCAASGSISSVDVMSWYRQAPGKQRELVAFITDRGRTNYKVSVKGRFTISRDNSKNMVYLQMNSLKPEDTADYLCRAESRTSWSSPSPLDVWGRGTQVTVSSGGTGGSDPETCPCPTGGSCTCSDKCKCKGCKCTNCKKSCCSCCPAGCEKCAKDCVCKGEEGAKAEAEKCSCCQGGGSGGGSGGSADPETCPCPTGGSCTCSDKCKCKGCKCTNCKKSCCSCCPAGCEKCAKDCVCKGEEGAKAEAEKCSCCQGSGPGYKLILNGKTLKGETTTEAVDAATAEKVFKQYANDNGVDGEWTYDDATKTFTVTEGDYKDDDDKGRRVG

SEQ ID NO: 178 - protein, 387 aa, synthetic construct
  REGION 1..387: note = Fusion of antiMoonTag intrabody with a EcCusF tandem, GB1 domain, a FLAG-Tag and C-terminal RXXG degron.
MEVQLVESGGGLVQPGGSLRLSCAASGSISSVDVMSWYRQAPGKQRELVAFITDRGRTNYKVSVKGRFTISRDNSKNMVYLQMNSLKPEDTADYLCRAESRTSWSSPSPLDVWGRGTQVTVSSGGTGGSDPETCPCPTGGSCTCSDKCKCKGCKCTNCKKSCCSCCPAGCEKCAKDCVCKGEEGAKAEAEKCSCCQMNEHHHETMSEAQPQVISATGEVKGIDLESKKITIHHDPIAAVNWPEMTMRFTITPQTKMSEIKTGDKVAFNFVQQGNLSLLQDIKVSQMNEHHHETMSEAQPQVISATGEVKGIDLESKKITIHHDPIAAVNWPEMTMRFTITPQTKMSEIKTGDKVAFNFVQQGNLSLLQDIKVSQDYKDDDDKGRRVG

SEQ ID NO: 179 - protein, 442 aa, synthetic construct
  REGION 1..442: note = Fusion of LifeAct-peptide to emFPv4
MGVADLIKKFESISKEEGTGSKNEQELLELDKWASLQQQKNEQELLELDKWASLQQQKNEQELLELDKWASLGQVSKGEELFTGVVPILVELDGDVNGHKFSVRGEGEGDATNGKLTLKFICTTGKLPVPWPTLVTTLGYGVACFARYPDHMKQHDFFKSAMPEGYVQERTISFKDDGTYKTRAEVKFEGDTLVNRIVLKGIDFKEDGNILGHKLEYNFNSHKVYITADKQKNGIKANFKTRHNVEGGKNEQELLELDKWASLQQQKNEQELLELDKWASLGGQQGGKNEQELLELDKWASLQQQKNEQELLELDKWASLGGDGGVQLADHYQQNTPIGDGPVLLPDNHYLSHQSKLSKDPNEKRDHMVLKERVTAAGITHDMDELYKGQGKNEQELLELDKWASLQQQKNEQELLELDKWASLQQQKNEQELLELDKWASL

SEQ ID NO: 180 - DNA, 1329 bp, synthetic construct
  misc_feature 1..1329: note = DNA sequence encoding QtEnc-FLAG followed by a TGA stop codon, followed by the read-through promoting sequence "RT20s" followed by an anti-GFPintrabody with a C-terminal HiBit peptide.
  source 1..1329: mol_type = other DNA
atgaacaagagccagctgtaccctgacagccctctgaccgaccaggacttcaaccagctggaccagaccgtgattgaagccgccagaaggcagctcgtcggcagaagattcatcgagctgtacggccctctcggcagaggcatgcagagcgtgttcaacgacatcttcatggaaagccacgaggccaagatggacttccagggcagcttcgacaccgaggtggaaagcagcagaagagtgaactacacaatccccatgctgtacaaggacttcgtgctgtactggcgcgacctggaacagagcaaggccctggacatccccatcgatttttccgtggccgccaacgccgctagagatgtggcctttctggaagatcagatgatctttcacggcagcaaagagttcgacatccccggcctgatgaacgtgaagggcagactgacacacctgatcggcaattggtacgagagcggcaacgccttccaggacatcgtggaagcccggaacaagctgctggaaatgaaccacaacggcccctacgctctggtgctgagccctgagctgtatagcctgctgcacagagtgcacaaggacaccaacgtcctggaaatcgagcacgtgcgcgagctgattaccgccggtgtctttcagagccctgtgctgaagggaaagtccggcgtgatcgtgaacaccggcagaaacaacctggacctggccatcagcgaggacttcgagacagcctatctgggcgaagagggcatgaatcaccccttccgggtgtacgagacagtggtgctgcggattaagaggcctgccgccatctgcaccctgatcgatcctgaagaaggctccggcgactacaaggacgacgacgacaaatgacaattagctcatccagacattccgggcggaggtggcagcggacctggtggctccggcgtgcagctggtcgagtccggaggagctctggttcagcccggcggctctcttcgactcagttgtgccgcatctggcttccccgtgaaccgatattcaatgagatggtaccggcaggctcccggaaaggagcgcgaatgggttgccggaatgtcctccgccggcgaccgatcatcttacgaagactctgtcaaaggcagattcaccatcagcagagatgacgcccggaataccgtctatctccagatgaactctcttaagcctgaggatacagcagtctactactgcaacgtgaacgtgggattcgagtattggggacaggggacccaggtcactgtgtcctctggcagcggcggcgtgagcggctggcgcctgtttaaaaaaattagctaa

SEQ ID NO: 181 - DNA, 2742 bp, synthetic construct
  misc_feature 1..2742: note = DNA sequence encoding QtEnc-FLAG followed by a TGA stop codon, followed by a read-through promoting sequence "RT20s", followed by an Ssp DnaE intein mutant (N159A) which exhibits hyperfast N-terminal cleavage, followed by a P2A peptide followed by a
  source 1..2742: mol_type = other DNA
atgaacaagagccagctgtaccctgacagccctctgaccgaccaggacttcaaccagctggaccagaccgtgattgaagccgccagaaggcagctcgtcggcagaagattcatcgagctgtacggccctctcggcagaggcatgcagagcgtgttcaacgacatcttcatggaaagccacgaggccaagatggacttccagggcagcttcgacaccgaggtggaaagcagcagaagagtgaactacacaatccccatgctgtacaaggacttcgtgctgtactggcgcgacctggaacagagcaaggccctggacatccccatcgatttttccgtggccgccaacgccgctagagatgtggcctttctggaagatcagatgatctttcacggcagcaaagagttcgacatccccggcctgatgaacgtgaagggcagactgacacacctgatcggcaattggtacgagagcggcaacgccttccaggacatcgtggaagcccggaacaagctgctggaaatgaaccacaacggcccctacgctctggtgctgagccctgagctgtatagcctgctgcacagagtgcacaaggacaccaacgtcctggaaatcgagcacgtgcgcgagctgattaccgccggtgtctttcagagccctgtgctgaagggaaagtccggcgtgatcgtgaacaccggcagaaacaacctggacctggccatcagcgaggacttcgagacagcctatctgggcgaagagggcatgaatcaccccttccgggtgtacgagacagtggtgctgcggattaagaggcctgccgccatctgcaccctgatcgatcctgaagaaggctccggcgactacaaggacgacgacgacaaatagcaattagctcatccagacattccgtgtctgtctttcggcaccgagatcctgaccgtggaatacggccctctgcctatcggcaagatcgtgtccgaagagatcaactgcagcgtgtacagcgtggacccagagggcagagtgtacacacaggctatcgcccagtggcacgacagaggcgaacaagaggtgctggaatacgagctggaagatggcagcgtgatcagagccaccagcgaccacagatttctgaccaccgactaccagctgctggccatcgaggaaatcttcgccagacagctggacctgctgaccctggaaaacatcaagcagaccgaggaagccctggacaaccacagactgccctttccactgctggatgccggcaccatcaagatggtcaaagtgatcggcagacgcagcctgggcgtgcagagaatcttcgatatcggactgccccaggaccacaactttctgctggctaacggcgctatcgccgctgcctgttcttgtggctctggatctagaggcggctctggcgccacaaacttcagcctgcttaaacaggccggcgacgtggaagagaaccccggacctaacaagagccagctgtaccctgacagccctctgaccgaccaggacttcaaccagctggaccagaccgtgattgaagccgccagaaggcagctcgtcggcagaagattcatcgagctgtacggccctctcggcagaggcatgcagagcgtgttcaacgacatcttcatggaaagccacgaggccaagatggacttccagggcagcttcgacaccgaggtggaaagcagcagaagagtgaactacacaatccccatgctgtacaaggacttcgtgctgtactggcgcgacctggaacagagcaaggccctggacatccccatcgatttttccgtggccgccaacgccgctagagatgtggcctttctggaagatcagatgatctttcacggcagcaaagagttcgacatccccggcctgatgaacgtgaagggcagactgacacacctgatcggcaattggtacgagagcggcaacgccttccaggacatcgtggaagcccggaacaagctgctggaaatgaaccacaacggcccctacgctctggtgctgagccctgagctgtatagcctgctgcacagagtgcacaaggacaccaacgtcctggaaatcgagcacgtgcgcgagctgattaccgccggtgtctttcagagccctgtgctgaagggaaagtccggcgtgatcgtgaacaccggcagaaacaacctggacctggccatcagcgaggacttcgagacagcctatctgggcgaagagggcatgaatcaccccttccgggtgtacgagacagtggtgctgcggattaagaggcctgccgccatctgcaccctgatcgatcctgaagaaggcggaggtggcagcggacctggtggctccggcgtgcagctggtcgagtccggaggagctctggttcagcccggcggctctcttcgactcagttgtgccgcatctggcttccccgtgaaccgatattcaatgagatggtaccggcaggctcccggaaaggagcgcgaatgggttgccggaatgtcctccgccggcgaccgatcatcttacgaagactctgtcaaaggcagattcaccatcagcagagatgacgcccggaataccgtctatctccagatgaactctcttaagcctgaggatacagcagtctactactgcaacgtgaacgtgggattcgagtattggggacaggggacccaggtcactgtgtcctctggcagcggcggcgtgagcggctggcgcctgtttaaaaaaattagctaa

SEQ ID NO: 182 - protein, 19 aa, Porcine tescho virus
ATNFSLLKQAGDVEENPGP

SEQ ID NO: 183 - protein, 558 aa, synthetic construct
  REGION 1..558: note = Tandem MT3 fused to QtEnc which has a C-terminal anti GFP intrabody
MDPETCPCPTGGSCTCSDKCKCKGCKCTNCKKSCCSCCPAGCEKCAKDCVCKGEEGAKAEAEKCSCCQGGGSGGGSGGSADPETCPCPTGGSCTCSDKCKCKGCKCTNCKKSCCSCCPAGCEKCAKDCVCKGEEGAKAEAEKCSCCQGSGPGNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGGGGSGPGGSGVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSS

SEQ ID NO: 184 - protein, 238 aa, Aequorea victoria
MSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYK

SEQ ID NO: 185 - protein, 235 aa, Anaplasma marginale
VSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 186 - protein, 114 aa, synthetic construct
  REGION 1..114: note = antiGFP-intrabody
VQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSS

SEQ ID NO: 187 - protein, 124 aa, synthetic construct
  REGION 1..124: note = antimCherry intrabody (LaM-4)
AQVQLVESGGSLVQPGGSLRLSCAASGRFAESSSMGWFRQAPGKEREFVAAISWSGGATNYADSAKGRFTLSRDNTKNTVYLQMNSLKPDDTAVYYCAANLGNYISSNQRLYGYWGQGTQVTVS

SEQ ID NO: 188 - protein, 953 aa, synthetic construct
  REGION 1..953: note = Fusion of sfGFP-SasG-G51-G54-mCherry
MSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGGTGGSAPKTITELEKKVEEIPFKKERKFNPDLAPGTEKVTREGQKGEKTITTPTLKNPLTGVIISKGEPKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETGSGATGSVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 189 - protein, 479 aa, synthetic construct
  REGION 1..479: note = MT3 fused to QtEnc with a C-terminal anti GFP intrabody
MDPETCPCPTGGSCTCSDKCKCKGCKCTNCKKSCCSCCPAGCEKCAKDCVCKGEEGAKAEAEKCSCCQGSGPGNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGGGGSGPGGSGVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSS

SEQ ID NO: 190 - protein, 402 aa, synthetic construct
  REGION 1..402: note = Fusion of BmTyr and synthetic coiled-coil domain P2 (partner of P1)
MGHSNKYRVRKNVLHLTDTEKRDFVRTVLILKEKGIYDRYIAWHGAAGKFHTPPGSDRNAAHMSSAFLPWHREYLLRFERDLQSINPEVTLPYWEWETDAQMQDPSQSQIWSADFMGGNGNPIKDFIVDTGPFAAGRWTTIDEQGNPSGGLKRNFGATKEAPTLPTRDDVLNALKITQYDTPPWDMTSQNSFRNQLEGFINGPQLHNRVHRWVGGQMGVVPTAPNDPVFFLHHANVDRIWAVWQIIHRNQNYQPMKNGPFGQNFRDPMYPWNTTPEDVMNHRKLGYVYDIELRKSKRSSGGGGSGGGGSTGSPEDEIQALEEENAQLEQENAALEEEIAQLEYGSGSETPGTSESATPESAISHGFPPEVEEQAAGTLPMSCAQESGMDRHPAACASARINV

SEQ ID NO: 191 - protein, 375 aa, synthetic construct
  REGION 1..375: note = Fusion of Brain FABP and superfolder GFP
VEAFCATWKLTNSQNFDEYMKALGVGFATRQVGNVTKPTVIISQEGDKVVIRTLSTFKNTEISFQLGEEFDETTADDRNCKSVVSLDGDKLVHIQKWDGKETNFVREIKDGKMVMTLTFGDVVAVRHYEKAPPPPRTGSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYK

SEQ ID NO: 192 - protein, 699 aa, synthetic construct
  REGION 1..699: note = Fusion of BM3h and superfolder GFP
TIKEMPQPKTFGELKNLPLLNTDKPVQALMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLPPPPRTGSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYK

SEQ ID NO: 193 - protein, 135 aa, Homo sapiens
FATRQVGNVTKPTVIISQEGDKVVIRTLSTFKNTEISFQLGEEFDETTADDRNCKSVVSLDGDKLVHIQKWDGKETNFVREIKDGKMVMTLTFGDVVAVRHYEKAGGGSGMVEAFCATWKLTNSQNFDEYMKALG

SEQ ID NO: 194 - protein, 955 aa, synthetic construct
  REGION 1..955: note = Fusion of six B-FABP flanked by DnaE split intein fragments C-intein (AA 103-137) on the N-term and N-Intein (AA 1-102)
MIKIATRKYLGKQNVYDIGVGEPHNFALKNGFIASNGGGVEAFCATWKLTNSQNFDEYMKALGVGFATRQVGNVTKPTVIISQEGDKVVIRTLSTFKNTEISFQLGEEFDETTADDRNCKSVVSLDGDKLVHIQKWDGKETNFVREIKDGKMVMTLTFGDVVAVRHYEKAGGGSGVEAFCATWKLTNSQNFDEYMKALGVGFATRQVGNVTKPTVIISQEGDKVVIRTLSTFKNTEISFQLGEEFDETTADDRNCKSVVSLDGDKLVHIQKWDGKETNFVREIKDGKMVMTLTFGDVVAVRHYEKAGGGSGVEAFCATWKLTNSQNFDEYMKALGVGFATRQVGNVTKPTVIISQEGDKVVIRTLSTFKNTEISFQLGEEFDETTADDRNCKSVVSLDGDKLVHIQKWDGKETNFVREIKDGKMVMTLTFGDVVAVRHYEKAGGGSGVEAFCATWKLTNSQNFDEYMKALGVGFATRQVGNVTKPTVIISQEGDKVVIRTLSTFKNTEISFQLGEEFDETTADDRNCKSVVSLDGDKLVHIQKWDGKETNFVREIKDGKMVMTLTFGDVVAVRHYEKAGGGSGVEAFCATWKLTNSQNFDEYMKALGVGFATRQVGNVTKPTVIISQEGDKVVIRTLSTFKNTEISFQLGEEFDETTADDRNCKSVVSLDGDKLVHIQKWDGKETNFVREIKDGKMVMTLTFGDVVAVRHYEKAGGGSGVEAFCATWKLTNSQNFDEYMKALGVGFATRQVGNVTKPTVIISQEGDKVVIRTLSTFKNTEISFQLGEEFDETTADDRNCKSVVSLDGDKLVHIQKWDGKETNFVREIKDGKMVMTLTFGDVVAVRHYEKAGGGCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 195 - protein, 651 aa, synthetic construct
  REGION 1..651: note = Fusion of three repeats of B-FABP fused to MmMT3 flanked by DnaE split intein fragments C-intein (AA 103-137) on the N-term and N-Intein (AA 1-102)
MIKIATRKYLGKQNVYDIGVGEPHNFALKNGFIASNGGGVEAFCATWKLTNSQNFDEYMKALGVGFATRQVGNVTKPTVIISQEGDKVVIRTLSTFKNTEISFQLGEEFDETTADDRNCKSVVSLDGDKLVHIQKWDGKETNFVREIKDGKMVMTLTFGDVVAVRHYEKAGGGMDPETCPCPTGGSCTCSDKCKCKGCKCTNCKKSCCSCCPAGCEKCAKDCVCKGEEGAKAEAEKCSCCQGGGVEAFCATWKLTNSQNFDEYMKALGVGFATRQVGNVTKPTVIISQEGDKVVIRTLSTFKNTEISFQLGEEFDETTADDRNCKSVVSLDGDKLVHIQKWDGKETNFVREIKDGKMVMTLTFGDVVAVRHYEKAGGGMDPETCPCPTGGSCTCSDKCKCKGCKCTNCKKSCCSCCPAGCEKCAKDCVCKGEEGAKAEAEKCSCCQVEAFCATWKLTNSQNFDEYMKALGVGFATRQVGNVTKPTVIISQEGDKVVIRTLSTFKNTEISFQLGEEFDETTADDRNCKSVVSLDGDKLVHIQKWDGKETNFVREIKDGKMVMTLTFGDVVAVRHYEKAGGGMDPETCPCPTGGSCTCSDKCKCKGCKCTNCKKSCCSCCPAGCEKCAKDCVCKGEEGAKAEAEKCSCCQGGG

SEQ ID NO: 196 - protein, 1094 aa, synthetic construct
  REGION 1..1094: note = Fusion of three repeats of 2x B-FABP intermixed by SpyTag003 and Spycatcher003 and flanked by DnaE split intein fragments C-intein (AA 103-137) on the N-term and N-Intein (AA 1-102)
MIKIATRKYLGKQNVYDIGVGEPHNFALKNGFIASNGGGVEAFCATWKLTNSQNFDEYMKALGVGFATRQVGNVTKPTVIISQEGDKVVIRTLSTFKNTEISFQLGEEFDETTADDRNCKSVVSLDGDKLVHIQKWDGKETNFVREIKDGKMVMTLTFGDVVAVRHYEKAGGGSGVEAFCATWKLTNSQNFDEYMKALGVGFATRQVGNVTKPTVIISQEGDKVVIRTLSTFKNTEISFQLGEEFDETTADDRNCKSVVSLDGDKLVHIQKWDGKETNFVREIKDGKMVMTLTFGDVVAVRHYEKAGGGSGRGVPHIVMVDAYKRYKGGGSGVEAFCATWKLTNSQNFDEYMKALGVGFATRQVGNVTKPTVIISQEGDKVVIRTLSTFKNTEISFQLGEEFDETTADDRNCKSVVSLDGDKLVHIQKWDGKETNFVREIKDGKMVMTLTFGDVVAVRHYEKAGGGSGVEAFCATWKLTNSQNFDEYMKALGVGFATRQVGNVTKPTVIISQEGDKVVIRTLSTFKNTEISFQLGEEFDETTADDRNCKSVVSLDGDKLVHIQKWDGKETNFVREIKDGKMVMTLTFGDVVAVRHYEKAGGGSGVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHTGGGSGVEAFCATWKLTNSQNFDEYMKALGVGFATRQVGNVTKPTVIISQEGDKVVIRTLSTFKNTEISFQLGEEFDETTADDRNCKSVVSLDGDKLVHIQKWDGKETNFVREIKDGKMVMTLTFGDVVAVRHYEKAGGGSGVEAFCATWKLTNSQNFDEYMKALGVGFATRQVGNVTKPTVIISQEGDKVVIRTLSTFKNTEISFQLGEEFDETTADDRNCKSVVSLDGDKLVHIQKWDGKETNFVREIKDGKMVMTLTFGDVVAVRHYEKAGGGCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 197 - protein, 317 aa, synthetic construct
  REGION 1..317: note = Fusion of QtEncapsulin and M13 a synthetic peptide binding Calmodulin
NKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEPPPPRPPPPRKRRWKKNFIAVSAANRFKKISSSGAL

SEQ ID NO: 198 - protein, 283 aa, Homo sapiens
VEAFCATWKLTNSQNFDEYMKALGVGFATRQVGNVTKPTVIISQEGDKVVIRTLSTFKNTEISFQLGEEFDETTADDRNCKSVVSLDGDKLVHIQKWDGKETNFVREIKDGKMVMTLTFGDVVAVRHYEKPPPPRADQLTDEQIAEFKEAFSLFDKDGDGTITTKELGTVMRSLGQNPTEAELQDMINEVDADGNGTIDFPEFLNLMARKMKDTDSEEKLKEAFRVFDKDGNGFISAAELRHVMTNLGEKLTDEEVDEMIREADVDGDGQVNYEEFVQVMMAK

SEQ ID NO: 199 - protein, 13 aa, synthetic construct
  REGION 1..13: note = Synthetic Silver mineralizing peptide 4
MNPSSLFRYLPSD

SEQ ID NO: 200 - protein, 1020 aa, Prevotella sp.
MKRTADGSEFESPKKKRKVNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNAYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDGSKRTADGSEFEPKKKRKV

SEQ ID NO: 201 - protein, 1186 aa, Homo sapiens
MSVACVLKRKAVLWQDSFSPHLKHHPQEPANPNMPVVLTSGTGSQAQPQPAANQALAAGTHSSPVPGSIGVAGRSQDDAMVDYFFQRQHGEQLGGGGSGGGGYNNSKHRWPTGDNIHAEHQVRSMDELNHDFQALALEGRAMGEQLLPGKKFWETDESSKDGPKGIFLGDQWRDSAWGTSDHSVSQPIMVQRRPGQSFHVNSEVNSVLSPRSESGGLGVSMVEYVLSSSPGDSCLRKGGFGPRDADSDENDKGEKKNKGTFDGDKLGDLKEEGDVMDKTNGLPVQNGIDADVKDFSRTPGNCQNSANEVDLLGPNQNGSEGLAQLTSTNGAKPVEDFSNMESQSVPLDPMEHVGMEPLQFDYSGTQVPVDSAAATVGLFDYNSQQQLFQRPNALAVQQLTAAQQQQYALAAAHQPHIGLAPAAFVPNPYIISAAPPGTDPYTAGLAAAATLGPAVVPHQYYGVTPWGVYPASLFQQQAAAAAAATNSANQQTTPQAQQGQQQVLRGGASQRPLTPNQNQQGQQTDPLVAAAAVNSALAFGQGLAAGMPGYPVLAPAAYYDQTGALVVNAGARNGLGAPVRLVAPAPVIISSSAAQAAVAAAAASANGAAGGLAGTTNGPFRPLGTQQPQPQPQQQPNNNLASSSFYGNNSLNSNSQSSSLFSQGSAQPANTSLGFGSSSSLGATLGSALGGFGTAVANSNTGSGSRRDSLTGSSDLYKRTSSSLTPIGHSFYNGLSFSSSPGPVGMPLPSQGPGHSQTPPPSLSSHGSSSSLNLGGLTNGSGRYISAAPGAEAKYRSASSASSLFSPSSTLFSSSRLRYGMSDVMPSGRSRLLEDFRNNRYPNLQLREIAGHIMEFSQDQHGSRFIQLKLERATPAERQLVFNEILQAAYQLMVDVFGNYVIQKFFEFGSLEQKLALAERIRGHVLSLALQMYGCRVIQKALEFIPSDQQNEMVRELDGHVLKCVKDQNGNHVVQKCIECVQPQSLQFIIDAFKGQVFALSTHPYGCRVIQRILEHCLPDQTLPILEELHQHTEQLVQDQYGNYVIQHVLEHGRPEDKSKIVAEIRGNVLVLSQHKFASNVVEKCVTHASRTERAVLIDEVCTMNDGPHSALYTMMKDQYANYVVQKMIDVAEPGQRKIVMHKIRPHIATLRKYTYGKHILAKLEKYYMKNGVDLGPICGPPNGII

SEQ ID NO: 202 - protein, 139 aa, Anguilla anguilla
MLEKFVGTWKIADSHNFGEYLKAIGAPKELSDGGDATTPTLYISQKDGDKMSVKIENGPPTFLDTQVKFKLGEEFDEFPSDRRKGVKSVVNLVGEKLVYVQKWDGKETTYVREIKDGKLVVTLTMGDVVAVRSYRRATE

SEQ ID NO: 203 - protein, 239 aa, Aequorea victoria
MVSKGEELFTGVVPILVELDGDVNGHKFSVRGEGEGDATNGKLTLKFICTTGKLPVPWPTLVTTLGYGVACFARYPDHMKQHDFFKSAMPEGYVQERTISFKDDGTYKTRAEVKFEGDTLVNRIVLKGIDFKEDGNILGHKLEYNFNSHKVYITADKQKNGIKANFKTRHNVEDGGVQLADHYQQNTPIGDGPVLLPDNHYLSHQSKLSKDPNEKRDHMVLKERVTAAGITHDMDELYK

SEQ ID NO: 204 - protein, 408 aa, synthetic construct
  REGION 1..408: note = Engineered fluorescent Calcium integrator
MGSDQLTEEQIAEFKEAFSLFDKDGDGTITTKELGTVMRSLGQNPTEAELQDMINEVDADGDGTIDFPEFLTMMARKMKDTDSEEEIREAFRVFDKDGNGYISAAELRHVMTNLGEKLTDEEVDEMIREADIDGDGQVNYEEFVVMMTAKLECEKIYVRDGVLTGDIHMILLLEGNAHYRCDFRTTYKAKEKGVKLPGVHYVDHTIEILSHDKDYNKVKIYEYAVAHSGLPDNARRGGTGGSMVSAIKPDMKIKLRMEGNVNGHHFVIDGDGTGKPYEGKQTMDLEVKEGGPLPFAFDILTTAFHYGNRVFVKYPDNIQDYFKQSFPKGYSWERSMTFEDGGICYARSDITMEGDTFYNKVRFYGTNFPANGPVMQKKTLKWMPSWTRSSRRKFNKTGHALRAIGRLS

SEQ ID NO: 205 - protein, 225 aa, Discosoma sp.
MASSEDVIKEFMRFKVRMEGSVNGHEFEIEGEGEGRPYEGTQTAKLKVTKGGPLPFAWDILSPQFQYGSKAYVKHPADIPDYLKLSFPEGFKWERVMNFEDGGVVTVTQDSSLQDGEFIYKVKLRGTNFPSDGPVMQKKTMGWEASTERMYPEDGALKGEIKMRLKLKDGGHYDAEVKTTYMAKKPVQLPGAYKTDIKLDITSHNEDYTIVEQYERAEGRHSTGA

SEQ ID NO: 206 - protein, 121 aa, synthetic construct
  REGION 1..121: note = phostphopetptide binding Forkhead-associated domain
FLTLKPLPDSIIQESLEIQQGVNPFFIGRSEDCNCKIEDNRLSRVHCFIFKKRHAVGKSMYESPAQGLDDIWYCHTGTNVSYLNNNRMIQGTKFLLQDGDEIKIIWDKNNKFVIGFKVEIN

SEQ ID NO: 207 - protein, 131 aa, synthetic construct
  REGION 1..131: note = Coat protein
MANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 208 - protein, 678 aa, synthetic construct
  REGION 1..678: note = Fusion of mEos4b together with APEX2 to a C-terminal minimal interaction domain (mINT)
MVSAIKPDMRIKLRMEGNVNGHHFVIDGDGTGKPYEGKQTMDLEVKEGGPLPFAFDILTTAFHYGNRVFVKYPDNIQDYFKQSFPKGYSWERSLTFEDGGICNARNDITMEGDTFYNKVRFYGTNFPANGPVMQKKTLKWEPSTEKMYVRDGVLTGDIEMALLLEGNAHYRCDFRTTYKAKEKGVKLPGAHFVDHAIEILSHDKDYNKVKLYEHAVAHSGLPDNARRGGGGSGGGGDYKDDDDKGKSYPTVSADYQDAVEKAKKKLRGFIAEKRCAPLMLRLAFHSAGTFDKGTKTGGPFGTIKHPAELAHSANNGLDIAVRLLEPLKAEFPILSYADFYQLAGVVAVEVTGGPKVPFHPGREDKPEPPPEGRLPDPTKGSDHLRDVFGKAMGLTDQDIVALSGGHTIGAAHKERSGFEGPWTSNPLIFDNSYFTELLSGEKEGLLQLPSDKALLSDPVFRPLVDKYAADEDAFFADYAEAHQKLSELGFADALQLPPLERLTLDGGGSGGGGSGGGDDEGRQSPVSWTSLFALQTEDGFWKLTPELGLILKLNVNALLNSLEEKGIRSLGTKGRERLLDLIATLLVLQFLYTKLEQEGMAAKSLIKMDDVLISRNIPWAFENIKKAREWARRTEGQYPSICQRLELGKDWESATKQLLGIQHQGNTSLHRILHYSQG

SEQ ID NO: 209 - protein, 880 aa, synthetic construct
  REGION 1..880: note = Fusion of mouse Gap43 palmitoylation signal to the N-terminus of the major vault protein (MVP).
MLCCMRRTKQVEKNDEDQKIATEEAIIRIPPYHYIHVLDQNSNVSRVEVGPKTYIRQDNERVLFAPVRMVTVPPRHYCIVANPVSRDTQSSVLFDITGQVRLRHADQEIRLAQDPFPLYPGEVLEKDITPLQVVLPNTALHLKALLDFEDKNGDKVMAGDEWLFEGPGTYIPQKEVEVVEIIQATVIKQNQALRLRARKECFDREGKGRVTGEEWLVRSVGAYLPAVFEEVLDLVDAVILTEKTALHLRALQNFRDLRGVLHRTGEEWLVTVQDTEAHVPDVYEEVLGVVPITTLGPRHYCVILDPMGPDGKNQLGQKRVVKGEKSFFLQPGERLERGIQDVYVLSEQQGLLLKALQPLEEGESEEKVSHQAGDCWLIRGPLEYVPSAKVEVVEERQAIPLDQNEGIYVQDVKTGKVRAVIGSTYMLTQDEVLWEKELPSGVEELLNLGHDPLADRGQKGTAKPLQPSAPRNKTRVVSYRVPHNAAVQVYDYRAKRARVVFGPELVTLDPEEQFTVLSLSAGRPKRPHARRALCLLLGPDFFTDVITIETADHARLQLQLAYNWHFELKNRNDPAEAAKLFSVPDFVGDACKAIASRVRGAVASVTFDDFHKNSARIIRMAVFGFEMSEDTGPDGTLLPKARDQAVFPQNGLVVSSVDVQSVEPVDQRTRDALQRSVQLAIEITTNSQEAAAKHEAQRLEQEARGRLERQKILDQSEAEKARKELLELEAMSMAVESTGNAKAEAESRAEAARIEGEGSVLQAKLKAQALAIETEAELERVKKVREMELIYARAQLELEVSKAQQLANVEAKKFKEMTEALGPGTIRDLAVAGPEMQVKLLQSLGLKSTLITDGSSPINLFSTAFGLLGLGSDGQPPAQK

SEQ ID NO: 210 - protein, 485 aa, synthetic construct
  REGION 1..485: note = N-terminal fusion of SNAP-tag connected via GS linker to QtEnc with a C-terminal FLAG epitope
MDKDCEMKRTTLDSPLGKLELSGCEQGLHRIIFLGKGTSAADAVEVPAPAAVLGGPEPLMQATAWLNAYFHQPEAIEEFPVPALHHPVFQQESFTRQVLWKLLKVVKFGEVISYSHLAALAGNPAATAAVKTALSGNPVPILIPCHRVVQGDLDVGGYEGGLAVKEWLLAHEGHRLGKPGLGGGTGGSGSGPGNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGSGDYKDDDDK

SEQ ID NO: 211 - protein, 600 aa, synthetic construct
  REGION 1..600: note = N-terminal fusion of Halo-tag connected via GS linker to QtEnc with a C-terminal FLAG epitope
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGGTGGSGSGPGNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGSGDYKDDDDK

SEQ ID NO: 212 - protein, 320 aa, synthetic construct
  REGION 1..320: note = Fusion of MT3 to AA 110-346 of Pyrococcus furiosus Encapsulin monomer with a C-terminal FLAG epitope.
MDPETCPCPTGGSCTCSDKCKCKGCKCTNCKKSCCSCCPAGCEKCAKDCVCKGEEGAKAEAEKCSCCQGSGPGVEYFEKLRSALLDGVNKGRSLLKHLPVTRIEGQSFRVDIIKFEDGVRVVKQEYKPIPLLKKKFYVGIRELNDGTYDVSIATKAGELLVKDEESLVIREILSTEGIKKMKLSSWDNPEEALNDLMNALQEASNASAGPFGLIINPKRYAKLLKIYEKSGKMLVEVLKEIFRGGIIVTLNIDENKVIIFANTPAVLDVVVGQDVTLQELGPEGDDVAFLVSEAIGIRIKNPEAIVVLEGSGDYKDDDDK

SEQ ID NO: 213 - protein, 320 aa, Pyrococcus furiosus; identical to SEQ ID NO: 212

SEQ ID NO: 214 - protein, 227 aa, synthetic construct
  REGION 1..227: note = mEos4b
MVSAIKPDMRIKLRMEGNVNGHHFVIDGDGTGKPYEGKQTMDLEVKEGGPLPFAFDILTTAFHYGNRVFVKYPDNIQDYFKQSFPKGYSWERSLTFEDGGICNARNDITMEGDTFYNKVRFYGTNFPANGPVMQKKTLKWEPSTEKMYVRDGVLTGDIEMALLLEGNAHYRCDFRTTYKAKEKGVKLPGAHFVDHAIEILSHDKDYNKVKLYEHAVAHSGLPDNARR

SEQ ID NO: 215 - protein, 227 aa, synthetic construct
  REGION 1..227: note = mEosEM
MVSAIKPDMRIKLRMEGNVNGHHFVIDGEGTGKPYEGKQTMDLEVKEGGPLPFAFDILTTAFHYGNRVFVKYPDNIQDYFKQSFPKGYSWERSMTFEDGGICNARNDITMEGDTFYNKVRFYGTNFPANGPVMQKKTLKWEPSTEKMYVRDGVLTGDIEMALLLEGGAHYRCDFRTTYKAKEKGVKLPGAHFVDHAIEILSHDKDYNKVKLYEHAVAHSGLPDNARR

SEQ ID NO: 216 - protein, 316 aa, synthetic construct
  REGION 1..316: note = iRFP713
MAEGSVARQPDLLTCDDEPIHIPGAIQPHGLLLALAADMTIVAGSDNLPELTGLAIGALIGRSAADVFDSETHNRLTIALAEPGAAVGAPITVGFTMRKDAGFIGSWHRHDQLIFLELEPPQRDVAEPQAFFRRTNSAIRRLQAAETLESACAAAAQEVRKITGFDRVMIYRFASDFSGEVIAEDRCAEVESKLGLHYPASTVPAQARRLYTINPVRIIPDINYRPVPVTPDLNPVTGRPIDLSFAILRSVSPVHLEFMRNIGMHGTMSISILRGERLWGLIVCHHRTPYYVDLDGRQACELVAQVLAWQIGVMEE

SEQ ID NO: 217 - protein, 132 aa, synthetic construct
  REGION 1..132: note = TeAPCalpha
MKTGEQRVKIATLLSENEKKIVDKASQDLWRRRPDFIAPGGNAFGQRERALCLRDYGWYLRLITYGLLAGDKDPIESIGLIGVREMYNSLGVPVPGMVESIRCLKEASLSLLDEEDAKETAPYFDYIIQAMS

SEQ ID NO: 218 - protein, 494 aa, Homo sapiens
DLSLSDINSTVEVPEGHSFWKTLLAYSGPGALVAVGYMDPGNWSTSITGGQNFQYLLLSIIVISSLLAMLLQNMAAKLGIVCQLDLAQAIRARTSRRLGFIFWILTELAIMATDIAEVIGAAIALYLLFKIPIFLAVVITVLDVFLLLLLNRIGFRKIEALVVCLIFVILFVFLYQIILSQPAWHQVAKGLIPSWASVQTSPKIGGQTPLSASLGIIGATIMPHNLFLHSAISQSRKIDRTDSSKVAEAVRFSNWDSNIQLSLAMVVNALLLIMGVAVFKSGAVQDPSFFGLYQALSNPDMVSNPVLAEAARSGVLSTLFAVALLASGQNSTITGTITGQVIMEGFIHLRLPLWLRRLVTRLIAIIPVVVCVAITSHQGSLDEHQALNNLMNNSQVFLALALPFSIVPLLMLTDSAAQMGNQFKNTRWVKVMGWLTVIILTLLNLISISSQIAGFFGDNPSSQDLLLSQVISIGIILAMIGLLIWTIIDIRRFT

SEQ ID NO: 219 - protein, 492 aa, Homo sapiens
MKLLLLHPAFQSCLLLTLLGLWRTTPEAHASSLGAPAISAASFLQDLIHRYGEGDSLTLQQLKALLNHLDVGVGRGNVTQHVQGHRNLSTCFSSGDLFTAHNFSEQSRIGSSELQEFCPTILQQLDSRACTSENQENEENEQTEEGRPSAVEVWGYGLLCVTVISLCSLLGASVVPFMKKTFYKRLLLYFIALAIGTLYSNALFQLIPEAFGFNPLEDYYVSKSAVVFGGFYLFFFTEKILKILLKQKNEHHHGHSHYASESLPSKKDQEEGVMEKLQNGDLDHMIPQHCSSELDGKAPMVDEKVIVGSLSVQDLQASQSACYWLKGVRYSDIGTLAWMITLSDGLHNFIDGLAIGASFTVSVFQGISTSVAILCEEFPHELGDFVILLNAGMSIQQALFFNFLSACCCYLGLAFGILAGSHFSANWIFALAGGMFLYISLADMFPEMNEVCQEDERKGSILIPFIIQNLGLLTGFTIMVVLTMYSGQIQIG

SEQ ID NO: 220 - protein, 425 aa, synthetic construct
  REGION 1..425: note = Fusion of Cystathionine gamma-lyase with QtSig
MQEKDASSQGFLPHFQHFATQAIHVGQDPEQWTSRAVVPPISLSTTFKQGAPGQHSGFEYSRSGNPTRNCLEKAVAALDGAKYCLAFASGLAATVTITHLLKAGDQIICMDDVYGGTNRYFRQVASEFGLKISFVDCSKIKLLEAAITPETKLVWIETPTNPTQKVIDIEGCAHIVHKHGDIILVVDNTFMSPYFQRPLALGADISMYSATKYMNGHSDVVMGLVSVNCESLHNRLRFLQNSLGAVPSPIDCYLCNRGLKTLHVRMEKHFKNGMAVAQFLESNPWVEKVIYPGLPSHPQHELVKRQCTGCTGMVTFYIKGTLQHAEIFLKNLKLFTLAESLGGFESLAELPAIMTHASVLKNDRDVLGISDTLIRLSVGLEDEEDLLEDLDQALKAAHPPSGSHSGGGGSGGGGSKGFTVGSLIQ

SEQ ID NO: 221 - protein, 1070 aa, synthetic construct
  REGION 1..1070: note = Fusion of Seap, a secreted form of Human embryonic alkaline phosphatase, and QtSig
MLGPCMLLLLLLLGLRLQLSLGIIPVEEENPDFWNREAAEALGAAKKLQPAQTAAKNLIIFLGDGMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTRVQHASPAGTYAHTVNRNWYSDADVPASARQEGCQDIATQLISNMDIDVILGGGRKYMFRMGTPDPEYPDDYSQGGTRLDGKNLVQEWLAKRQGARYVWNRTELMQASLDPSVTHLMGLFEPGDMKYEIHRDSTLDPSLMEMTEAALRLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDEETHAGEDVAVFARGPQAHLVHGVQEQTFIAHVMAFAACLEPYTACDLAPPAGTTDAAHPGRSVVPALLPLLAGTLLLLETATAPMLGPCMLLLLLLLGLRLQLSLGIIPVEEENPDFWNREAAEALGAAKKLQPAQTAAKNLIIFLGDGMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTRVQHASPAGTYAHTVNRNWYSDADVPASARQEGCQDIATQLISNMDIDVILGGGRKYMFRMGTPDPEYPDDYSQGGTRLDGKNLVQEWLAKRQGARYVWNRTELMQASLDPSVTHLMGLFEPGDMKYEIHRDSTLDPSLMEMTEAALRLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDEETHAGEDVAVFARGPQAHLVHGVQEQTFIAHVMAFAACLEPYTACDLAPPAGTTDAAHPGRSVVPALLPLLAGTLLLLETATAP

SEQ ID NO: 222 - protein, 1237 aa, synthetic construct
  REGION 1..1237: note = Fusion of MnxG, component of the Bacillus sp. Multimeric multicopper oxidase Mnx, and QtSig
MKRCFHVVAIPIRIVVNNFGDYNPNGMMYVLKENEHKVKRLVKKNPFSVVDLVQPLIIRANEGDEVEVLFENQLPFNTSMHFQEAEYDVLTSDGANVGFNPDTTVAPGCKILYRLRLPKEGAYIFSDLGNPSSSEQGSNSNGLFGALFVERRFSWWTDPVTGGPLNSGLYADVHHPILPSFREYAWLFSDEMEINDLTGNNPIDPVTGEPTESFHGVNYRYEPLHRRKQLIDEGVVCPDCDGEEVHHDSWVFGDPATPIFRGYVGDPAKIRLIHAGVKETHVFHYHVHQWFNDPDNLESEIFDSQAASPQSHYDIEPLYGLGSLQRAIGDAIIHCHLYPHFGIGMWGMNRVFDTLQDGSQSYPNGVKIDALQPLPDRPAPPRPTKEKPGFPNFIPGKPGFKAPRPPLGIVGGRGLTELEKNAAVPKARPGAVFADPCIENANVIEYNISLIELPLVYNKQGWHDPKGRIYILDEDIDDVCSGRKEPEPLIIHQPANTCIRINYTNRLPHILDGDAFQLVTRSYEAGMHIHFVKFDVLVNDGANVGWNYDSSILPGETMRYEYYADVELKAWFFHDHLFAVQHQQHGVFGSGVVHPRFTKFIDSNGGGEVGAGAQVTAVNPLIPDYRDFSLMVQDFSLLFDKKGNPIQPPEFPGSQDDPGLFGVNYKNEPLQFRLGPDCDPAYTFSSFVHGDPCTPILRAYEGDPIRIRLLQGAQEESHSFNVHGLRWSKERSDLDSMFREQQHIGISESFTFESYIPRAGDYLWAFETEEDLWNGLWGLIRAYDEVVPDLIPLTDRPRPLKRSRLLPYRTGKPPRKGDCFKDDLIIPHKRCVKEFDIVAFQVPIIYNEFGDVDQNGIVFSLREDMDDILSGLKNPEPLIIRANVGDEVRVKLTSLLEFDKFPFKDGIYPYPTVKEQAFYPPSLRISLHPQLIQYDVKTSSGETVGYNYDQTVGPGESITYKWYVDFPVGACGMWDMADIRNHKSQGAFGAFIAEPRGTEYLDPHTLKPVKTGASVVLRNNLLPDKREFVIIMHDGVRLHDKDGILITDPIDGILLGPDNPFEGDLVDTYDNGSRGFNYRSERLINRYRKHPVLHDLFSSKLFGDPSTPLFESYAGDPVTLRLIAPAERRRSHTFHLHGHYWRKDISDINSVTASFEGFNLAGSKQDFELFGGAGGFNQFPGDYMYRSGNIQWDIEQGMWGIMRVHGKRQPHLPPLEKGGGGSGGGGSKGFTVGSLIQ

SEQ ID NO: 223 - protein, 1237 aa, Bacillus sp.; identical to SEQ ID NO: 222

SEQ ID NO: 224 - protein, 103 aa, Bacillus sp.
MEALFPMSTDYSKMTDVNEIHDSAILEHFRNGIGHKTLVISPSYPYMFVGIIKELIGDTVMIDVETTHFAQLENREWYIHIHNIEVFYIERPGAPKIPKLEDY

SEQ ID NO: 225 - protein, 82 aa, synthetic construct
  REGION 1..82: note = Designed uranyl binder with femtomolar affinity
SNALDCRERIEKDLEDLEKELMEMKSIKLSDDEEAVVERALNYRDDSVYYLEKGDHITSFGCITYAEGLTDSLRMLHRIIEG

SEQ ID NO: 226 - protein, 388 aa, synthetic construct
  REGION 1..388: note = Coiled-coil adapter to recruit an encapsulated cargo, such as the fluorescent protein mEosEM fused to P4 to an encapsulin already harboring an MT3 for dual EM and fluorescence detection.
SPEDEIQQLEEEIAQLEQKNAALKEKNQALKYGSGMDPETCPCPTGGSCTCSDKCKCKGCKCTNCKKSCCSCCPAGCEKCAKDCVCKGEEGAKAEAEKCSCCQGSGGNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEE

SEQ ID NO: 227 - protein, 497 aa, synthetic construct
  REGION 1..497: note = Fusion of the fluorescent protein eUnaG to the outer surface of encapsulin
MDPETCPCPTGGSCTCSDKCKCKGCKCTNCKKSCCSCCPAGCEKCAKDCVCKGEEGAKAEAEKCSCCQGSGGNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGGGSGMLEKFVGTWKIADSHNFGEYLKAIGAPKELSDGGDATTPTLYISQKDGDKMSVKIENGPPTFLDTQVKFKLGEEFDEFPSDRRKGVKSVVNLVGEKLVYVQKWDGKETTYVREIKDGKLVVTLTMGDVVAVRSYRRATE

SEQ ID NO: 228 - protein, 312 aa, synthetic construct
  REGION 1..312: note = CGU for attaching to the outer surface of encapsulin via anti-GFP antibody
MDPETCPCPTGGSCTCSDKCKCKGCKCTNCKKSCCSCCPAGCEKCAKDCVCKGEEGAKAEAEKCSCCQPPPPRTGSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYK

SEQ ID NO: 229 - protein, 418 aa, synthetic construct
  REGION 1..418: note = Fusion of QtEnc and Brain FABP, embodiment of concentric barcodes added to the outer surface of encapsulins
NKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGGGGSGVEAFCATWKLTNSQNFDEYMKALGVGFATRQVGNVTKPTVIISQEGDKVVIRTLSTFKNTEISFQLGEEFDETTADDRNCKSVVSLDGDKLVHIQKWDGKETNFVREIKDGKMVMTLTFGDVVAVRHYEKA

SEQ ID NO: 230 - protein, 742 aa, synthetic construct
  REGION 1..742: note = Fusion of QtEnc and Bm3h, embodiment of concentric barcodes added to the outer surface of encapsulins
NKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGGGGSGTIKEMPQPKTFGELKNLPLLNTDKPVQALMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPL

SEQ ID NO: 231 - protein, 355 aa, synthetic construct
  REGION 1..355: note = Fusion of QtEnc and MT3, embodiment of concentric barcodes added to the outer surface of encapsulins
NKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGGGGSGMDPETCPCPTGGSCTCSDKCKCKGCKCTNCKKSCCSCCPAGCEKCAKDCVCKGEEGAKAEAEKCSCCQ

SEQ ID NO: 232 - protein, 380 aa, synthetic construct
  REGION 1..380: note = Fusion of Brain FABP and superfolder GFP, for attachment via anti-GFP intrabody attachment points, e.g. to the outer surface of Encapsulins.
VEAFCATWKLTNSQNFDEYMKALGVGFATRQVGNVTKPTVIISQEGDKVVIRTLSTFKNTEISFQLGEEFDETTADDRNCKSVVSLDGDKLVHIQKWDGKETNFVREIKDGKMVMTLTFGDVVAVRHYEKAPPPPRTGPPPPRSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYK

SEQ ID NO: 233 - protein, 704 aa, synthetic construct
  REGION 1..704: note = Fusion of Bm3h and superfolder GFP, or attachment via anti-GFP intrabody attachment points, e.g. to the outer surface of Encapsulins.
TIKEMPQPKTFGELKNLPLLNTDKPVQALMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLPPPPRTGPPPPRSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYK

SEQ ID NO: 234 - protein, 204 aa, synthetic construct
  REGION 1..204: note = Fusion of MT3 and Lam4, for attachment to mCherry, e.g. on SasG
MDPETCPCPTGGSCTCSDKCKCKGCKCTNCKKSCCSCCPAGCEKCAKDCVCKGEEGAKAEAEKCSCCQPPPPRTGPPPPRAQVQLVESGGSLVQPGGSLRLSCAASGRFAESSSMGWFRQAPGKEREFVAAISWSGGATNYADSAKGRFTLSRDNTKNTVYLQMNSLKPDDTAVYYCAANLGNYISSNQRLYGYWGQGTQVTVS

SEQ ID NO: 235 - protein, 267 aa, synthetic construct
  REGION 1..267: note = Fusion of Brain FABP and Lam4, , for attachment to mCherry, e.g. on SasG
VEAFCATWKLTNSQNFDEYMKALGVGFATRQVGNVTKPTVIISQEGDKVVIRTLSTFKNTEISFQLGEEFDETTADDRNCKSVVSLDGDKLVHIQKWDGKETNFVREIKDGKMVMTLTFGDVVAVRHYEKAPPPPRTGPPPPRAQVQLVESGGSLVQPGGSLRLSCAASGRFAESSSMGWFRQAPGKEREFVAAISWSGGATNYADSAKGRFTLSRDNTKNTVYLQMNSLKPDDTAVYYCAANLGNYISSNQRLYGYWGQGTQVTVS

SEQ ID NO: 236 - protein, 591 aa, synthetic construct
  REGION 1..591: note = Fusion of Bm3h and Lam4, , for attachment to mCherry, e.g. on SasG
TIKEMPQPKTFGELKNLPLLNTDKPVQALMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLPPPPRTGPPPPRAQVQLVESGGSLVQPGGSLRLSCAASGRFAESSSMGWFRQAPGKEREFVAAISWSGGATNYADSAKGRFTLSRDNTKNTVYLQMNSLKPDDTAVYYCAANLGNYISSNQRLYGYWGQGTQVTVS

SEQ ID NO: 237 - protein, 4 aa, synthetic construct
  REGION 1..4: note = General linker used for protein fusions, including direct fusions of CGUs to achieve the nanometer sizes of a structural element
GGSG

SEQ ID NO: 238 - protein, 201 aa, synthetic construct
  REGION 1..201: note = myristoyl switch motif for calicum-rependent relocalization a structural element such as vaults to the membrane
GNSKSGALSKEILEELQLNTKFTEEELSSWYQSFLKECPSGRITRQEFQTIYSKFFPEADPKAYAQHVFRSFDANSDGTLDFKEYVIALHMTSAGKTNQKLEWAFSLYDVDGNGTISKNEVLEIVTAIFKMISPEDTKHLPEDENTPEKRAEKIWGFFGKKDDDKLTEKEFIEGTLANKEILRLIQFEPQKVKEKLKEKKL

SEQ ID NO: 239 - protein, 15 aa, synthetic construct
  REGION 1..15: note = synthide-2 binding for FHA2
PLARALTVAGLPGKK

SEQ ID NO: 240 - protein, 599 aa, synthetic construct
MDPETCPCPTGGSCTCSDKCKCKGCKCTNCKKSCCSCCPAGCEKCAKDCVCKGEEGAKAEAEKCSCCQGSGPGNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGGGGSGPGGSGDPETCPCPTGGSCTCSDKCKCKGCKCTNCKKSCCSCCPAGCEKCAKDCVCKGEEGAKAEAEKCSCCQGSGLEKFVGTWKIADSHNFGEYLKAIGAPKELSDGGDATTPTLYISQKDGDKMSVKIENGPPTFLDTQVKFKLGEEFDEFPSDRRKGVKSVVNLVGEKLVYVQKWDGKETTYVREIKDGKLVVTLTMGDVVAVRSYRRATEGSSEGGRRKWQKTGHAVRAIGRLSSS

SEQ ID NO: 241 - protein, 521 aa, synthetic construct
MDPETCPCPTGGSCTCSDKCKCKGCKCTNCKKSCCSCCPAGCEKCAKDCVCKGEEGAKAEAEKCSCCQGSGPGNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGSGLEKFVGTWKIADSHNFGEYLKAIGAPKELSDGGDATTPTLYISQKDGDKMSVKIENGPPTFLDTQVKFKLGEEFDEFPSDRRKGVKSVVNLVGEKLVYVQKWDGKETTYVREIKDGKLVVTLTMGDVVAVRSYRRATEGSSEGGRRKWQKTGHAVRAIGRLSSS

SEQ ID NO: 242 - protein, 448 aa, synthetic construct
NKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGSGLEKFVGTWKIADSHNFGEYLKAIGAPKELSDGGDATTPTLYISQKDGDKMSVKIENGPPTFLDTQVKFKLGEEFDEFPSDRRKGVKSVVNLVGEKLVYVQKWDGKETTYVREIKDGKLVVTLTMGDVVAVRSYRRATEGSSEGGRRKWQKTGHAVRAIGRLSSS

SEQ ID NO: 243 - protein, 857 aa, synthetic construct
MDPETCPCPTGGSCTCSDKCKCKGCKCTNCKKSCCSCCPAGCEKCAKDCVCKGEEGAKAEAEKCSCCQGSGPGNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGSGLEKFVGTWKIADSHNFGEYLKAIGAPKELSDGGDATTPTLYISQKDGDKMSVKIENGPPTFLDTQVKFKLGEEFDEFPSDRRKGVKSVVNLVGEKLVYVQKWDGKETTYVREIKDGKLVVTLTMGDVVAVRSYRRATEGSSEGGRRKWQKTGHAVRAIGRLSSSGSGATNFSLLKQAGDVEENPGPADQLTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAKSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 244 - protein, 935 aa, synthetic construct
MDPETCPCPTGGSCTCSDKCKCKGCKCTNCKKSCCSCCPAGCEKCAKDCVCKGEEGAKAEAEKCSCCQGSGPGNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGGGGSGPGGSGDPETCPCPTGGSCTCSDKCKCKGCKCTNCKKSCCSCCPAGCEKCAKDCVCKGEEGAKAEAEKCSCCQGSGLEKFVGTWKIADSHNFGEYLKAIGAPKELSDGGDATTPTLYISQKDGDKMSVKIENGPPTFLDTQVKFKLGEEFDEFPSDRRKGVKSVVNLVGEKLVYVQKWDGKETTYVREIKDGKLVVTLTMGDVVAVRSYRRATEGSSEGGRRKWQKTGHAVRAIGRLSSSGSGATNFSLLKQAGDVEENPGPADQLTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAKSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 245 - protein, 1539 aa, synthetic construct
MKEELDAFHQIFTTTKEAIERFMAMLTPVIENAEDDHERLYYHHIYEEEEQRLSRLDVLIPLIEKFQDETDEGLFSPSNNAFNRLLQELNLEKFGLHNFIEHVDLALFSFTDEERQTLLKELRKDAYEGYQYVKEKLAEINARFDHDYADPHAHHDEHRDHLADMPSAGSSHEEVQPVAHKKKGFTVGSLIQGSGATNFSLLKQAGDVEENPGPMNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGSGGGGGSGPGGSGLEKFVGTWKIADSHNFGEYLKAIGAPKELSDGGDATTPTLYISQKDGDKMSVKIENGPPTFLDTQVKFKLGEEFDEFPSDRRKGVKSVVNLVGEKLVYVQKWDGKETTYVREIKDGKLVVTLTMGDVVAVRSYRRATEGSSEGGRRKWQKTGHAVRAIGRLSSSGSGATNFSLLKQAGDVEENPGPMADQLTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAKSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGSGATNFSLLKQAGDVEENPGPMKRLHPALPSCLLLVLFGIWRTAPQTHASSAGLPPLSATSFLEDLMDRYGKNDSLTTQLKSLLDHLHVGVGRDNVSQPKEGPRNLSTCFSSGDLFAAHNLSERSQIGASEFQEFCPTILQQLDSQACTSENQKSEENEQTEEGKPSAIEVWGFGFLSVSLINLASLLGVLVLPCTEKAFFSRVLTYFIALSIGTLLSNALFQLIPEAFGFNPQDNYVSKSAVVFGGFYLFFFTEKILKMLLKQKNEHHHGHNHFTSETLPSKKDQEEGVTEKLQNGDLDHMIPQHCNSELDGKAPGTDEKVIVNSMSVQDLQASQSACYWLKGVRYSDIGTLAWMITLSDGLHNFIDGLAIGASFTVSVFQGISTSVAILCEEFPHELGDFVILLNAGMSIQQALFFNFLSACCCYLGLAFGILAGSHFSANWIFALAGGMFLYIALADMFPEMNEVCQEDEKNDSFLVPFVIQNLGLLTGFSIMLVLTMYSGQIQIGGGGGSGGGGSDYKDDDDK

SEQ ID NO: 246 - protein, 477 aa, synthetic construct
MDPETCPCPTGGSCTCSDKCKCKGCKCTNCKKSCCSCCPAGCEKCAKDCVCKGEEGAKAEAEKCSCCQGSGPGVEYFEKLRSALLDGVNKGRSLLKHLPVTRIEGQSFRVDIIKFEDGVRVVKQEYKPIPLLKKKFYVGIRELNDGTYDVSIATKAGELLVKDEESLVIREILSTEGIKKMKLSSWDNPEEALNDLMNALQEASNASAGPFGLIINPKRYAKLLKIYEKSGKMLVEVLKEIFRGGIIVTLNIDENKVIIFANTPAVLDVVVGQDVTLQELGPEGDDVAFLVSEAIGIRIKNPEAIVVLEGSGDYKDDDDKGGGGSGPGGSGANLDKMLNTTVTEVRQFLQVDRVCVFQFEEDYSGVVVVEAVDDRWISILKTQVRDRYFMETRGEEYSHGRYQAIADIYTANLTECYRDLLTQFQVRAILAVPILQGKKLWGLLVAHQLAAPRQWQTWEIDFLKQQAVVVGIAIQQS

SEQ ID NO: 247 - protein, 445 aa, synthetic construct
MDPETCPCPTGGSCTCSDKCKCKGCKCTNCKKSCCSCCPAGCEKCAKDCVCKGEEGAKAEAEKCSCCQGSGPGVEYFEKLRSALLDGVNKGRSLLKHLPVTRIEGQSFRVDIIKFEDGVRVVKQEYKPIPLLKKKFYVGIRELNDGTYDVSIATKAGELLVKDEESLVIREILSTEGIKKMKLSSWDNPEEALNDLMNALQEASNASAGPFGLIINPKRYAKLLKIYEKSGKMLVEVLKEIFRGGIIVTLNIDENKVIIFANTPAVLDVVVGQDVTLQELGPEGDDVAFLVSEAIGIRIKNPEAIVVLEGSGDYKDDDDKGGGGSGPGGSGVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSS

SEQ ID NO: 248 - protein, 131 aa, synthetic construct
VEAFCATWKLTNSQNWDEYMKALGVGFATRQVGNVTKPTVIISQEGDKVVIRRLSTFKNTEISFQLGEEFDETTADDRNCKSVVSLDGDKLVHIQKWDGKETNMVMEIKDGKMVMTLTFGDVVAVSHWEKA

SEQ ID NO: 249 - protein, 464 aa, synthetic construct
MTIKEMPQPKTFGELKNLPLLNTDKPVQALMKIADELGEIFKFEAPGRVTRYLSSQRLIKEACDESRFDKNLSQAPKFVRDLAGDGLFTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLRRANPDDPAYDENKRQFQEDIKVMNDLVDKIIADRKASGEQSDDLLTHMLNGKDPETGEPLDDENIRYQIITFLAAGHEATSGLLSFALYFLVKNPHELQKAAEEAARVLVDPVPSHKQVKQLKYVGMVLNEALRLWPTAPAFSLYAKEDTVLGGEYPLEKGDELMVLIPQLHRDKTVWGDDVEEFRPERFENPSAIPQHAFKPFGNGQRACIGQQFALHEATLVLGMMLKHFDFEDHTNYELDIKETLTLKPEGFVVKAKSKKIPLGGIPSPST

SEQ ID NO: 250 - protein, 415 aa, synthetic construct
MNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGGVEAFCATWKLTNSQNWDEYMKALGVGFATRQVGNVTKPTVIISQEGDKVVIRRLSTFKNTEISFQLGEEFDETTADDRNCKSVVSLDGDKLVHIQKWDGKETNMVMEIKDGKMVMTLTFGDVVAVSHWEKA

SEQ ID NO: 251 - protein, 297 aa, synthetic construct
VEAFCATWKLTNSQNFDEYMKALGVGFATRQVGNVTKPTVIISQEGDKVVIRTLSTFKNTEISFQLGEEFDETTADDRNCKSVVSLDGDKLVHIQKWDGKETNFVREIKDGKMVMTLTFGDVVAVRHYEKASGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 252 - protein, 298 aa, synthetic construct
MVEAFCATWKLTNSQNWDEYMKALGVGFATRQVGNVTKPTVIISQEGDKVVIRRLSTFKNTEISFQLGEEFDETTADDRNCKSVVSLDGDKLVHIQKWDGKETNMVMEIKDGKMVMTLTFGDVVAVSHWEKASGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 253 - protein, 621 aa, synthetic construct
TIKEMPQPKTFGELKNLPLLNTDKPVQALMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 254 - protein, 629 aa, synthetic construct
TIKEMPQPKTFGELKNLPLLNTDKPVQALMKIADELGEIFKFEAPGRVTRYLSSQRLIKEACDESRFDKNLSQAPKFVRDLAGDGLFTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLRRANPDDPAYDENKRQFQEDIKVMNDLVDKIIADRKASGEQSDDLLTHMLNGKDPETGEPLDDENIRYQIITFLAAGHEATSGLLSFALYFLVKNPHELQKAAEEAARVLVDPVPSHKQVKQLKYVGMVLNEALRLWPTAPAFSLYAKEDTVLGGEYPLEKGDELMVLIPQLHRDKTVWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 255 - protein, 142 aa, synthetic construct
VEAFCATWKLTNSQNFDEYMKALGVGFATRQVGNVTKPTVIISQEGDKVVIRTLSTFKNTEISFQLGEEFDETTADDRNCKSVVSLDGDKLVHIQKWDGKETNFVREIKDGKMVMTLTFGDVVAVRHYEKAGSGDYKDDDDK

SEQ ID NO: 256 - protein, 142 aa, synthetic construct
VEAFCATWKLTNSQNWDEYMKALGVGFATRQVGNVTKPTVIISQEGDKVVIRRLSTFKNTEISFQLGEEFDETTADDRNCKSVVSLDGDKLVHIQKWDGKETNMVMEIKDGKMVMTLTFGDVVAVSHWEKAGSGDYKDDDDK

SEQ ID NO: 257 - protein, 466 aa, synthetic construct
TIKEMPQPKTFGELKNLPLLNTDKPVQALMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGSGDYKDDDDK

SEQ ID NO: 258 - protein, 474 aa, synthetic construct
TIKEMPQPKTFGELKNLPLLNTDKPVQALMKIADELGEIFKFEAPGRVTRYLSSQRLIKEACDESRFDKNLSQAPKFVRDLAGDGLFTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLRRANPDDPAYDENKRQFQEDIKVMNDLVDKIIADRKASGEQSDDLLTHMLNGKDPETGEPLDDENIRYQIITFLAAGHEATSGLLSFALYFLVKNPHELQKAAEEAARVLVDPVPSHKQVKQLKYVGMVLNEALRLWPTAPAFSLYAKEDTVLGGEYPLEKGDELMVLIPQLHRDKTVWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTGSGDYKDDDDK

SEQ ID NO: 259 - protein, 260 aa, synthetic construct
MEVQLVESGGGLVQPGGSLRLSCAASGSISSVDVMSWYRQAPGKQRELVAFITDRGRTNYKVSVKGRFTISRDNSKNMVYLQMNSLKPEDTADYLCRAESRTSWSSPSPLDVWGRGTQVTVSSGGTGGSVEAFCATWKLTNSQNFDEYMKALGVGFATRQVGNVTKPTVIISQEGDKVVIRTLSTFKNTEISFQLGEEFDETTADDRNCKSVVSLDGDKLVHIQKWDGKETNFVREIKDGKMVMTLTFGDVVAVRHYEKA

SEQ ID NO: 260 - protein, 541 aa, synthetic construct
MDPETCPCPTGGSCTCSDKCKCKGCKCTNCKKSCCSCCPAGCEKCAKDCVCKGEEGAKAEAEKCSCCQGSGPGNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGGTGGSDKDCEMKRTTLDSPLGKLELSGCEQGLHRIIFLGKGTSAADAVEVPAPAAVLGGPEPLMQATAWLNAYFHQPEAIEEFPVPALHHPVFQQESFTRQVLWKLLKVVKFGEVISYSHLAALAGNPAATAAVKTALSGNPVPILIPCHRVVQGDLDVGGYEGGLAVKEWLLAHEGHRLGKPGLG

SEQ ID NO: 261 - protein, 541 aa, synthetic construct
MDPETCPCPTGGSCTCSDKCKCKGCKCTNCKKSCCSCCPAGCEKCAKDCVCKGEEGAKAEAEKCSCCQGSGPGNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGGTGGSDKDCEMKRTTLDSPLGKLELSGCEQGLHEIIFLGKGTSAADAVEVPAPAAVLGGPEPLIQATAWLNAYFHQPEAIEEFPVPALHHPVFQQESFTRQVLWKLLKVVKFGEVISESHLAALVGNPAATAAVNTALDGNPVPILIPCHRVVQGDSDVGPYLGGLAVKEWLLAHEGHRLGKPGLG

SEQ ID NO: 262 - protein, 620 aa, synthetic construct
MGVKVLFALICIAVAEAKRGTQPTFGFTVNWKFSESTTVFTGQCFIDRNGKEVLKTMWLLRSSVNDIGDDWKATRVGINIFTRLRTQKEGGSGGSARKCSLTGKWTNDLGSNMTIGAVNSRGEFTGTYITAVTATSNEIKESPLHGTQNTINKSGGSTTVFTGQCFIDRNGKEVLKTMWLLRSSVNDIGDDWKATRVGINIFTRLRTQKEGGSGGSARKCSLTGKWTNDLGSNMTIGAVNSRGEFTGTYITAVTATSNEIKESPLHGTQNTINKRTQPTFGFTVNWKFSEGGGGSGGGGSGENLYFQSGGGGSGGGGSGGGGSAHHFSEPEITLIIFGVMAGVIGTILLISYGIRRLIKKSPSGGGGSGGGGSGGGGSGGGGSTGVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 263 - protein, 872 aa, synthetic construct
MGVKVLFALICIAVAEAKRGTQPTFGFTVNWKFSESTTVFTGQCFIDRNGKEVLKTMWLLRSSVNDIGDDWKATRVGINIFTRLRTQKEGGSGGSARKCSLTGKWTNDLGSNMTIGAVNSRGEFTGTYITAVTATSNEIKESPLHGTQNTINKSGGSTTVFTGQCFIDRNGKEVLKTMWLLRSSVNDIGDDWKATRVGINIFTRLRTQKEGGSGGSARKCSLTGKWTNDLGSNMTIGAVNSRGEFTGTYITAVTATSNEIKESPLHGTQNTINKRTQPTFGFTVNWKFSEGGSGSGSGSGSGRTQPTFGFTVNWKFSESTTVFTGQCFIDRNGKEVLKTMWLLRSSVNDIGDDWKATRVGINIFTRLRTQKEGGSGGSARKCSLTGKWTNDLGSNMTIGAVNSRGEFTGTYITAVTATSNEIKESPLHGTQNTINKSGGSTTVFTGQCFIDRNGKEVLKTMWLLRSSVNDIGDDWKATRVGINIFTRLRTQKEGGSGGSARKCSLTGKWTNDLGSNMTIGAVNSRGEFTGTYITAVTATSNEIKESPLHGTQNTINKRTQPTFGFTVNWKFSEGGGGSGGGGSGGGGSGGGGSAHHFSEPEITLIIFGVMALVIGTILLISYGIRRLIKKSPSGGGGSTGSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANAKTTYRSKKPAKNLKMPGVYYVDYRLERIKEANNETYVEQHEVAVARYCDLPSKLGHKLN

SEQ ID NO: 264 - protein, 227 aa, synthetic construct; identical to SEQ ID NO: 215

SEQ ID NO: 265 - protein, 187 aa, synthetic construct
LPFAFDILTTAFHYGNRVFVKYPDNIQDYFKQSFPKGYSWERSMTFEDGGICNARNDITMEGDTFYNKVRFYGTNFPANGPVMQKKTLKWEPSTEKMYVRDGVLTGDIEMALLLEGGAHYRCDFRTTYKAKEKGVKLPGAHFVDHAIEILSHDKDYNKVKLYEHAVAHSGLPDNARRKGFTVGSLIQ

SEQ ID NO: 266 - protein, 57 aa, synthetic construct
GDHYDDELESEVDDIRTALAKIHEDNQKIISKLESLLLLKGEVESIKKQINRQNISI

SEQ ID NO: 267 - protein, 552 aa, synthetic construct
MDKDCEMKRTTLDSPLGKLELSGCEQGLHRIIFLGKGTSAADAVEVPAPAAVLGGPEPLMQATAWLNAYFHQPEAIEEFPVPALHHPVFQQESFTRQVLWKLLKVVKFGEVISYSHLAALAGNPAATAAVKTALSGNPVPILIPCHRVVQGDLDVGGYEGGLAVKEWLLAHEGHRLGKPGLGGGTGGSDPETCPCPTGGSCTCSDKCKCKGCKCTNCKKSCCSCCPAGCEKCAKDCVCKGEEGAKAEAEKCSCCQGSGPGNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGSGDYKDDDDK

SEQ ID NO: 268 - protein, 30 aa, synthetic construct
GEIAQTIEEIADTIREIAYTIKEIAQTIKG

SEQ ID NO: 269 - protein, 32 aa, synthetic construct
VSRLEEDVRNLEAEVDKLRERLDRLEETVQAK

SEQ ID NO: 270 - protein, 32 aa, synthetic construct
VSRLEERVRDLEAEVDKLRERLDRLEETVQAK

SEQ ID NO: 271 - protein, 61 aa, synthetic construct
QLEDKVEELLSKVYHLENEVDRLRKLIENKADMKQLEDKVEELESEVDHLRNEVARLKKLV

SEQ ID NO: 272 - protein, 485 aa, synthetic construct
NKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEPPPPRPPPPRVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSPPPPRPPPPRKRRWKKNFIAVSAANRFKKISSSGAL

SEQ ID NO: 273 - protein, 300 aa, synthetic construct
VTGYRLFEEILPPPPRVEAFCATWKLTNSQNFDEYMKALGVGFATRQVGNVTKPTVIISQEGDKVVIRTLSTFKNTEISFQLGEEFDETTADDRNCKSVVSLDGDKLVHIQKWDGKETNFVREIKDGKMVMTLTFGDVVAVRHYEKAPPPPRADQLTDEQIAEFKEAFSLFDKDGDGTITTKELGTVMRSLGQNPTEAELQDMINEVDADGNGTIDFPEFLNLMARKMKDTDSEEKLKEAFRVFDKDGNGFISAAELRHVMTNLGEKLTDEEVDEMIREADVDGDGQVNYEEFVQVMMAK

SEQ ID NO: 274 - protein, 300 aa, synthetic construct
VTGYRLFEEILPPPPRADQLTDEQIAEFKEAFSLFDKDGDGTITTKELGTVMRSLGQNPTEAELQDMINEVDADGNGTIDFPEFLNLMARKMKDTDSEEKLKEAFRVFDKDGNGFISAAELRHVMTNLGEKLTDEEVDEMIREADVDGDGQVNYEEFVQVMMAKPPPPRVEAFCATWKLTNSQNFDEYMKALGVGFATRQVGNVTKPTVIISQEGDKVVIRTLSTFKNTEISFQLGEEFDETTADDRNCKSVVSLDGDKLVHIQKWDGKETNFVREIKDGKMVMTLTFGDVVAVRHYEKA

SEQ ID NO: 275 - protein, 602 aa, synthetic construct
NKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEPPPPRPPPPRVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSPPPPRADQLTDEQIAEFKEAFSLFDKDGDGTITTKELGTVMRSLGQNPTEAELQDMINEVDADGNGTIDFPEFLNLMARKMKDTDSEEKLKEAFRVFDKDGNGFISAAELRHVMTNLGEKLTDEEVDEMIREADVDGDGQVNYEEFVQVMMAK

SEQ ID NO: 276 - protein, 2841 aa, synthetic construct
RGVPHIVMVDAYKRYKGGSGGGSGADQLTDEQIAEFKEAFSLFDKDGDGTITTKELGTVMRSLGQNPTEAELQDMINEVDADGNGTIDFPEFLNLMARKMKDTDSEEKLKEAFRVFDKDGNGFISAAELRHVMTNLGEKLTDEEVDEMIREADVDGDGQVNYEEFVQVMMAKGGSGGGSGAPKTITELEKKVEEIPFKKERKFNPDLAPGTEKVTREGQKGEKTITTPTLKNPLTGVIISKGEPKEEITKDPINELTEYGPETIAPGHRDEFDPKLPTGEKEEVPGKPGIKNPETGDVVRPPVDSVTKYGYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPEGGGSGGGSSGGGGSGGSSGGGSGPKTITELEKKVEEIPFKKERKFNPDLAPGTEKVTREGQKGEKTITTPTLKNPLTGVIISKGEPKEEITKDPINELTEYGPETIAPGHRDEFDPKLPTGEKEEVPGKPGIKNPETGDVVRPPVDSVTKYGYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPEGGSGVEAFCATWKLTNSQNFDEYMKALGVGFATRQVGNVTKPTVIISQEGDKVVIRTLSTFKNTEISFQLGEEFDETTADDRNCKSVVSLDGDKLVHIQKWDGKETNFVREIKDGKMVMTLTFGDVVAVRHYEKAGGSGGSSGKRRWKKNFIAVSAANRFKKISSSGAL

SEQ ID NO: 277 - protein, 434 aa, synthetic construct
NKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGGGSGADQLTDEQIAEFKEAFSLFDKDGDGTITTKELGTVMRSLGQNPTEAELQDMINEVDADGNGTIDFPEFLNLMARKMKDTDSEEKLKEAFRVFDKDGNGFISAAELRHVMTNLGEKLTDEEVDEMIREADVDGDGQVNYEEFVQVMMAK

SEQ ID NO: 278 - protein, 445 aa, synthetic construct
KRRWKKNFIAVSAANRFKKISSSGALGGGSGVEAFCATWKLTNSQNFDEYMKALGVGFATRQVGNVTKPTVIISQEGDKVVIRTLSTFKNTEISFQLGEEFDETTADDRNCKSVVSLDGDKLVHIQKWDGKETNFVREIKDGKMVMTLTFGDVVAVRHYEKAGGGSGSELIKENMHMKLYMEGTVNNHHFKCTSEGEGKPYEGTQTMRIKVVEGGPLPFAFDILATSFMYGSRTFINHTQGIPDFFKQSFPEGFTWERVTTYEDGGVLTATQDTSLQDGCLIYNVKIRGVNFPSNGPVMQKKTLGWEANTEMLYPADGGLEGRSDMALKLVGGGHLICNFKTTYRSKKPAKNLKMPGVYYVDHRLERIKEADKETYVEQHEVAVARYCDLPSKLGHKSRGGSGNEKEELKSKKAELRNRIEQLKQKREQLKQKIANLRKEIEAYK

SEQ ID NO: 279 - protein, 284 aa, synthetic construct
MSGGEELFAGIVPVLIELDGDVHGHKFSVRGEGEGDADYGKLEIKFICTTGKLPVPWPTLVTTLCYGIQCFARYPEHMKMNDFFKSAMPEGYIQERTIQFQDDGKYKTRGEVKFEGDTLVNRIELKGKDFKEDGNILGHKLEYSFNSHNVYIRPDKANNGLEANFKTRHNIEGGGVQLADHYQTNVPLGDGPVLIPINHYLSTQTKISKDRNEARDHMVLLESFSACCHTHGMDELYRGGGSGFYAEERELKALDRELNAIDKELRANENELRALDNELTAAIS

SEQ ID NO: 280 - protein, 720 aa, synthetic construct
ESLFKGPRDYNPISSTICHLTNESDGHTTSLYGIGFGPFIITNKHLFRRNNGTLLVQSLHLEDQLTEEQIAEFKELFSLFDKDGDGTITTKELGTVMRSLGQNPTEAELQDMINEVDADGDGTIDFPEFLTMMARKMKDTGGVKLIPSWTTVILVKSMLRKRSFGNPFGGDSEEEIREAFGVFDKDGNGYISAAELRHVMTNLGEKLTDEEVDEMIREADIDGDGQVNYEEFVQMMTAKAAAGVFKVKNTTTLQQHLIDGRDMIIIRMPKDFPPFPQKLKFREPQREERICLVTTNFQTKSMSSMVSDTSCTFPSSDGIFWKHWIQTKDGQCGNPLVSTRDGFIVGIHSASNFTNTNNYFTSVPKNFMELLTNQEAQQWVSGWRLNADSVLWGGHKVFMVELAEKLAGLDINGGASEFRATTLERIEKSFVITDPRLPDNPIIFVSDSFLQLTEYSREEILGRNCRFLQGPETDRATVRKIRDAIDNQTEVTVQLINYTKSGKKFWNVFHLQPMRDYKGDVQYFIGVQLDGTERLHGAAEREAVCLVKKTAFQIAENLYFQMGGGSGGRGVPHIVMVDAYKRYKGGGSGVEAFCATWKLTNSQNFDEYMKALGVGFATRQVGNVTKPTVIISQEGDKVVIRTLSTFKNTEISFQLGEEFDETTADDRNCKSVVSLDGDKLVHIQKWDGKETNFVREIKDGKMVMTLTFGDVVAVRHYEKA

SEQ ID NO: 281-283 - skipped (3 entries)

SEQ ID NO: 284 - protein, 1179 aa, synthetic construct
MADIEQELERLKASIRRLEQEVNQERARMAALQTLLAKGGSGGATEEAIIRIPPYHYIHVLDQNSNVSRVEVGPKTYIRQDNERVLFAPVRMVTVPPRHYCIVANPVSRDTQSSVLFDITGQVRLRHADQEIRLAQDPFPLYPGEVLEKDITPLQVVLPNTALHLKALLDFEDKNGDKVMAGDEWLFEGPGTYIPQKEVEVVEIIQATVIKQNQALRLRARKECFDREGKGRVTGEEWLVRSVGAYLPAVFEEVLDLVDAVILTEKTALHLRALQNFRDLRGVLHRTGEEWLVTVQDTEAHVPDVYEEVLGVVPITTLGPRHYCVILDPMGPDGKNQLGQKRVVKGEKSFFLQPGERLERGIQDVYVLSEQQGLLLKALQPLEEGESEEKVSHQAGDCWLIRGPLEYVPSAKVEVVEERQAIPLDQNEGIYVQDVKTGKVRAVIGSTYMLTQDEVLWEKELPSGVEELLNLGGGGSPEDKLQAIKYELAQNEEELAQIEEKLAANKEGGSGGGGSGSKTIVLSVGEATRTLTEIQSTADRQIFEEKVGPLVGRLRLTASLRQNGAKTAYRVNLKLDQADVVDSGLPKVRYTQVWSHDVTIVANSTEASRKSLYDLTKSLVATSQVEDLVVNLVPLGRGPGSGGGGSGGPEDENAALEEKIAQLKQKNAALKEEIQALEYSAPRNKTRVVSYRVPHNAAVQVYDYRAKRARVVFGPELVTLDPEEQFTVLSLSAGRPKRPHARRALCLLLGPDFFTDVITIETADHARLQLQLAYNWHFELKNRNDPAEAAKLFSVPDFVGDACKAIASRVRGAVASVTFDDFHKNSARIIRMAVFGFEGSPEDELAANEEELQQNEQKLAQIKQKLQAIKYGGGGSGGGGSGGGGHHHHHHGGGGSGGGGSGGGGSPEDEIQQLEEEIAQLEQKNAALKEKNQALKYGKARDQAVFPQNGLVVSSVDVQSVEPVDQRTRDALQRSVQLAIEITTNSQEAAAKHEAQRLEQEARGRLERQKILDQSEAEKARKELLELEAMSMAVESTGNAKAEAESRAEAARIEGEGSVLQAKLKAQALAIETEAELERVKKVREMELIYARAQLELEVSKAQQLANVEAKKFKEMTEALGPGTIRDLAVAGPEMQVKLLQSLGLKSTLITDGSSPINLFSTAFGLLGLGSDGQPPAQK

SEQ ID NO: 285 - protein, 1137 aa, synthetic construct
MATEEAIIRIPPYHYIHVLDQNSNVSRVEVGPKTYIRQDNERVLFAPVRMVTVPPRHYCIVANPVSRDTQSSVLFDITGQVRLRHADQEIRLAQDPFPLYPGEVLEKDITPLQVVLPNTALHLKALLDFEDKNGDKVMAGDEWLFEGPGTYIPQKEVEVVEIIQATVIKQNQALRLRARKECFDREGKGRVTGEEWLVRSVGAYLPAVFEEVLDLVDAVILTEKTALHLRALQNFRDLRGVLHRTGEEWLVTVQDTEAHVPDVYEEVLGVVPITTLGPRHYCVILDPMGPDGKNQLGQKRVVKGEKSFFLQPGERLERGIQDVYVLSEQQGLLLKALQPLEEGESEEKVSHQAGDCWLIRGPLEYVPSAKVEVVEERQAIPLDQNEGIYVQDVKTGKVRAVIGSTYMLTQDEVLWEKELPSGVEELLNLGGGGSPEDKLQAIKYELAQNEEELAQIEEKLAANKEGGSGGGGSGSKTIVLSVGEATRTLTEIQSTADRQIFEEKVGPLVGRLRLTASLRQNGAKTAYRVNLKLDQADVVDSGLPKVRYTQVWSHDVTIVANSTEASRKSLYDLTKSLVATSQVEDLVVNLVPLGRGPGSGGGGSGGPEDENAALEEKIAQLKQKNAALKEEIQALEYSAPRNKTRVVSYRVPHNAAVQVYDYRAKRARVVFGPELVTLDPEEQFTVLSLSAGRPKRPHARRALCLLLGPDFFTDVITIETADHARLQLQLAYNWHFELKNRNDPAEAAKLFSVPDFVGDACKAIASRVRGAVASVTFDDFHKNSARIIRMAVFGFEGSPEDELAANEEELQQNEQKLAQIKQKLQAIKYGGGGSGGGGSGGGGHHHHHHGGGGSGGGGSGGGGSPEDEIQQLEEEIAQLEQKNAALKEKNQALKYGKARDQAVFPQNGLVVSSVDVQSVEPVDQRTRDALQRSVQLAIEITTNSQEAAAKHEAQRLEQEARGRLERQKILDQSEAEKARKELLELEAMSMAVESTGNAKAEAESRAEAARIEGEGSVLQAKLKAQALAIETEAELERVKKVREMELIYARAQLELEVSKAQQLANVEAKKFKEMTEALGPGTIRDLAVAGPEMQVKLLQSLGLKSTLITDGSSPINLFSTAFGLLGLGSDGQPPAQK

SEQ ID NO: 286 - protein, 1167 aa, synthetic construct
MATEEAIIRIPPYHYIHVLDQNSNVSRVEVGPKTYIRQDNERVLFAPVRMVTVPPRHYCIVANPVSRDTQSSVLFDITGQVRLRHADQEIRLAQDPFPLYPGEVLEKDITPLQVVLPNTALHLKALLDFEDKNGDKVMAGDEWLFEGPGTYIPQKEVEVVEIIQATVIKQNQALRLRARKECFDREGKGRVTGEEWLVRSVGAYLPAVFEEVLDLVDAVILTEKTALHLRALQNFRDLRGVLHRTGEEWLVTVQDTEAHVPDVYEEVLGVVPITTLGPRHYCVILDPMGPDGKNQLGQKRVVKGEKSFFLQPGERLERGIQDVYVLSEQQGLLLKALQPLEEGESEEKVSHQAGDCWLIRGPLEYVPSAKVEVVEERQAIPLDQNEGIYVQDVKTGKVRAVIGSTYMLTQDEVLWEKELPSGVEELLNLGGGGSPEDKLQAIKYELAQNEEELAQIEEKLAANKEGGSGGGGSGSKTIVLSVGEATRTLTEIQSTADRQIFEEKVGPLVGRLRLTASLRQNGAKTAYRVNLKLDQADVVDSGLPKVRYTQVWSHDVTIVANSTEASRKSLYDLTKSLVATSQVEDLVVNLVPLGRGPGSGGGGSGGPEDENAALEEKIAQLKQKNAALKEEIQALEYSAPRNKTRVVSYRVPHNAAVQVYDYRAKRARVVFGPELVTLDPEEQFTVLSLSAGRPKRPHARRALCLLLGPDFFTDVITIETADHARLQLQLAYNWHFELKNRNDPAEAAKLFSVPDFVGDACKAIASRVRGAVASVTFDDFHKNSARIIRMAVFGFEGSPEDELAANEEELQQNEQKLAQIKQKLQAIKYGGGGSGGGGSGGGGCGLCVCPCNKLKSSDAYKKAWGNNQDGVVASQPARVGGGGSGGGGSGGGGSPEDEIQQLEEEIAQLEQKNAALKEKNQALKYGKARDQAVFPQNGLVVSSVDVQSVEPVDQRTRDALQRSVQLAIEITTNSQEAAAKHEAQRLEQEARGRLERQKILDQSEAEKARKELLELEAMSMAVESTGNAKAEAESRAEAARIEGEGSVLQAKLKAQALAIETEAELERVKKVREMELIYARAQLELEVSKAQQLANVEAKKFKEMTEALGPGTIRDLAVAGPEMQVKLLQSLGLKSTLITDGSSPINLFSTAFGLLGLGSDGQPPAQK

SEQ ID NO: 287 - protein, 423 aa, synthetic construct
MEKSFVITDPRLPDNPIIFASDGFLELTEYSREEILGRNGRFLQGPETDQATVQKIRDAIRDQREITVQLINYTKSGKKFWNLLHLQPMRDQKGELQYFIGVQLDGGGGGSGGGGSGSGPGNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGSGDYKDDDDKGLPPLERLTL

SEQ ID NO: 288 - protein, 424 aa, synthetic construct
MEKSFVITDPRLPDNPIIFASDGFLELTEYSREEILGRNGRFLQGPETDQATVQKIRDAIRDQREITVQLINYTKSGKKFWNLLHLQPMRDQKGELQYFIGVQLDGGGGGSGGGGSGSGPGNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGSGDYKDDDDKGGPAAKRVKLD

SEQ ID NO: 289 - protein, 375 aa, synthetic construct
MDPETCPCPTGGSCTCSDKCKCKGCKCTNCKKSCCSCCPAGCEKCAKDCVCKGEEGAKAEAEKCSCCQGSGPGNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGSGDYKDDDDKGLPPLERLTL

SEQ ID NO: 290 - protein, 457 aa, synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKYSDLEMGTRDDEYDYLFKVVLIGDSGVGKSNLLSRFTRNEFNLESKSTIGVEFATRSIQVDGKTIKAQIWDTAGQERYRAITSAYYRGAVGALLVYDIAKHLTYENVERWLKELRDHADSNIVIMLVGNKSDLRHLRAVPTDEARAFAEKNGLSFIETSALDSTNVEAAFQTILTEIYRIVSQKQMSDRRENDMSPSNNVVPIHVPPTTENKPKVQCCQNI

SEQ ID NO: 291 - protein, 850 aa, synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKYSDLELKLRILMSAAVTAGKLARAPADPGKAGVPGVAAPGAPAAAPPAKEIPEVLVDPRSRRRYVRGRFLGKGGFAKCFEISDADTKEVFAGKIVPKSLLLKPHQREKMSMEISIHRSLAHQHVVGFHGFFEDNDFVFVVLELCRRRSLLELHKRRKALTEPEARYYLRQIVLGCQYLHRNRVIHRDLKLGNLFLNEDLEVKIGDFGLATKVEYDGERKKTLCGTPNYIAPEVLSKKGHSFEVDVWSIGCIMYTLLVGKPPFETSCLKETYLRIKKNEYSIPKHINPVAASLIQKMLQTDPTARPTINELLNDEFFTSGYIPARLPITCLTIPPRFSIAPSSLDPSNRKPLTVLNKGLENPLPERPREKEEPVVRETGEVVDCHLSDMLQQLHSVNASKPSERGLVRQEEAEDPACIPIFWVSKWVDYSDKYGLGYQLCDNSVGVLFNDSTRLILYNDGDSLQYIERDGTESYLTVSSHPNSLMKKITLLKYFRNYMSEHLLKAGANITPREGDELARLPYLRTWFRTRSAIILHLSNGSVQINFFQDHTKLILCPLMAAVTYIDEKRDFRTYRLSLLEEYGCCKELASRLRYARTMVDKLLSSRSASNRLKAS

SEQ ID NO: 292 - protein, 2090 aa, synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKSGLRSSSSMTYSELYSRYTRVWIPDPDEVWRSAELTKDYKDGDESLQLRLEDDTILDYPIDVQNNQVPFLRNPDILVGENDLTALSHLHEPAVLHNLKVRFLESNHIYTYCGIVLVAINPYEQLPIYGQDVIYAYSGQNMGDMDPHIFAVAEEAYKQMARDEKNQSIIVSGESGAGKTVSAKYAMRYFATVGGSASDTNIEEKVLASSPIMEAIGNAKTTRNDNSSRFGKYIEIGFDKKYHIIGANMRTYLLEKSRVVFQADDERNYHIFYQLCAAASLPEFKELALTCAEDFFYTAHGGNTTIEGVDDAEDFEKTRQALTLLGVRESHQISIFKIIASILHLGSVEIQAERDGDSCSISPQDEHLSNFCRLLGIEHSQMEHWLCHRKLVTTSETYVKTMSLQQVVNARNALAKHIYAQLFSWIVEHINKALQTSLKQHSFIGVLDIYGFETFEINSFEQFCINYANEKLQQQFNSHVFKLEQEEYMKEQIPWTLIDFYDNQPCIDLIEAKLGILDLLDEECKVPKGTDQNWAQKLYERHSNSQHFQKPRMSNTAFIVIHFADKVEYLSDGFLEKNRDTVYEEQINILKASKFPLVADLFRDDEDSVPATNTAKSRSSSKINVRSSRPLMKAPNKEHKKSVGYQFRTSLNLLMETLNATTPHYVRCIKPNDEKLPFHFDPKRAVQQLRACGVLETIRISAAGYPSRWTYHDFFNRYRVLMKKRELANTTDKKNICKSVLESLIKDPDKFQFGRTKIFFRAGQVAYLEKLRADKFREATIMIQKTVRGWLQRVKYRRLRAATLTLQRFCRGYLARRLTEHLRRTRAAIVFQKQYRMLKARRAYCRVRRAAVIIQSYTRGHVCTQKLPPVLTEHKATIIQKYARGWMARRHFQRQRDAAIVIQCAFRRLKARQALKALKIEARSAEHLKRLNVGMENKVVQLQRKIDDQNKEFKTLSEQLSAVTSTHAMEVEKLKKELARYQQNQEADPSLQLQEEVQSLRTELQKAHSERRVLEDAHNRENGELRKRVADLEHENALLKDEKEHLNHQILRQSKAESSQSSVEENLLIKKELEEERSRYQNLVKEYSQLEQRYENLRDEQQTPGHRKNPSNQSSLESDSNYPSISTSEIGDTEDALQQVEEIGIEKAAMDMTVFLKLQKRVRELEQERKKLQVQLEKEQQDSKKVQVEQQNNGLDVDQDADIAYNSLKRQELESENKKLKNDLNERWKAVADQAMQDNSTHSSPDSYSLLLNQLKLANEELEVRKEEVLILRTQIMNADQRRLSGKNMEPNINARTSWPNSEKHVDQEDAIEAYHGVCQTNSQTEDWGYLNEDGELGLAYQGLKQVARLLEAQLQAQNLKHEEEVEHLKAQVEAMKEEMDKQQQTFCQTLLLSPEAQVEFGVQQEISRLTNENLDFKELVEKLEKNEKKLKKQLKIYMKKVQDLEAAQALAQSDRRHHELTRQVTVQRKEKDFQGMLEYHKEDEALLIRNLVTDLKPQMLSGTVPCLPAYILYMCIRHADYTNDDLKVHSLLSSTINGIKKVLKKHNEDFEMTSFWLSNTCRLLHCLKQYSGDEGFMTQNTAKQNEHCLKNFDLTEYRQVLSDLSIQIYQQLIKIAEGLLQPMIVSAMLENESIQGLSGVRPTGYRKRSSSMVDGENSYCLEAIIRQMNFFHTVLCDQGLDPEIILQVFKQLFYMINAVTLNNLLLRKDACSWSTGMQLRYNISQLEEWLRGKNLQQSGAVQTMEPLIQAAQLLQLKKKTQEDAEAICSLCTSLSTQQIVKILNLYTPLNGFEERVTVSFIRTIQAQLQERSDPQQLLLDSKHMFPVLFPFNPSALTMDSIHIPACLNLEFLNEV

SEQ ID NO: 293 - protein, 365 aa, synthetic construct; identical to SEQ ID NO: 165

SEQ ID NO: 294 - protein, 444 aa, synthetic construct; identical to SEQ ID NO: 143

SEQ ID NO: 295 - protein, 511 aa, synthetic construct
MDPETCPCPTGGSCTCSDKCKCKGCKCTNCKKSCCSCCPAGCEKCAKDCVCKGEEGAKAEAEKCSCCQGGTGGSTSTTLVKCACEPCLCNVDPSKAIDRNGLYYCSEACADGHTGGSKGCGHTGCNCHGSAGGCDDKCGCAVPCPGGTGCRCTSARSGAAAGEHTTCGCGEHCGCNPCACGREGTPSGRANRRANCSCGAACNCASCGSATAPGGSGPGNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGSGDYKDDDDK

SEQ ID NO: 296 - protein, 370 aa, synthetic construct; identical to SEQ ID NO: 166

SEQ ID NO: 297 - protein, 449 aa, synthetic construct
MDPETCPCPTGGSCTCSDKCKCKGCKCTNCKKSCCSCCPAGCEKCAKDCVCKGEEGAKAEAEKCSCCQGGGSGGGSGGSADPETCPCPTGGSCTCSDKCKCKGCKCTNCKKSCCSCCPAGCEKCAKDCVCKGEEGAKAEAEKCSCCQGSGPGPDFLGHAENPLREEEWARLNETVIQVARRSLVGRRILDIYGPLGAGVQTVPYDEFQGVSPGAVDIVGEQETAMVFTDARKFKTIPIIYKDFLLHWRDIEAARTHNMPLDVSAAAGAAALCAQQEDELIFYGDARLGYEGLMTANGRLTVPLGDWTSPGGGFQAIVEATRKLNEQGHFGPYAVVLSPRLYSQLHRIYEKTGVLEIETIRQLASDGVYQSNRLRGESGVVVSTGRENMDLAVSMDMVAAYLGASRMNHPFRVLEALLLRIKHPDAICTLEGAGATERRGSGDYKDDDDK

SEQ ID NO: 298 - protein, 353 aa, synthetic construct
MDPETCPCPTGGSCTCSDKCKCKGCKCTNCKKSCCSCCPAGCEKCAKDCVCKGEEGAKAEAEKCSCCQGSGPGEFLKRSFAPLTEKQWQEIDNRAREIFKTQLYGRKFVDVEGPYGWEYAAHPLGEVEVLSDENEVVKWGLRKSLPLIELRATFTLDLWELDNLERGKPNVDLSSLEETVRKVAEFEDEVIFRGCEKSGVKGLLSFEERKIECGSTPKDLLEAIVRALSIFSKDGIEGPYTLVINTDRWINFLKEEAGHYPLEKRVEECLRGGKIITTPRIEDALVVSERGGDFKLILGQDLSIGYEDREKDAVRLFITETFTFQVVNPEALILLKGGGSGPDRKAAVSHWQQ

SEQ ID NO: 299 - protein, 470 aa, synthetic construct
MDPETCPCPTGGSCTCSDKCKCKGCKCTNCKKSCCSCCPAGCEKCAKDCVCKGEEGAKAEAEKCSCCQGSGPGEFLKRSFAPLTEKQWQEIDNRAREIFKTQLYGRKFVDVEGPYGWEYAAHPLGEVEVLSDENEVVKWGLRKSLPLIELRATFTLDLWELDNLERGKPNVDLSSLEETVRKVAEFEDEVIFRGCEKSGVKGLLSFEERKIECGSTPKDLLEAIVRALSIFSKDGIEGPYTLVINTDRWINFLKEEAGHYPLEKRVEECLRGGKIITTPRIEDALVVSERGGDFKLILGQDLSIGYEDREKDAVRLFITETFTFQVVNPEALILLKGSGSGSETTGAQVQLVESGGSLVQPGGSLRLSCAASGRFAESSSMGWFRQAPGKEREFVAAISWSGGATNYADSAKGRFTLSRDNTKNTVYLQMNSLKPDDTAVYYCAANLGNYISSNQRLYGYWGQGTQVTVS

SEQ ID NO: 300 - protein, 479 aa, synthetic construct; identical to SEQ ID NO: 189

SEQ ID NO: 301 - protein, 700 aa, synthetic construct
MSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGGGTGGSAPKTITELEKKVEEIPFKKERKFNPDLAPGTEKVTREGQKGEKTITTPTLKNPLTGVIISKGEPKEEITKDPINELTEYGPETIAPGHRDEFDPKLPTGEKEEVPGKPGIKNPETGDVVRPPVDSVTKYGYGPVKGDSIVEKEEIPFEKERKFNPDLAPGTEKVTREGQKGEKTITTPTLKNPLTGEIISKGESKEEITKDPINELTEYGPETGSGATGSVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 302 - protein, 827 aa, synthetic construct
MSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGGGTGGSAPKTITELEKKVEEIPFKKERKFNPDLAPGTEKVTREGQKGEKTITTPTLKNPLTGVIISKGEPKEEITKDPINELTEYGPETIAPGHRDEFDPKLPTGEKEEVPGKPGIKNPETGDVVRPPVDSVTKYGYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPTGSGATGSVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 303 - protein, 956 aa, synthetic construct
MSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGGGTGGSAPKTITELEKKVEEIPFKKERKFNPDLAPGTEKVTREGQKGEKTITTPTLKNPLTGVIISKGEPKEEITKDPINELTEYGPETIAPGHRDEFDPKLPTGEKEEVPGKPGIKNPETGDVVRPPVDSVTKYGYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETGSGATGSVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 304 - protein, 1084 aa, synthetic construct
MSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGGGTGGSAPKTITELEKKVEEIPFKKERKFNPDLAPGTEKVTREGQKGEKTITTPTLKNPLTGVIISKGEPKEEITKDPINELTEYGPETIAPGHRDEFDPKLPTGEKEEVPGKPGIKNPETGDVVRPPVDSVTKYGYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETGSGATGSVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 305 - protein, 1212 aa, synthetic construct
MSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGGGTGGSAPKTITELEKKVEEIPFKKERKFNPDLAPGTEKVTREGQKGEKTITTPTLKNPLTGVIISKGEPKEEITKDPINELTEYGPETIAPGHRDEFDPKLPTGEKEEVPGKPGIKNPETGDVVRPPVDSVTKYGYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETGSGATGSVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 306 - protein, 1340 aa, synthetic construct
MSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGGGTGGSAPKTITELEKKVEEIPFKKERKFNPDLAPGTEKVTREGQKGEKTITTPTLKNPLTGVIISKGEPKEEITKDPINELTEYGPETIAPGHRDEFDPKLPTGEKEEVPGKPGIKNPETGDVVRPPVDSVTKYGYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETGSGATGSVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 307 - protein, 1468 aa, synthetic construct
MSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGGGTGGSAPKTITELEKKVEEIPFKKERKFNPDLAPGTEKVTREGQKGEKTITTPTLKNPLTGVIISKGEPKEEITKDPINELTEYGPETIAPGHRDEFDPKLPTGEKEEVPGKPGIKNPETGDVVRPPVDSVTKYGYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETGSGATGSVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 308 - protein, 1596 aa, synthetic construct
MSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGGGTGGSAPKTITELEKKVEEIPFKKERKFNPDLAPGTEKVTREGQKGEKTITTPTLKNPLTGVIISKGEPKEEITKDPINELTEYGPETIAPGHRDEFDPKLPTGEKEEVPGKPGIKNPETGDVVRPPVDSVTKYGYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETGSGATGSVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 309 - protein, 1724 aa, synthetic construct
MSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGGGTGGSAPKTITELEKKVEEIPFKKERKFNPDLAPGTEKVTREGQKGEKTITTPTLKNPLTGVIISKGEPKEEITKDPINELTEYGPETIAPGHRDEFDPKLPTGEKEEVPGKPGIKNPETGDVVRPPVDSVTKYGYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETGSGATGSVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 310 - protein, 532 aa, synthetic construct
MDPETCPCPTGGSCTCSDKCKCKGCKCTNCKKSCCSCCPAGCEKCAKDCVCKGEEGAKAEAEKCSCCQGSGPGNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGSGDYKDDDDKTGACAATTAGCTCATCCAGACATTCCGGGGGSGPGGSGVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGSGGVSGWRLFKKIS

SEQ ID NO: 311 - protein, 629 aa, synthetic construct
MVSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSKLSKDPNEKRDHMVLLEFVTAAGITHGMDELYKSGLRSRAQMGDWSALGKLLDKVQAYSTAGGKVWLSVLFIFRILLLGTAVESAWGDEQSAFRCNTQQPGCENVCYDKSFPISHVRFWVLQIIFVSVPTLLYLAHVFYVMRKEEKLNKKEEELKVAQTDGVNVEMHLKQIEIKKFKYGIEEHGKVKMRGGLLRTYIISILFKSVFEVAFLLIQWYIYGFSLSAVYTCKRDPCPHQVDCFLSRPTEKTIFIIFMLVVSLVSLALNIIELFYVFFKGVKDRVKGRSDPYHATTGPLSPSKDCGSPKYAYFNGCSSPTAPLSPMSPPGYKLVTGDRNNSSCRNYNKQASEQNWANYSAEQNRMGQAGSTISNSHAQPFDFPDDNQNAKKVAAGHELQPLAIVDQRPSSRASSRASSRPRPDDLEI

SEQ ID NO: 312 - protein, 630 aa, synthetic construct
MGDWSALGKLLDKVQAYSTAGGKVWLSVLFIFRILLLGTAVESAWGDEQSAFRCNTQQPGCENVCYDKSFPISHVRFWVLQIIFVSVPTLLYLAHVFYVMRKEEKLNKKEEELKVAQTDGVNVEMHLKQIEIKKFKYGIEEHGKVKMRGGLLRTYIISILFKSVFEVAFLLIQWYIYGFSLSAVYTCKRDPCPHQVDCFLSRPTEKTIFIIFMLVVSLVSLALNIIELFYVFFKGVKDRVKGRSDPYHATTGPLSPSKDCGSPKYAYFNGCSSPTAPLSPMSPPGYKLVTGDRNNSSCRNYNKQASEQNWANYSAEQNRMGQAGSTISNSHAQPFDFPDDNQNAKKVAAGHELQPLAIVDQRPSSRASSRASSRPRPDDLEISARDPPVATMVSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSKLSKDPNEKRDHMVLLEFVTAAGITHGMDELYK

SEQ ID NO: 313 - protein, 379 aa, synthetic construct
MYPYDVPDYAGTGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKPEGSGMVSKGEELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANAKTTYRSKKPAKNLKMPGVYYVDYRLERIKEANNETYVEQHEVAVARYCDLPSKLGHKLNGGGGSGGGGSKGFTVGSLIQ

SEQ ID NO: 314 - protein, 373 aa, synthetic construct
MYPYDVPDYAGTGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKPEGSGVSKGEAVIKEFMRFKVHMEGSMNGHEFEIEGEGEGRPYEGTQTAKLKVTKGGPLPFSWDILSPQFMYGSRAFIKHPADIPDYYKQSFPEGFKWERVMNFEDGGAVTVTQDTSLEDGTLIYKVKLRGTNFPPDGPVMQKKTMGWEASTERLYPEDGVLKGDIKMALRLKDGGRYLADFKTTYKAKKPVQMPGAYNVDRKLDITSHNEDYTVVEQYERSEGRHSTGGMDELYKGGGGSGGGGSKGFTVGSLIQ

SEQ ID NO: 315 - protein, 503 aa, synthetic construct
MDPETCPCPTGGSCTCSDKCKCKGCKCTNCKKSCCSCCPAGCEKCAKDCVCKGEEGAKAEAEKCSCCQGSGPGNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGGGGSGPGGSGLEKFVGTWKIADSHNFGEYLKAIGAPKELSDGGDATTPTLYISQKDGDKMSVKIENGPPTFLDTQVKFKLGEEFDEFPSDRRKGVKSVVNLVGEKLVYVQKWDGKETTYVREIKDGKLVVTLTMGDVVAVRSYRRATE

SEQ ID NO: 316 - protein, 632 aa, synthetic construct
MLEKFVGTWKIADSHNFGEYLKAIGAPKELSDGGDATTPTLYISQKDGDKMSVKIENGPPTFLDTQVKFKLGEEFDEFPSDRRKGVKSVVNLVGEKLVYVQKWDGKETTYVREIKDGKLVVTLTMGDVVAVRSYRRATEGGTGGSDPETCPCPTGGSCTCSDKCKCKGCKCTNCKKSCCSCCPAGCEKCAKDCVCKGEEGAKAEAEKCSCCQGSGPGNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGSGSGSETTGAQVQLVESGGSLVQPGGSLRLSCAASGRFAESSSMGWFRQAPGKEREFVAAISWSGGATNYADSAKGRFTLSRDNTKNTVYLQMNSLKPDDTAVYYCAANLGNYISSNQRLYGYWGQGTQVTVS

SEQ ID NO: 317 - protein, 622 aa, synthetic construct
MLEKFVGTWKIADSHNFGEYLKAIGAPKELSDGGDATTPTLYISQKDGDKMSVKIENGPPTFLDTQVKFKLGEEFDEFPSDRRKGVKSVVNLVGEKLVYVQKWDGKETTYVREIKDGKLVVTLTMGDVVAVRSYRRATEGGTGGSDPETCPCPTGGSCTCSDKCKCKGCKCTNCKKSCCSCCPAGCEKCAKDCVCKGEEGAKAEAEKCSCCQGSGPGNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGSGSGSETTGVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSS

SEQ ID NO: 318 - protein, 516 aa, synthetic construct
MLEKFVGTWKIADSHNFGEYLKAIGAPKELSDGGDATTPTLYISQKDGDKMSVKIENGPPTFLDTQVKFKLGEEFDEFPSDRRKGVKSVVNLVGEKLVYVQKWDGKETTYVREIKDGKLVVTLTMGDVVAVRSYRRATEGGTGGSDPETCPCPTGGSCTCSDKCKCKGCKCTNCKKSCCSCCPAGCEKCAKDCVCKGEEGAKAEAEKCSCCQGSGPGNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGSGSGSETTGDYKDDDDK

SEQ ID NO: 319 - protein, 711 aa, synthetic construct
MLEKFVGTWKIADSHNFGEYLKAIGAPKELSDGGDATTPTLYISQKDGDKMSVKIENGPPTFLDTQVKFKLGEEFDEFPSDRRKGVKSVVNLVGEKLVYVQKWDGKETTYVREIKDGKLVVTLTMGDVVAVRSYRRATEGGTGGSDPETCPCPTGGSCTCSDKCKCKGCKCTNCKKSCCSCCPAGCEKCAKDCVCKGEEGAKAEAEKCSCCQGGGSGGGSGGSADPETCPCPTGGSCTCSDKCKCKGCKCTNCKKSCCSCCPAGCEKCAKDCVCKGEEGAKAEAEKCSCCQGSGPGNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGSGSGSETTGAQVQLVESGGSLVQPGGSLRLSCAASGRFAESSSMGWFRQAPGKEREFVAAISWSGGATNYADSAKGRFTLSRDNTKNTVYLQMNSLKPDDTAVYYCAANLGNYISSNQRLYGYWGQGTQVTVS

SEQ ID NO: 320 - protein, 701 aa, synthetic construct
MLEKFVGTWKIADSHNFGEYLKAIGAPKELSDGGDATTPTLYISQKDGDKMSVKIENGPPTFLDTQVKFKLGEEFDEFPSDRRKGVKSVVNLVGEKLVYVQKWDGKETTYVREIKDGKLVVTLTMGDVVAVRSYRRATEGGTGGSDPETCPCPTGGSCTCSDKCKCKGCKCTNCKKSCCSCCPAGCEKCAKDCVCKGEEGAKAEAEKCSCCQGGGSGGGSGGSADPETCPCPTGGSCTCSDKCKCKGCKCTNCKKSCCSCCPAGCEKCAKDCVCKGEEGAKAEAEKCSCCQGSGPGNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGSGSGSETTGVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSS

SEQ ID NO: 321 - protein, 595 aa, synthetic construct
MLEKFVGTWKIADSHNFGEYLKAIGAPKELSDGGDATTPTLYISQKDGDKMSVKIENGPPTFLDTQVKFKLGEEFDEFPSDRRKGVKSVVNLVGEKLVYVQKWDGKETTYVREIKDGKLVVTLTMGDVVAVRSYRRATEGGTGGSDPETCPCPTGGSCTCSDKCKCKGCKCTNCKKSCCSCCPAGCEKCAKDCVCKGEEGAKAEAEKCSCCQGGGSGGGSGGSADPETCPCPTGGSCTCSDKCKCKGCKCTNCKKSCCSCCPAGCEKCAKDCVCKGEEGAKAEAEKCSCCQGSGPGNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGSGSGSETTGDYKDDDDK

SEQ ID NO: 322 - protein, 376 aa, synthetic construct; identical to SEQ ID NO: 168

SEQ ID NO: 323 - protein, 381 aa, synthetic construct; identical to SEQ ID NO: 167

SEQ ID NO: 324 - protein, 529 aa, synthetic construct
MDPETCPCPTGGSCTCSDKCKCKGCKCTNCKKSCCSCCPAGCEKCAKDCVCKGEEGAKAEAEKCSCCQGGTGGSTSTTLVKCACEPCLCNVDPSKAIDRNGLYYCSEACADGHTGGSKGCGHTGCNCHGSAGGCDDKCGCAVPCPGGTGCRCTSARSGAAAGEHTTCGCGEHCGCNPCACGREGTPSGRANRRANCSCGAACNCASCGSATAPGGGGSGGGSGGSANKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGSGDYKDDDDKGGPAAKRVKLD

SEQ ID NO: 325 - protein, 698 aa, synthetic construct
MVSKGEAVIKEFMRFKVHMEGSMNGHEFEIEGEGEGRPYEGTQTAKLKVTKGGPLPFSWDILSPQFMYGSRAFIKHPADIPDYYKQSFPEGFKWERVMNFEDGGAVTVTQDTSLEDGTLIYKVKLRGTNFPPDGPVMQKKTMGWEASTERLYPEDGVLKGDIKMALRLKDGGRYLADFKTTYKAKKPVQMPGAYNVDRKLDITSHNEDYTVVEQYERSEGRHSTGGMDELYKGSGATNFSLLKQAGDVEENPGPMDPETCPCPTGGSCTCSDKCKCKGCKCTNCKKSCCSCCPAGCEKCAKDCVCKGEEGAKAEAEKCSCCQGGGSGGGSGGSADPETCPCPTGGSCTCSDKCKCKGCKCTNCKKSCCSCCPAGCEKCAKDCVCKGEEGAKAEAEKCSCCQGSGPGNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGSGDYKDDDDK

SEQ ID NO: 326 - protein, 293 aa, synthetic construct; identical to SEQ ID NO: 142

SEQ ID NO: 327 - protein, 297 aa, synthetic construct
PDFLGHAENPLREEEWARLNETVIQVARRSLVGRRILDIYGPLGAGVQTVPYDEFQGVSPGAVDIVGEQETAMVFTDARKFKTIPIIYKDFLLHWRDIEAARTHNMPLDVSAAAGAAALCAQQEDELIFYGDARLGYEGLMTANGRLTVPLGDWTSPGGGFQAIVEATRKLNEQGHFGPYAVVLSPRLYSQLHRIYEKTGVLEIETIRQLASDGVYQSNRLRGESGVVVSTGRENMDLAVSMDMVAAYLGASRMNHPFRVLEALLLRIKHPDAICTLEGAGATERRGSGDYKDDDDK

SEQ ID NO: 328 - protein, 282 aa, synthetic construct
EFLKRSFAPLTEKQWQEIDNRAREIFKTQLYGRKFVDVEGPYGWEYAAHPLGEVEVLSDENEVVKWGLRKSLPLIELRATFTLDLWELDNLERGKPNVDLSSLEETVRKVAEFEDEVIFRGCEKSGVKGLLSFEERKIECGSTPKDLLEAIVRALSIFSKDGIEGPYTLVINTDRWINFLKEEAGHYPLEKRVEECLRGGKIITTPRIEDALVVSERGGDFKLILGQDLSIGYEDREKDAVRLFITETFTFQVVNPEALILLKGGGSGPGPDRKAAVSHWQQ

SEQ ID NO: 329 - protein, 247 aa, synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKGSGDYKDDDDK

SEQ ID NO: 330 - protein, 488 aa, synthetic construct
MDPETCPCPTGGSCTCSDKCKCKGCKCTNCKKSCCSCCPAGCEKCAKDCVCKGEEGAKAEAEKCSCCQGSGPGNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGSGSGSETTGAQVQLVESGGSLVQPGGSLRLSCAASGRFAESSSMGWFRQAPGKEREFVAAISWSGGATNYADSAKGRFTLSRDNTKNTVYLQMNSLKPDDTAVYYCAANLGNYISSNQRLYGYWGQGTQVTVS

SEQ ID NO: 331 - protein, 365 aa, synthetic construct
MDPETCPCPTGGSCTCSDKCKCKGCKCTNCKKSCCSCCPAGCEKCAKDCVCKGEEGAKAEAEKCSCCQGSGPGNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGSGWSHPQFEK

SEQ ID NO: 332 - protein, 481 aa, synthetic construct
MSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGGTGATGSVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 333 - protein, 390 aa, synthetic construct
MGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKPEGSGGKSYPTVSADYQDAVEKAKKKLRGFIAEKRCAPLMLRLAFHSAGTFDKGTKTGGPFGTIKHPAELAHSANNGLDIAVRLLEPLKAEFPILSYADFYQLAGVVAVEVTGGPKVPFHPGREDKPEPPPEGRLPDPTKGSDHLRDVFGKAMGLTDQDIVALSGGHTIGAAHKERSGFEGPWTSNPLIFDNSYFTELLSGEKEGLLQLPSDKALLSDPVFRPLVDKYAADEDAFFADYAEAHQKLSELGFADALQLPPLERLTLDGGGGSGGGGSLTVGSLRR

SEQ ID NO: 334 - protein, 298 aa, synthetic construct
MNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGSGAHIVMVDAYKPTK

SEQ ID NO: 335 - protein, 231 aa, synthetic construct
MVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGGGGSGGGGSGDYDSATHIKFSKRDEDGKELAGATMELRDSSGKTISTWISDGQVKDFYLYPGKYTFVETAAPDGYEVATAITFTVNEQGQVTVNGKATKGDAHIGSGDYKDDDDK

SEQ ID NO: 336 - protein, 326 aa, synthetic construct
MNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGGGGSGPGGSGSPEDKIAQLKQKIQALKQENQQLEEENAALEYG

SEQ ID NO: 337 - protein, 260 aa, synthetic construct
VSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGLRSRAQASNSGCMSCKCVLS

SEQ ID NO: 338 - protein, 302 aa, synthetic construct
MSPEDEIQQLEEEIAQLEQKNAALKEKNQALKYGGGGGSGPGVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGLRSRAQASNSGCMSCKCVLS

SEQ ID NO: 339 - protein, 922 aa, synthetic construct
MNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGSGDYKDDDDKTAGCTAGGCGCTCGAAGACLSFGTEILTVEYGPLPIGKIVSEEINCSVYSVDPEGRVYTQAIAQWHDRGEQEVLEYELEDGSVIRATSDHRFLTTDYQLLAIEEIFARQLDLLTLENIKQTEEALDNHRLPFPLLDAGTIKMVKVIGRRSLGVQRIFDIGLPQDHNFLLANGAIAAACSCGSGSRGGSGATNFSLLKQAGDVEENPGPNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGGGGSGPGGSGVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGSGGVSGWRLFKKIS

SEQ ID NO: 340 - protein, 451 aa, synthetic construct
MNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGSGDYKDDDDKTAGCTAGGCGCTCGAAGAGGGGSGPGGSGVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGSGGVSGWRLFKKIS

SEQ ID NO: 341 - protein, 451 aa, synthetic construct
MNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGSGDYKDDDDKTAACTAGGCGCTCGAAGAGGGGSGPGGSGVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGSGGVSGWRLFKKIS

SEQ ID NO: 342 - protein, 451 aa, synthetic construct
MNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGSGDYKDDDDKTGACTAGGCGCTCGAAGAGGGGSGPGGSGVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGSGGVSGWRLFKKIS

SEQ ID NO: 343 - protein, 442 aa, synthetic construct
MNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGSGDYKDDDDKTAGCTATCCGGGGSGPGGSGVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGSGGVSGWRLFKKIS

SEQ ID NO: 344 - protein, 442 aa, synthetic construct
MNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGSGDYKDDDDKTAACTATCCGGGGSGPGGSGVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGSGGVSGWRLFKKIS

SEQ ID NO: 345 - protein, 442 aa, synthetic construct
MNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGSGDYKDDDDKTGACTATCCGGGGSGPGGSGVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGSGGVSGWRLFKKIS

SEQ ID NO: 346 - protein, 459 aa, synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKSGLRSRAMGTRDDEYDYLFKVVLIGDSGVGKSNLLSRFTRNEFNLESKSTIGVEFATRSIQVDGKTIKAQIWDTAGQERYRAITSAYYRGAVGALLVYDIAKHLTYENVERWLKELRDHADSNIVIMLVGNKSDLRHLRAVPTDEARAFAEKNGLSFIETSALDSTNVEAAFQTILTEIYRIVSQKQMSDRRENDMSPSNNVVPIHVPPTTENKPKVQCCQNI

SEQ ID NO: 347 - protein, 855 aa, synthetic construct
MSAAVTAGKLARAPADPGKAGVPGVAAPGAPAAAPPAKEIPEVLVDPRSRRRYVRGRFLGKGGFAKCFEISDADTKEVFAGKIVPKSLLLKPHQREKMSMEISIHRSLAHQHVVGFHGFFEDNDFVFVVLELCRRRSLLELHKRRKALTEPEARYYLRQIVLGCQYLHRNRVIHRDLKLGNLFLNEDLEVKIGDFGLATKVEYDGERKKTLCGTPNYIAPEVLSKKGHSFEVDVWSIGCIMYTLLVGKPPFETSCLKETYLRIKKNEYSIPKHINPVAASLIQKMLQTDPTARPTINELLNDEFFTSGYIPARLPITCLTIPPRFSIAPSSLDPSNRKPLTVLNKGLENPLPERPREKEEPVVRETGEVVDCHLSDMLQQLHSVNASKPSERGLVRQEEAEDPACIPIFWVSKWVDYSDKYGLGYQLCDNSVGVLFNDSTRLILYNDGDSLQYIERDGTESYLTVSSHPNSLMKKITLLKYFRNYMSEHLLKAGANITPREGDELARLPYLRTWFRTRSAIILHLSNGSVQINFFQDHTKLILCPLMAAVTYIDEKRDFRTYRLSLLEEYGCCKELASRLRYARTMVDKLLSSRSASNRLKASGSGSASGGSGSGPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 348 - protein, 423 aa, synthetic construct; identical to SEQ ID NO: 287

SEQ ID NO: 349 - protein, 424 aa, synthetic construct; identical to SEQ ID NO: 288

SEQ ID NO: 350 - protein, 375 aa, synthetic construct; identical to SEQ ID NO: 289

SEQ ID NO: 351 - DNA, 596 bp, synthetic construct
  source 1..596: mol_type = other DNA
ccccttcaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgctcgcttcggcagcacatatactagtcgacgggccgcactcgccggtcccaagcccggataaaatgggagggggcgggaaaccgcctaaccatgccgagtgcggccgcgagacggtcgggtccagatagaattccgaccagaagatatggcttcggttgggttctatctgtcgagtagagtgtgggctcgtggccgcggtcggcgtggactgtagaacactgccaatgccggtcccaagcccggataaaagtggagggtacagtccacgctctagagcggacttcggtccgcgcggttctatctagttacgcgttaaaccaactagaatttttt

SEQ ID NO: 352 - DNA, 660 bp, synthetic construct
  source 1..660: mol_type = other DNA
ccccttcaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgctcgcttcggcagcacatatactagtcgacgggccgcactcgccggtcccaagcccggataaaatgggagggggcgggaaaccgcctaaccatgccgagtgcggccgcgagacggtcgggtccagataggcactaacctaagacaggagggccgggaaacctgcctaatccaatgacgggtaatagtgtcgaattccgaccagaagatatggcttcggttgggttcgttatctgtcgagtagagtgtgggctcgtggccgcggtcggcgtggactgtagaacactgccaatgccggtcccaagcccggataaaagtggagggtacagtccacgctctagagcggacttcggtccgcgcggttctatctagttacgcgttaaaccaactagaatttttt

SEQ ID NO: 353 - DNA, 658 bp, synthetic construct
  source 1..658: mol_type = other DNA
ccccttcaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgctcgcttcggcagcacatatactagtcgacgggccgcactcgccggtcccaagcccggataaaatgggagggggcgggaaaccgcctaaccatgccgagtgcggccgcgagacggtcgggtccagataggcactaacctaagacaggagggccgggagaattccgaccagaagatatggcttcggttgggttcaacctgcctaatccaatgacgggtaatagtgtctatctgtcgagtagagtgtgggctcgtggccgcggtcggcgtggactgtagaacactgccaatgccggtcccaagcccggataaaagtggagggtacagtccacgctctagagcggacttcggtccgcgcggttctatctagttacgcgttaaaccaactagaatttttt

SEQ ID NO: 354 - protein, 1179 aa, synthetic construct; identical to SEQ ID NO: 284

SEQ ID NO: 355 - protein, 1137 aa, synthetic construct; identical to SEQ ID NO: 285

SEQ ID NO: 356 - protein, 1167 aa, synthetic construct; identical to SEQ ID NO: 286

SEQ ID NO: 357 - protein, 1257 aa, synthetic construct
MKEELDAFHQIFTTTKEAIERFMAMLTPVIENAEDDHERLYYHHIYEEEEQRLSRLDVLIPLIEKFQDETDEGLFSPSNNAFNRLLQELNLEKFGLHNFIEHVLALFSFTDEERQTLLKELRKDAYEGYQYVKEKLAEINARFDHDYADPHAHHDEHRDHLADMPSAGSSHEEVQPVAHKKKGFTVGSLIQGSGATNFSLLKQAGDVEENPGPMNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGSGGGGGSGPGGSGMKFNKEKWQAIKEIAFLPNLNQPQHLAFIVSLLDDPSQSANLLAEAKKLNDAQAPKGSGATNFSLLKQAGDVEENPGPMSRDPLPFFPPLYLGGPEITTENCEREPIHIPGSIQPHGALLTADGHSGEVLQMSLNAATFLGQEPTVLRGQTLAALLPEQWPALQAALPPGCPDALQYRATLDWPAAGHLSLTVHRVGELLILEFEPTEAWDSTGPHALRNAMFALESAPNLRALAEVATQTVRELTGFDRVMLYKFAPDATGEVIAEARREGLHAFLGHRFPASDIPAQARALYTRHLLRLTADTRAAAVPLDPVLNPQTNAPTPLGGAVLRATSPMHMQYLRNMGVGSSLSVSVVVGGQLWGLIACHHQTPYVLPPDLRTTLEYLGRLLSLQVQVKEAADVAAFRQSLREHHARVALAAAHSLSPHDTLSDPALDLLGLMRAGGLILRFEGRWQTLGEVPPAPAVDALLAWLETQPGALVQTDALGQLWPAGADLAPSAAGLLAISVGEGWSECLVWLRPELRLEVAWGGATPDQAKDDLGPRHSFDTYLEEKRGYAEPWHPGEIEEAQDLRDTLTGALLESGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 358 - protein, 1019 aa, synthetic construct
MKEELDAFHQIFTTTKEAIERFMAMLTPVIENAEDDHERLYYHHIYEEEEQRLSRLDVLIPLIEKFQDETDEGLFSPSNNAFNRLLQELNLEKFGLHNFIEHVDLALFSFTDEERQTLLKELRKDAYEGYQYVKEKLAEINARFDHDYADPHAHHDEHRDHLADMPSAGSSHEEVQPVAHKKKGFTVGSLIQGSGATNFSLLKQAGDVEENPGPMNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGSGGSGGSGGSGGTGGSGGSGGSGMHTLYAPGGYDIMGYLRQIRNRPNPQVELGPVDTSCALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGATNFSLLKQAGDVEENPGPMGHTLYAPGGYDIMGYLDQIGNRPNPQVELGPVDTSCALILCDLKQKDTPIVYASEAFLYMTGYSNAEVLGRNCRFLQSPDGMVKPKSTRKYVDSNTINTIRKAIDRNAEVQVEVVNFKKNGQRFVNFLTIIPVRDETGEYRYSMGFQCETEGGGGSGGGGSGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 359 - protein, 255 aa, synthetic construct
MVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGGGGSGGGGSGENLYFQSGGGGSGGGGSGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 360 - protein, 459 aa, synthetic construct
MDPETCPCPTGGSCTCSDKCKCKGCKCTNCKKSCCSCCPAGCEKCAKDCVCKGEEGAKAEAEKCSCCQGSGPGNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGGGGSGPGGSGMILWHEMWHEGLEEASRLYFGERNVKGMFEVLEPLHAMMERGPQTLKETSFNQAYGRDLMEAQEWCRKYMKSGNVKDLTQAWDLYYHVFRRISK

SEQ ID NO: 361 - protein, 517 aa, synthetic construct
MDPETCPCPTGGSCTCSDKCKCKGCKCTNCKKSCCSCCPAGCEKCAKDCVCKGEEGAKAEAEKCSCCQGSGPGNKSQLYPDSPLTDQDFNQLDQTVIEAARRQLVGRRFIELYGPLGRGMQSVFNDIFMESHEAKMDFQGSFDTEVESSRRVNYTIPMLYKDFVLYWRDLEQSKALDIPIDFSVAANAARDVAFLEDQMIFHGSKEFDIPGLMNVKGRLTHLIGNWYESGNAFQDIVEARNKLLEMNHNGPYALVLSPELYSLLHRVHKDTNVLEIEHVRELITAGVFQSPVLKGKSGVIVNTGRNNLDLAISEDFETAYLGEEGMNHPFRVYETVVLRIKRPAAICTLIDPEEGGGGSGPGGSGMGHTLYAPGGYDIMGYLDQIGNRPNPQVELGPVDTSCALILCDLKQKDTPIVYASEAFLYMTGYSNAEVLGRNCRFLQSPDGMVKPKSTRKYVDSNTINTIRKAIDRNAEVQVEVVNFKKNGQRFVNFLTIIPVRDETGEYRYSMGFQCETE

SEQ ID NO: 362 - protein, 435 aa, synthetic construct
MHTLYAPGGYDIMGYLRQIRNRPNPQVELGPVDTSCALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGGTGGSGGSGGSGVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGLRSRAQASNSGCMSCKCVLS

SEQ ID NO: 363 - DNA, 596 bp, synthetic construct; identical to SEQ ID NO: 351
  source 1..596: mol_type = other DNA

SEQ ID NO: 364 - DNA, 660 bp, synthetic construct; identical to SEQ ID NO: 352
  source 1..660: mol_type = other DNA

SEQ ID NO: 365 - DNA, 653 bp, synthetic construct
  misc_feature 1..405: note = DNA
  source 1..653: mol_type = other DNA
  misc_feature 406..444: note = RNA
  misc_feature 445..653: note = DNA
ccccttcaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgctcgcttcggcagcacatatactagtcgacggccgcactcgccggtcccaagcccggataaaatgggagggggcgggaaaccgcctaaccatgccgagtgcggccgcgagacggtcgggtccagatagggggagaaggacgggtccagtgcgaaacacgcactgttgagtagagtgtgagctcccgaattccgaccagaagatatggcttcggttgggttctatctgtcgagtagagtgtgggctcgtggccgcggtcggcgtggactgtagaacactgccaatgccggtcccaagcccggataaaagtggagggtacagtccacgctctagagcggacttcggtccgcgcggttctatctagttacgcgttaaaccaactagaatttttt